SEQUENCE LISTING (117 sequences)

SEQ ID NO: 1 - protein, 531 aa, Rattus norvegicus
  Pyruvate kinase M1 subunit
MPKPDSEAGTAFIQTQQLHAAMADTFLEHMCRLDIDSAPITARNTGIICTIGPASRSVEMLKEMIKSGMNVARLNFSHGTHEYHAETIKNVRAATESFASDPILYRPVAVALDTKGPEIRTGLIKGSGTAEVELKKGATLKITLDNAYMEKCDENILWLDYKNICKVVEVGSKIYVDDGLISLQVKEKGADYLVTEVENGGSLGSKKGVNLPGAAVDLPAVSEKDIQDLKFGVEQDVDMVFASFIRKAADVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAVFHRLLFEELARASSQSTDPLEAMAMGSVEASYKCLAAALIVLTESGRSAHQVARYRPRAPIIAVTRNPQTARQAHLYRGIFPVLCKDAVLDAWAEDVDLRVNLAMNVGKARGFFKKGDVVIVLTGWRPGSGFTNTMRVVPVP

SEQ ID NO: 2 - protein, 531 aa, Rattus norvegicus
  Pyruvate kinase M2 subunit
MPKPDSEAGTAFIQTQQLHAAMADTFLEHMCRLDIDSAPITARNTGIICTIGPASRSVEMLKEMIKSGMNVARLNFSHGTHEYHAETIKNVRAATESFASDPILYRPVAVALDTKGPEIRTGLIKGSGTAEVELKKGATLKITLDNAYMEKCDENILWLDYKNICKVVEVGSKIYVDDGLISLQVKEKGADYLVTEVENGGSLGSKKGVNLPGAAVDLPAVSEKDIQDLKFGVEQDVDMVFASFIRKAADVHEVRKVLGEKGKNIKIISKIENHEGVRRFDEILEASDGIMVARGDLGIEIPAEKVFLAQKMMIGRCNRAGKPVICATQMLESMIKKPRPTRAEGSDVANAVLDGADCIMLSGETAKGDYPLEAVRMQHLIAREAEAAIYHLQLFEELRRLAPITSDPTEAAAVGAVEASFKCCSGAIIVLTKSGRSAHQVARYRPRAPIIAVTRNPQTARQAHLYRGIFPVLCKDAVLDAWAEDVDLRVNLAMNVGKARGFFKKGDVVIVLTGWRPGSGFTNTMRVVPVP

SEQ ID NO: 3 - DNA, 11788 bp, Rattus norvegicus
  Pyruvate kinase
cccgggcgagcgccgggagggtggagagtcaccgggcggggctggaggaatgtccgggacctataaatctgggcaacgccctggtaggccagggcagatggggcacctgggcagaattccaaaatgggattatgtagcctctgaggtcctaaagcaacaggtggcggaccacccggggatctaggggtggtggcggcggtggacccgagggcgggtcctgcctcctcaccacttccccattggccatcagaatgacccatgcgcaattttggtttgcaatgtccttccgccacggaaggtagtccccctcaaaagggcaacctgcttgtcccgcctaccctgcgactctctcagaaggtgcgggtgcctgttgagaggcggggctctgctagctgctgcccggattgggcgaggggcggggctgcggagggattgcggcggcccgcagcagtgataaccttgaggcccagtctgcgcagccccgcacagcagcgacccgtcctaagtcgacagacgtcctctttaggtattgcaacaggatctgaagtacgcccgaggtgagcggggagaacctttgccattctgtggcccagagccaatgccatagtggatcactagtgggtggctgcaatgtcaggtggcagagccaggcttgggtgcatgttctttaggtatccagaaaagccgatgtccagggtagagtgcgggtctttgggaaggcagggaagagcaaggagatgtggagtgcaggctgtgtgctcaggagtttgaaaggagcctcgtggcctgaaaattgattcaagtctctggtattctgtgaggtacatcccaggttctcgcttactcagccaactatccagctcagtgagtgcttagctgtaagtcgtaataaagtaaaagcaaaggtcaggacagctgttgggattgtgacctgggtgattaattgcctggaacagatgttcagactactgtgtcgtggtccctatcagacagagaaggagactctagatagccagaactggggctgggaaagctgggtctaatgtcagctcttgggaagagtagtgactttcaaggtcaacctcggttgcacagtggatttcaggccagcatagactagctactgtgtgtgattccatctcctaggttactgaagactccttggtgaggttgtaatgactttggagccatttcaatgtccttgtgcaaaatttgctttgtgtgtcttgtacatggtgctgcttttttgcttcttgtccagaaaaaaacttgttgcctgtaatttggaccttattcctgtgtcggatagaggctaaggaatacatttaggtgatacctcaaacctaggactttaaaaagaaaggacttgggacagggttcgtctcttggcagcaggatcgtttaggtcatggcgaacatgataaagaggctatgtcagtgccaaagcaggtttgacattgtcgtgataggcttcccagatgttcgtgtgctgacacattagctacgtgctctgcaggagtggggaggtttccttgtgtgggaaggtatgttctctctagggttggtgtgaagctcttcaatagatggcagccgttgtaagaagctctgcttaaactggcagccagggtgtgtatgtgcactgggaaggaagctacttttgttcaccaaggtctttgctgttcgttcatagtcacaggagccctttgcccaatttgagtagtacccacataaccagcacgtgccaggcctctgagtgacttgtgcacaaagcagtcttattgggttaatgtgtaagcgggagacagtctcctatgccaaatggttcattaagcactgcctagtctaattgaactcttcaaatctccgggatccaggatctcagaaaccatgcccaagccagacagcgaagcagggactgccttcattcagacccagcagctccatgcagccatggctgacaccttcctggaacacatgtgccgcctggacattgactccgcacccatcacggcccgcaacactgggatcatctgtaccattggtgagtgagtgtggcccccttcgcagggcttctgcctggtttgaaaggcgtaataaccattgcagggctaacctaggttctgagagacatgtccacacttttagaggaacgtattggtgtacttcttctgcacttggtgaggttcagggtggtctagtggcctttgcaggagtagttcagggacttcagatctttttcctgtgctgtcactccactttctgcagaatttatagaaaatgatagtgtccttccttacatataaattaagagagtccttccttacatataaatcaagagagttgactagcacagtagtctttctggactttggaaagacctttctctaaatttgtggaggtattaaaaacaaaggacaaggagttgcctttgatagtagcagatttcatatactttcctacctgagaatcatgattttcttcctgtggctctaaatgtttcttggtaggccctgcttcccgatctgtggagatgctgaaggagatgattaagtctgggatgaatgtggctcggctgaatttctctcatggaacccatgaggtgagtggcagcttgatccaggaggttcgggacttgctgctgctgtgctccaggcttctcagtgaatggacgttgctctacttacatttagatcccctctctccctcttcccctctggccaacttcattggcatcaacaacctgcctgctttgtcgccggccaggttatccctaccatcaagcttcaccagggctttcttattctatgtgcaccttcgatgaggggctgtggctgtctgtcttggatgaggggctgtggctgtctgtcttggttttaaccattcctggagacggtggaatgaatcgtagctccctcgagctcttggcctaattgatcatctttggctccagggtggctcagggcatgggaactggacttgggtgtggagaacacctacctgtcaatctccccttctttctctccagtaccatgcagagactatcaagaatgtccgtgcagccacagaaagctttgcatctgatcccattctctaccgacctgttgcggtggctctggatacaaagggacctgagatccggactggactcatcaagggcgtgagtatccaggagtttaggtctttaaatgagaatatttttcatctgcctggtaggaattattatagatgcacatttttggtatgtgaataacatacttaagtctcactctggggacctggtttgttgtttgttttgtttgtttccctcaataaacaaattcaggatttacagaaaggtgatcggtttcttggggctttgagccagagtttgagcgccgccatcagggtgttggcgtccacagtcacacgcctctgctgtctttaatctagagcggcaccgcagaggtggagctgaagaagggagccacactgaagatcaccctggacaacgcctacatggagaagtgcgacgagaacatcctgtggctggactataagaacatctgcaaggtggtggaggtgggcagcaagatctacgtggacgatgggctcatctccctgcaggtgaaggagaaaggtatgtgtggtgtacagtccacggcccaatgccactcccatccccagaactctggtaagcacttaacctagcatgtatgaattggtctcccaaggtcaaaagtttaaggtggttgttggtctgcatccctggcctgtctgaaacactgcctgagaaaaaatagacaaataacctacaaaggcctatgtgtacacctctaccctttagttccagcactcgggaatcagcaggtgtgtgagttctcatgtgtaaggactactcctgtatgcctagaatgagtctagagttctcttggcttctcacaaactgagatagatggtcttgatccctttcacacaggtgctgactacctggtgacagaagtggaaaatggtggctccttgggcagcaagaagggcgtgaacctgcctggtgctgctgtggacctccctgctgtgtcagaaaaggacatccaggacctgaagtttggggtggagcaggacgtggacatggtgtttgcgtctttcatccgcaaggcggctgacgtgcatgaggttaggaaggtcctgggagagaagggcaagaacatcaagatcatcagcaaaatcgagaaccatgaaggtgtccgcaggtgaaggtccactcctaccgtgtgcctggggtgtggccttgagggcacctctgtcagggcccaggaaagctctgtccattctttggttgtacttcctgttctatagcatctttctttttatcaggtttgatgagattttggaggccagcgatggaatcatggtagctcgtggtgacctgggcattgagattccggcagagaaggtcttcctagctcagaagatgatgattggacgatgcaaccgagctgggaagccagtcatctgcgccacccaggcatgtgctatcccttccttctgtgttctccacctaggagacctggtcttgacctggcctttaggtacacgtacccgcacatagctatgacctgcgtacctgtgcaagcttcggggaattgccctggaatcatcactgaagatgtcctgctcttccattatttagtgactttcatttagcggtggtctcttacttaataaaaacccttgtttgtcccctcctagatgctggagagcatgatcaagaagccacgccccacccgtgctgaaggcagtgacgtggccaatgcagtcctagatggagctgactgcatcatgctgtccggagaaacagccaaaggggactaccctctggaggctgttcgcatgcagcacctggtgagtaagtcctcagagtcctggggtagaagagctctgctggagaggcctctgtccagtcttgttacattgctcccgtcacagcaaggagagtgaggtttgtggagtgtgcttgagtttcattgtgctttcactgcctgcacctgcccctttgtcctgctctggggattacataggccacatctggctaaaatatcaaggtcctaggatgcagtcaagggatgccttccttgtggacacccagagggcctgggtacctctattctaaaggagccaagagtttgttcagctattcgccttgttacctctcatttggtctcctgtggtctgcccatgggcaatgcttctctccactgcagctgtagccatactgagctgctttaagctggccgtgcatggtgcctgtgacatgggacttcctcccttgctgtgccagacccaactcggggctacaaatagctctgggggtggggaatgggtgctaatttagcaggttctgtctggtctaggaattataaagacttctcaggcatattatgtgctcttttgattaagtcttctggtttcagtaagatagggtctggcgcaagcttgtaattccaacaatcaaatcaagcctgcggcaggaagatggcttgagattaggctggactagaatgagacatcttgtagaagcaacaggcgtcagtgacctgtccctgcggactttccacccaggcctccgtcttctgtgttctgctccagaacttcttccggagcagaacaattatctggggcatcagttagggaccgttgcctgataaaggcatggtggattagctttttgggggttttctattcccttttcagccaacaaagaaccacagtatcttttgtggctccagtttcccacaagcccccccaaacttaagacaagtgaaagaaaagaaggaagccccttctgggttaggcatcttaccttgctttatatgtatatgagagttgagtagattccatgatctttccatggatttatctgaattatagctgtagccagatgtttgcctatattgaaacagaccagcgctttgactcggctggtcatgacctgttctgattggaattgtgactggtcctcatagcttctcatagctgcaggcattctctcccagaagacttttcccttccttcaatctcccccacagtagctgccttcctggtttacaccaaaaccccttttctacttacctggcctcacagtggtcaggaacaggactttgaccaggtattctaaagcatgtagtcacataaatgtatttttggatagctcaagaagaccttgcccaaagtggcggttgatgagattaattacaattaattatatgtgtatgagttagttttggtgtcctgtccttttctcatgccctggtctttcctatgaacagacaccttagaacctcgaggctgggattgcatggccctgctcagaagatgagtcacagagtccgggttagactgtggctaccccctcagggatacaaatttctctatcagttaacacttaggacagcttctcccctttctttatctgtttgctgtttcctcctgtgtctaactgattcagttcaaaacctgtaattaaaagctagacatcccagctgtggttgcttcctgtccatgcctcttgtcctttgggcttgcctgcattccatgcttaaccaaaagattactttccacttgcaaatctgctaatgctaccaataaaatcgagtgctggtttcatatcattcctggaattgcactctctaaaactttatttctttatttctaatgcttggttttacttgaacaggtggtctgtatataacacatttgctatcctgtaaccgttttaattattgcagtttgaatctgtgtcttgaaaggcctgtgtgctttcccaggcgtctgcctcctcacatggctgttcagtgtaccgtgttcaagtgagccagtcgaccactgttctgtttagaacttgtgcactgcaactttggctcttttgaccctcacccccagctttcagagctgcccgagtgtttccactgtaagccaagtgcaagcgctcactcttgtgtgtaggcggagttggatgccttgtaaccacatagccatgtgaggaggggacgcctttttcttcctgtaagctgtgtcaggaggcagtgtggtcaagcggaagtgtagttggctccaccttggcatctttccatgccagggtccctttctcagctttaatcaaaaacaaaagaaatgatggatggtgcatgcctagcacttgggaggctgaggcaaagaaaatagtggtgttttgaggctgaccatggcaagttcaaggccagtaggtgctatggtaagatcctatctcaaacctgtgtggggttgagaattggctttttttgtttgtttgtttttagaaacaggaaatcccaaaaggatactatctttccagaagggaaggaaggatctgtccgtgtgtttgaacagaaaagagagccacccaaaatcccacatataataccattccaggctttgaccatcctgcctctgtatctgcatggagaagaaaagattaacctaagagttccttcctctcattagttccctgtctttccatgtgttgtctcttgtttttgccttcatccttcttccttatccttcctaccctaaaccttacagatagctcgagaggctgaggcagccgtgttccaccgcctgctgtttgaagagcttgcgcgagcctccagtcaatccacagaccccctggaggccatggccatgggcagcgtggaggcctcttataaatgtttagcagcagctttgatagttctgacggagtccggcaggtagggccctgagggcaggtatcattataagataaccagcttctcacacaactagggcccactgtgtgcctgagcctgggcatagcctctctcctgcaggaaggcagccaaggaggtcacgatagggcaggaccaaaggattccctagtgggtacagtggaagtcacaggcactggttcaggatggttccctgtggagtttctaatcttgctcaagtttcagaaacgtgttagtgaactcatctttctcctggctttttgtgcccaggacatgttccttcccagttgcctgtgactcttctccttcatttgtgacaaagctctgacaaagccctgtcccccttcctcgtccctctggacggatgttgctcccctagattgcccgagaggcagaggctgccatctaccacttgcagttattcgaggaactccgccgcctggcgcccattaccagcgaccccacagaagctgccgccgtgggtgccgtggaggcctccttcaagtgctgcagtggggccattatcgtgctcaccaagtctggcaggtaggaggcggcagtggctccctggggatcccagagcaactctgggctgattttaagacccctggctgccatcaaaggactccaggaagcactcccaggtacatcagattaggtggctccagtgccagtcagtgcaggcctgcctcagggcctgaagcatatatacagttttgcttagttacggtgtggaagctgggcatgggtacatgcctttaatcctcccagcattcgggcagaagaggtgggcaaatgtctgcattcgtggacagtgctggtctacaaagcaagttccaggacagagccctagctcacaaaaattaactcagctggtaaatactacttgctacaccaagttagacggatggaggagggagaaaggtaacttagcaagcgtgcccaaggacatatacagtaacactttttctcatcccacttggagggagctcaatggataaagtgttggcaatctggatttggatctggcacctgtgggtttttgttgttgtttgggtttttgtttttttttttttttttaagtcacctggagaagtacgttaggcctttaggagcatggggttttggtttttaagatttatttattatatatgaactacactctccctatcttcagtcgcaccagaagagggtgattacagatgattgtgtaagccaccatgtggttgctgggagttgaactcaggacctctggaagagcagttagtgctcttaaccactgagccatctctccagccagcactgttctttcagaagcccaacacatacatgacagctcacactatctaactgcagtcctggggggaatctaatgccctcttctaacttccagccgtacatgtttatatgatgcatgtggtcaggatacccagacagctggagggaaaggaaggtgcccaacacacctgtgtgcctgtaatcccagcactcatggtacagaggtagaaagatcacaagataggtctaatctgagcaaattctgcaacagcctagatacagaaagccaaacacataaaaatcagtgtgggctgaggatgttgacctttgtgtgaattggccatgaacattgtatgcagctggaagcaagggatggggtgctgaaaaatggggaccttaaaactaattttctgaagtcctgaagtttcgagagctccaagatacaagttctatgctaatcctggactttctctgaagagttcctggttccttcaaagcccatgtaggccagctagaagcagggtattttctgcctgtggaatggccacatcccctaataccttgtgtgtcctcaagatgcctagttaattcagtaagcctgaaacagatctgatagaagtaaacatatcatttgctatcggttgaggagagaggggctccctacattcccaaaggatcttgattggccagatattaaccatgcctggtcacacctagattttccaaaaaaccatagaaattcagaggtttcttttagcaccaagttcagctgataagactcccaaaggagcagtatagtactctctggcaaatactgtccactccagggtctctgcagataccagaagtcaggagctaggcacatggtgcttggattgtaaaagttgctggcagctacaggatggttctggtgagattaggccagagctgcctcactgcctgatggaagggttcacagtgtgggagggatcctgccagccgtggtcctatgggactgcccacactgagatcaggacaatgagttaaaaaggacaggacaggtctgagggggtggccaggcactaaacagtaattgtaagtgggcaaacctgtggcctagaggtgaattagagggtgctcctttggctgactgaaagggtctcgtgacacaatggccattcttcccaccttctcaggagtgctcaccaagtggcccggtaccgcccaagggctcctatcattgctgtgacacgcaatccccagacagcccgccaggcccatctgtaccgtggcatcttccctgtgctgtgtaaggatgccgtactggatgcctgggctgaggacgttgatcttcgtgtgaacttggccatgaatgttggtatgtagctggaagcaagggatggggtgctgggaaatggggagcctaaaactaccatttcctgaagccctcacccaattttgggcccaaggcaaaattaattgcctcattaggctcttgtaagattaattggagctactgccctgtggggtggggcacacaagctgttgtggtctttgttcctatataaggttggatatcaagagacaaggaagcagctgaccctgaacttgggcaaggctggccactctagagtctaaactgaatggtgtccaaaggtccctgcagtctgagttttgcctcagcccttgtttaagctaggagacttgcaccaccttgtggtaaaaagaagtaactggcaacctgctctcctacctgaattagaagcaatagggcatttgttatcctgcctggttaggcttgtgtctgggctgggagaaagcaccaccttgtggtaggagagagtactggttgctcagttaggactgggtagggctttgcagttatccccaagtgttatatacctctactcaccaacctccttctcccctcaggcaaggcccgaggcttcttcaagaagggagatgtggtcattgtgctgactggatggcgccctggctctggcttcaccaacaccatgcgtgtagtgcctgtaccatgatgatcctctggagcttctcttctagcccctgtcccttcccctcccctatcctatccattaggccagcaacgttgtagtgctcactctgggccatagtgtggcgctggtgggctgggacaccaggaaaaattaatgcctctgaaacatgcaatagagcccagctatttttcatggccctacttgagccaggggtgaaggaggaatgcaggattggaaaccctctgactttatcacagaagggcagcattatctctgtgttctttgctcctgtagaaagttttccagagaattcccagccctggcctggaatcaggagacagcaagaacagaggctgggggcccagggttcccatgtagatgacttttggccctgtccctgacttgctttcccaacagctttggcctctctcctcgtgcactccactgctgtccctgcagatgttccactctccacctcgtactctgcagcgtctccaggcctgttgctatagtgcccacctgaatgtcaataaacagcagcggaacgacctgtgttcttttcttcctggggggcgtggttgaggctatcctctgagcagtcgaaaatgacaaccggcctaaaggctcagcctggagccgagtatataggttgcctcctccaacagcaccaagtggggctatctatgtgttggaaagctcactgttttatttcaggacagactggaaaggtctgggacaggttcagctagcacctgtaatggtgggtagaccggaggg

SEQ ID NO: 4 - protein, 446 aa, Rattus norvegicus
  Dopamine receptor D1
MAPNTSTMDEAGLPAERDFSFRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPFQYERKMTPKAAFILISVAWTLSVLISFIPVQLSWHKAKPTWPLDGNFTSLEDTEDDNCDTRLSRTYAISSSLISFYIPVAIMIVTYTSIYRIAQKQIRRISALERAAVHAKNCQTTAGNGNPVECAQSESSFKMSFKRETKVLKTLSVIMGVFVCCWLPFFISNCMVPFCGSEETQPFCIDSITFDVFVWFGWANSSLNPIIYAFNADFQKAFSTLLGCYRLCPTTNNAIETVSINNNGAVVFSSHHEPRGSISKDCNLVYLIPHAVGSSEDLKKEEAGGIAKPLEKLSPALSVILDYDTDVSLEKIQPVTHSGQHST

SEQ ID NO: 5 - protein, 960 aa, Mus musculus
  GABA-B1a receptor
MLLLLLLLLFLRPLGAGGAQTPNVTSEGCQIIHPPWEGGIRYRGLIRDQVKAINFLPVDYEIEYVCRGEREVVGPKVRKCLANGSWTDMDTPSRCVRICSKSYLTLENGKVFLTGGDLPALDGARVDFRCDPDFHLVGSSRSICSQGQWSTPKPHCQVNRTPHSERRAVYIGALFPMSGGWPGGQACQPAVEMALEDVNSRRDILPDYELKLIHHDSKCDPGQATKYLYELLYNDPIKIILMPGCSSVSTLVAEAARMWNLIVLSYGSSSPALSNRQRFPTFFRTHPSATLHNPTRVKLFEKWGWKKIATIQQTTEVFTSTLDDLEERVKEAGIEITFRQSFFSDPAVPVKNLKRQDARIIVGLFYETEARKVFCEVYKERLFGKKYVWFLIGWYADNWFKTYDPSINCTVEEMTEAVEGHITTEIVMLNPANTRSISNMTSQEFVEKLTKRLKRHPEETGGFQEAPLAYDAIWALALALNKTSGGGGRSGVRLEDFNYNNQTITDQIYRAMNSSSFEGVSGHVVFDASGSRMAWTLIEQLQGGSYKKIGYYDSTKDDLSWSKTDKWIGGSPPADQTLVIKTFRFLSQKLFISVSVLSSLGIVLAVVCLSFNIYNSHARYIQNSQPNLNNLTAVGCSLALAVVFPLGLDGYHIGRSQFPFVCQARLWLLGLGFSLGYGSMFTKIWWVHTVFTKKEEKKEWRKTLEPWKLYATVGLLVGMDVLTLAIWQIVDPLHRTIETFAKEEPKEDIDVSILPQLEHCSSKKMNTWLGIFYGYKGLLLLLGIFLAYETKSVSTEKINDHRAVGMAIYNVAVLCLITAPVTMILSSQQDAAFAFASLAIVFSSYITLVVLFVPKMRRLITRGEWQSETQDTMKTGSSTNNNEEEKSRLLEKENRELEKIIAEKEERVSELRHQLQSRQQLRSRRHPPTPPDPSGGLPRGPSEPPDRLSCDGSRVHLLYK

SEQ ID NO: 6 - DNA, 4365 bp, Mus musculus
  GABA-B1a receptor
taacttgcggccgccgtcgccccggggaaggagagacgggggtggggtttgcgggtagcgatcgcgaaggggagagaccccggccgggctggagcctggattcctgtggaagaagaacagggggaggggaagctggaggaccgggagggagaacggggagccgcggccgggcctgggcccttgaggcccggggagagccgcggagcgggaccggccgccgagatgctgctgctgctgcttctgcttctcttcctccgccccctgggcgctggcggggctcagacccccaacgtcacctcggaaggttgccagattatacatccgccctgggaaggtggcatcaggtaccgtggcttgattcgcgaccaggtgaaggccatcaatttcctgcctgtggactatgagattgaatatgtgtgccggggcgaacgcgaggtggtggggcccaaggtgcgcaagtgcctggccaacggctcctggacggatatggacacacccagtcgctgtgtccgaatctgctccaagtcttatttgaccctggaaaatgggaaggttttcctgacgggtggggacctcccagctctggatggagcccgggtggatttccgatgtgaccctgacttccatctggtgggcagctcccggagcatctgtagtcagggccagtggagcacccccaagccccactgccaggtgaatcgaacgccacactcagaacggcgtgcagtatacatcggggcgctgtttcccatgagcgggggctggccggggggccaggcctgccagcctgcggtggagatggcgctggaggacgttaacagccgcagagacatcctgccggactacgagctcaagcttatccaccacgacagcaagtgcgacccagggcaagccaccaagtacttgtatgaactactctacaacgaccccatcaagatcatcctcatgcccggctgcagctctgtgtccacactggtagccgaggctgcccggatgtggaaccttattgtgctctcatatggctccagctcaccagccttgtcaaaccgacagcggtttccaacgttctttcggacacatccatccgccacactccacaatcccacccgggtgaaactcttcgaaaagtggggctggaagaagattgccaccatccagcagactaccgaggtcttcacctcaacactggatgacctggaggagcgagtgaaagaggctgggattgagatcacttttcgacagagtttcttctcagatccagctgtgcctgttaaaaacctgaagcgtcaagatgctcgaatcatcgtgggacttttctatgagaccgaagcccggaaagttttttgtgaggtctataaggaacggctctttgggaagaagtatgtctggtttctcatcgggtggtatgctgacaactggttcaaaacctatgacccgtcaatcaattgtacagtagaagagatgactgaggcggtggagggccatatcaccacggagattgtcatgctgaaccctgccaacacccgaagcatttccaacatgacatcacaggaatttgtggagaaactaaccaagcggctgaaaagacaccctgaggagactggaggcttccaggaggcaccactggcctatgatgctatttgggccttggctttggccttgaacaagacctctggaggaggtggccgttcaggagtgcgcctggaggactttaactacaacaaccagaccattacagaccaaatctaccgggccatgaactcctcctcctttgagggtgtttctggccacgtggtctttgatgccagcggctcccggatggcatggacgcttatcgagcagctacagggcggcagctacaagaagatcggctactacgacagcaccaaggatgatctttcctggtccaaaacagacaagtggatcggagggtctcccccagccgaccagaccttggtcatcaagacattccgtttcctgtcacagaaactctttatctccgtctcagttctctccagcctgggcattgttcttgctgttgtctgtctgtcctttaacatctacaactcccacgctcgttatatccagaattcccagcccaacctgaacaatctgactgctgtgggctgctcactggcactggctgttgtcttccctctcgggctggatggttaccacatagggagaagccagttcccgtttgtctgccaggcccgcctttggctcttgggcttgggctttagtctgggctatggctctatgttcaccaagatctggtgggtccacacagtcttcacgaagaaggaggagaagaaggagtggaggaagaccctagagccctggaaactctatgccactgtgggcctgctggtgggcatggatgtcctgactcttgccatctggcagattgtggaccccttgcaccgaaccattgagacttttgccaaggaggaaccaaaggaagacatcgatgtctccattctgccccagttggagcactgcagctccaagaagatgaatacgtggcttggcattttctatggttacaaggggctgctgctgctgctgggaatctttcttgcttacgaaaccaagagcgtgtccactgaaaagatcaatgaccacagggccgtgggcatggctatctacaatgtcgcggtcctgtgtctcatcactgctcctgtgaccatgatcctttccagtcagcaggacgcagcctttgcctttgcctctctggccatcgtgttctcttcctacatcactctggttgtgctctttgtgcccaagatgcgcaggctgatcacccgaggggaatggcagtctgaaacgcaggacaccatgaaaacaggatcatccaccaacaacaacgaggaagagaagtcccgactgttggagaaggaaaaccgagaactggaaaagatcatcgctgagaaagaggagcgcgtctctgaactgcgccatcagctccagtctcggcagcaactccgctcacggcgccaccccccaacacccccagatccctctgggggccttcccaggggaccctctgagccccctgaccggcttagctgtgatgggagtcgagtacatttgctttacaagtgagggggcatggagaaggatcaagccagtaggggagggaagggtctgggaagagggtgggggcctgggaggagggtaaggactcctatctccaacctggagagcacacgctccaatccccctcttataaatacatgtcgctctgtgcatctggggttatttgggtctccagtactctgggaaacagactgttttctttctcccctataattttatatctccacttcacaggttttgtttgaaccctgcttggagttattattcactcatggctccagaggggcatctcatttttctccggtagcctgtcttgtacagttaccacagcaactcctgtcatttcagcagcaggggtcttcctacactagcagggctctcgctctctccatttttcagcctcagaatctccttccattattcttctccttctacatgtctccatggcttcctctcccaggggactcgttctacacacatacacacacacacacacacacacacacacacacacacacacacacaccccgcatcctgccctctcctaggcagctgcatgtcgtcctgtacaaatgtgctcgcttctgagtgctttgtgcggccgttcacttgtgctgtctgcataagctgcgtctgtgagtgcacggtggtttgtgggtgcgtgaagtggcatgctccggtaggtgtgtgtgtgtgtgcccacgcgtgcgcccgtgtgcatgcgttcgtgttgccctgactggctgtctcagccttctgagtaattgggattccagttgtctgtctagctcatgtcctgtcttcttccagtagagccgtgaacacccaacacacacagttaatcgggctccccccagtccatgttttctgagccatccaaaaactctccttggccttaggttcatctgcaaatgttccctctgttctttgctctcgtgcgtccaccttcattctcttcagtcatttctcagatctgctgcgtcgtggtttcctttccttcattatcatcgtcattatttttcagaacttaagggaaaaagaaatggggacaggttggaggctgtttccagtggaatagtgggtgcgcgtcctgaccaaatgaaggcacggacagatggactgacggggcgggaggcggcgtccctttcacaccgtggtgtctcttgggggggaaggatctccctgaatctcaataaagcagtgaacagtaaaaaaaaaaaaaaaaaaaaaaaaaaaaaaaaaaaaaaaaaaaaaaaaaaaaaaaaa

SEQ ID NO: 7 - protein, 582 aa, Rattus norvegicus
  Differentiation-associated Na-dependent inorganic phosphate cotransporter
MESVKQRILAPGKEGIKNFAGKSLGQIYRVLEKKQDNRETIELTEDGKPLEVPEKKAPLCDCTCFGLPRRYIIAIMSGLGFCISFGIRCNLGVAIVDMVNNSTIHRGGKVIKEKAKFNWDPETVGMIHGSFFWGYIITQIPGGYIASRLAANRVFGAAILLTSTLNMLIPSAARVHYGCVIFVRILQGLVEGVTYPACHGIWSKWAPPLERSRLATTSFCGSYAGAVIAMPLAGILVQYTGWSSVFYVYGSFGMVWYMFWLLVSYESPAKHPTITDEERRYIEESIGESANLLGAMEKFKTPWRKFFTSMPVYAIIVANFCRSWTFYLLLISQPAYFEEVFGFEISKVGMLSAVPHLVMTIIVPIGGQIADFLRSKQILSTTTVRKIMNCGGFGMEATLLLVVGYSHTRGVAISFLVLAVGFSGFAISGFNVNHLDIAPRYASILMGISNGVGTLSGMVCPIIVGAMTKNKSREEWQYVFLIAALVHYGGVIFYALFASGEKQPWADPEETSEEKCGFIHEDELDEETGDITQNYINYGTTKSYGATSQENGGWPNGWEKKEEFVQESAQDAYSYKDRDDYS

SEQ ID NO: 8 - DNA, 3982 bp, Rattus norvegicus
  Differentiation-associated Na-dependent inorganic phosphate cotransporter
agacagtaaggttcttttgcttttttcccttacacaaggattcgatgacgtttttggtcaatctgattaaaagacagcggatttggttgcgttaagacttcaaaaccgggaatttacgttgtttttcggtgaggtgacttccagaacggggactcatcagcacccgcccaaataccacggcactgcgcgcgccctcggccaccggatcctccccttccaatgagactttgtgactgtgtgtaccaattctcctattaggaaacccgtgggctgaatgcagctattccgttgtactctctttctcgctctccctcccctctccaactcacagccttgctgaaaagctcatctctgctgagaagaaaacgttctaccttaacctattaagactatgcgcagaactcgcctttcatagccatcacaatttaaatctggtaaggctggacacgagtctttacaagaatggagtcggtaaaacaaaggattttggccccggggaaagaggggataaagaattttgctggaaaatccctcggacagatctacagggtgctggagaagaagcaggataaccgagagaccatcgagctgacagaggacggcaagcccctggaggtgcctgagaagaaggctccgctatgcgactgtacgtgcttcggcctgccgcgccgctacatcatagccatcatgagcggcctcggcttctgcatctcctttggtatccgctgtaacctgggtgtggccattgtggacatggtcaacaacagcaccatccaccggggtggcaaagttatcaaggagaaagccaagtttaactgggaccccgagactgtggggatgattcacgggtcgttcttctggggctatatcatcacgcagattccgggcggatacatcgcatcgcgactggctgctaaccgggtctttggggctgccatactgcttacctctaccctcaatatgctgatcccatctgcagccagagtgcattatggatgcgtcatctttgttagaatattgcaaggacttgtggagggcgtcacctacccagcctgtcacgggatatggagcaagtgggcccctcctttggagaggagtaggttggctaccacctccttctgtggttcctatgctggagcagtcattgcaatgcccctagctggtatcctggtgcagtacactggatggtcttcagtattttacgtatatggaagctttggtatggtctggtatatgttctggcttctggtgtcttacgagagccccgcaaagcatccaaccataacagacgaagaacgtaggtacatagaagagagcatcggggagagcgcaaatctgttaggagcaatggagaaattcaagaccccatggaggaagtttttcacatccatgcccgtctatgcgataattgttgcaaacttctgcaggagttggactttttatttactgctcatcagtcaaccagcttatttcgaggaggtttttggatttgaaatcagcaaggttggcatgttgtctgcggtcccacacctggtcatgacaatcattgtgcctatcggggggcaaattgcagactttctaaggagcaagcaaattctttcaacaactacagtgcgaaagatcatgaactgcgggggttttggcatggaagccacactgcttctggttgttggctactctcatactagaggggtggccatctccttcttggtgcttgcagtgggattcagtggatttgctatctctggtttcaatgtgaaccacttggatattgccccgagatatgccagtatcttaatgggcatttcaaatggtgttggcacgctgtcgggaatggtctgcccgatcattgttggtgcaatgacgaagaacaagtcccgtgaagaatggcagtatgtcttcctcatcgctgcactggtccactatggtggagtcatattttatgcactatttgcctcaggagagaagcaaccttgggcagaccctgaggaaacaagcgaagaaaagtgtggcttcattcatgaagatgaactggatgaagaaacgggggacatcactcagaattacataaattacggtaccaccaaatcctacggcgccacctcacaggagaacggaggctggcctaacggctgggagaaaaaggaagaatttgtgcaagaaagtgcgcaagacgcgtactcctataaggaccgagatgattattcataacgaagctagttgctggattcctttgtagtgtttgtgattaaattaattgtgattgcacaaaatcattttaagaaatgtggtgtaaacatgtaaacacatcaaccaagcaagtcttgctgttcaaaaaataataataatatgaattcaaaacagaccgtgagagtcccatcaagtgcaatctgtggcggcagtcacgtgacgccatttccattcaggccattcgtccttttcgtttgtgatttaaaggtttcctgtagaaataagtaggtattcgttggatccatcaccacgttagagagtacaactacaacagttggcacatgtcatcctacggaagttaggaagccaaagctactggattatgtgaactgcatttatttatttattacactggactgcaaaatatcccagggaaatcctgtctagagacatagtagaactggaaagatggctagattgggtactgacgataatcattgtgtgtatatcatggagtggctatatcttttaattggagaactatattgtatagctagcaaaattgtactgaattattactaggagtgcacagtgtgtgatattttgtgatcttccaaaagcttatcttgcagtgttttgtgaaacgcttgggcacaaacacttatttttatgaacaagagcttgtaaagggaggagtatgctccatgctctcccattcactacctgacagtatcaaaccttcacatttcaatgaaatccaacgtccatgtaacatatcacatgactttttttgcaaaaaagaatataagaagaaatagacttcaatgtattttttattacaactttgtactggttgtaacttgcattaggaaaaatgattaatatatgtataatcgtaaagaatctaataaaaatttactatgaagatatagcccttaaaatgcaatattaacaacaaaaatatatagaaaatttagataatcttccttgataactagagactatatggaactcacaccacaaagctatatataatatgaaaagataaacaatagagattgtatatgtagacgattttatgacctaatgtcccatttaagaggtatttgtcttgagtatatagtacaaagtatattaaaattatatctacatccctgtatatcttatacatatccactcacacaaacataacaaatacttttcacacagaaccaaaaacaagcatacacctaatgttgggtttggggattgcaatttctactttcatagagtcatagaattttagatgggaaaaaaaaaggcattttgctcgtcatttcttaatataattaattcaacaggaactgcaacatttgtgtaccaagcaataagtgcgaagcataaacctgctgtgtgtaaactatccccatactgcttgtggtagcactgatttctttcttttaaagaacttaacatcggagctctttacaatgttttgcgctgataagaatgcacatcccaatttaacgcaaagtgtcacctggtgtgtttacctgtctgttttgggtatttggtctgtttggtgtcctgtgctcttgactggaggccctgctactgcgaatataaaacgtgaagtttgtttctaaatgcaaaccactcctgaccttaagaaactaaagtccctctctgctttgtgtctccaagtactatcatgtgaccataacccttgctgtgctgagtaaaaagatgtgaactgtcattttgttgctgcgaagcaagtgttaataaaatgttctatttaaaaaaaaaaaaa

SEQ ID NO: 9 - protein, 854 aa, Homo sapiens
  Vacuolar assembly protein VSP41 gene
MAEAVEQETGSLEESTDESEEEESEEEPKLKYERLSNGVTEILQKDAASCMTVHDKFLALGTHYGKVYLLDVQGNITQKFDVSPVKINQISLDESGEHMGVCSEDGKVQVFGLYSGEEFHETFDCPIKIIAVHPHFVRSSCKQFVTGGKKLLLFERSWMNRWKSAVLHEGEGNIRSVKWRGHLIAWANNMGVKIFDIISKQRITNVPRDDISLRPDMYPCSLCWKDNVTLIIGWGTSVKVCSVKERHASEMRDLPSRYVEIVSQFETEFYISGLAPLCDQLVVLSYVKEISEKTEREYCARPRLDIIQPLSETCEEISSDALTVRGFQENECRDYHLEYSEGESLFYIVSPRDVVVAKERDQDDHIDWLLEKKKYEEALMAAEISQKNIKRHKILDIGLAYINHLVERGDYDIAARKCQKILGKNAALWEYEVYKFKEIGQLKAISPYLPRGDPVLKPLIYEMILHEFLESDYEGFATLIREWPGDLYNNSVIVQAVRDHLKKDSQNKTLLKTLAELYTYDKNYGNALEIYLTLRHKDVFQLIHKHNLFSSIKDKIVLLMDFDSEKAVDMLLDNEDKISIKKVVEELEDRPELQHVYLHKLFKRDHHKGQRYHEKQISLYAEYDRPNLLPFLRDSTHCPLEKALEICQQRNFVEETVYLLSRMGNSRSALKMIMEELHDVDKAIEFAKEQDDGELWEDLILYSIDKPPFITGLLNNIGTHVDPILLIHRIKEGMEIPNLRDSLVKILQDYNLQILLREGCKKILVADSLSLLKKMHRTQMKGVLVDEENICESCLSPILPSDAAKPFSVVVFHCRHMFHKECLPMPSMNSAAQFCNICSAKNRGPGSAILEMKK

SEQ ID NO: 10 - protein, 770 aa, Rattus norvegicus
  Git 1
MSRKGPRAEVCADCSAPDPGWASISRGVLVCDECCSVHRSLGRHISIVKHLRHSAWPPTLLQMVHTLASNGANSIWEHSLLDPAQVQSGRRKANPQDKVHPIKSEFIRAKYQMLAFVHKLPCRDDDGVTAKDLSKQLHSSVRTGNLETCLRLLSLGAQANFFHPEKGTTPLHVAAKAGQTLQAELLVVYGADPGSPDVNGRTPIDYARQAGHHELAERLVECQYELTDRLAFYLCGRKPDHKNGHYIIPQMADRSRQKCMSQSLDLSELAKAAKKKLQALSNRLFEELAMDVYDEVDRRENDAVWLATQNHSTLVTERSAVPFLPVNPEYSATRNQGRQKLARFNAREFATLIIDILSEAKRRQQGKSLSSPTDNLELSARNQSDLDDQHDYDSVASDEDTDQEPLPSAGATRNNRARSMDSSDLSDGAVTLQEYLELKKALATSEAKVQQLMKVNSSLSDELRKLQREIHKLQAENLQLRQPPGPVPVPSLPSERAEHTLMGPGGSTHRRDRQAFSMYEPGSALKPFGGAPGDELATRLQPFHSTELEDDAIYSVHVPAGLYRIRKGVSASSVTFTPSSPLLSSSQEGSRHASKLSRHGSGAESDYENTQSGEPLLGLEGKRFLELSKEDELHAELESLDGDPDPGLPSTEDVILKTEQVTKNIQELLRAAQEFKHDSFVPCSEKIHLAVTEMASLFPKRPALEPVRSSLRLLNASAYRLQSECRKTVPPEPGAPVDFQLLTQQVIQCAYDIAKAAKQLVTITTREKKQ

SEQ ID NO: 11 - DNA, 3236 bp, Rattus norvegicus
  Git 1
gcccgcgccccccacgctggcccagagggctgcggccgcttcgccgctgaggatgtcccggaaggggccgcgagcggaggtgtgtgcggactgcagcgcccctgaccctggctgggcatctatcagcagaggtgtgctggtttgtgacgagtgctgcagtgtgcaccggagcctgggacgacacatctccattgtcaaacaccttcgccacagcgcctggcctcctacgctgctacagatggtgcacacgcttgccagcaacggggccaactctatctgggagcattccctgctggaccctgcacaagtgcaaagtggccggcgcaaagccaacccccaagacaaagtccaccccatcaagtcagagttcatcagggcaaagtaccagatgctggcgtttgtgcacaagcttccctgccgtgatgatgatggggtcaccgccaaagacctcagcaagcaactgcactcgagtgtgcgaacaggcaacctggagacctgtctgcgcctgctttccctgggtgcccaggccaacttcttccacccagaaaagggcactacacctctacacgtggctgccaaggcagggcagacactgcaggctgagctgctagtagtgtatggggctgacccaggctcccctgatgtcaatggccgcacacccattgactatgccaggcaggcggggcaccatgaactggcagaaaggctagttgagtgccagtatgagctcactgaccggttggccttctacctctgtggacggaagcctgatcacaagaatgggcattacatcataccacagatggctgacagatctcggcaaaagtgcatgtctcagagtttggatctgtctgaattggccaaagctgccaagaagaagctacaagcactcagcaaccggctctttgaggagctcgccatggatgtgtatgacgaagtggaccggagagaaaatgatgcagtgtggctagctacccagaatcatagcaccctggtgacagaacgcagtgctgtgcccttcctgccagtcaaccctgaatactcagccactcggaatcagggacgacagaaattggctcgttttaatgcccgagagtttgccaccttgatcatcgacatcctcagtgaggccaaacggaggcagcagggcaagagtctgagcagccccacagacaacctcgagctgtctgcacggaaccaaagtgacctggacgaccagcacgactacgatagtgtggcttctgacgaagacacagaccaggagcccttgcccagtgctggcgccacgcggaacaatcgtgccaggagcatggactcctcagatctgtcggatggggctgtgacgctgcaggagtacctggagctgaagaaggctctggctacctccgaggccaaagtgcagcagctcatgaaagtcaacagcagtctgagtgacgagcttcggaagctgcagagggagatccacaaactgcaggcagagaacctgcagctccggcagccaccaggaccagtgcctgtaccctcacttcccagtgaacgggcagaacacacactcatgggccctggtggaagtacccatcgcagggaccgccaggccttctctatgtatgagccaggctccgccctgaagccctttggaggtgcacctggggatgagctcgccacacggctccagcctttccacagcacagagctggaagatgatgccatctattcagtacatgtccctgctggcctttaccggatccggaagggggtgtctgcctcttctgtgaccttcactccctcctccccactgctgtcaagctcccaagaaggaagtcgccatgcgagcaagctttcacgccacggcagtggcgcagagagtgactatgagaacacacagagtggagagcctctgctgggacttgaagggaagcgattcctagagctgagcaaggaggatgagctgcacgctgagctggagagcttagatggagacccagaccccgggctccccagcacagaagatgtcatcctaaagacagagcaggtcaccaagaacattcaggagctattgcgggcagcccaggagttcaaacatgacagctttgtgccctgttcagaaaagatccatttggctgtgactgagatggcctctctcttcccaaagaggccagccctggagcctgtgcgcagttcactgcggctgctcaacgccagcgcctaccggctgcagagtgaatgccggaagacagtgcctccagaacctggcgcccctgtggacttccagctactgactcagcaggtgatccagtgcgcctatgacatcgccaaggctgccaagcagctggtcaccatcaccacccgagagaagaaacagtgaccactttccctgcccttacccacaccctgggacctcactggccatgggagctgggccactccagacactaacccacccccaacagagccactggcacaagtgcccttagtgccaccattctcctctggcatccaggtgccctggtgtccacacccccttaagccccaaggatgggaatgtgaggtgacaggaccatctgtcccccacattccacgcccctccctctgtacatagcatccctgccctgctgatacgcaggagcctcaaggagtccctcccagctcttcacctgccttccagggccccctcagcaaagggtgggacagggacactccaagtggggcagccccgcctactgcaccctatccccatgagccagttcagccctggggggtcgagcagaggcaccccctcctttgtgcattatctmtgggtgtytytgccccgtagccagcagcccccccgctgctctttcatgacaaatggcccctgccagagcacaggccccaacttgtgtgccagggtccccagcagcaaacactggaacgtctttctctcccctaccccttaattttaactttgtggtaactgagtgtccctgcgttcttacaactgagtgtgtgagcggcagtgccgtgccagagacctggtccacctggagctctgaggggcaggggaaccagagtagtgggaccagcattggaagtcagcttccctctacccaacctgggacggggactgtcagggtcaccagctgtggtccagttgctacccctacttgtatccctatcctataaacttaaaaggaaaaccacttccctccatctctccccatcccctgtgggaagtgtgctgaccaggcagaggacag

SEQ ID NO: 12 - protein, 136 aa, Homo sapiens
  Histone H3.3
MARTKQTARKSTGGKAPRKQLATKAARKSAPSTGGVKKPHRYRPGTVALREIRRYQKSTELLIRKLPFQRLVREIAQDFKTDLRFQSAAIGALQEASEAYLVGLFEDTNLCAIHAKRVTIMPKDIQLARRIRGERA

SEQ ID NO: 13 - DNA, 1039 bp, Homo sapiens
  Histone H3.3
tgttcgcagccgccgccgcgccgccgtcgctctccaacgccagcgccgcctctcgctcgcgtaagtaaggaggtctctgcgagctccagccgaagagaagggggtaccatggctcgtacaaagcagactgcccgcaaatcgaccggtggtaaagcacccaggaagcaactggctacaaaagccgctcgcaagagtgcgccctctactggaggggtgaagaaacctcatcgttacaggcctggtactgtggcgctccgtgaaattagacgttatcagaagtccactgaacttctgattcgcaaacttcccttccagcgtctggtgcgagaaattgctcaggactttaaaacagatctgcgcttccagagcgcagctatcggtgctttgcaggaggcaagtgaggcctatctggttggcctttttgaagacaccaacctgtgtgctatccatgccaaacgtgtaacaattatgccaaaagacatccagctagcacgccgcatacgtggagaacgtgcttaagaatccactatgatgggaaacatttcattctcaaaaaaaaaaaaaaaatttctcttcttcctgttattggtagttctgaacgttagatattttttttccatggggtcaaaggtacctaagtatatgattgcgagtggaaaaataggggacagaaatcaggtattggcagtttttccattttcatttgtgtgtgaatttttaatataaatgcggagacgtaaagcattaatgcaagttaaaatgtttcagtgaacaagtttcagcggttcaactttataataattataaataaacctgttaaatttttctggacaatgccagcatttggatttctttaaaacaagtaaatttcttattgatggcaactaaatggtgtttgtagcatttttatcatacagtagattccatccattcactatacttttctaactgagttgtcctacatgcaagtacatgtttttaatgttgtctgtcttctgtgctgttcctgtaagtttgctattaaaatacattaaactat

SEQ ID NO: 14 - protein, 520 aa, Rattus norvegicus
  Cytosolic 3-hydroxy 3-methylglutaryl coenzyme A synthase
MPGSLPLNAEACWPKDVGIVALEIYFPSQYVDQAELEKYDGVDAGKYTIGLGQARMGFCTDREDINSLCLTVVQKLMERNSLSYDCIGRLEVGTETIIDKSKSVKSNLMQLFEESGNTDIEGIDTTNACYGGTAAVFNAVNWIESSSWDGRYALVVAGDIAIYASGNARPTGGVGAVALLIGPNAPVIFDRGLRGTHMQHAYDFYKPDMLSEYPVVDGKLSIQCYLSALDRCYSVYRKKIRAQWQKEGKDKDFTLNDFGFMIFHSPYCKLVQKSLARMFLNDFLNDQNRDKNSIYSGLEAFGDVKLEDTYFDRDVEKAFMKASAELFNQKTKASLLVSNQNGNMYTSSVYGSLASVLAQYSPQQLAGKRIGVFSYGSGLAATLYSLKVTQDATPGSALDKITASLCDLKSRLDSRTCVAPDVFAENMKLREDTHHLANYIPQCSIDSLFEGTWYLVRVDEKHRRTYARRPSTNDHSLDEGVGLVHSNTATEHIPSPAKKVPRLPATSGEPESAVISNGEH

SEQ ID NO: 15 - DNA, 3275 bp, Rattus norvegicus
  Cytosolic 3-hydroxy 3-methylglutaryl coenzyme A synthase
cccagggtccgatcgcgtttggtgcctgaaggaggaaccggtgacagacctggagactacagttctctgtccttcacacagctctttcaccatgcctgggtcacttcctttgaatgcagaggcttgctggccaaaagatgtgggaatcgttgcccttgaaatctactttccttctcagtatgttgatcaagctgagttggaaaaatacgatggtgtagatgctggaaagtataccattggcctgggccaggccaggatgggcttctgcacggatcgcgaagacatcaactctctctgcctgactgtggttcagaaactgatggagagaaatagcctttcctatgactgcattgggcggctggaagtcgggacagagacaatcatcgacaaatcaaaatccgtgaagtctaatttgatgcagctgtttgaggagtctgggaatacagatatagaaggaatagatacaactaatgcatgctatgggggcacagccgcagtcttcaatgctgtgaactggatcgaatccagctcttgggatggacgatacgctttggtagttgcaggagacatcgctatatatgcctcaggaaacgccaggcctacaggtggagttggagctgtggctctgctaattgggccaaatgctcctgtaatttttgaccgagggcttcgtgggacacacatgcagcatgcctacgacttttacaagcctgacatgctctctgaataccctgtggtagatggaaaactctccatacagtgctacctcagcgcattggaccgctgctattctgtctaccgcaaaaagatccgggcccagtggcagaaagagggaaaggataaagattttaccctgaatgattttggcttcatgatctttcactcgccatactgtaaactggtgcagaaatctctagctaggatgttcctgaatgactttcttaacgatcaaaacagagacaaaaacagtatttacagtgggctggaagcctttggggatgtgaaattagaagatacttacttcgacagagatgtggaaaaggcatttatgaaggctagtgctgagctattcaaccagaaaacaaaggcatctttgcttgtatcgaatcaaaatggaaacatgtacacatcctctgtatacggttcccttgcttctgttctggcacagtactcacctcaacagttggccgggaagaggattggagtgttctcttacggttctggcttggctgccacactctactcccttaaagtcacacaagatgccacaccaggatctgctcttgacaaaataacagcaagtttatgtgaccttaagtcaaggcttgactcaagaacgtgtgtggcaccagacgtctttgctgaaaacatgaagctcagagaggacacacatcacttagccaactatattccccagtgttcaatagattcactcttcgaaggaacatggtatctagtcagagtggatgaaaagcacagaagaacttacgcccggcgtccctccacaaatgaccacagtttggatgaaggagtgggacttgtgcattcaaacacagctacagagcatattccaagcccggctaagaaagtgccaagacttcctgcgacctcgggcgaacctgagtcggctgtcatcagtaacggggagcactgagagccgtggccttcacagaggctcggggctggatggggtatgggaaacgttagaggaatggatgtcttgggacaattttacagattacgtgttgcttaaaaatgtaatgtaactgacacagagcccagaaaactgttgtgtttttggagaagtctcgctgagctcctaacacacttcctgctgtgggctggccaatggtgaatgtactgcgatggtgttaagggctctgcagaacgtcatacctcgctgcatgtttacacgcatgcgggttaggcttcaaactcggtctgaactgagtgcttctgactgcaaaggcagaggtactgctgtccagtttaaaaaattgttttttttttttttaatgtgtaagaatttttatacttaaataaaaaaaagtacctgtagcttttgggggaaaaaaaaaaacctttttctaggttggggattgtggaatttaaatgttacacataaactctgcttaatggcaaggcaaacatttatctttttcgaagatttataaatcctgaagagaaaaaaagagggtatggttctaggatctggatgaaccatcagtgagaaaggttagtcataatcaagtgagcagaaggatgctggcgttgagcaggcctctgtcacagcaaccagggctctgtgggcacagctgagggaactttctggccaggtgcccgtgactgctgctcagctgcactgagatgcagtggagctgctgcacggaagcttgctgtggtgctgaacgccttacctgcggataaagtgtaaagtaggagggatgggcagggcactattaggttacagtgttacagacccaggttatagacttgacagctcaaactcaccagacacctttttccctttgtggtttgtgtattttttgtgttttgtttgtttttttttatattgttcaatttaaaaaatttagaaaattttaaccttacgttttcacatagtgtgattagccaaaaggaatttcacttcaagatctagaaatagaattcataacattttttcctaaactttgacttttaaaacaacgaaaattaccacatgagatgaacaagaaaattcattagaaagttctctgggtgatttttggtgctgaactgacatgagcctcatagactgtaaaacagaggtagttgaaactaatgtacagaactacattttttaattttatttgcatttaattctgtgaagtttcagttatctaaaataaacacataaacgtgtaatgtttcagattgcaaggtgagatgtaatgtagcatttgtaagatattcttgtcaatattaactggtaggattttgatttgtacagttttaattggttaaaatgatctcattttaacatccactgctatagatgaatgatgtaagctcagatttaatgaatggtggggaaatggtgcatgtaattttttcgcaagtatcgagagttctgtatgttttgaaaagaataatttaacgtttgggttgccaggaagggggctttcccagagtccattgccaggcgttgggcaagcctcgcaatgttggcacggagcgttaaccacaccttactaatagcaaggggaataactttgaaataaagttttagacaaata

SEQ ID NO: 16 - protein, 353 aa, Rattus norvegicus
  farnesyl pyrophosphate synthetase
MNGDQKLDVHNQEKQNFIQHFSQIVKVLTEDELGHPEKGDAITRIKEVLEYNTVGGKYNRGLTVVQTFQELVEPRKQDAESLQRALTVGWCVELLQAFFLVLDDIMDSSHTRRGQICWYQKPGIGLDAINDALLLEAAIYRLLKFYCREQPYYLNLLELFLQSSYQTEIGQTLDLITAPQGQVDLGRYTEKRYKSIVKYKTAFYSFYLPIAAAMYMAGIDGEKEHANALKILLEMGEFFQIQDDYLDLFGDPSVTGKVGTDIQDNKCSWLVVQCLLRATPQQRQILEENYGQKDPEKVARVKALYEELDLRSVFFKYEEDSYNRLKSLIEQCSAPLPPSIFLELANKIYKRRK

SEQ ID NO: 17 - DNA, 1271 bp, Rattus norvegicus
  farnesyl pyrophosphate synthetase
ttatatttgggttctgcctactgagccgggagtctgggaactacaactcccagagtgctgagcggatgcacgctctgcttttaggtgtaagccgcaaacatcttggaccccgggagaatccgcgttgaagcacagagcatttagctcctctgtcagaatgaatggggaccagaaactggatgttcataaccaagaaaagcagaatttcatccagcacttctcccagattgtcaaggtgctgactgaggatgaactgggacacccagagaagggagatgctattacccggatcaaagaggtcctggagtacaacactgtaggaggcaagtacaatcggggtctgacggtggtacagaccttccaggaactggtggaaccaaggaaacaggatgctgagagcctacagcgggccctgacggtgggctggtgtgtagaactgctccaggctttcttcctcgtgttagatgacatcatggactcttcccacactcgccgggggcagatctgctggtatcagaagccgggcataggcttggatgccatcaacgatgctctgcttctggaagccgctatctaccgcctgcttaagttctactgcagggagcagccctactacctcaacctgctggagctctttctacagagttcctatcagactgagatcgggcagactctcgacctcatcacagcaccccagggccaagtggatcttggtagatacactgaaaagaggtacaaatctatcgtcaagtacaagacagctttctactctttctacctgcctatcgcggctgccatgtacatggctggaattgatggggagaaggaacacgctaatgccctgaagatcctgctggagatgggcgagttcttccagatccaggacgactaccttgatctctttggagaccccagtgtgaccggaaaggtcggcactgacatccaggacaacaaatgcagctggctggtggttcagtgtctgctacgagccactcctcagcagcgccagatcttagaggagaattatgggcagaaggacccagaaaaagtggcgcgggtgaaagcactgtacgaggagctggatctgcggagtgtgttcttcaagtacgaggaagacagttacaaccgcctcaagagtctcatagagcagtgctccgcgcccctgcccccatccatcttcctggaactagcaaacaagatctacaagcggagaaagtaacctcgaattgtagaggctgcgagggaggggtctcaataaattattgttcaac

SEQ ID NO: 18 - protein, 152 aa, Rattus norvegicus
  Bendless protein
MAGLPRRIIKETQRLLAEPVPGIKAEPDESNARYFHVVIAGPQDSPFEGGTFKLELFLPEEYPMAAPKVRFMTKIYHPNVDKLGRICLDILKDKWSPALQIRTVLLSIQALLSAPNPDDPLANDVAEQWKSNEAQAIETARAWTRLYAMNNI

SEQ ID NO: 19 - DNA, 459 bp, Rattus norvegicus
  Bendless protein
atggccgggctgccccgcaggatcatcaaggaaacccagcgtttgctggcagaaccagttcctggcattaaagcagaaccagatgagagcaacgcccgttattttcatgtggtcattgctggtccccaggattccccctttgagggagggacttttaaacttgaactattccttccagaagaatacccaatggcagcacctaaagtacgtttcatgaccaaaatttatcatcctaatgtagacaagttgggaagaatatgtttagatattttgaaagataagtggtccccagcactgcagatccgaacagttctgctatcaatccaggctttgctaagtgctcctaatccagatgatccattagcaaatgatgtagctgagcagtggaagagcaacgaagcccaagccatagaaacagcaagagcatggactaggctatatgccatgaataatatttaa

SEQ ID NO: 20 - protein, 2505 aa, Rattus norvegicus
  Fatty acid synthase
MEEVVIAGMSGKLPESENLQEFWANLIGGVDMVTDDDRRWKAGLYGLPKRSGKLKDLSKFDASFFGVHPKQAHTMDPQLRLLLEVSYEAIVDGGINPASLRGTNTGVWVGVSGSEASEALSRDPETLLGYSMVGCQRAMMANRLSFFFDFKGPSIALDTACSSSLLALQNAYQAIRSGECPAAIVGGINLLLKPNTSVQFMKLGMLSPDGTCRSFDDSGNGYCRAEAVVAVLLTKKSLARRVYATILNAGTNTDGCKEQGVTFPSGEAQEQLIRSLYQPGGVAPESLEYIEAHGTGTKVGDPQELNGITRSLCAFRQSPLLIGSTKSNMGHPEPASGLAALTKVLLSLENGVWAPNLHFHNPNPEIPALLDGRLQVVDRPLPVRGGIVGINSFGFGGANVHVILQPNTQQAPAPAPHAALPHLLHASGRTMEAVQGLLEQGRQHSQDLAFVSMLNDIAATPTAAMPFRGYTVLGVEGHVQEVQQVPASQRPLWFICSGMGTQWRGMGLSLMRLDSFRESILRSDEALKPLGVKVSDLLLSTDEHTFDDIVHSFVSLTAIQIALIDLLTSMGLKPDGIIGHSLGEVACGYADGCLSQREAVLAAYWRGQCIKDANLPAGSMAAVGLSWEECKQRCPPGVVPACHNSEDTVTISGPQAAVNEFVEQLKQEGVFAKEVRTGGLAFHSYFMEGIAPTLLQALKKVIREPRPRSARWLSTSIPEAQWQSSLARTSSAEYNVNNLVSPVLFQEALWHVPEHAVVLEIAPHALLQAVLKRGVKPSCTIIPLMKRDHKDNLEFFLTNLGKVHLTGIDINPNALFPPVEFPVPRGTPLISPHIKWDHSQTWDIPVAEDFPNGSSSSSATVYNIDASSESSDHYLVDHCIDGRVLFPGTGYLYLVWKTLARSLSLSLEETPVVFENVTFHQATILPRTGTVPLEVRLLEASHAFEVSDSGNLIVSGKVYQWEDPDSKLFDHPEVPIPAESESVSRLTQGEVYKELRLRGYDYGPHFQGVYEATLEGEQGKLLWKDNWVTFMDTMLQISILGFSKQSLQLPTRVTAIYIDPATHLQKVYMLEGDTQVADVTTSRCLGVTVSGGVYISRLQTTATSRRQQEQLVPTLEKFVFTPHVEPECLSESAILQKELQLCKGLAKALQTKATQQGLKMTVPGLEDLPQHGLPRLLAAACQLQLNGNLQLELGEVLARERLLLPEDPLISGLLNSQALKACIDTALENLSTLKMKVVEVLAGEGHLYSHISALLNTQPMLQLEYTATDRHPQALKDVQTKLQQHDVAQGQWDPSGPAPTNLGALDLVVCNCALATLGDPALALDNMVAALKDGGFLLMHTVLKGHALGETLACLPSEVQPGPSFLSQEEWESLFSRKALHLVGLKKSFYGTALFLCRRLSPQDKPIFLPVEDTSFQWVDSLKSILATSSSQPVWLTAMNCPTSGVVGLVNCLRKEPGGHRIRCILLSNLSSTSHVPKLDPGSSELQKVLESDLVMNVYRDGAWGAFRHFQLEQDKPEEQTAHAFVNVLTRGDLASIRWVSSPLKHMQPPSSSGAQLCTVYYASLNFRDIMLATGKLSPDAIPGKWASRDCMLGMEFSGRDKCGRRVMGLVPAEGLATSVLLSPDFLWDVPSSWTLEEAASVPVVYTTAYYSLVVRGRIQHGETVLIHSGSGGVGQAAISIALSLGCRVFTTVGSAEKRAYLQARFPQLDDTSFANSRDTSFEQHVLLHTGGKGVDLVLNSLAEEKLQASVRCLAQHGRFLEIGKFDLSNNHPLGMAIFLKNVTFHGILLDALFEGANDSWREVAELLKAGIRDGVVKPLKCTVFPKAQVEDAFRYMAQGKHIGKVLVQVREEEPEAMLPGAQPTLISAISKTFCPEHKSYIITGGLGGFGLELARWLVLRGAQRLVLTSRSGIRTGYQAKHVREWRRQGIHVLVSTSNVSSLEGARALIAEATKLGPVGGVFNLAMVLRDAMLENQTPELFQDVNKPKYNGTLNLDRATREACPELDYFVAFSSVSCGRGNAGQSNYGFANSTMERICEQRRHDGLPGLAVQWGAIGDVGIILEAMGTNDTVVGGTLPQRISSCMEVLDLFLNQPHAVLSSFVLAEKKAVAHGDGEAQRDLVKAVAHILGIRDLAGINLDSSLADLGLDSLMGVEVRQILEREHDLVLPIREVRQLTLRKLQEMSSKAGSDTELAAPKSKNDTSLKQAQLNLSILLVNPEGPTLTRLNSVQSSERPLFLVHPIEGSITVFHSLAAKLSVPTYGLQCTQAAPLDSIPNLAAYYIDCIKQVQPEGPYRVAGYSFGACVAFEMCSQLQAQQGPAPAHNNLFLFDGSHTYVLAYTQSYRAKLTPGCEAEAEAEAICFFIKQFVDAEHSKVLEALLPLKSLEDRVAAAVDLITRSHQSLDRRDLSFAAVSFYYKLRAADQYKPKAKYHGNVILLRAKTGGTYGEDLGADYNLSQVCDGKVSVHIIEGDHRTLLEGRGLESIINIIHSSLAEPRVSVREG

SEQ ID NO: 21 - DNA, 9143 bp, Rattus norvegicus
  Fatty acid synthase
agagagacggcagcagcgtcccgtccagttcgcctgccgcgctcctcgcttgtcgtctgcctccagatcccagacagagaagagccatggaggaggtggtgatagccggtatgtccgggaaattgcccgagtcagagaacctgcaggagttctgggccaacctcattggcggtgtggacatggtcacagacgatgacaggaggtggaaggctgggctctatgggttgcctaagcggtctggaaagctgaaggatctgtccaagttcgacgcctccttttttggggtccaccccaagcaggcacacacaatggacccgcagctccggctgctgctggaagtcagctatgaagctattgtggacggaggtatcaacccggcctcactccgaggaacaaacactggtgtctgggtgggtgtgagtggttccgaggcgtcggaggccctgagcagagatcctgagactcttctgggctacagcatggtgggctgccagagagcaatgatggccaaccggctctctttcttcttcgacttcaaaggacccagcattgccctggacacagcctgctcctctagcctactggcactacagaatgcctatcaggctatccgcagtggggagtgccctgctgccattgtgggcgggatcaacctgctgctaaagcctaacacctctgtgcagttcatgaagctaggcatgctcagccccgatggcacctgcagatcctttgatgattcagggaacgggtattgccgtgctgaggctgtcgtggcagttctgctgactaagaagtccttggctcggcgagtctatgccactattctgaatgccgggacgaacacagatggctgcaaggagcaaggcgtgacattcccctctggagaagcccaggaacaactcatccgttctctgtatcagccgggcggtgtggcccccgagtctcttgaatatattgaagcccatggcacgggcaccaaggtgggggacccccaggaactgaacggcattactcggtccctgtgtgctttccgccagagccctttgttaattggctccaccaaatccaacatgggacaccctgagcctgcctcggggcttgcagccctgaccaaggtgctgttatccctagaaaatggggtttgggcccccaacctgcatttccacaaccccaaccctgaaatcccagcacttcttgatgggcggctgcaggtggtcgataggcccctgcctgttcgtggtggcatcgtgggcatcaactcgtttggcttcggaggtgccaatgttcacgtcatcctccagcccaacacacagcaggccccagcacctgccccacatgctgccctaccgcatttgctgcatgccagtggacggaccatggaggcagtgcagggcctgctggaacagggccgccagcacagtcaggacttggcctttgtgagcatgctcaatgacattgcagcaacccctacagcagccatgcccttcagaggttacactgtgttaggtgttgagggccatgtccaggaagtgcagcaagtgcctgccagccagcgcccactctggttcatctgctcagggatgggcacacagtggcgtggaatggggctgagccttatgcgcctggacagtttccgtgagtccatcctgcgctctgatgaggctctgaagcccttgggagtcaaagtgtcagacctgctgctgagcactgatgagcacacctttgatgacatcgtgcattcctttgtgagcctcaccgccatccagattgccctcatcgacctgctgacgtctatggggctgaaacctgatggcatcattgggcactccttgggagaggttgcctgtggctatgcagatggctgtctctcccagagagaggctgtgcttgcagcctactggagaggccagtgcattaaggatgccaaccttccggctggatccatggcagctgttggtttgtcctgggaagaatgtaaacaacgctgccctcctggtgtggtgcctgcctgccacaactctgaggacactgtgaccatctctggacctcaggctgcagtgaatgaatttgtggagcagctaaagcaagagggcgtgtttgccaaggaggtgcgaacaggtggcctggccttccactcctacttcatggaaggaattgcccccacgctgctgcaggctctcaagaaggtgatccgggagccacggccacgctcagcacgctggctcagcacctctatccctgaggcccagtggcagagcagcctggcccgcacatcttctgctgagtacaacgtcaacaacctggtgagccctgtgctcttccaggaagcactgtggcacgtccccgagcacgccgtggtgctggagattgcaccccatgcactgttgcaggctgtcctgaagcgaggcgtgaagcctagctgcaccatcatccccttgatgaagagggaccataaagataacttggagttcttcctcaccaacctcggcaaggtgcacctcacaggcatcgacatcaaccctaatgccttgttcccacctgtggaattcccggttccccgagggactcctctcatctcccctcacatcaagtgggaccacagtcagacttgggatatcccagttgctgaagacttccccaacggttccagctcctcctcagctacagtctacaacattgacgccagttccgagtcatctgaccactacctggtcgaccactgcattgacggccgtgtcctcttccctggcactggctacctgtacctggtgtggaagacactggctcgaagcctgagcttgtccctagaagagacccctgtggtgtttgagaacgtgacatttcatcaggccaccatcctgcccaggacaggaaccgtgcctctggaggtgcggctgctagaggcctcacatgcatttgaggtgtctgacagtggcaacctgatagtgagcgggaaagtgtaccagtgggaagaccctgactccaagttattcgaccacccagaagtcccgatccccgccgagtccgagtctgtctcccgcttgacgcagggagaagtatacaaggagctgcggctacgtggctatgactatggccctcatttccagggcgtctatgaggccaccctcgaaggtgagcaaggcaagctgctctggaaagacaactgggtgaccttcatggacacaatgctgcagatatccatcctgggcttcagcaagcagagtctgcagctacccacccgtgtgactgccatctatattgaccctgcaacccacctgcagaaggtgtacatgctggagggagacactcaagtggctgacgtgaccacgagccgctgtctgggcgtgaccgtctctggtggtgtctacatttcgagactacagacaacagcaacctcacggcggcagcaggaacagctggtccccaccctggagaagtttgtcttcacaccccatgtggagcctgagtgcctgtctgagagtgctatcctgcagaaagagctgcagctgtgcaagggtctggcaaaggctctgcagaccaaggccacccagcaagggctgaagatgacagtgcctgggctagaggaccttccccagcatggactgcctcgactcttggctgctgcctgccagctgcagctcaacgggaacctgcaactggagttaggtgaggtactggctcgagagaggctcctgctgccagaagaccctctgatcagtggcctccttaactcccaggccctcaaggcctgcatagacacagccctggagaacttgtctactctcaagatgaaggtggtggaggtgctggctggagaaggccacttgtattcccacatctcagcactgctcaacacccagcctatgctgcaactggagtatacagccaccgaccggcacccccaggccctgaaggatgttcagaccaagctgcagcagcatgatgtagcacagggccagtgggacccttctggtcctgctcctaccaacctgggtgctcttgaccttgtggtgtgcaactgtgcgttagccaccctgggggatccagccctggccctggacaacatggtagctgccctcaaggatggtggtttcctgctaatgcacacagtgctcaaaggacatgcccttggggagaccctggcctgcctcccttctgaggtgcagcctgggcccagcttcttaagccaggaagagtgggagagcctgttctcaaggaaggcactgcacctggtgggccttaaaaagtcattctacggtactgcgctgttcctgtgccgccgtctcagcccacaggacaagcccatcttcctgcctgtggaggatactagtttccagtgggtggactctctgaagagcattctggccacatcctcctcccagcctgtgtggctaacagccatgaactgccccacctcaggtgtggtaggcttggtgaactgtctccgaaaagagccgggtggacaccggattcggtgtatcctgctgtccaacctcagcagcacatctcacgtccccaagctggaccctggctcttcagagctacagaaggtgctagagagtgatctggtgatgaacgtgtacagggacggtgcctggggtgccttccgtcacttccagttagagcaggacaagcccgaggagcagacagcacatgcctttgtaaacgtccttacccgaggggaccttgcctccatccgctgggtctcttctcccctgaaacacatgcagccgccctcgagctcaggagcacagctctgcactgtctactatgcctcactgaacttccgagatatcatgctggccacgggcaagctgtcccctgatgccattccaggtaaatgggccagccgggactgcatgcttggcatggagttctcaggccgtgataagtgcggccggcgtgtgatggggctggtacccgcagaaggcctggccacctcagtcctgttatcacccgacttcctctgggatgtaccctctagctggaccctggaggaggcggcttctgtgcctgttgtctacaccaccgcctactactccttagtagtgcgtggtcgtattcagcacggggaaactgtgctcattcactcgggctccggtggtgtgggccaagcggccatttccattgcccttagcctgggctgccgagtcttcaccactgtgggctccgctgagaagcgagcttacctccaggccagattccctcagctggatgacaccagctttgctaactctcgagacacatcgtttgagcagcatgtgttactgcacacaggtggcaaaggggtggacctggtcctcaactccctggcagaagagaagctgcaggccagtgtgcggtgcttggctcagcatggccgcttcctagagatcggcaaatttgatctttctaacaaccaccctctgggcatggccatcttcttgaagaacgtcactttccatgggatcctgctggatgcactttttgagggggccaacgacagctggcgggaggtggcagagctgctgaaggccggcatccgtgatggggttgtgaagcctctcaagtgtacagtgtttcccaaggcccaggtggaggacgccttccgatacatggctcaaggaaaacatattggcaaagtccttgtccaggtacgggaggaggagcccgaggctatgctgccaggggctcagcccaccctgatttccgccatctccaagaccttctgcccagagcataagagttacatcatcactggtggcctaggtggctttggcctggaactggcccggtggcttgtgcttcgtggggcccaaaggcttgtactaacttcccgatctggaatccgcacaggctaccaagccaagcacgttcgggagtggaggcgccagggcatccatgtgctagtgtcgacaagcaatgtcagttcactggagggggcccgtgctctcatcgctgaagccacaaagcttgggcccgttggaggtgtcttcaacctggccatggttttaagggatgccatgctggagaaccagactccagaactcttccaggatgtcaacaagcccaagtacaatggcaccctgaaccttgacagggcgacccgggaagcctgtcctgagctggactactttgtggccttctcctctgtaagctgcgggcgtggtaatgctggccaatccaactatggcttcgccaactctaccatggagcgtatttgcgaacagcgccggcacgatggcctcccaggtcttgccgtgcaatggggtgccattggtgacgtgggcattatcttggaagcgatgggtaccaatgacacagtcgttggcggcacactgccacagcgcatctcctcctgcatggaggtgctggacctcttcctgaatcagccccacgcagtcctgagcagttttgtgctggctgagaagaaagctgtggcccatggtgatggtgaagcccagagggatctggtgaaagcagtggcacacatcctaggcatccgcgacctcgcagggattaacctggacagctcgctggcagacctcggcctggactcgctcatgggtgtggaagtgcgccagatcctggaacgtgaacatgatctggtgctacccattcgtgaagtacggcaactcacactgcggaagcttcaggaaatgtcctccaaggctggctcagacactgagttggcagcccccaagtccaagaatgatacatccctgaagcaggcccagctgaatctgagtatcctgctggtgaaccctgagggccctaccttaacacgactcaactcagtgcagagctctgagcggcctctgttcctggtgcaccccattgaaggttccatcactgtgttccacagcctggctgccaagctcagtgtgcccacctacggtctgcagtgcacccaagcggcccccctggacagcattccaaacctggctgcctactacattgattgcatcaagcaggtgcagcctgaggggccctaccgagtggctgggtattcttttggagcttgtgtagccttcgagatgtgctcccagctgcaggcccagcagggcccagcccccgcccacaacaacctcttcttgtttgatggctcacacacctacgtattggcgtacacccagagctaccgggcaaagctgaccccaggctgtgaggctgaggctgaagctgaagccatatgcttcttcattaagcagtttgttgatgcagagcatagcaaggtgctagaggccctgctaccactgaagagcctggaggaccgggttgctgctgctgtggacctcatcactagaagccaccagagcctggaccgccgtgacctgagctttgctgccgtgtccttctactacaagcttcgagccgccgaccagtataaacccaaggccaagtaccacggcaatgtgatcctgctgcgggccaagacaggtggcacctacggcgaggacttgggtgccgattacaacctgtcccaggtgtgtgatgggaaggtgtctgtgcacatcattgagggtgaccaccgtacgctgctggagggcaggggcctggagtctatcatcaacatcatccacagctccctggctgagcctcgagtgagtgtacgggagggctagacctgcctaccatgaagccacgacccacaccggccaccagagatgctccgatccccaccacaccctgagtgcagggactggggagggtcctgctggtgggaccccctcaccccagtggcccagcaccaccccctcccctggtggctgctacaaacaggaccatcacatgtgtcccagccacttagtggggttcccagagccactgacttggaggcaccctggtctgtgaagagtcagtggaggccagcaagagccaaactgagccttttctgccaagtgacatttgtcacactggttgtttctccattaaattctcatatttattgcattgctgggaaagaccgcccaccccagggttaactcattccagaacccctaaagtgggaaaagccatgtggggaaggctgctggctggagcccctttttgtcttagccctgtacccgctcactgcagggcagggtatggagagggctggttcgcggggaacgaggaccccagcagacactgtagcccatggcccttggtccccagcactcccggctgcacccatgatgcagggcctaccagactctgcggaccgcaccgggcactcactgtatttgttttccaagattcaaattgctgcttgggttttgaatttactgcagctgtcagtgtaaagaaacatgtctgaactgtgtcctttttacaccaacctggtaaaaatgctcttgatgctgtcccgttgccacaattaaactgcacgtgagctctggcttccgttcagtctctttccagtcccagacctgagtccccagagcctccacagctcttacagtgagaatcaaattggcccactccttggaaggcgtggcattctgtcagagtaaaaggaaagtagagtgtgctgattcacgttcagcgtgtggggctggctagagaccttggcactgtagtgaacagaatgtgtccacctttaagtcaccctgaaggcatcaccatagctacagcctcacccaggggtagagaatagtactgtctacttgttgactacctggcagttggtgccagcccctatagaggaaaacagcagtgtgtggccactgtgagaagcatatccctggaaacaggtgaccagagcagagggctaacgcctacctgagtcacacaaaactgaccaggcttgagtgtccagaagagtctatcagaaggccacagcattcagtcctatccacagagagcagcagactaagttgtctccttgccagcttagaaaactgcagtgctggggtacaggtagggtgttcaggaggtccgggccccagtgattagtctaagactgaagcatctggttggctgtggtcccacctagaaaattcttaaagctcttgtcatgtacttcctgggaaggacctaccctgtctcaataatgtctctagctcgttggagtctactgactcaaacatttataaagtgtcctagaaaggcctgactcccctacaaggctgtgtgatccttcaaactcacatatgtgagccaataaaaccttgagactctag

SEQ ID NO: 22 - protein, 373 aa, Rattus norvegicus
  Glutamine synthetase
MATSASSHLNKGIKQMYMNLPQGEKIQLMYIWVDGTGEGLRCKTRTLDCDPKCVEELPEWNFDGSSTFQSEGSNSDMYLHPVAMFRDPFRRDPNKLVFCEVFKYNRKPAETNLRHSCKRIMDMVSSQRPWFGMEQEYTLMGTDGHPFGWPSNGFPGPQGPYYCGVGADKAYGRDIVEAHYRACLYAGIKITGTNAEVMPAQWEFQIGPCEGIRMGDHLWVARFILHRVCEDFGVIATFDPKPIPGNWNGAGCHTNFSTKAMREENGLRCIEEAIDKLSKRHQYHIRAYDPKGGLDNARRLTGFHETSNINDFSAGVANRSASIRIPRIVGQEKKGYFEDRRPSANCDPYAVTEAIVRTCLLNETGDEPFQYKN

SEQ ID NO: 23 - DNA, 2793 bp, Rattus norvegicus
  Glutamine synthetase
acagccgagaatgggagtagggcggagtgtttgagcagcacacccatttcctctccgctcttcgtctcgttctcgtggcctgtccacccatccatcatctgccggccaccgctctgaacaccttccaccatggccacctcagcaagttcccacttgaacaaaggcatcaagcagatgtacatgaacctgccccagggcgagaagatccaactcatgtatatctgggttgatggtaccggggaagggctacgctgcaagacccgtactctggactgtgaccccaagtgtgtagaagagttacccgagtggaactttgatggttctagtacgtttcagtctgaaggctccaacagcgacatgtacctccatcctgtggccatgtttcgagaccccttccgcagagaccccaacaagctggtgttctgcgaagtattcaagtataaccggaagcccgcagagaccaacctgaggcacagctgtaagcgtataatggacatggtgagcagccagcgcccctggtttggaatggaacaggagtatactctcatgggaacagacggccaccctttcggctggccttctaatggcttccctggaccccaaggaccctattactgcggtgtgggagctgacaaggcttatggccgagatatcgtggaggctcactaccgggcctgcttgtatgctggaatcaagatcacagggacaaatgccgaggttatgcctgcccagtgggaattccagataggaccctgcgaagggatccgcatgggagatcatctctgggtagcccgttttatcttgcatcgggtatgcgaagactttggggtgatagcaacctttgaccccaagcccattccagggaactggaatggggcaggctgccacaccaactttagcaccaaggccatgcgggaggagaatggtctgaggtgcattgaggaggccattgataaactgagcaagaggcaccagtaccacatccgtgcctacgaccccaaggggggcctggacaacgcccgccgtctgactggattccacgaaacctccaacatcaacgacttttccgctggcgttgccaaccgcagcgccagtatccgcattccccggattgtcggccaggagaagaagggttactttgaagaccgtcggccttctgccaattgcgacccctatgcggtgacggaagccatcgtccgcacgtgtctcctcaacgaaactggcgacgagcccttccaatacaagaactaagcggactcgacttccagtgatcttgagcccttcctagttcaccccactcccaactgttccctctcccactggtccccactgtaactcaaaaggatggaataccaaggtctttttattccttgcgcccagttaatttttgcctttattggtcagaatagaggggtcaggttcttaatctctacacacccaaccccttctttcctagctagctttccagtgggggaacgggagggggtggggaagggtaacccaccgcttcatctcagcgggaatgcatgtcctgtaggcatagctgtcacaaatcgggtgtacttgtggtgagggaggactggttttttttttccttcaggataattgaaagggcaggcccaacagcttagattaacattttctctgtcagtagagagctgttatttcttccggtgaaaccagctttctattgaagtctggtgaggagttggaggttggtctcttggcttccttagcttagggaaggggagttcaccctcccttcatgaaacacagttcacctgacaaatggccctactgtaaaggaagaaaaaagtttcttggtcctccatttataactcaaagcagagtagtatttttatatttaaatgttaaaaacaaaaaagttatatatatgggtgtgtggatatatatgtcttttctaattgagaaaaccatcctattccctgggtgccaagtttgagtgaggagctcggtgtagaagtgaggcactcttgaggtaggggtggggatgcagtactgggaaagttggttatcttgggggttcagcttcattactacttagggtttccctgcccactctgcaggagcagatgttggacaggtagccagtgggatgccactgcttgccgccactgtccctgggcttagtttaaggggacgtgtatacctaatccacacacgagttagaagtatgagttggctggtcaacttgaacattgttacaggcgggtgggtgttagtggggggttattttttggtgggactagcatgtcactaaagcgggccttttgatatattaaattttttaaagcaaaacaagtttagattttaatcaagttcgtagggtttctaactttacagaattgcctgtttgtttcaatgactccttccacttggctcttaggggaactgaggacaggcctggagttaatacacttgtcattctgtgtcctagtgtcctcttcctccggcagactgtccccttccttctgaaaaagccgatagagtcttgttttatttttcttttataataaacacaccccacctccatcccagcttgttgccttgcagttttctggatgtttgtgtcggcagcaggcagctgtggtttttttctcttgccacgatgactctaattaccatgtatagtatgttcagttagataactcactgtaaacagactgtaactgagagcagagcttgtaaatcaacctaacgtttataagatttcctctgacttgtttctttgtggttccaaaaaaaaaaaaaaaaaaaaaaacctcaaaaact

SEQ ID NO: 24 - protein, 514 aa, Homo sapiens
  Seryl t-RNA synthetase
MVLDLDLFRVDKGGDPALIRETQEKRFKDPGLVDQLVKADSEWRRCRFRADNLSKLKNLCSKTIGEKMKKKEPVGDDESVPENVLSFDDLTADALANLKVSQIKKVRLLIDEAILKCDAERIKLEAERFENLREIGNLLHPSVPISNDEDVDNKVERIWGDCTVRKKYSHVDLVVMVDGFEGEKGAVVAGSRGYFLKGVLVFLEQALIQYALRTLGSRGYIPIYTPFFMRKEVMQEVAQLSQFDEELYKVIGKGSEKSDDNSYDEKYLIATSEQPIAALHRDEWLRPEDLPIKYAGLSTCFRQEVGSHGRDTRGIFRVHQFEKIEQFVYSSPHDNKSWEMFEEMITTAEEFYQSLGIPYHIVNIVSGSLNHAASKKLDLEAWFPGSGAFRELVSCSNCTDYQARRLRIRYGQTKKMMDKVEFVHMLNATMCATTRTICAILENYQTEKGITVPEKLKEFMPPGLQELIPFVKPAPIEQEPSKKQKKQHEGSKKKAAARDVTLENRLQNMEVTDA

SEQ ID NO: 25 - DNA, 1846 bp, Homo sapiens
  Seryl t-RNA synthetase
gcagtgcggcggtcacaggctgagtgctgcggcgcgatccttgcttccctgagcgttggcccgggaggaaagaagatggtgctggatctggatttgtttcgggtggataaaggaggggacccagccctcatccgagagacgcaggagaagcgcttcaaggacccgggactagtggaccagctggtgaaggcagacagcgagtggcgacgatgtagatttcgggcagacaacttgagcaagctgaagaacctatgcagcaagacaatcggagagaaaatgaagaaaaaagagccagtgggagatgatgagtctgtcccagagaatgtgctgagtttcgatgaccttactgcagacgctttagctaacctgaaagtctcacaaatcaaaaaagtccgactcctcattgatgaagccatcctgaagtgtgacgcggagcggataaagttggaagcagagcggtttgagaacctccgagagattgggaaccttctgcacccttctgtacccatcagtaacgatgaggatgtggacaacaaagtagagaggatttggggcgattgtacagtcaggaagaagtactctcatgtggacctggtggtgatggtagatggctttgaaggcgaaaagggggccgtggtggctgggagtcgagggtacttcttgaagggggtcctggtgttcctggaacaggctctcatccagtatgcccttcgcaccttgggaagtcggggctacattcccatttataccccctttttcatgaggaaggaggtcatgcaggaggtggcacagctcagccagtttgatgaagaactttataaggtgattggcaaaggcagtgaaaagtctgatgacaactcctatgatgagaagtacctgattgccacctcagagcagcccattgctgccctgcaccgggatgagtggctccggccggaggacctgcccatcaagtatgctggcctgtctacctgcttccgtcaggaggtgggctcccatggccgtgacacccgtggcatcttccgagtccatcagtttgagaagattgaacagtttgtgtactcatcaccccatgacaacaagtcatgggagatgtttgaagagatgattaccaccgcagaggagttctaccagtccctggggattccttaccacattgtgaatattgtctcaggttctttgaatcatgctgccagtaagaagcttgacctggaggcctggtttccgggctcaggagccttccgtgagttggtctcctgttctaattgcacggattaccaggctcgccggcttcgaatccgatatgggcaaaccaagaagatgatggacaaggtggagtttgtccatatgctcaatgctaccatgtgcgccactacccgtaccatctgcgccatcctggagaactaccagacagagaagggcatcactgtgcctgagaaattgaaggagttcatgccgccaggactgcaagaactgatcccctttgtgaagcctgcgcccattgagcaggagccatcaaagaagcagaagaagcaacatgagggcagcaaaaagaaagcagcagcaagagacgtcaccctagaaaacaggctgcagaacatggaggtcaccgatgcttgaacattcctgcctccctatttgccaggctttcatttctgtctgctgagatctcagagcctgcccaacagcagggaagccaagcacccattcatccccctgcccccatctgactgcgtagctgagaggggaacagtgccatgtaccacacagatgttcctgtctcctcgcatgggcatagggacccatcattgatgactgatgaaaccatgtaataaagcatctctgg

SEQ ID NO: 26 - protein, 434 aa, Homo sapiens
  Alpha-enolase
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 27 - DNA, 1755 bp, Homo sapiens
  Alpha-enolase
acggagatctcgccggctttacgttcacctcggtgtctgcagcaccctccgcttcctctcctaggcgacgagacccagtggctagaagttcaccatgtctattctcaagatccatgccagggagatctttgactctcgcgggaatcccactgttgaggttgatctcttcacctcaaaaggtctcttcagagctgctgtgcccagtggtgcttcaactggtatctatgaggccctagagctccgggacaatgataagactcgctatatggggaagggtgtctcaaaggctgttgagcacatcaataaaactattgcgcctgccctggttagcaagaaactgaacgtcacagaacaagagaagattgacaaactgatgatcgagatggatggaacagaaaataaatctaagtttggtgcgaacgccattctgggggtgtcccttgccgtctgcaaagctggtgccgttgagaagggggtccccctgtaccgccacatcgctgacttggctggcaactctgaagtcatcctgccagtcccggcgttcaatgtcatcaatggcggttctcatgctggcaacaagctggccatgcaggagttcatgatcctcccagtcggtgcagcaaacttcagggaagccatgcgcattggagcagaggtttaccacaacctgaagaatgtcatcaaggagaaatatgggaaagatgccaccaatgtgggggatgaaggcgggtttgctcccaacatcctggagaataaagaaggcctggagctgctgaagactgctattgggaaagctggctacactgataaggtggtcatcggcatggacgtagcggcctccgagttcttcaggtctgggaagtatgacctggacttcaagtctcccgatgaccccagcaggtacatctcgcctgaccagctggctgacctgtacaagtccttcatcaaggactacccagtggtgtctatcgaagatccctttgaccaggatgactggggagcttggcagaagttcacagccagtgcaggaatccaggtagtgggggatgatctcacagtgaccaacccaaagaggatcgccaaggccgtgaacgagaagtcctgcaactgcctcctgctcaaagtcaaccagattggctccgtgaccgagtctcttcaggcgtgcaagctggcccaggccaatggttggggcgtcatggtgtctcatcgttcgggggagactgaagataccttcatcgctgacctggttgtggggctgtgcactgggcagatcaagactggtgccccttgccgatctgagcgcttggccaagtacaaccagctcctcagaattgaagaggagctgggcagcaaggctaagtttgccggcaggaacttcagaaaccccttggccaagtaagctgtgggcaggcaagccttcggtcacctgttggctacacagacccctcccctcgtgtcagctcaggcagctcgaggcccccgaccaacacttgcaggggtccctgctagttagcgccccaccgccgtggagttcgtaccgcttccttagaacttctacagaagccaagctccctggagccctgttggcagctctagcttttgcagtcgtgtaatgggcccaagtcattgtttttctcgcctcactttccaccaagtgtctagagtcatgtgagcctcgtgtcatctccggggtggccacaggctagatccccggtggttttgtgctcaaaataaaaagcctcagtgacccatgag

SEQ ID NO: 28 - protein, 316 aa, Rattus norvegicus
  Aldose reductase
MASHLELNNGTKMPTLGLGTWKSPPGQVTEAVKVAIDMGYRHIDCAQVYQNEKEVGVALQEKLKEQVVKRQDLFIVSKLWCTFHDQSMVKGACQKTLSDLQLDYLDLYLIHWPTGFKPGPDYFPLDASGNVIPSDTDFVDTWTAMEQLVDEGLVKAIGVSNFNPLQIERILNKPGLKYKPAVNQIECHPYLTQEKLIEYCHCKGIVVTAYSPLGSPDRPWAKPEDPSLLEDPRIKEIAAKYNKTTAQVLIRFPIQRNLVVIPKSVTPARIAENFKVFDFELSNEDMATLLSYNRNWRVCALMSCAKHKDYPFHAEV

SEQ ID NO: 29 - DNA, 1337 bp, Rattus norvegicus
  Aldose reductase
ctcttgcgggtcgttgtgcgtaacttgcagcaatcatggctagccatctggaactcaacaacggcaccaagatgcccaccctgggtctgggcacctggaagtctcctcctggccaggtgaccgaggctgtgaaggttgctatcgacatggggtatcgccacattgactgcgcccaggtgtaccagaatgagaaggaggtgggggtggccctccaggagaagctcaaggagcaggtggtgaagcgccaggatctcttcattgtcagcaagctgtggtgcacgttccacgaccagagcatggtgaaaggggcctgccagaagacgctgagcgacctgcagctggactacctggacctctaccttattcactggccaactggcttcaagcctgggcctgactatttccccctggatgcatcgggaaacgtgattcctagtgacaccgattttgtggacacttggacggctatggagcaactagtggatgaaggtttggtaaaagcaatcggagtctccaacttcaaccctcttcagattgagaggatcttgaacaaacctggcttaaagtataagcctgctgttaaccagatcgagtgccacccatacctaactcaggagaagctgattgagtactgccattgcaaaggcatcgtggtgactgcatacagtccccttggttctcctgacaggccctgggccaagcctgaggacccctctctcctggaggatcccaggatcaaggaaattgcagccaagtacaataaaactacagcccaggtgctgatccggttccccatccaaaggaacctggtcgtgatccccaagtctgtgacaccagcacgtattgctgagaactttaaggtctttgactttgagctgagcaatgaggacatggccactctactcagctacaacaggaactggagggtgtgcgccttgatgagctgtgccaaacacaaggattaccccttccacgcagaagtctgaagctgtggtggacgaatcctgctcctccccaagcgacttaacacatgttctttctgcctcatctgcccttgcaagtgtccctctgcactgggtggcaccttgcagaccagatggtgagagtttgttagtttgacgtagaatgtggagggcagtaccagtagctgaggagtttcttcggcctttcttggtcttcttcccacctggaggactttaacacgagtaccttttccaaccaaagagaaagcaagatttatagcccaagtcatgccactaacacttaaatttgagtgcttagaactccagtcctatgggggtcagactttttgcctcaaataaaaactgcttttgtcg

SEQ ID NO: 30 - protein, 299 aa, Rattus norvegicus
  Cytochrome C oxidase, subunit 1
EFPFFDPAGGGDPILYQHLFWFFGHPEVYILILPGFGIISHVVTYYSGKKEPFGYMGMVWAMMSIGFLGFIVWAHHMFTVGLDVDTRAYFTSATMIIAIPTGVKVFSWLATLHGGNIKWSPAMLWALGFIFLFTVGGLTGIVLSNSSLDIVLHDTYYVVAHFHYVLSMGAVFAIMACFVHWFPLFSGYTLNDTWAKAHFAIMFVGVNMTFFPQHFLGLAGMPRRYSDYPDAYTTWNTVSSMGSFISLTAVLVMIFMIWEAFASKREVLSISYSSTNLEWLHGCPPPYHTFEEPSYVKVK

SEQ ID NO: 31 - DNA, 987 bp, Rattus norvegicus
  Cytochrome C oxidase, subunit 1
gaattccctttcttcgaccccgctggaggtggagacccaattctttatcaacacctattctgattcttcggccacccagaagtgtacatcttaattcttccagggtttggaattatttcacatgtagttacctattactctggaaaaaaagaacccttcggatatataggtatggtatgagccataatatctattggcttcctaggatttattgtatgagcacatcacatattcacagtaggcctagatgtagacacccgagcctactttacatctgccactataattatcgcaattcctacaggcgtaaaagtattcagctgactcgctacactacatggaggaaatatcaaatgatcccccgccatattatgagccttagggtttatcttcttattcacagtagggggcctaacagggatcgtactatctaactcatcccttgacattgtacttcatgatacatactacgtagtagctcacttccactatgtcttatctataggagcagtattcgccatcatagcttgcttcgtccactgattcccactattctcaggctataccctaaatgacacatgagcaaaagcccactttgccattatatttgtaggtgtaaacataacattttttcctcaacacttcctaggattagcagggatacctcgtcgttactctgattatccagatgcttataccacatgaaatacagtctcctctataggctcattcatctcacttacggccgtccttgtaatgatcttcatgatttgagaagccttcgcatcaaaacgagaagtgctctcaatttcctactcttcaactaacctagaatgactgcatggatgccccccaccttaccacacattcgaagaaccttcctatgtaaaagttaaataagaaaggaaggattcgaaccccctacaactggtttcaagccaatttcataaccattatgtctttctcaataaaaaaaaaaaggaattc

SEQ ID NO: 32 - protein, 334 aa, Rattus norvegicus
  LDH-B
MATLKEKLIAPVADDETAVPNNKITVVGVGQVGMACAISILGKSLADELALVDVLEDKLKGEMMDLQHGSLFLQTPKIVADKDYSVTANSKIVVVTAGVRQQEGESRLNLVQRNVNVFKFIIPQIVKYSPDCTIIVVSNPVDILTYVTWKLSGLPKHRVIGSGCNLDSARFRYLMAEKLGIHPSSCHGWILGEHGDSSVAVWSGVNVAGVSLQELNPEMGTDNDSENWKEVHKMVVDSAYEVIKLKGYTNWAIGLSVADLIESMLKNLSRIHPVSTMVKGMYGIENEVFLSLPCILNARGLTSVINQKLKDDEVAQLRKSADTLWDIQKDLKDL

SEQ ID NO: 33 - DNA, 1217 bp, Rattus norvegicus
  LDH-B
cagagcctcctcttgtctggacaagatggcaacccttaaggaaaagctcattgcgccagtcgcagacgacgagactgccgtcccgaacaacaagattactgtagtaggcgttggacaagttggaatggcttgtgctatcagcattctggggaagtctctggctgatgagcttgccctggtggatgtcttggaagacaagctcaaaggagaaatgatggatctgcagcacgggagcttatttctccagactccgaaaatcgtggctgataaagattactccgtgacagccaattctaagattgtggtggtgaccgcgggagtccgccagcaggagggggagagtcggctcaacctggtgcagagaaacgtcaatgtattcaagttcattattcctcagatcgtcaagtacagccccgactgcaccatcatcgtggtttccaacccagtggatattcttacctatgtcacctggaagctgagcgggctacctaagcaccgcgtgattggaagtggatgcaatctggattctgctcggtttcgttacctcatggccgaaaagcttggtattcatcccagcagctgccacggctggatcctgggcgagcacggggactccagtgtggcagtgtggagcggggtgaatgtggcaggagtctccctccaggaactgaacccagagatgggaacggacaatgacagcgagaactggaaggaggtgcataagatggtggtggacagtgcctatgaagtcatcaagctaaaaggctacaccaactgggccatcggcctaagtgtggctgacctcatcgaatccatgctgaaaaacctctctcggattcaccccgtgtctacaatggtgaagggaatgtacggcatcgagaacgaagtcttcctcagtctcccgtgcatccttaatgctcggggactgaccagcgtcatcaaccagaagctgaaggacgatgaggtcgctcagctcaggaagagtgcggacaccctgtgggatatccagaaagacctcaaggacctgtgactgccaggcgccaggctgtagaaatccaaacctccaatgtgactaagtgaacctttagtcttcggccttgtacgtaggtcacagtttgcttcttccctaacatgtgataatgagctcacagatcaaaaccaggagtgtttgatgtttgcactaggagctcctgaacaaataaagtttagcaattgca

SEQ ID NO: 34 - protein, 86 aa, Homo sapiens
  Cytochrome C oxidase subunit VIb
MAEDMETKIKNYKTAPFDSRFPNQNQTRNCWQNYLDFHRCQKAMTAKGGDISVCEWYQRVYQSLCPTSWVTDWDEQRAEGTFPGKI

SEQ ID NO: 35 - DNA, 439 bp, Homo sapiens
  Cytochrome C oxidase subunit VIb
ttgagctgcaggttgaatccggggtgcctttaggattcagcaccatggcggaagacatggagaccaaaatcaagaactacaagaccgccccttttgacagccgcttccccaaccagaaccagactagaaactgctggcagaactacctggacttccaccgctgtcagaaggcaatgaccgctaaaggaggcgatatctctgtgtgcgaatggtaccagcgtgtgtaccagtccctctgccccacatcctgggtcacagactgggatgagcaacgggctgaaggcacgtttcccgggaagatctgaactggctgcatctccctttcctctgtcctccatccttctcccaggatggtgaagggggacctggtacccagtgatccccaccccaggatcctaaatcatgacttacctgctaataaaaactcattggaaaagtg

SEQ ID NO: 36 - protein, 172 aa, Homo sapiens
  NADH ubiquinone oxidoreductase PGIV subunit
MPGIVELPTLEELKVDEVKISSAVLKAAAHHYGAQCDKPNKEFMLCRWEEKDPRRCLEEGKLVNKCALDFFRQIKRHCAEPFTEYWTCIDYTGQQLFRHCRKQQAKFDECVLDKLGWVRPDLGELSKVTKVKTDRPLPENPYHSRPRPDPSPEIEGDLQPATHGSRFYFWTK

SEQ ID NO: 37 - DNA, 700 bp, Homo sapiens
  NADH ubiquinone oxidoreductase PGIV subunit
ggggagttcaaggagacgggggcgacgcggctgagggcttctcgtcggggtcggggctgcagccgtcatgccggggatagtggagctgcccactctagaggagctgaaagtagatgaggtgaaaattagttctgctgtgcttaaagctgcggcccatcactatggagctcaatgtgataagcccaacaaggaatttatgctctgccgctgggaagagaaagatccgaggcggtgtttagaggaaggcaaactggtcaacaagtgtgctttggacttctttaggcagataaaacgtcactgtgcagagccttttacagaatattggacttgcattgattatactggccagcagttatttcgtcactgtcgcaaacagcaggcaaagtttgacgagtgtgtgctggacaaactgggctgggtgcggcctgacctgggagaactgtcaaaggtcaccaaagtgaaaacagatcgacctttaccggagaatccctatcactcaagaccaagaccggatcccagccctgagatcgagggagatctgcagcctgccacacatggcagccgcttttatttctggaccaagtaaagatgggtccgtggcccacactcggtcatgtgctcagacaacgactgatgaaaacgcccatgcggtttgcatcgactgatagtgtgttctttccgggatcacaaacattaacaa

SEQ ID NO: 38 - protein, 532 aa, Mus musculus
  succinate dehydrogenase Fp subunit
LRAAFGLSEAGFNTACLTKLFPTRSHTVAAQGGINAALGNMEEDNWRWHFYDTVKGSDWLGDQDAIHYMTEQAPASVVELENYGMPFSRTEDGKIYQRAFGGQSLKFGKGGQAHRCCCVADRTGHSLLHTLYGRSLRYDTSYFVEYFALDLLMENGECRGVIALCIEDGSIHRIRAKNTVIATGGYGRTYFSCTSAHTSTGDGTAMVTRAGLPCQDLEFVQFHPTGIYGAGCLITEGCRGEGGILINSQGERFMERYAPVAKDLASRDVVSRSMTLEIREGRGCGPEKDHVYLQLHHLPPEQLATRLPGISETAMIFAGVDVTKEPIPVLPTVHYNMGGIPTNYKGQVLKHVNGQDQIVPGLYACGEAACASVHGANRLGANSLLDLVVFGRACALSIAESCRPGDKVPSIKANAGEESVMNLDKLRFADGSIRTSELRLNMQKSMQNHAAVFRVGSVLQEGCEKISQLYGDLKHLKTFDRGMVWNTDLVETLELQNLMLCALQTIYGAEARKESRGAHAREDYKVRVDEYD

SEQ ID NO: 39 - DNA, 1596 bp, Mus musculus
  succinate dehydrogenase Fp subunit
ttgcgagctgcatttggcctttctgaggcagggtttaatactgcatgccttacaaagctctttcctacccgatcacatactgttgcagcacagggaggtatcaatgctgctctggggaacatggaagaggacaactggagatggcatttctatgacactgtgaaaggctccgactggctgggggatcaggatgccatccattacatgacagagcaagctcctgcctctgtggttgagctagaaaattatggtatgccatttagcagaactgaagatgggaagatttatcagcgtgcatttggtggacagagcctcaagtttgggaaaggcgggcaggctcatcggtgttgctgtgtggctgatcggacaggccactcactcttacacaccttgtatggaagatctctgcgatatgacaccagttattttgtggaatattttgcactggatcttctgatggaaaatggggagtgccgtggtgtcattgcactgtgcatagaagatgggtccatacaccgaataagagcaaaaaacactgttattgctactgggggctacgggcgaacctacttcagctgtacatctgcccataccagcacaggggacggcacagccatggtcactagggctggtttgccttgccaggacttagaatttgttcagttccaccccacaggtatatacggtgctggctgcctcatcacagaagggtgtcgtggagagggggggattctcatcaacagtcaaggtgaaaggttcatggagagatacgcccctgttgccaaggacctggcatcaagagatgttgtgtctcgatccatgactcttgagatccgtgaaggaagaggctgtggccctgagaaagatcacgtctacctgcagttgcatcatctgcctcctgagcaactggccacacgtctgcccggaatttcagagacagccatgatcttcgctggtgtggatgtcactaaggagcccattccagtcctccccactgtgcattacaacatgggtgggattcccactaactacaagggacaggtgctgaagcatgtgaatggccaggatcagattgtgcctggactgtatgcctgtggggaggctgcctgcgcctcagtgcatggtgccaaccggcttggagcaaattctctcttggaccttgtagtctttggcagagcctgtgccctgagcattgcagaatcttgcaggcctggagataaagttccttcaattaaggcaaatgctggagaagaatcggttatgaatcttgacaagttgagatttgccgatggaagtataagaacatcagaactacgcctaaacatgcagaagtcgatgcagaaccatgctgcagtgttccgtgtggggagtgtattgcaagaaggctgtgaaaaaatcagtcagctctatggagacctaaagcatctaaagacattcgacaggggaatggtttggaacacagacctggtggagaccctggagctgcagaatctgatgctgtgcgcactgcagaccatatatggtgcagaagctcggaaggagtcccggggagcccatgccagggaagattacaaagtgcgggtcgatgagtatgat

SEQ ID NO: 40 - protein, 453 aa, Homo sapiens
  Core protein II of human mitochondrial cytochrome bc-1 complex
MKLLTRAGSFSRFYSLKVAPKVKATAAPAGAPPQPQDLEFTKLPNGLVIASLENYSPVSRIGLFIKAGSRYEDFSNLGTTHLLRLTSSLTTKGASSFKITRGIEAVGGKLSVTATRENMAYTVECLRGDVDILMEFLLNVTTAPEFRRWEVADLQPQLKIDKAVAFQNPQTHVIENLHAAAYQNALANPLYCPDYRIGKVTSEELHYFVQNHFTSARMALIGLGVSHPVLKQVAEQFLNMRGGLGLSGAKANYRGGEIREQNGDSLVHAAFVAESAVAGSAEANAFSVLQHVLGAGPHVKRGSNTTSHLHQAVAKATQQPFDVSAFNASYSDSGLFGIYTISQATAAGDVIKAAYNQVKRIAQGNLSNTDVQAAKNKLKAGYLMSVESSECFLEEVGSQALVAGSYMPPSTVLQQIDSVANADIINAAKKFVSGQKSMAASGNLGHTPFVDEL

SEQ ID NO: 41 - DNA, 1588 bp, Homo sapiens
  Core protein II of human mitochondrial cytochrome bc-1 complex
atcttgctttcctttaatccggcagtgaccgtgtgtcagaacaatcttgaatcatgaagctactaaccagagccggctctttctcgagattttattccctcaaagttgcccccaaagttaaagccacagctgcgcctgcaggagcaccgccacaacctcaggaccttgagtttaccaagttaccaaatggcttggtgattgcttctttggaaaactattctcctgtatcaagaattggtttgttcattaaagcaggcagtagatatgaggacttcagcaatttaggaaccacccatttgctgcgtcttacatccagtctgacgacaaaaggagcttcatctttcaagataacccgtggaattgaagcagttggtggcaaattaagtgtgaccgcaacaagggaaaacatggcttatactgtggaatgcctgcggggtgatgttgatattctaatggagttcctgctcaatgtcaccacagcaccagaatttcgtcgttgggaagtagctgaccttcagcctcagctaaagattgacaaagctgtggcctttcagaatccgcagactcatgtcattgaaaatttgcatgcagcagcttaccagaatgccttggctaatcccttgtattgtcctgactataggattggaaaagtgacatcagaggagttacattacttcgttcagaaccatttcacaagtgcaagaatggctttgattggacttggtgtgagtcatcctgttctaaagcaagttgctgaacagtttctcaacatgaggggtgggcttggtttatctggtgcaaaggccaactaccgtggaggtgaaatccgagaacagaatggagacagtcttgtccatgctgcttttgtagcagaaagtgctgtcgcgggaagtgcagaggcaaatgcatttagtgttcttcagcatgtcctcggtgctgggccacatgtcaagaggggcagcaacaccaccagccatctgcaccaggctgttgccaaggcaactcagcagccatttgatgtttctgcatttaatgccagttactcagattctggactctttgggatttatactatctcccaggccacagctgctggagatgttatcaaggctgcctataatcaagtaaaaagaatagctcaaggaaacctttccaacacagatgtccaagctgccaagaacaagctgaaagctggatacctaatgtcagtggagtcttctgagtgtttcctggaagaagtcgggtcccaggctctagttgctggttcttacatgccaccatccacagtccttcagcagattgattcagtggctaatgctgatatcataaatgcggcaaagaagtttgtttctggccagaagtcaatggcagcaagtggaaatttgggacatacaccttttgttgatgagttgtaatactgatgcacacattacaggagagagctgaacgttctctcacccagagcagcaaacacatgaaagtcagaagtctctaatatatcatttgtcttttttccagtgaggtaaaataaggcataaatgcaggtaattattcccagctgacctaaagtcaataaaacattctgttt

SEQ ID NO: 42 - protein, 358 aa, Rattus norvegicus
  Stearoyl-CoA desaturase-2
MPAHILQEISGSYSATTTITAPPSGGQQNGGEKFEKNPHHWGADVRPEIKDDLYDPSYQDEEGPPPKLEYVWRNIILMALLHIGALYGITLVPSCKVYTCLFAYLYYVISALGITAGAHRLWSHRTYKARLPLRLFLIIANTMAFQNDVYEWARDHRAHHKFSETHADPHNSRRGFFFSHVGWLLVRKHPAVKEKGGKLDMSDLKAEKLVMFQRRYYKPGLLLMCFILPTLVPWYCWGETFVNSLCVSTFLRYAVVLNATWLVNSAAHLYGYRPYDKNISSRENILVSMGAVGEGFHNYHHAFPYDYSASEYRWHINFTTFFIDCMALLGLAYDRKRVSKAAVLARIKRTGEESCKSG

SEQ ID NO: 43 - DNA, 5055 bp, Rattus norvegicus
  Stearoyl-CoA desaturase-2
ctgggctagggatgggcacaatgcggtgttggtgtctgcagtattctactcctggacaccggtggcctgcaagtgcgcttttggcgtcctctcatttttctatccttatctccgtccgcggctgccttggccagccagtttttgattttttatctgttccttgatcgatacaaagaacctaaggacatacaggacattaatacccaactgccagctctggcccagggcttgtactgtgcagcgggctggctgcagaaacttaagtcataacactctttgtcgctgaggtctgaagctctctgcacgttctcatccctgggaacgtgaccccagcatccgacgccaagatgccggctcacatactgcaagagatctctggctcttactcggccaccaccacaatcacagcgccaccttctgggggacagcagaatggaggagagaagtttgaaaagaatcctcaccactggggagcagatgttcgccctgaaattaaagatgacctatacgaccccagctaccaggatgaggaggggcccccgcccaagctggagtacgtctggaggaacatcatcctcatggccctgctgcacattggagccctgtacgggatcacactggtcccctcctgcaaggtctacacctgcctctttgcatatttgtactacgtaatcagcgccctgggcatcacagccggggctcatcgcctgtggagccacaggacttacaaggctcggctgcccctgaggctctttctcatcatcgccaacaccatggcattccagaacgatgtgtacgaatgggcccgggatcaccgcgcccaccacaagttctcagagacacacgccgaccctcacaactcccgccgtggctttttcttctctcacgtgggttggctgcttgtgcgcaaacacccggctgtcaaagagaagggcggaaagctggacatgtctgacctgaaagctgagaagctggtgatgttccagaggaggtattacaagcccggcctcctgctcatgtgcttcatcctgcccacgctggtgccctggtactgctggggtgagacgtttgtaaacagcctgtgcgtcagcactttcttacggtacgctgtggtgctcaacgccacctggctggtgaacagcgccgcccacctctatggatatcgcccctacgacaagaacatcagctctcgggagaacatcctggtgtcgatgggagctgtgggcgagggcttccacaactaccatcacgccttcccctatgactactctgccagtgagtaccgctggcacatcaacttcaccacgttcttcatcgactgcatggccctcctgggcctggcttatgaccggaagagggtgtccaaggccgctgtcttagccaggattaagagaactggggaggagagctgcaagagtggctgagttcggggtcttccagttcctgttccaagagccggcttggcagaggcttactgttctgttaattaactactgagtattgctacccagatgctaaaatgatgaagttaacccattctggtacagtattgtaaaacggacaaatatcggaagtcaacagctctaccttttgatgctaagctgttctccccttctatcttcttttcccgttgtctttttcttcgctttgtccccaatgacctcacccctcatcgcctcccctaagccagcagccactccgctgtagttcagagttcaccttccagagcgtaccagcttttcaagggtctacagtaaaggttcacgccccagccccaacttaacgttgctccaagttacagatagaagaaaaatcttgagaaatttctcctgttattttagtccagactttgccagatggaatgggagaaaaaatagttttatttggcagagagcttgcgaagtgggtaaatcttaaggggccaaattcaacactcgggctgtgattcatcaaaattgtgggagagggggatctcctgtgatcaggctccgctccgtctggccgaggtgctggcgtctcttgacacagcgtgcctcctctcctaattctgattgggtgacagccgtggaactttgtgacactaaaatatggcaacacatcggtatctcagtgtactttaggctaaagatttaaaaaaatagagaagtgaaaaacaatttgtaggaaaagtggtctttcctaaaataagattttcttttcctttcataacaaggacctggggccaggcgtcgtggcacccgcctcaaaacagttgagaggagaggggcaggtgggtctctgagtttgaagccagccagggctagatagtgaggccttgtctcaaatagaaaagccaacaattagcaggacacacggagccgggggtcctcttgcgaggcgtgtctggaatccatgagggcggtcgctcgtggtttttgagcttcctgtgagctgcacccccagacacttgtaattcctgttcttccgtctttcggatgcaggttgctcccacaaattcaggatagctatggcgtttccatgcatcccacaatggaaggagtgtaagacttgagcttgagtcacagctaaaacccagaagacacaaagtcagtctgtcctggatcctacaaacactcaacgagtgcctccctctggaaaagatggtgaacaccttccttccactgttgggcagtggacagggaagttggaggtcgctgtaggcagctccaaggtttctaggggcaccttgccaggggacctcacaactttggcggtctctagtggagcaggttaggttttagctccctttggttcccgctgtgctttgtggtaaggcttttccctgggctgttgcctcattcctcactagctctgtgaccaggtaggactctgggatacatggctgaaagctcttttgctctaccacatctgcctctctttccctacccatttttttgtgtttcggggtggtatttttgtggtggttgttttgttttatttcttctttcctctttgacgtttagtcttgctctaggcagaaacgtcctggatatttacggtcagtaaagatgtccggtcccagcagagatacaacctctatctgcagtaggtagagaaggctgtccctttccggtggctttcagattctccacctctatccctcccaggcctctgatattctttaaactttaaaggaaaagtttggccgactttctagaaacatccctggctcagagaggctctctgtctcctcggttgttctaaaacaaatggtccgggtggtgtgggtgctggcccaagactcgattcctctgagttagggcagagcatgctccctgcactcagcaaagcctctcgggctgatcagtgctcctgggtgatgctgtggtgcttttaggaggaagacagtgttggctggctggaaagcatgacagttaaggttgtgttcttaagtgcctcagtttgaaagctttggctagggtaggagatctgagatgccgtggactttgcagaagagattttgtgcagcaacgcagaagcctggctgtgttaagattggctgtacaggatagcgaatgctgtggttggagggcacagtcttcccgtgttagttaaatagagaggctttaggacgtctctgcgcccacttgaaggtagccttatcctttagctgttcgctaaaatagaatccttgagagattgacaatatatttgctggcctgtcgccctattccttggagaggccttttgaatggttttaattccactggttatccggtgcccacttaatcaaatgtgaaaaataaaaagcagctctcttcttagcctgcccccgtggatctgaggaccctgttagatacagctgtcggtaagggtgtaccagctcactaagaaacactactgtcaagattttaaagctcagaccaagtgacacgttaataagaaactaagatctgattgaattttctaacagtccttgcctcgtggacacgctaaaattcgacttgggtgccttacctcttttctaactagtgttactcatgggcctgcgctcccttcctctgcggattccttagaaccggagagcctgtaagtgtgacaggctcaaaactggcctggctggagggttaggtggactttgatctgcagtgttccctctggggtcttcgtttggggaatatttcttggggctgttttgctgaagtgcccgtagctgatggatggtggaagtaatttgaacgtacgcgatttgtaggttttgttatttgttttaagatttaaaggctggttgtagcatttaaaagtgaaagctttttcttcctggtttgctagtgttctgcgtgtcttctttagttgaaataggttagtggagaagttaagaaagcggtggccgtgttacgctggtggtgaaggccagggccgcctacaaactctcagttttgttttgttttgtttttttataacagtcccactctgacaataatgagttcactccttggataaggttgttaatgagggaagagcccttcagacctaatcagctcttactggacccagctgtcaacactgttgcattggggattaagtttccaaccccgtgaatcatgggacagactcaaaccgtagcactgggcatgtcgcctaacaatataatgtgactcagtattctgtgagacatgggataatattcctgacctacctccacgggtaggtgcgagcatgagcaaatgctctttataaaacatttggggatcctcagcagaggagtatttaagtcctgcacatttttatagtaaccgtccaccatggacttgacacgtgcctatgtcccagacgacattgttaatctacatcattcttctcagaggttgaatggatgcattagataatgtatactcattgctatggaaacctgcaggactccttttccttccttgggtgggatttttttttcctttattatgtgcaacagtagctatttgtaatctaataattttcattaataccaactctgaaagtatttctactcatctgaggttgtgtttgttcataataaaatgaaatggatctgactgc

SEQ ID NO: 44 - protein, 605 aa, Rattus norvegicus
  Ribophorin 1
MEAPIVLLLLLWLALAPTPGSASSEAPPLVNEDVKRTVDLSSHLAKVTAEVVLAHPGGGSTARASSFVLALEPELESRLAHLGVQVKGEDEEDNNLEVRETKMKGKSGRFFTVKLPVALDPGSKISIVVETVYTHVLHPYPTQITQSEKQFVVFEGNHYFYSPYPTKTQTMRVRLASRNVESHTKLGNPSRSEDILDYGPFKDIPAYSQDTFKVHYENNSPFLTITSMTRVIEVSHWGNIAVEENVDLKHTGAVLKGPFSRYDYQRQPDSGISSIRSFKTILPAAAQDVYYRDEIGNVSTSHLLILDDSVEMEIRPRFGLFGGWKTHYIVGYNLPSYEYLYNLGDQYALKMRFVDHVFDEQVIDSLTVKIILPEGAKNIQVDSPYDISRAPDELHYTYLDTFGRPVIVAYKKNLVEQHIQDIVVHYTFNKVLMLQEPLLVVAAFYILFFTVIIYVRLDFSITKDPAAEARMKVACITEQVLTLVNKRLGLYRHFDETVNRYKQSRDISTLNSGKKSLETEHKAVTSEIAVLQSRLKTEGSDLCDRVSEMQKLDAQVKELVLKSAVEAERLVAGKLKKDTYIENEKLSSGKRQELVTKIDHILDAL

SEQ ID NO: 45 - DNA, 2214 bp, Rattus norvegicus
  Ribophorin 1
tcgcggtcatggaggcgccgatcgtcttgctgctgctgctatggctcgccttggccccgacgcctggcagcgcctcttcggaggctccgccgctggtcaacgaggacgtgaagcgcacggtggacctgagcagccacctagccaaggtgacggctgaggtggtcctggcgcacccgggcggcggctctacagcacgagccagctctttcgttctggccctggagcccgaactggagtcgcgacttgcacacctaggcgtgcaggtaaagggagaagatgaggaagacaacaacctagaagtacgagaaaccaaaatgaaggggaaaagtgggaggtttttcaccgtcaagctcccagttgctcttgatcctgggtccaagatctcaatcgttgtggaaactgtctacacccatgtgcttcatccatacccgactcagataactcagtcagagaaacagtttgtggtgtttgagggcaaccattacttctactctccctatccaacaaagacccagaccatgcgagtgagacttgcttcccgaaatgtggagagccataccaagctggggaacccctcaaggtctgaggacatcctggactatgggccttttaaagacatccctgcctacagtcaggatactttcaaagtacattatgagaacaatagccctttcctgaccatcaccagtatgacccgggtcattgaggtttctcactggggcaatattgctgtggaagagaacgtggacttgaagcatacaggtgcggtgctgaagggacctttctcccgctacgattaccagaggcagcctgacagtgggatctcctccattcgttcttttaagaccatccttcctgctgctgcccaggatgtgtattaccgggatgaaatcggtaatgtttccaccagccacctccttattttggatgactccgtggaaatggaaatccggcctcgatttggtctctttggagggtggaagacacactacatcgttggttacaacctcccaagctatgagtacctctataatctgggtgaccagtatgcactgaagatgcggtttgtagaccacgtgtttgatgagcaagtgatagattctctgacagtgaagatcatccttcctgagggagccaagaacatccaggtagacagtccctacgatattagccgggccccagatgagctgcattacacctacctagacacattcggccgcccggtgattgttgcttacaagaagaatctagttgaacagcacattcaggacattgtggtgcactacacattcaacaaggtgctcatgctgcaggagcctctgctggttgtggccgccttctacatcctgttcttcaccgtcatcatctacgtccgtctagacttttccatcaccaaggacccagcagcagaagccaggatgaaagtggcctgtatcacagagcaggtcttaaccctggtcaacaagaggttaggcctctaccgtcactttgatgagactgtcaatagatacaagcagtcccgggacatctctaccctcaatagtggcaagaagagcctagagacagagcacaaagctgtgaccagtgagattgctgtgctgcagtctaggctgaagacggagggctctgacctgtgtgacagggtgagcgaaatgcagaagctggacgcgcaggtcaaggagctggtactgaagtctgcggtggaggcagagaggctggtggctggcaagctcaagaaggacacatacatcgagaatgaaaagctcagctcaggaaaacgccaggagctggtcaccaagatcgaccacatcctagacgctctgtagctcttgtctccagagcaggcctgggtggcctacacttctggatggaataggtagagggaggaagttgtgattagcctaagacctcaagaaaaaagctgaggcccatccatggctgtgtaagaagcttgtcccttatttctgaatggttctcttttttacaaaaacaaaaacctaaacaaaaatcctgcccaaaaaacaaacttggttgtgttttgagcaaaagcattttgagtagttgattgctgttttgttttgatacctgggtattttttctgtctgtaagccctgttttatggccttctgattcctaatgtttggttcttttgtgagcgtatgcatggttttttaaaagtgtgtatgaccaaataaaaataaaggagggactgtg

SEQ ID NO: 46 - protein, 284 aa, Rattus norvegicus
  Sulfotransferase-like protein
MAESEAETPGTPGEFESKYFEFHGVRLPPFCRGKMEDIADFPVRPSDVWIVTYPKSGTSLLQEVVYLVSQGADPDEIGLMNIDEQLPVLEYPQPGLDIIKELTSPRLIKSHLPYRFLPSDLHNGDSKVIYMARNPKDLVVSYYQFHRSLRTMSYRGTFQEFCRRFMNDKLGYGSWFEHVQEFWEHRMDANVLFLKYEDMHRDLVTMVEQLARFLGVSCDKAQLESLIEHCHQLVDQCCNAEALPVGRGRVGLWKDIFTVSMNEKFDLVYKQKMGKCDLTFDFYL

SEQ ID NO: 47 - DNA, 2239 bp, Rattus norvegicus
  Sulfotransferase-like protein
ggcacgagccctggagctgcgggccgggccgggcggcagcatggcggagagcgaagcggagaccccgggcaccccgggcgagttcgagagcaagtacttcgagttccatggcgtgcggctgcctcccttctgccgcgggaagatggaggacatcgccgacttcccagtgcggcccagcgatgtgtggatcgtcacctaccccaagtcaggtacgagcttgctgcaagaggtggtctacttggtgagccagggggctgaccctgatgagattggcctcatgaacattgacgagcagctgccagtactggagtacccacagcccgggctggacatcatcaaggaactgacgtctccccgcctcatcaagagccaccttccctaccgcttcctgccctctgacctccacaacggggactccaaggtcatctacatggctcgcaacccaaaggacctggtggtatcttactatcagttccaccgctcgctgcggaccatgagttaccgaggaaccttccaggagttctgtcggaggttcatgaacgacaaattgggctatggctcctggtttgagcacgttcaggaattctgggaacatcgaatggacgcaaatgtgcttttcctcaagtatgaagatatgcaccgggacctggtgaccatggtggagcagctggccagattcctgggtgtgtcctgtgataaggcccagctggagtccttgatcgagcactgccaccagctggtggaccagtgctgcaatgctgaggccctgcctgtgggccggggaagagtcgggctgtggaaggacatcttcactgtctccatgaacgagaagtttgatctagtgtataaacagaagatggggaagtgtgacctcacgtttgacttttatttataataccagaatccgcaaacttgcatgctcacggtccccagacgctctactagctaaaagtcctgtttgcgttcatttattccttgctggacactccggaagttgcttggaaacagcagggggcgagcctggtggagagagccgaggagtgagatgatgctgtctacagcagccgcctggccgtagcgtttgagtctccacgtgctggccataccacacgcacgggcaggcaaatgtctgcccctgtaccccatgttattcactgtattttatagcgcttttcactgaaaatcgaaataaatatgtcaaccaaatgcaagtccatttccaagggggagcaggagcgagctgtgccgggatgctaggaagctctcaggggtcactgtaatttttatagtcttctctctaggcacagccatcccaccttgtcccgagatcaaaggacaggttggggggagctggagaggtgggtgtagcctgtggagttcagttttctggaaccatcctgacaccaagccaggtcctgtcccaggtctctgagcccagtacagaatgttctggaagggaagctggcctgaggctgcggatggcatctgaaggcatcagtggtgtgtgaagcgtacccacagacaccagactgcaccaagaactagtatatctcatagccttcacttgctccccaaggccgtgactggcacactcccaatggcaagagacatgacataccatggttctgaagtccctagtcatcgtcacgtctgtgccacctgtgccaaggctctcgctcgggagacccagtctgcaggttgactgtcctccatgcagataggattggcaagcccacctgtctgagggggcacttagccctcaaggatgtctgagggagcaatggccccatgtactgtccaccctgacacctaccggctctgtcttccagctccaatttggctcaagtgatgaccagatgatgggtggaccaggagattgctgcttgtggggaccaacttattctgttccttaccaggatgggaagagtcttaacgtggtggggggaggctctggctgtggaaggtgcctgctgtgcgccatctctgaagcaggtttggttgtagcagcgttacctctgtctagaggaaagcttgtgtgggtctggtgcatggttctctgaggtgggcatggcagtgcctttcctgtgtgtctggggaggaaggctgctttctgtgaaattttctttctatttttctatttttagtactgtacggatgttctggaaccacacacggtactctgtgcttgcttgcatctttaataaaagacacctcccctgtcaaaaaaaaaaaaaaaaaa

SEQ ID NO: 48 - protein, 235 aa, Rattus norvegicus
  F1-ATPase alpha subunit
VADRQMSLLLRRPPGREAYPGDVFYLHSRLLERAAKMNDSFGGGSLTALPVIETQAGDVSAYIPTNVISITDGQIFLETELFYKGIRPAINVGLSVSRVGSAAQTRAMKQVAGTMKLELAQYREVAAFAQFGSDLDAATQQLLSRGVRLTELLKQGQYSPMAIEEQVAVIYAGVRGYLDKLEPSKITKFESAFLSHVVSQHQSLLGNIRSDGKISEQSDAKLKEIVTNFLAGFEP

SEQ ID NO: 49 - DNA, 1066 bp, Rattus norvegicus
  F1-ATPase alpha subunit
gtggctgaccgccagatgtctctgctgctccgccgacccccgggtcgagaggcctatcccggtgatgtgttttacctacactctcgcctgctggagagagcagccaagatgaacgattcctttggtggtggctctttgactgccttaccagtcattgaaacacaggctggtgatgtgtccgcctacattccaacaaacgttatttccatcaccgatggacagatcttcttggaaacagaattgttctataaaggcatccgccctgccattaatgtgggcttgtctgtgtcccgtgtcggatctgccgcccagaccagagccatgaagcaggtggcaggcaccatgaagctggagttggcccagtaccgggaggtcgctgcttttgcccagtttggttctgatctggatgctgccactcagcagctcttgagccgtggcgtgcgcctgaccgagctgctaaagcaaggacagtactctcccatggctattgaagaacaggtggctgtcatctatgcaggcgtccggggttatcttgataaactggagcccagtaagatcacaaagttcgagagtgctttcttgtctcatgttgtgagccagcaccagagcctcttgggcaatatcaggtctgatgggaaaatctcagaacagtcggatgcaaagctgaaggaaatcgtaacaaacttcttggctgggtttgaaccttaaagccctgccactgtcaccagacactgctttggttttgtcatttattgtggtaaaatcagcaccatttgtaaaggtttactcttgtactccctgatgtacagaaatcacatgaataaaagttccatattgagtgctagtttgtgtgggacgggctcagatgtatcctgggacttgtggaggtcagagagcagcctgtaggagtcagtgccctcctttccctgggaatttaactcaggttattatttaaagctcgacttcaggggttggggatttagctcagtggtagagcgcttgcctaggaagcgcaaggccctgggttcggtccccagctccggaaaaaaaagaaccaacccgaatt

SEQ ID NO: 50 - protein, 168 aa, Rattus norvegicus
  F1F0 ATPase delta subunit
MLPAALLRHPGLRRLVLQARTYAQAAASPAPAAGPGQMSFTFASPTQVFFDGANVRQVDVPTLTGAFGILASHVPTLQVLRPGLVMVHAEDGTTTKYFVSSGSVTVNADSSVQLLAEEVVTLDMLDLGAARANLEKAQSELSGAADEAARAEIQIRIEANEALVKALE

SEQ ID NO: 51 - DNA, 811 bp, Rattus norvegicus
  F1F0 ATPase delta subunit
ggaattccgctgctgtcgctaagctaaagtcactgacgtttccgccaccatgctgcccgccgcattgcttcgtcacccaggtctgcgccgtctggtgctccaggcgcgtacgtacgcccaggccgccgcctcacctgcccccgccgctgggcctggacagatgtccttcaccttcgcctccccgacgcaggtgttctttgatggtgccaatgtccggcaagtggatgtgcctacgctgactggagcctttggcatcctggcatcccatgtccccacactacaggtcctacggcctgggctggtaatggttcatgcggaagatggcaccacaactaagtattttgtgagcagcggctccgtcactgtgaatgcggactcctctgtgcaattactagctgaagaagtcgtgacactggacatgctggacctcggggcagcccgggcaaacctggagaaggcgcagtcagagctgtcaggggcagcagatgaggcagcacgggctgagatccaaatccggatcgaggccaatgaagccctggtgaaggccctggagtaggcgtactttgtctgtcacccacagggtaacagaggcaggtcctggggggctgaagtcgccaccagggggcagcagtgctccggttactggcttaaagcttccctggtgctgcctgccaggtcatggagggcttccccaggcttccagagtctgggatccccaggatgcctctggagagctggccttgattgcccctcaaagccacccggacagtcagctggcccagcctatctgcattaaataccacggaattcc

SEQ ID NO: 52 - protein, 572 aa, Homo sapiens
  Dihydropyrimidinase-related protein
MSYQGKKNIPRITSDRLLIKGGKIVNDDQSFYADIYMEDGLIKQIGENLIVPGGVKTIEAHSRMVIPGGIDVHTRFQMPDQGMTSADDFFQGTKAALAGGTTMIIDHVVPEPGTSLLAAFDQWREWADSKSCCDYSLHVDISEWHKGIQEEMEALVKDHGVNSFLVYMAFKDRFQLTDCQIYEVLSVIRDIGAIAQVHAENGDIIAEEQQRILDLGITGPEGHVLSRPEEVEAEAVNRAITIANQTNCPLYITKVMSKSSAEVIAQARKKGTVVYGEPITASLGTDGSHYWSKNWAKAAAFVTSPPLSPDPTTPDFLNSLLSCGDLQVTGSAHCTFNTAQKAVGKDNFTLIPEGTNGTEERMSVIWDKAVVTGKMDENQFVAVTSTNAAKVFNLYPRKGRIAVGSDADLVIWDPDSVKTISAKTHNSSLEYNIFEGMECRGSPLVVISQGKIVLEDGTLHVTEGSGRYIPRKPFPDFVYKRIKARSRLAELRGVPRGLYDGPVCEVSVTPKTVTPASSAKTSPAKQQAPPVRNLHQSGFSLSGAQIDDNIPRRTTQRIVAPPGGRANITSLG

SEQ ID NO: 53 - DNA, 4459 bp, Homo sapiens
  Dihydropyrimidinase-related protein
gtttctctctctccttctctctctctctctctctctcttttttttccgccctagctggggctgtgttggaggagaggaagaaagagagacagaggattgcattcatccgttacgttcttgaaatttcctaatagcaagaccagcgaagcggttgcacccttttcaatcttgcaaaggaaaaaaacaaaacaaaacaaaaaaaacccaagtccccttcccggcagtttttgccttaaagctgccctcttgaaattaattttttcccaggagagagatgtcttatcaggggaagaaaaatattccacgcatcacgagcgatcgtcttctgatcaaaggaggtaaaattgttaatgatgaccagtcgttctatgcagacatatacatggaagatgggttgatcaagcaaataggagaaaatctgattgtgccaggaggagtgaagaccatcgaggcccactcccggatggtgatccccggaggaattgacgtccacactcgtttccagatgcctgatcagggaatgacgtctgctgatgatttcttccaaggaaccaaggcggccctggctgggggaaccactatgatcattgaccacgttgttcctgagcctgggacaagcctgctcgctgcctttgaccagtggagggaatgggccgacagcaagtcctgctgtgactactctctgcatgtggacatcagcgagtggcataagggcatccaggaggagatggaagcgcttgtgaaggatcacggggtaaattccttcctcgtgtacatggctttcaaagatcgcttccagctaacggattgccagatttatgaagtactgagtgtgatccgggatattggcgccatagcccaagtccacgcagaaaatggcgacatcattgcagaggagcagcagaggatcctggatctgggcatcacgggccccgagggacatgtgctgagccgacctgaggaggtcgaggccgaagccgtgaatcgtgccatcaccatcgccaaccagaccaactgcccgctgtatatcaccaaggtgatgagcaaaagctctgctgaggtcatcgcccaggcacggaagaagggaactgtggtgtatggcgagcccatcactgccagcttgggaacggacggctcccattactggagcaagaactgggccaaggctgctgcctttgtcacctccccacccttgagccctgatccaaccactccagactttctcaactccttgctgtcctgtggagacctccaggtcacgggcagtgcccattgcacgtttaacactgcccagaaggctgtaggaaaggacaacttcaccctgattccggagggcaccaatggcactgaggagcggatgtccgtcatctgggacaaggctgtggtcactgggaagatggatgagaaccagtttgtggctgtgaccagcaccaatgcagccaaagtcttcaacctttacccccggaaaggccgcattgctgtgggatccgatgccgacctggtcatctgggaccccgacagcgttaaaaccatctctgccaagacacacaacagctctctcgagtacaacatctttgaaggcatggagtgccgcggctccccactggtggtcatcagccaggggaagattgtcctggaggacggcaccctgcatgtcaccgaaggctctggacgctacattccccggaagcccttccctgattttgtttacaagcgtatcaaggcaaggagcaggctggctgagctgagaggggttcctcgtggcctgtatgacggacctgtgtgtgaagtgtctgtgacgcccaagacagtcactccagcctcctcggccaagacgtctcctgccaagcagcaggccccacctgtccggaacctgcaccagtctggattcagtttgtctggtgctcagattgatgacaacattccccgccgcaccacccagcgtatcgtggcgccccccggtggccgtgccaacatcaccagcctgggctagagctcctgggctgtgcgtccactggggactggggatgggacacctgaggacattctgagacttctttcttccttcctttttttttttttgtttttttttttaagagcctgtgatagttactgtggagcagccagttcatggggtcccccttggggccccacaccccgtctctcaccaagagttactgattttgctcatccacttccctacacatctatgggtatcacacccaagactacccaccaagctcatacagggaaccacacccaacacttagacatgcgaacaagcagcccccagcgagggtctccttcgccttcaacctcctagtgtctgttagcatcttccttttcatggggggagggaagataaagtgaattgcccagagctgcctttttcttttctttttaaaaattttaagaagttttccttgtggggctggggaggggccggggtcagggagagtctttttttttttttttttaaatactaaattggaacatttaattccatattaatacaaggggtttgaactggacatcctaatgatgcaattacgtcatcacccagctgattccgggtggttggcaaactcatcgtgtctgtcctgagaggctccacaatgcccacccgcatcgccattctgtagtcttcagggtcagctgttgataaaggggcaggcttgcgttattggcctagattttgctgcagattaaatcctttgaggattctcttctcttttaccatttttctgcgtgctctcactctctctttctctctctagctttttaattcatgaatattttcgtgtctgtctctctctctctctgtgtttcctccagcccttgtctcggagacggtgttttcctcccttgccccattatcttttcacctcccaggtctaccatttcatggtggtcgttgggtccgcctaaaggatttgagcgtttgccattgcaagcatagtgctgtgtcatcctggtccatgtaggactggtgctaaccacctgccatcatgaggatgtgtgctagagtgtgggaccctggccaagtgcaggaatgggccatgccgtctcacccacagtatcacacgtggaaccgcagacagggcccagaagctttagaggtatgaggctgcagaaccggagagattttcctctgtgcagtgctctctggctaaagtcacggtcaaacctaaacaccgagcctcattaacccaagtgaaccaaccaaagtcaccagttcagaagtgctaagctaataggagtctgacccgagggcctgctgcttcctggttaagtatcttttgagattctagaacacatgggagctttttattttcggggaaaaaccgtatttttttcttgtccaattatttctaaagacacactacatagaaagaggccctataaactcaaaaagtcattgggaaacttaaagtctattctactttgcaagaggagaaatgtgttttatgaacgatagatcacatcagaactcctgtggggaggaaaccttataaattaaacacatggcccccttagagaccacaggtgatgtctgtctccatccttccctctccttttctgtcacctttccccctagctggctcctttggacctacccctgtccttgctgacttgtgttgcattgtattccaaacgtgtttacaggttctcttaagcaatgttgtatttgcaggcttttctgaataccaaatctgctttttgtaaagcgtaaaaacatcacaaagtaggtcattccatcaccacccttgtctctctacacattttgcctttggggatctggttggggttttgggttttttgttgttgttgtttatttgttattttaaaggtaaattgcacttttaaaaaaataattggttgacttaatatatttgctttttttctcacctgcacttagaggaaatttgaacaagttggaaaaaaacaatttttgtttcaattctaagaaacacttgcagctctagtattcacttgagtcttcctgtttttcctgtaccgggtcatggtaatttttggttgttttggttgttttcttaaaaaacaagttaaaacctgacgatttctgcaggctgtgtaagcatgtttacctgttggcttgctttgtgtgtctgttaaatgaatgtcatatgtaaatgctaaaataaatcgacagtgtctcagaactgaataactgcagtgacttgatgctctaaaacagtgtaggatttaagaatagatggtttttaatcctggaaattgtgattgtgacccatgagtggaggaactttcagttctaaagctgataaagtgtgtagccagaagagtacttttttttttgtaaccactgtcttgatggcaaaataattatggtaaaaaacaagtctcgtgtttattattccttaagaactctgtgttatattaccatggaacgcctaataaagcaaaatgtggttgtttc

SEQ ID NO: 54 - DNA, 1410 bp, Rattus norvegicus
  Myelin basic protein S
ttcggcttccgaaggcctgatgtgatggcatcacagaagagaccctcacagcgacacggatccaagtacttggccacagcaagtaccatggaccatgcccggcatggcttcctcccaaggcacagagacacgggcatccttgactccatcgggcgcttctttagcggtgacaggggtgcgcccaagcggggctctggcaaggactcacacacaagaactacccactacggctccctgccccagaagtcgcagaggacccaagatgaaaacccagtagtccacttcttcaagaacattgtgacacctcgtacaccccctccatcccaaggaaaggggagaggcctgtccctcagcagatttagctggggaggaagagacagccgctctggatctcccatagcaagacgctgagagcctccctgctcagccttcccgaatcctgccctcggcttcttaatataactgccttaaacgtttaattctacttgcaccaaatagctagttagagcagaccctctcttaatcccgtggggctgtgaacgcggcgggccagcccacggcaccctgactggctaaaactgtttgtccctttttatttgaagattgagtttcctcggggtcttctctgccccgacttgctccccgtgtaccttggtcgactccggaggttcaggtgcacggacaccctttcaagttcacccctactccatcctcagactttcttttcacggcgaggcgcacccctccagcttccgtgggcactgcggatagacaggcacaccgccaaggagccagagagcatggcgcaggggactatgtggtccaggcttcctttgttttctttcccctaaagagctttgtttttcctgacaggatcagacagtcttggagtggcttacacaacgggggcttgtggtatgtgagcacaggtcgggcagctgtgagagtccagagtggggtggccctggggacgcttccaggccagcggttccctgcaccccaccagctgatttcgagcgtggcagagggaaggaaaggggcgagcgggctgggcaatggacccgacaggaaacggggacttaggggaacacgctggagatgccatgtgtggctgccgaaggtcaccatctctcctcagtgctccccagagcaggtgcttttaagaaccctgtttcctctcagagcctagggagagtccaaggacatggcgcatcaggaagtgggactgcaggagttctctggtggcctcgtgctgtccctctggccacttctcactttagggtggtcacggcagctcgccatggcagtgccattggtgcacactaacctcatggaaaagtaaccattccctgcctcttagaaagaactcattcttagttttaggagggttcctgtcgctgaatcaagtcg

SEQ ID NO: 55 - protein, 698 aa, Rattus norvegicus
  Transferrin
MRFAVGALLACAALGLCLAVPDKTVKWCAVSEHENTKCISFRDHMKTVLPADGPRLACVKKTSYQDCIKAISGGEADAITLDGGWVYDAGLTPNNLKPVAAEFYGSLEHPQTHYLAVAVVKKGTDFQLNQLQGKKSCHTGLGRSAGWIIPIGLLFCNLPEPRKPLEKAVASFFSGSCVPCADPVAFPQLCQLCPGCGCSPTQPFFGYVGAFKCLRDGGGDVAFVKHTTIFEVLPQKADRDQYELLCLDNTRKPVDQYEDCYLARIPSHAVVARNGDGKEDLIWEILKVAQEHFGKGKSKDFQLFGSPLGKDLLFKDSAFGCYGVPPRMDYRLYLGHSYVTAIRNQREGVCPEASIDSAPVKWCALSHQERAKCDEWSVTSNGQIECESAESTEDCIDKIVNGEADAMSLDGGHAYIAGQCGLVPVMAENYDISSCTNPQSDVFPKGYYAVAVVKASDSSINWNNLKGKKSCHTGVDRTAGWNIPMGLLFSRINHCKFDEFFSQGCAPGYKKNSTLCDLCIGPAKCAPNNREGYNGYTGAFQCLVEKGDVAFVKHQTVLENTNGKNTAAWAKDLKQEDFQLLCPDGTKKPVTEFATCHLAQAPNHVVVSRKEKAARVSTVLTAQKDLFWKGDKDCTGNFCLFRSSTKDLLFRDDTKCLTKLPEGTTYEEYLGAEYLQAVGNIRKCSTSRLLEACTFHKS

SEQ ID NO: 56 - DNA, 2293 bp, Rattus norvegicus
  Transferrin
cgtccgctcctcgccacacacaccgagaggatgaggttcgctgtgggtgccctgctggcttgtgccgccctgggactgtgtctggctgtccctgacaaaacggtcaaatggtgcgcagtgtctgagcatgagaacaccaagtgtatcagtttccgtgaccacatgaaaaccgtccttccagctgatggcccccggcttgcctgtgtgaagaaaacctcctatcaagattgcatcaaggccatttctggaggtgaagctgatgccattaccttggatgggggttgggtgtacgatgcaggcctgactcccaacaacctgaagcctgtggcagcagagttttatggatcacttgaacatccacagacccactacttggctgtggccgtggtgaagaagggaacagacttccagctgaaccagctccagggcaagaagtcctgccacactggcctgggcaggtctgcaggctggattatccccattggcttacttttctgtaacttgccagagccccgcaagcctcttgagaaagctgtggccagtttcttctcgggcagttgtgtcccctgtgcagatccagtggccttcccccagctgtgtcaactgtgtccaggctgtggctgctccccgactcaaccgttctttggctacgtaggcgccttcaagtgtctgagagatggaggtggagatgtggcctttgtcaagcatacaaccatatttgaggtcttgccacagaaggctgacagggatcaatatgagctgctctgccttgacaatacccgcaagccagtggatcagtatgaggactgctacctagcccggatcccttctcatgctgttgtggctcgaaatggagatggcaaagaggacttgatctgggagatcctcaaagtggctcaggaacactttggcaaaggcaaatcaaaagacttccaactgttcggctctcctcttgggaaagacctgctgtttaaggattctgcctttggctgttacggggtgcccccaaggatggactacaggctgtacctcggccacagctatgtcactgccattcgaaatcagcgggaaggcgtgtgcccggaggcctccatcgacagcgcgccagtgaaatggtgtgcactgagtcaccaagagagagccaagtgtgatgagtggagcgtcacgagcaatgggcagatagagtgtgagtcagcagagagcactgaggactgcattgacaagattgtgaatggagaagcagatgccatgagcttggatggaggtcatgcctacatagcaggccagtgtggactagtgcccgtcatggcagagaactatgatatctcttcgtgtacaaacccacaatcagatgtctttcctaaagggtattatgccgtggctgtggtgaaggcatcagactccagcatcaactggaacaacctgaaaggcaagaagtcctgccatactggagtagacagaaccgccggctggaacatccctatgggcctgctgttcagcaggatcaaccactgcaagttcgatgaatttttcagtcaaggctgtgctcctggctataagaagaattccaccctctgtgacctgtgtattggcccagcaaaatgtgctccgaacaacagagagggatataatggttatacaggggctttccagtgcctcgttgagaagggagacgtagcctttgtgaagcaccagactgtcctggaaaacacgaacggaaagaacactgctgcatgggctaaggatctgaagcaggaagacttccagctgctgtgccctgatggtaccaagaagcctgtaaccgagttcgccacctgccacctggcccaagctccaaaccatgttgtggtctcacgaaaagagaaggcagcccgggttagcactgtgctgactgcccagaaggatttattttggaaaggtgacaaggactgcactggcaatttctgtttgttccggtcttccaccaaggaccttctgttcagagatgacaccaagtgtttgactaaacttccagaaggtaccacatatgaagagtacttaggagcagagtacttgcaagctgttggaaacataaggaagtgttcaacctcacgactcctagaagcctgcactttccacaaaagttaaaatccaagaggtgggtgccactgtggtggaggaggatgcccccgtgatccatgggcttctcctggcctccatagccctgagcggctggggctaactgtgtccgtcttactgcttggtgttgttaccacatacacagagcacaaaataaaaaatgactgttgacttt

SEQ ID NO: 57 - protein, 542 aa, Rattus norvegicus
  NF-L
MSSFSYEPYFSTSYKRRYVETPRVHISSVRSGYSTARSAYSSYSAPVSSSLSVRRSYSSSSGSLMPSLENLDLSQVAAISNDLKSIRTQEKAQLQDLNDRFASFIERVHELEQQNKVLEAELLVLRQKHSEPSRFRALYEQEIRDLRLAAEDATNEKQALQGEREGLEETLRNLQARYEEEVLSREDAEGRLMEARKGADEAALARAELEKRIDSLMDEIAFLKKVHEEEIAELQAQIQYAQISVEMDVSSKPDLSAALKDIRAQYEKLAAKNMQNAEEWFKSRFTVLTESAAKNTDAVRAAKDEVSESRRLLKAKTLEIEACRGMNEALEKQLQELEDKQNADISAMQDTINKLENELRSTKSEMARYLKEYQDLLNVKMALDIEIAAYRKLLEGEETRLSFTSVGSITSGYSQSSQVFGRSAYSGLQSSSYLMSARAFPAYYTSHVQEEQSEVEETIEATKAEEAKDEPPSEGEAEEEEKEKEEGEEEEGAEEEEAAKDESEDAKEEEGGEGEEEDTKESEEEEKKEESAGEEQAAKKKD

SEQ ID NO: 58 - DNA, 2032 bp, Rattus norvegicus
  NF-L
gaattcccccggttcttctctctaggtcccctatctccgccccgggcctgaggggcgcaccgaccgccaccatgagttcgttcagctacgagccgtacttttcgacctcctacaagcggcgctacgtggagacgccccgggtgcacatctccagcgtgcgcagcggctacagcacggcgcgctctgcgtactccagctactccgcgcccgtctcctcctctctgtcggtgcgccgcagctactcatccagctccggctctttgatgcccagcctggagaacctcgatctgagccaggtagccgccatcagcaacgacctcaagtctatccgcacacaggagaaggcacagctgcaggacctcaacgatcgcttcgccagcttcatcgagcgcgtgcacgagctggagcagcagaacaaggtcctggaagccgagctgttggtgctgcgccagaagcactcagagccttcccgcttccgcgccctgtatgagcaggagatccgtgatctgcgactggcggccgaagacgccactaacgagaagcaggcgctgcagggcgagcgcgaggggctggaggagactctgcgcaacctgcaggctcgctacgaggaggaggtgctgagccgcgaggacgccgagggccggctgatggaagcccgcaaaggcgcggatgaggctgcgctcgcccgcgccgagctggagaagcgcatcgacagcctgatggacgagatagccttcctgaaaaaggtgcacgaggaagagatcgccgagctgcaggctcagatccagtatgctcagatctccgtggagatggacgtgtcctccaagcccgacctctccgccgctctcaaggacatccgcgctcagtacgagaagctggccgccaagaatatgcagaatgccgaagagtggttcaagagccgcttcacggtgctaaccgagagcgccgccaagaacaccgacgcagtgcgcgctgccaaggacgaggtgtcggaaagccgccgcctacttaaggctaagaccctggagatcgaagcctgccggggtatgaacgaagctctagagaagcagctgcaggagctggaggacaagcagaatgcagacatcagcgccatgcaggacacaatcaacaaactggagaatgagctgcgaagcacgaagagcgagatggccaggtacctgaaggagtaccaggacctcctcaatgtcaagatggcattggacattgagattgcagcttacaggaaactcttggaaggcgaagaaaccaggctcagtttcaccagcgtgggtagcataaccagcggctactctcagagctcgcaggtctttggccgttctgcttacagtggcttgcagagcagctcctacttgatgtctgctcgagcattcccagcctactataccagccacgtccaggaagagcagtcagaggtggaggagaccattgaggctacgaaagctgaggaggccaaggatgagcccccctctgaaggagaagcagaagaggaggagaaggagaaagaggagggggaggaagaggaaggtgctgaggaggaagaagctgccaaggatgagtctgaagatgccaaagaagaagaaggtggtgagggtgaagaggaagacaccaaagagtcagaagaggaagagaagaaagaggagagtgctggggaggagcaagctgctaagaagaaagattgagccccattcccaactatcccaggaaaaaagtctccccaaatcaggtcaacctcatcaccaaaccaaccagttgagttccagatcctatacagattaagaagtcaatatatgtataattctgagatgacttaggttggacattcaatgttgtgctatgactttcctccttatgcagagtatctgtttgcttgcagagtggctttctggcttgctgccaacctgtgcatggtccatgcttatgagttcaggatctacggcaatgtgaatcacacagatgtttacaataataataataaaaaaaccacacatacacaacatgaataaatgaattc

SEQ ID NO: 59 - protein, 626 aa, Rattus norvegicus
  MAG
MIFLTTLPLFWIMISASRGGHWGAWMPSSISAFEGTCVSIPCRFDFPDELRPAVVHGVWYFNSPYPKNYPPVVFKSRTQVVHESFQGRSRLLGDLGLRNCTLLLSTLSPELGGKYYFRGDLGGYNQYTFSEHSVLDIINTPNIVVPPEVVAGTEVEVSCMVPDNCPELRPELSWLGHEGLGEPTVLGRLREDEGTWVQVSLLHFVPTREANGHRLGCQAAFPNTTLQFEGYASLDVKYPPVIVEMNSSVEAIEGSHVSLLCGADSNPPPLLTWMRDGMVLREAVAESLYLDLEEVTPAEDGIYACLAENAYGQDNRTVELSVMYAPWKPTVNGTVVAVEGETVSILCSTQSNPDPILTIFKEKQILATVIYESQLQLELPAVTPEDDGEYWCVAENQYGQRATAFNLSVEFAPIILLESHCAAARDTVQCLCVVKSNPEPSVAFELPSRNVTVNETEREFVYSERSGLLLTSILTLRGQAQAPPRVICTSRNLYGTQSLELPFQGAHRLMWAKIGPVGAVVAFAILIAIVCYITQTRRKKNVTESPSFSAGDNPHVLYSPEFRISGAPDKYESEKRLGSERRLLGLRGEPPELDLSYSHSDLGKRPTKDSYTLTEELAEYAEIRVK

SEQ ID NO: 60 - DNA, 2475 bp, Rattus norvegicus
  MAG
cagaagccagaccatccaaccttctgtatcagtgctcctcgtcgcctcactgtacttcacggaagagacttggttgactggccacttggagcggaatcaggagacattcccaactcagggagactgaggtgagggccctagctcgcccacttgctggacaagatgatattccttaccaccctgcctctgttttggataatgatttcagcttctcgaggggggcactggggtgcctggatgccctcgtccatctcagccttcgagggcacgtgtgtctccatcccctgccgtttcgacttcccggatgagctcagaccggctgtggtacatggcgtctggtatttcaacagtccctaccccaagaactacccgccagtggtcttcaagtcccgcacacaagtggtccacgagagcttccagggccgtagccgcctgttgggagacctgggcctacgaaactgcaccctgcttctcagcacgctgagccctgagctgggagggaaatactatttccgaggtgacctgggcggctacaaccagtacaccttctcggagcacagcgtcctggacatcatcaacacccccaacatcgtggtgcccccagaagtggtggcaggaacggaagtagaggtcagctgcatggtgccggacaactgcccagagctgcgccctgagctgagctggctgggccacgaggggctaggggagcccactgttctgggtcggctgcgggaggatgaaggcacctgggtgcaggtgtcactgctacacttcgtgcctactagagaggccaacggccaccgtctgggctgtcaggctgccttccccaacaccaccttgcagttcgagggttacgccagtctggacgtcaagtaccccccggtgattgtggagatgaattcctctgtggaggccattgagggctcccatgtcagcctgctctgtggggctgacagcaacccgccaccgctgctgacttggatgcgggatgggatggtgttgagggaggcagttgctgagagcctgtacctggatctggaggaggtgaccccagcagaggacggcatctatgcttgcctggcagagaatgcctatggccaggacaaccgcacggtggagctgagcgtcatgtatgcaccttggaagcccacagtgaatgggacggtggtggcggtagagggggagacagtctccatcctgtgttccacacagagcaacccggaccctattctcaccatcttcaaggagaagcagatcctggccacggtcatctatgagagtcagctgcagctggaactccctgcagtgacgcccgaggacgatggggagtactggtgtgtagctgagaaccagtatggccagagagccaccgccttcaacctgtctgtggagtttgctcccataatccttctggaatcgcactgtgcagcggccagagacaccgtgcagtgcctgtgtgtggtaaaatccaacccggaaccctccgtggcctttgagctgccttcccgcaacgtgactgtgaacgagacagagagggagtttgtgtactcagagcgcagcggcctcctgctcaccagcatcctcacgctccggggtcaggcccaagccccaccccgcgtcatttgtacctccaggaacctctacggcacccagagcctcgagctgcctttccagggagcacaccgactgatgtgggccaaaatcggccctgtgggtgctgtggtcgcctttgccatcctgattgccattgtctgctacatcacccagacaagaagaaaaaagaacgtcacagagagccccagcttctcagcgggagacaaccctcatgtcctgtacagccccgaattccgaatctctggagcacctgataagtatgagagtgagaagcgcctggggtccgagaggaggctgctgggccttaggggggaacccccagaactggacctcagttattcccactcagacctggggaaacgacccaccaaggacagctacaccctgacagaggagctggctgagtacgcagaaatccgagtcaagtgaggaagctgggggctggccctgtggctcaccccccatcaggaccctcgcttggcccccactggccgtgggctccctttctcttgagagtggtaggggtgggggcgggaaggggcggggcaggaaacagtgaggtcttaggggcccggcctcccctccttcccggctgctcctctctgccaacatcctgcacctatgttacagctccctctcccctccttttaacctcagctgttgagaggggtgctctgtctgtccatgttatttattgttatcctggtctcctgtccccttacccggccccaggacctgtacaaaagggacatgaaataaatgtcctaatgacaagtgccagtctagacccatcctttggaggaaaggggcatattagtaatacttttctcgttgctgtaacaaaatactggacaaaaacac

SEQ ID NO: 61 - protein, 845 aa, Rattus norvegicus
  NF-M protein
MSYTLDSLGNPSAYRRVPTETRSSFSRVSGSPSSGFRSQSWSRGSPSTVSSSYKRSALAPRLAYSSAMLSSAESSLDFSQSSSLLNGGSGGDYKLSRSNEKEQLQGLNDRFAGYIEKVHYLEQQNKEIEAEIHALRQKQASHAQLGDAYDQEIRELRATLEMVNHEKAQVQLDSDHLEEDIHRLKERFEEEARLRDDTEAAIRAVRKDIEESSMVKVELDKKVQSLQDEVAFLRSNHEEEVADLLAQIQASHITVERKDYLKTDISTALKEIRSQLECHSDQNMHQAEEWFKCRYAKLTEAAEQNKEAIRSAKEEIAEYRRQLQSKSIELESVRGTKESLERQLSDIEERHNHDLSSYQDTIQQLENELRGTKWEMARHLREYQDLLNVKMALDIEIAAYRKLLEGEETRFSTFSGSITGPLYTHRQPSVTISSKIQKTKVEAPKLKVQHKFVEEIIEETKVEDEKSEMEDALTVIAEELAASAKEEKEEAEEKEEEPEVKSPVKSPEAKEEEEGEKEEEEEGQEEEEEEDEGVKSDQAEEGGSEKEGSSEKDEGEQEEEGETEAEGEGEEAEAKEEKKTEGKVEEMAIKEEIKVEKPEKAKSPVPKSPVEEVKPKPEAKAGKDEQKEEEKVEEKKEVAKESPKEEKVEKKEEKPKDVPDKKKAESPVKEKAVEEMITITKSVKVSLEKDTKEEKPQQQEKVKEKAEEEGGSEEEVGDKSPQESKKEDIAINGEVEGKEEEEQETQEKGSGQEEEKGVVTNGLDVSPAEEKKGEDRSDDKVVVTKKVEKITSEGGDGATKYITKSVTVTQKVEEHEETFEEKLVSTKKVEKVTSHAIVKEVTQGD

SEQ ID NO: 62 - DNA, 2717 bp, Rattus norvegicus
  NF-M protein
ggcctccaagatgagctacacgctggactcgctgggcaacccgtccgcctaccggcgcgttccaaccgagacccggtccagcttcagtcgtgtgagcggttccccgtccagcggcttccgctcgcagtcctggtcccgcggctcgcccagcaccgtgtcctcctcctacaagcgcagcgcgctcgccccgcgcctcgcctacagctcggctatgctcagctcggccgagagcagcctcgacttcagccagtcctcttcgctgcttaacggcggctccggcggcgactacaagctgtcccgctcaaacgagaaagagcagctgcaggggctgaacgaccgtttcgccggctacatcgagaaagtgcactacttggaacaacagaacaaggagatcgaggcagagatccacgcgctgcggcagaagcaggcctcgcacgcccagctgggtgacgcttacgaccaggagatccgagagctgcgcgccaccctggagatggtgaatcacgagaaggctcaagtgcagctggactctgatcacttggaggaagacatccaccggctcaaggagcgcttcgaggaggaggcgcggctgcgggacgacacggaggctgccatccgggcggtgcgcaaagacatagaggagtcgtcgatggttaaggtggagctggacaagaaggttcagtcgctgcaggatgaggtggccttcctgcggagcaatcacgaagaggaggtggccgacctgctggcccagatccaggcgtcgcacatcaccgtagagcgcaaagactacctgaagacagacatctccacggcgctgaaagagatccgctcccagctcgagtgtcactccgaccagaacatgcaccaggccgaagagtggttcaaatgccgctacgccaagctcaccgaggcggccgagcagaacaaggaggccatccgctccgctaaagaagagatcgccgagtaccggcgccagctgcagtccaagagcattgagctcgagtcggtgcgaggcactaaggagtccctggaacggcagctcagcgacatcgaggagcgccacaaccacgacctcagcagctaccaggacaccatccagcagctggaaaatgagcttcggggaacaaagtgggaaatggctcgtcatttgcgagaataccaggatctccttaacgtcaagatggctctggacatcgagatcgccgcatataggaaactactggagggtgaagagaccagatttagcacattttcaggaagcatcactgggcctctgtacacacaccgacagccctcagtcacaatatccagtaagattcagaagaccaaagtcgaggcccccaagctcaaggtccaacacaaatttgtggaggagatcattgaggagactaaagtggaagatgagaagtcagaaatggaagacgccctcacagtcattgcagaggaattggcagcctctgccaaagaggagaaagaagaggcagaagaaaaggaagaggaaccggaagtgaagtctcccgtgaagtctcctgaggctaaggaagaggaggaaggggaaaaggaggaagaagaggaaggccaagaggaagaagaggaggaagatgaaggtgtcaagtcagaccaggcagaagagggaggatctgagaaggaaggctcgagtgaaaaggatgaaggtgagcaagaagaagaaggggaaactgaggcagaaggtgaaggagaggaagcagaagctaaggaggaaaagaaaacagagggaaaggtcgaggaaatggctatcaaggaggaaatcaaggtcgagaagcccgagaaagccaagtcccctgtgccaaaatcaccggtggaagaagtaaagccaaaaccagaagccaaagccggaaaggatgagcagaaggaggaagagaaagttgaggagaagaaggaggtagccaaggaatcacccaaggaagagaaggtggagaaaaaggaggagaagccaaaagatgtcccagataaaaagaaggctgagtccccagtgaaagaaaaggccgtagaggaaatgatcaccattactaagtcggtaaaggtgagcctggagaaagacaccaaagaggagaagcctcagcagcaggagaaggtgaaggagaaggcagaggaggaggggggtagtgaggaggaagtgggtgacaaaagcccgcaagaatccaagaaggaagacatagctatcaatggggaggtggaaggaaaagaggaggaggagcaggaaactcaggagaagggcagtgggcaagaggaggagaaaggggtggtcactaatggcttagatgtgagccctgcggaggaaaagaaaggggaggatagaagtgatgacaaagtggtggtgaccaagaaggtagaaaaaatcaccagcgagggaggcgatggtgctaccaaatacatcaccaaatctgttactgtcactcaaaaggttgaagagcatgaggagacctttgaggagaagctggtgtcaactaaaaaggtagaaaaggtcacttcacatgccatagtcaaggaagtcacccagggtgactaagatcggagtccattgcaaaaggttaagccatacaacaatttcaaaatgcatgtgattgacagcttcaaaacagaatgggttctcccatgagggctccagacattgtattttcctttgtgcaatatgagggaactgcatgcaagctcagggtgcctcctcagtccttggg

SEQ ID NO: 63 - protein, 707 aa, Rattus norvegicus
  Neurodegeneration associated protein 1
MSQYTEKEPSVMDQDSSKAAWPRAAGGYQTITGRRYGRRHAYVSFKPCMTRHERSLGRAGDDYEVLELDDVAKENTAGSSSLDQVHPSLPSETTVEKSETEIPTCGPALNQSTESNPSVATVCHSEEVRETLDSSTNLQNHAERECTPAVCNASSVQNGIVLVHTDSYDPDSKHDENDSLQLCAQAVEGGRRQKVLGNAVFELENGEVERYADLCPSVPSLSGEIREESEELGSALLEKNSAGDAEAVHQDGQEFQRSSEDGIVRKRRQDDTDQGRQTENSTEDADCVPGHVEQNTSERANHHGSSPEQVVRPKVRKVISSSQVDQESGFNRHEAKQRSVQRWREALEVEECSSDDPIIKCDDYDGDHDCMFLTPSYSRVTPREAERHRATAENGATASGRQEARENAFWNACGEYYQLFDKDEDSSECSDGEWSASLPHRFSGTEKDQSSSDESWETLPGKDENEPELQSDSSGPEEENQELSLQEGEQTSLEEGEIPWLQYNEVNESSSDEGNEPANEFAQPEAFMLDGNNNLEDDSSVSEDLDVDWSLFDGFADGLGVAEAISYVDPQFLTYMALEERLAQAMETALAHLESLAVDVEVANPPASKESIDGLPETLVLEDHTAIGQEQCCPICCSEYIKDDIATELPCHHFFHKPCVSIWLQKSGTCPVCRRHFPPAVIDASAAASSEPDLDASPANDNAEEAP

SEQ ID NO: 64 - DNA, 4758 bp, Rattus norvegicus
  Neurodegeneration associated protein 1
tggcgggtggaggatcgagcgctgttctcgctctggagccgctgcacatctcggaatcatttgcagcttattcactgtgtaaggaaaaaccaaattacatccataattctattgaactggaagcatagacgctgccatatatgtcacagtatacagaaaaagaaccatcagtaatggatcaagactccagcaaggctgcgtggcctagagcagcaggaggataccagacgattacaggcaggaggtacggaaggagacatgcgtatgtcagttttaagccatgtatgaccaggcatgaacggagcttgggtcgggctggcgatgactatgaagttctggaactggatgacgttgccaaggaaaacaccgcaggttccagttcattggatcaagtccatccttctttacccagtgaaactacagttgaaaaaagtgaaacagaaattcctacttgtggtccagcactgaatcaaagcacggagagcaacccatccgttgccacagtgtgtcatagtgaggaagtcagggagaccttagacagcagtacgaatcttcagaatcacgctgagagagagtgtacgccagcagtttgtaatgcctcaagtgtccagaatggaattgtgttggttcatactgactcttatgatccagacagcaagcatgatgagaatgactctcttcaactttgtgcccaagctgtggaaggtggtagacgtcagaaggtattaggcaatgcagtctttgagctggaaaatggagaggtagagagatatgctgatctgtgtccctcagttccctctctcagtggtgaaataagggaggagtctgaagagctaggttcagcactcttagagaaaaattctgctggcgatgcggaggctgtccatcaggatgggcaggaatttcagaggtcttctgaagacggcattgttagaaagaggcgacaagatgataccgatcagggaagacagacagaaaattcaactgaagatgcagactgtgttccaggccatgttgaacaaaatactagtgagagagccaatcaccatggaagttctcctgaacaggtagtgaggcccaaagttagaaaagtgatcagttcaagccaggtggaccaagagagcggttttaataggcacgaggctaagcaaagaagtgttcagaggtggagagaggctctggaagttgaggaatgcagttcagatgaccctataatcaagtgtgacgattatgatggagaccatgactgcatgttcctaaccccatcctactcaagagttacgccaagggaagcagaacgtcaccgtgcgacagcagaaaatggagccacagcttcaggaaggcaagaggctcgggaaaatgccttttggaatgcctgtggagagtattaccagctctttgacaaagacgaagacagttcagagtgcagtgatggggaatggtctgcttctctgcctcaccgattttctggcacagaaaaagaccagtcctcaagcgatgaaagctgggaaactctgccaggaaaagatgagaatgaacctgagctacagagtgatagtagtggccctgaggaagaaaaccaagaattgtctcttcaggagggagaacagacgtccttggaggagggggagattccctggttacagtacaatgaggtcaatgagagcagcagcgatgaagggaacgagcctgccaatgagtttgcacaaccagaagctttcatgttggatgggaacaacaacctggaggacgactcgagcgtgagtgaagacctggatgtggactggagcctatttgatggttttgcggatggacttggtgttgctgaagctatttcctacgtggatcctcagttcctcacctacatggcactagaagaacgcttagcccaggctatggagactgcactggcacacttagagtctcttgctgtagacgttgaggtggctaacccacctgccagtaaggaaagcatcgacggacttccagaaacccttgtcctagaagatcacaccgctattggtcaggagcagtgctgtcccatctgctgcagtgagtacattaaggatgacatcgcaacagagctgccctgccatcacttctttcataagccttgtgtctccatttggctacagaagtccggaacgtgccctgtgtgccgccgccacttcccacctgcagtgattgacgcatctgcagctgcttcctctgaaccagaccttgatgcctcgcctgcaaacgacaatgctgaggaagccccgtaagccgtcacggggaaatgagatcgaaattctatcacagtaaatctgcaaattccttctaaatctgatgtgcaaataattatatataaatatatttaaaatgctctatatagtatatgccgtagtttagaaagagaatattaacctttctaaactgaatttaggtttgcagaagatactaaacattttcaagctgaatgttgaagcagtgcatccattttctttagttgaacatggtgtccattgtaacgtcagtagtcatctctgtggcatacgtttctgttttgtctgaagtgctgccactaagagatcagaattaagctctcctttccacatcagatgtgaaaggagagcttaattctgatctctgtggcatacgtttctgttttgtctgaagtgctgccactaagagatcagaattaagctctcctttccacatcagatgtgaaaactgtgatcacaacagtagtacagtttggtttcattgaaaataaactgaattctaaagcatgctttttcactggtccctttgcttttgctacttcgagacctcttggtttatataacactgaggttaagattaaagcttttcagaatgccaggcaaagactagagttttgacccgaacacacacacaaaataaaaaaataaaaaataaattacatcttagatgtaaacttcctgaaactgtcatgtaataaactattcagtgaacgttggatgtttaaaggctagtaaagcatgtgaattatctgatgtgcttcatgctcggcacagtctcatgagcttggcctatgaaacactgtctaagccctcatgcagtctgcttctacttgttaaacactgaaggtccagaccagtcttggcatctgattaagaaattggatgtagttagctaaggtagtttgggttctactttaggagatctgaagatttgcacatggccatgtctgttacagcatcaccccagattagaaaggcacagcttttaggtggtcacacgagtaggcaagtctgtgcagacgctggtgtgataggtagcttacaggtgagccctgacatcagcacagggacaacggctcagcaggcccagcactggagcccatccgcgtgcacatctacctgccctgctcatccagttcgggaatatttctatagcttcccaaatcagcttcatctgatttgtgtaatatatctgagtatttatagtctttcttttttctccagtcttaaaaataaatgaattttcactgttggcacatttgaggcttaaatataagaaacctaacacttgcattctgatttttgcatatattgtaaatgtggctggtatttacagcgaatcctgtgtgtccttttatgggtaaaacaaaagtgaacattgcatgcaagtaatgtggtgaatttgtaatttaggggttttctggggcttctgtgactgtggatgcttaaattcaggccttaatgttgctctagctagcatgtttccttctaatgtacatagtcagcctgtatccactaatctctgcttgtcttctcctccgtggtcttcacaccgtatccactgatggtcagttagtgtcagaggtcacccaggttagcatcagttcccatgtgtgccgtgcggattgtgagccgtgtggttgcttggttggttttcttttatgtactttgattagctgtggcacttactgagtaaacttacgttgctgcagactgctgtgtaacaagccacgttttgtgtttagtcagtattacacacgggaccttgttttattcttctagatagcagctgtcccaaagaaaatatttcttctttgcctattaagatttagctattatctgccagttgttaaggggtttgattccaaactcaaccagagttgaacgtaagaaagctgctgtactcttgctgtccatctgttacgtcctccgttgttggctcctgactacgtacccacggctgctgaaggagacttgttttaatttgttttaaaaaggaaaatgaagttttaggactttgatggtggggaagctgacaagttcagaccccagtctgtcatttgctcttagcttggatccttttgaaaagttaagaatatatgaaggtaaattagaaaaagtaaaaatattgataaaatgtcatttatcttatgtctattttatgttgttatttggcctattaattttaattttttgaacattttcttatttcttgtaatatgaatgccgatatttaaagtttgactcacttgggtttcttttgtgtttcttagttgttatctctaagcctaactaacttttaagattatttgtgatatggatttatataagctgttaaatatatatgagctgttaaaatggaatgcaaggttttccaaagaccaggtctaactgtaatgattggtttattgttctacaatcccagcccggcattttccgtgtaaatcataaacaataaacaggatatactcagtgttcatttctaaaaaaaaaa

SEQ ID NO: 65 - DNA, 1488 bp, Rattus norvegicus
  Brain S-100 protein beta-subunit
aagtccacacccagtcctctctggaggaagaaaagggagcttctctgtctaccctcctagtccttggacaccgaagccagagaggactccggcggcaaaaggtgaccaggagcctccgggatgtctgagctggagaaggccatggttgccctcattgatgtcttccatcagtattcagggagagagggtgacaagcacaagctgaagaagtcagaactgaaggagctcatcaacaacgagctctctcacttcctggaggaaatcaaagagcaggaagtggtggacaaagtgatggagacgctggacgaagatggggatggggagtgtgacttccaggagtttatggccttcgtctccatggtgaccacagcctgtcatgagttctttgaacatgagtgagacaaaaaaaaaaaaaaaaaagtggctgagccgtttccccgtggcagacatgagggccacgagaggaggcacggcagaaggctcgtgggctggaaggagctgcgctctctagacgcatataactaattaggaagcttgatttgcttcagggatgaaactctgaccccgttcccaagggctgctttaagttagcacttcgtttctgctacactaggtattcctgtgagctgaatggtcccgggaactattgataagagtcactgagggacgaaatcaacactctgtgggtatagcactggttgtagaccaccatgctcctgtggaagggtcacctgtaagaatcaaggcagactaccaatagcacctccgttggacagctttcttaggtgtaatgtatgctgtccatgcatctacagacccacagctggatccactgccacccgaagaggttggctcgcccttacaactgcttgtcctctgtgcaaacgatgccccggaaagttagacctatcacccacaccctcccaccacccccaggccaaaaggacagcccacccaagtcccctcccccacagcgaatcgcggtttgttaccaagtacgtatttgacatcaacagttccaactggtggaacgattagatctcgcacactaagtattagcaccctaactcacgaccgagaatcaaaattctgctcagtagacgtctcctttcaggatgacaccattgtccccataggacacggacagaggagggcactggagagagtgtcaggtctttttctagctgtatcttcctctctccctctgctgcccataatgtgagtgaccctctagggtgagacttgcagggtgagctgctgaggaatgaagggccactgagatgtgtcctttagctgctgggtgtcatgtctgacctgctggtgcctagggcctgcttaacactcggcaaggctgcgagccgaggactgtgggaagccggacttgatgctttctaacctgcatatttgaatgccgaaggtcaaacaatccaagttacagataaataaaaaccgcattgcaagtattaaaaagccattctaggaaaattc

SEQ ID NO: 66 - protein, 2364 aa, Rattus norvegicus
  Microtubule associated protein IB
GQKILHHRSDVLETVVLINPSDEAVSTEVRLMITDAARHKLLVLTGQCFENTGELILQSGSFSFQNFIEIFTDQEIGELLSTTHPANKASLTLFCPEEGDWKNSNLDRHNLQDFINIKLNSASILPEMEGLSEFTEYLSESVEVPSPFDILEPPTSGGFLKLSKPCCYIFPGGRGDSALFAVNGFNMLINGGSERKSCFWKLIRHLDRVDSILLTHIGDDNLPGINSMLQRKIAELEEESQGSTSNSDWMKNLISPDLGVVFLNVPENLKNPEPNIKMKRSTEEACFTLQYLNKLSMKPEPLFRSVGNAIEPVILFQKMGVGKLKMYVLNPVKSSKEMQYFMQQWTGTNKDKAELILPNGQEVDIPISYLASVSSLIVWHPANPAEKIIRVLFPGNSTQYNILEGLEKLKHLDFLKQPLATQKDLTGQVSTPPVKQVKLKQRADSRESLKPATKPLSSKSVRKESKEEAPEATKASQVEKTPKVESKEKVIVKKDKPGKVESKPSVTEKEVPSKEEQSPVKAEVAEKAATESKPKVTKDKVVKKEIKTKPEEKKEEKPKKEVAKKEDKTPLKKDEKPKKEEAKKEIKKEIKKEEKKELKKEVKKETPLKDAKKEVKKDEKKEVKKEEKEPKKEIKKISKDIKKSTPLSDTKKPAALKPKVAKKEEPTKKEPIAAGKLKDKGKVKVIKKEGKTTEAAATAVGTAAVAAAAGVAASGPAKELEAERSLMSSPEDLTKDFEELKAEEIDVAKDIKPQLELIEDEEKLKETEPGEAYVIQKETEVSKGSAESPDEGITTTEGEGECEQTPEELEPVEKQGVDDIEKFEDEGAGFEESSEAGDYEEKAETEEAEEPEEDGEDNVSGSASKHSPTEDEEIAKAEADVHIKEKRESVASGDDRAEEDMDEALEKGEAEQSEEEGEEEEDKAEDAREEDHEPDKTEAEDYVMAVVDKAAEAGVTEDQYDFLGTPAKQPGVQSPSREPASSIHDETLPGGSESEATASDEENREDQPEEFTATSGYTQSTIEISSEPTPMDEMSTPRDVMTDETNNEETESPSQEFVNITKYESSLYSQEYSKPVVASFNGLSDGSKTDATDGRDYNASASTISPPSSMEEDKFSKSALRDAYRPEETDVKTGAELDIKDVSDERLSPAKSPSLSPSPPSPIEKTPLGERSVNFSLTPNEIKASAEGEATAVVSPGVTQAVVEEHCASPEEKTLEVVSPSQSVTGSAGHTPYYQSPTDEKSSHLPTEVTENAQAVPVSFEFTEAKDENERSSISPMDEPVPDSESPIEKVLSPLRSPPLIGSESAYEDFLSADDKALGRRSESPFEGKNGKQGFSDKESPVSDLTSDLYQDKQEEKRAGFIPIKEDFSPEKKASDAEIMSSQSALALDERKLGGDGSPTQVDVSQFGSFKEDTKMSISEGTVSDKSATPVDEGAEDTYSHMEGVASVSTASVATSSFPEPTTDDVSPSLHAEVGSPHSTEVDDSLSVSVVQTPTTFQETEMSPSKEECPRPMSISPPDFSPKTAKSRTPVQDHRSEQSSMSIEFGQESPEHSLAMDFSRQSPDHPTVGAGMLHITENGPTEVDYSPSDIQDSSLSHKIPPTEEPSYTQDNDLSELISVSQVEASPSTSSAHTPSQIASPLQEDTLSDVVPPRDMSLYASLASEKVQSLEGEKLSPKSDISPLTPRESSPTYSPGFSDSTSGAKESTAAYQTSSSPPIDAAAAEPYGFRSSMLFDTMQHHLALSRDLTTSSVEKDNGGKTPGDFNYAYQKPESTTESPDEEDYDYESHEKTIQAHDVGGYYYEKTERTIKSPCDSGYSYETIEKTTKTPEDGGYSCEITEKTTRTPEEGGYSYEISEKTTRTPEVSGYTYEKTERSRRLLDDISNGYDDTEDGGHTLGDCSYSYETTEKITSFPESESYSYETTTKTTRSPDTSAYCYETMEKITKTPQASTYSYETSDRCYTPERKSPSEARQDVDLCLVSSCEFKHPKTELSPSFINPNPLEWFAGEEPTEESERPLTQSGGAPPPSGGKQQGRQCDETPPTSVSESAPSQTDSDVPPETEECPSITADANLDSEDESETIPTDKTVTYKHMDPPPAPMQDRSPSPRHPDVSMVDPEALAIEQNLGKALKKDLKEKAKTKKPGTKTKSSSPVKKGDGKSKPSAASPKPGALKESSDKVSRVASPKKKESVEKAMKTTTTPEVKATRGEEKDKETKNAANASASKSVKTATAGPGTTKTAKSSTVPPGLPVYLDLCYIPNHSNSKNVDVEFFKRVRSSYYVVSGNDPAAEEPSRAVLDALLEGKAQWGSNMQVTLIPTHDSEVMREWYQETHEKQQDLNIMVLASSSTVVMQDESFPACKIEL

SEQ ID NO: 67 - DNA, 7095 bp, Rattus norvegicus
  Microtubule associated protein IB
ggacaaaagatcctccatcaccgaagtgacgtcttagaaactgtagttctgatcaacccttcggatgaagcagtcagcaccgaggtgcgtttgatgatcactgacgccgcccgccataaactgctggtgctcaccggacagtgctttgagaacactggagagctcatcctccagtcaggctctttctccttccagaacttcatagagattttcaccgaccaagagattggggagctcctaagcaccacccatcctgccaacaaagccagcctcaccctcttctgccctgaggaaggagactggaagaactccaaccttgacagacacaatctccaagacttcatcaacatcaagctcaactcagcttctatcttgccagaaatggagggactttctgagttcaccgagtacctctcggagtctgtcgaagtcccctccccctttgacatcctggagcccccgacctcgggcggatttctgaagctctccaagccttgttgttacatttttccggggggccgcggggactctgccctgttcgcagtgaacggattcaacatgctcattaacggaggatcagaaagaaagtcctgcttctggaagctcattcggcacttggaccgggtggactccatcctgctcacccacattggggatgacaacttgcccgggatcaacagcatgttgcaacgcaagattgcagagctggaagaggagtcccagggctccaccagcaacagtgactggatgaaaaacctcatctcccctgacttgggggttgtgtttctcaatgtacctgaaaatctgaaaaacccagaacccaacatcaagatgaagagaagtacagaagaagcatgcttcaccctccagtacctaaacaaactgtccatgaaaccagagcctttatttagaagtgtaggcaatgccattgagcctgtcatcctgttccaaaaaatgggagtgggtaaactgaagatgtatgtgcttaacccagtcaaaagcagcaaggaaatgcagtatttcatgcagcagtggactggaaccaacaaagacaaggctgaacttatcctgcccaatggtcaagaagtagacatcccgatttcctacctagcttccgtctcgtctttgattgtgtggcacccagccaaccctgctgagaaaatcatccgggttctgtttcctggaaacagcacccagtacaacatcctagaagggctggaaaaactcaaacatctagacttcctaaagcagccactggccacccaaaaagatctcactggccaggtgtccacccccccagtgaaacaggtcaagttgaaacagcgggctgacagccgagagagtctgaagccagccacaaaaccactttccagtaaatcagtgaggaaggagtccaaagaggaggcccctgaagccacaaaagccagccaagtggaaaaaacacccaaagttgaaagcaaagagaaagtgatagtgaaaaaagacaagccaggaaaggtagaaagtaagccatcggtgacggaaaaggaggtgcccagcaaagaggagcagtcgcccgtcaaagctgaggtggctgagaaggcggccacggagagcaaacccaaagtcaccaaagacaaagtggtaaaaaaggaaataaagacaaaacccgaagaaaagaaagaggagaagcccaagaaggaagtggctaaaaaggaagacaaaactcccctcaagaaagacgagaagcccaaaaaggaagaggcgaagaaggagatcaagaaagaaatcaaaaaggaagagaaaaaggagctgaagaaagaggtgaagaaggaaacgcccctgaaggacgccaagaaggaggtgaagaaagacgagaagaaagaagttaaaaaggaagagaaggaacccaaaaaggagattaagaagatctccaaggacataaagaaatccactcctctgtcagacacaaagaaaccggctgcattgaaaccaaaagtagcaaagaaagaagagcccaccaagaaggagcctattgctgctgggaaactcaaggacaaggggaaggtcaaagtcattaaaaaggaaggcaagaccacagaggccgctgccacagctgttggcactgctgccgtggctgcagcagccggagtagcggccagcggtcctgccaaagaacttgaagctgagcggtccctcatgtcgtcccctgaggatctaaccaaggactttgaggagctaaaggctgaggagatcgatgtagcgaaggacatcaagcctcagctggagctcattgaagatgaagagaaactgaaggaaaccgagccgggagaagcctacgtcattcagaaagagacggaagtcagcaaaggttctgctgagtcacctgatgaagggatcaccaccactgagggggaaggggagtgcgagcaaacccccgaggagctggagccagttgagaagcagggcgtggatgacatcgagaagttcgaggatgaaggcgctggttttgaagaatcctcagaggccggagactacgaagagaaggcagaaactgaggaggccgaggagccggaagaagacggggaagacaatgtgagcgggagcgcctcgaagcacagccccacagaagacgaagaaatcgctaaggctgaggcggacgtacacatcaaggagaagagggagtctgtggccagcggcgatgaccgggccgaagaagacatggatgaagcgcttgagaaaggagaagctgaacagtctgaggaggagggtgaggaggaggaggacaaagcagaggacgccagagaggaagaccatgagcccgacaaaactgaggctgaagattatgtgatggctgtggttgacaaggccgcggaggccggagtcaccgaggatcagtatgatttcctggggacaccggccaagcaacctggagtccagtctcctagccgagaacccgcgtcttcaattcatgatgagaccctacccggaggctccgagagcgaggccactgcttcagatgaggagaatcgagaagaccagcctgaggaattcactgctacctccggatatactcagtccaccatcgagatatctagtgagccgactccaatggatgagatgtccactcctcgagatgtgatgaccgacgagaccaacaatgaggagacagagtccccgtctcaggagttcgtgaacattaccaaatacgagtcttcgctgtactctcaggagtactccaaacctgtggttgcatcattcaatggattgtcagacgggtcaaagacagacgccactgacggtagggattacaacgcttccgcctccaccatatcaccaccttcgtccatggaagaagacaaattcagcaagtctgctcttcgtgacgcttaccgcccagaagagacggacgtgaaaaccggtgccgagttggacatcaaagatgtttcggatgagagacttagcccagccaagagtccatccctgagtccttctccaccatcacccatagagaagactcccctgggtgaacgtagcgtgaatttctctctgacacccaacgagatcaaagcctctgcagagggagaggcaacagcagtagtgtcccccggagtgacccaagcagtagttgaagaacactgtgccagtcctgaggagaagaccttggaggtagtgtcaccgtctcagtctgtgacaggcagtgcgggccacacaccttactaccaatctcccaccgacgaaaagtccagtcacctacctacagaagtcactgagaacgcgcaggcagtcccggtgagctttgaattcactgaggccaaagatgagaacgagaggtcgtccatcagccccatggatgaacctgtgcctgactcagagtctcctatcgagaaagttctgtctccgttacgcagccctccccttattggatccgagtccgcatatgaagacttcctgagtgcggatgacaaggctcttggcagacgttcagaaagcccctttgaagggaagaatggaaagcaaggcttctcagacaaagaaagcccagtttctgacctgacttccgatctttaccaagacaagcaggaagagaaaagggcgggcttcataccgataaaggaagactttagtccagaaaagaaagccagcgatgctgaaatcatgagttctcaatcagctctggctttggatgaaaggaaactgggaggagatggatctccaacgcaagtagatgtcagtcagtttggctctttcaaagaagacaccaagatgtccatttcggaaggcaccgtttcagacaagtccgccacgcctgtggatgagggcgcggaagacacctattcacacatggaaggtgtggcctcagtgtcaacggcctctgtggctaccagctcgtttccagagccaaccacagatgacgtgtctccttctctccacgctgaagtgggctctccacattccacagaggtggatgactccctgtcggtgtcggtggtgcaaacaccaactactttccaggaaacagaaatgtctccgtctaaagaagagtgcccaagaccaatgtcgatttctcctcctgacttctcccctaagacagccaaatccaggacaccagttcaagatcaccgatccgaacagtcttcaatgtctattgaattcggtcaggaatcccccgagcattctcttgctatggactttagtcggcagtctccagaccaccctactgtgggtgctggtatgcttcacatcaccgaaaatgggccaactgaggtggactacagtccctccgatatccaggactctagtttgtcacataagattcctcctacagaagagccatcctacacccaggataatgatctgtccgagctcatctctgtgtctcaggtggaggcttccccatccacctcttctgctcacactccttctcagatagcctctcctcttcaggaagacactctctctgatgtcgttcctcccagagatatgtccttatatgcctcgcttgcgtctgagaaagtgcagagcctggaaggagagaaactctctccaaaatccgatatttctccgctcacccctcgagagtcctcacctacatattcacctggcttttcagattctacctctggagctaaagagagtacagcggcttaccaaacctcctcttccccaccaatagatgcagcagccgcagagccctacggcttccgctcctcaatgttatttgatacaatgcagcatcacctggccttgagtagagatttgaccacatctagtgtggagaaggacaatggagggaagacacccggtgactttaactatgcctatcaaaagcccgagagcaccaccgaatccccagatgaagaagattatgactatgaatctcacgagaaaaccatccaggcccacgatgtgggtggttactactatgagaagacagagagaaccataaaatccccatgtgacagtggatactcctatgagaccattgagaagaccaccaagaccccagaagatggtggctactcctgtgaaattaccgagaaaaccactcggacccctgaagagggcgggtactcgtatgagatcagcgagaagacaacacgaacccctgaagtaagtggctacacctatgagaagaccgagaggtccagaaggctcctcgatgacattagcaatggctacgatgacactgaggatggtggccacacacttggcgactgtagctattcctacgaaaccactgagaaaattaccagctttcctgaatctgaaagctattcctatgagacaactacaaaaacaacacggagtccagacacctctgcatactgttacgagaccatggagaagatcaccaagaccccacaggcatccacatactcctatgagacctcagaccgatgctacactccagaaaggaagtccccctcggaggcacgccaggatgttgacttgtgtctggtgtcctcctgtgaattcaagcatcccaagaccgagctctcaccttccttcattaatccaaaccctctcgagtggtttgctggggaagagcccactgaagaatctgagaggcctctcactcagtctggaggagcccccccaccttcaggaggaaaacaacagggcagacaatgcgatgaaactccacccacctcagtcagtgagtcagctccatcccagacggactctgatgttcccccagagacagaagagtgcccctccatcacagctgatgccaaccttgactctgaagatgagtcagaaaccatccccacagacaaaacggttacgtacaaacacatggacccgcctccagcccccatgcaagaccgaagcccttctcctcgccaccctgatgtgtccatggtggatccagaggccttggctattgagcagaacctaggcaaggctctgaaaaaggatctgaaggagaaggccaagaccaagaaaccaggcacaaagaccaagtcctcttcacctgtcaaaaagggtgatgggaagtccaagccttcagcagcttcccccaaaccaggagccttgaaggaatcctctgacaaggtgtccagagtggcttctcccaagaagaaagagtctgtggagaaagctatgaagaccaccaccactcctgaggtcaaagccacacgaggggaagagaaggacaaggaaactaagaatgcagccaatgcttctgcatccaagtcagtgaagactgcaacagcaggaccaggaaccactaagacggccaagtcgtccaccgtgcctcccggcctccctgtgtatttggacctctgctatattcccaaccacagcaacagtaagaatgtcgatgttgagtttttcaagagagtgaggtcatcttactacgtggtgagtgggaacgaccctgccgcggaggagcccagccgggctgtcctggatgccttgttggaagggaaggctcagtggggaagcaacatgcaggtgactctgatcccaacacatgactctgaggtgatgagggagtggtaccaggagacccacgagaagcagcaagacctcaacatcatggtcctagcaagcagtagtacagtggtcatgcaagacgagtccttccctgcatgcaagatagaactgtag

SEQ ID NO: 68 - protein, 379 aa, Rattus norvegicus
  cdc 37 homolog
MVDYSVWDHIEVSDDEDETHPNIDTASLFRWRHQARVERMEQFQKEKEELDRGCRECKRKVAEFQRKLKELEVAEGGGQVELERLRAEAQQLRKEERTGSRSWRTCGKKEKNMPWNVDTLSKDGFSKSMVNTKPEKAEEDSEEAREQKHKTFVEKYEKQIKHFGMLHRWDDSQKYLSDNVHLVCEETANYLVIWCIDLEVEEKCALMEQVAHQTMVMQFILELAKSLKVDPRACFRQFFTKIKTADQQYMEGFKYELEAFKERVRGRAKLRIEKAMKEYEEEERKKRLGPGGLDPVEVYESLPEELQKCFDVKDVQMLQDAISKMDPTDAKYHMQRCIDSGLWVPNSKSGEAKEGEEAGPGDPLLEAVPKAGFEKDISA

SEQ ID NO: 69 - DNA, 1604 bp, Rattus norvegicus
  cdc 37 homolog
cgagcggactctctagcccttgtgggtccctgttcagcgcccggccgtgtggagacccgggagccagtccgggcctccgaggcgaagatggtggactacagcgtttgggatcacatcgaggtgtcggacgatgaggacgagacgcaccccaacatagacacggccagcctgttccgctggcggcaccaggcccgggtggaacgcatggaacagtttcagaaggagaaagaagaactggaccggggctgccgggaatgcaagcgcaaggtagccgagttccagcgcaagctgaaggaactggaagtggctgagggtggtggccaggtggagcttgagcggcttcgagcggaggcacagcagctgcgcaaggaggagcgaactgggagcagaagctggaggacatgcggcaaaaaggagaagaacatgccctggaatgtggacacactcagcaaagatggcttcagcaagagcatggtcaataccaagcctgagaaggcagaggaagactcagaggaggcaagggagcagaaacacaagaccttcgttgaaaaatacgagaaacagatcaaacatttcggcatgctccaccgctgggatgacagccagaagtacctgtcagacaatgtccacctggtgtgcgaggagacagccaactacctggttatctggtgcattgacctggaagtcgaggagaagtgtgccctgatggagcaggtggctcaccagaccatggtcatgcagttcatcctggagttggccaagagtctgaaggtcgacccccgggcctgcttccggcagtttttcaccaagatcaagactgctgaccagcagtacatggagggcttcaagtatgaactggaggcctttaaggagcgtgtgcggggccgcgccaagctgcgaatagagaaggccatgaaggagtacgaggaggaggagcgcaagaagaggctaggccctggtggcctggaccctgtggaggtctacgaatctctgcctgaggagctgcagaagtgctttgatgtgaaggatgtacagatgctgcaagacgccatcagcaagatggatcctactgatgccaagtatcacatgcagcgttgcatcgattctggcctctgggtccccaactccaagtctggtgaggccaaggagggggaggaggcgggccccggggacccattgctggaagccgtccccaaagcgggcttcgagaaagacatcagtgcgtgacccttctccagaaaaggaaacccagccaccatgtccctagctcccgacttcttcacttctgctgctcctcacgtcctggcccatggcctgccccatgtcccctctcctccggcctgtccccagcactcatcagattctctgcttcgggttgaggggctctgctgtctgcagcccccccacccccatcagcgttatgccaaagtcctgggggtctggggaggggcagcagctgccaggttggtccaccgggttggggatacacatgtccccatctgggcagggtctgtccctatgggtctatggtcactgtttctcttctgtctagctttctgtcctgcaactggcaacaatgtccaataaacagttccaggtgctgg

SEQ ID NO: 70 - protein, 216 aa, Homo sapiens
  Rab5c-like protein
MAGRGGARRPNGPAAGNKICQFKLVLLGESAVGKSSLVLRFVKGQFHEYQESTIGAAFLTQTVCLDDTTVKFEIWDTAGQERYHSLAPMYYRGAQAAIVVYDITNTDTFARAKNWVKELQRQASPNIVIALAGNKADLASKRAVEFQEAQAYADDNSLLFMETSAKTAMNVNEIFMAIAKKLPKNEPQNATGAPGRNRGVDLQENNPASRSQCCSN

SEQ ID NO: 71 - DNA, 1529 bp, Homo sapiens
  Rab5c-like protein
cttacactaagtgcctctttgcatagcaccagtccccacccgcacgctctctggaccactacagctggacgggcaatggcgggtcggggaggcgcacgacgacccaatggaccagctgctgggaacaagatctgtcaatttaagctggttctgctgggggagtctgcggtaggcaaatccagcctcgtcctccgctttgtcaagggacagtttcacgagtaccaggagagcacaattggagcggccttcctcacacagactgtctgcctggatgacacaacagtcaagtttgagatctgggacacagctggacaggagcggtatcacagcctggcccccatgtactatcggggggcccaggctgccatcgtggtctatgacatcaccaacacagatacatttgcacgggccaagaactgggtgaaggagctacagaggcaggccagccccaacatcgtcattgcactcgcgggtaacaaggcagacctggccagcaagagagccgtggaattccaggaagcacaagcctatgcagacgacaacagtttgctgttcatggagacatcagcaaagactgcaatgaacgtgaacgaaatcttcatggcaatagctaagaagcttcccaagaacgagccccagaatgcaactggtgctccaggccgaaaccgaggtgtggacctccaggagaacaacccagccagccggagccagtgctgcagcaactgagccccccttgcctgcccgctgcccccgcctcctccgcctgaatgacccgactggaatccactctaaccaatcgcacttaacgactcgggccaccactgggggggcagggggaggggtccaccatgatttctccatataattttgatcataggccggagtgagtcattccacctgcacctttctgtacaaatactaattcaattttaagtcttaagtcacttttttaatatatatgatcttctgctcttcccacttcctcccctttctactgctctcccattttcccttgctgggagtagccacatgctcttgccccccaacccttgtatatggggacagtggggtcagtgcagctaccctttctttccctctgcggaacagcggacccagcaagagcatccacatcctcactttgttcggagtggtctttggtttgggcggtggggcagaccttgggaaggggcttaggaagggagaggcagctcttccttcagctggctctcatcaggctgcagccccctccccgctcccacctccctgctgggaaaccacagcattatcacagcattattgtgacagccacgaacccattgcccacaacccctccaccctcggtcaccccaacctctggctctgagccctgttctgaccaaatcatgatgatgagtatttgggggtgggtgggtaagggggggagtgggaggggacggaaccaactttttctgtattttgtattgtatgttttcttcaacatgtaaccaatcagtatcttgtcaatatagtcagccgatcgatcgacctca

SEQ ID NO: 72 - protein, 731 aa, Mus musculus
  Gelsolin
MVVEHPEFLKAGKEPGLQIWRVEKFDLVPVPPNLYGDFFTGDAYVILKTVQLRNGNLQYDLHYWLGNECSQDESGAAAIFTVQLDDYLNGRAVQHREVQGFESSTFSGYFKSGLKYKKGGVASGFKHVVPNEVVVQRLFQVKGRRVVRATEVPVSWDSFNNGDCFILDLGNNIYQWCGSGSNKFERLKATQVSKGIRDNERSGRAQVHVSEEETEPEAMLQVLGPKPALPEGTEDTAKEDAANRKLAKLYKVSNGAGSMSVSLVADENPFAQGPLRSEDCFILDHGRDGKIFVWKGKQANMEERKAALKTASDFISKMQYPRQTQVSVLPEGGETPLFKQFFKNWRDPDQTDGPGLGYLSSHIANVERVPFDAGTLHTSTAMAAQHGMDDDGTGQKQIWRIEGSNKVPVDPATYGQFYGGDSYIILYNYRHGGRQGQIIYNWQGAQSTQDEVAASAILTAQLDEELGGTPVQSRVVQGKEPAHLMSLFGGKPMIIYKGGTSRDGGQTAPASIRLFQVRASSSGATRAVEVMPKSGALNSNDAFVLKTPSAAYLWVGAGASEAEKTAAQELLKVLRSQHVQVEEGSEPDGFWEALGGKTSYRTSPRLKDKKMDAHPPRLFACSNRIGRFVIEEVPGELMQEDLATDDVMLLDTWDQVFVWVGKDSQEEEKTEALTSAKRYIETDPANRDRRTPITVVRQGFEPPSFVGWFLGWDNNYWSVDPLDRALAELAA

SEQ ID NO: 73 - DNA, 2447 bp, Mus musculus
  Gelsolin
tgagcgcggcccagcactatggtggtggagcaccccgaattcctgaaggcagggaaggagcctggcctgcagatctggcgtgtggagaagtttgacctggtgcctgtgccccccaacctctatggagacttcttcacgggtgatgcctatgtcatcctgaagactgtgcagctgaggaatgggaatctgcagtatgacctccactattggctgggcaatgaatgcagccaggatgagagcggggctgctgccatctttactgtgcaactggatgactacctgaacggccgggctgtgcagcaccgtgaggttcagggctttgagtcgtccaccttctccggctacttcaagtctggacttaagtacaagaaaggaggtgtggcatctggattcaaacacgtggtacccaatgaggtggtggtccagaggctcttccaggtcaaaggacgccgtgtagtccgtgctactgaggtacctgtgtcctgggacagtttcaacaatggcgactgcttcattctggacctgggaaacaatatctatcagtggtgtggctctggcagcaacaaatttgaaaggctgaaggccacacaggtgtccaagggcatccgggacaacgagaggagtggccgtgctcaagtacacgtgtctgaagaggagactgagcccgaggcgatgctgcaggtgctgggccccaagccggctctgcctgaaggtaccgaggacacagccaaggaagatgcagccaaccgcaagctggccaagctctacaaggtctccaacggtgcaggtagcatgtcagtctccctagtggctgatgagaaccccttcgcccaggggcccctgagatctgaggactgcttcatcctggaccatggcagagatgggaaaatctttgtttggaaaggcaagcaggccaacatggaggagcggaaggctgccctcaaaacagcctctgacttcatctccaagatgcagtaccccaggcagacccaggtttcagttctcccagagggcggtgagacccctctctttaagcagttcttcaagaactggcgggacccagaccagacagatggccccggcctgggctacctctccagccacattgccaacgtggagcgcgtacctttcgatgccggcacgctgcacacctccaccgccatggccgctcagcacggcatggatgatgatggaactggccagaaacagatctggagaattgaaggttccaacaaggtgccagtggaccctgccacatacggacagttctatggaggcgacagctacatcattctgtacaactaccgccacggtggccgccagggacagatcatctacaactggcagggtgctcagtctacccaggatgaggttgctgcttctgccatcctgactgcccagctggatgaggagctgggaggaactcctgtccagagccgagtggtccaaggcaaagagcctgcacacctcatgagcttgtttggcgggaagcccatgatcatctacaagggtggcacctcccgtgatggtgggcagacagctcctgccagtatccgcctcttccaagtgcgtgccagcagctctggagccaccagggctgtggaggtgatgcctaagtctggtgctctgaactccaacgatgcctttgtgctgaaaaccccctccgctgcctacctgtgggtgggcgcaggagccagtgaggcagagaagacggcggcccaggagcttctgaaggtccttcggtcccagcatgtgcaggtggaagaaggcagtgagccagatggcttctgggaggctctgggcgggaagacgtcctaccgcacatcccccaggcttaaggacaagaagatggatgcccatcctcctcgactctttgcctgctccaacaggatcggacgctttgtgatcgaagaggttcctggcgagcttatgcaggaagacctggctactgatgacgtcatgctcctggacacctgggaccaggtctttgtctgggttggaaaagactcccaggaagaagaaaagacggaagccttgacttctgctaagcggtacatcgagacagatccagcaaatcgggacaggcggacccccatcacagtcgttaggcagggctttgagcctccttccttcgtgggctggttcctcggctgggacaacaactactggtcggtggatcctttggaccgggccttggctgagctggctgcctgagtaaggaccaagccatcaatgtcaccaatcagtgcctttgagggttgtccatctcccaaagacatcatatggcaagcaggaaaactatgatgtgtgcgcgcgtgtttttgtttttgttttttacggtagccaaaacaagcccttgtggaaactcagggtctttacagaattgcttcaaatgtctgtactttggaaatgaaagccaataaaagctttttgaagtgaaaaaaaaa

SEQ ID NO: 74 - protein, 236 aa, Rattus norvegicus
  Cd81 antigen
MGVEGCTKCIKYLLFVFNFVFWLAGGVILGVALWLRHDPQTTTLLYLELGDKPAPSTFYVGIYILIAVGAVMMFVGFLGCYGAIQESQCLLGTFFTCLVILFACEVAAGIWGFVNKDQIAKDVKQFYDQALQQAVMDDDANNAKAVVKTFHETLNCCGSNTLTTLTTAVLRNSLCPSSSNSFTQLLKEDCHQKIDELFSGKLYLIGIAAIVVAVIMIFEMILSMVLCCGIRNSSVY

SEQ ID NO: 75 - DNA, 1303 bp, Rattus norvegicus
  Cd81 antigen
gtggcggtctgagcgagcgcgcccttgcttcaaagcggtagcgactctagcgcctccggctaggctccaacccttctctaccctacgtctcaatagccacaccgcagtctccggcctcaagcgacccaggctatcttccagtcccgggactccgggtactgcggtccccagaaccgcccgtccagggaccaacccaagttccgcaggccgcggcggccgccaatgggggtggagggctgcaccaaatgcatcaaatacctgctcttcgtcttcaatttcgtcttctggctggctggaggtgtgatcctaggtgtagctctgtggttgcgccatgatccacagaccaccaccttgctctacctggaactgggagacaaaccagcacctagcaccttctatgtgggcatctacattctcattgctgtgggagctgtgatgatgtttgtaggcttcctggggtgctatggggccatccaggagtcccagtgcctgctggggacgttcttcacttgccttgtgatcctgtttgcctgtgaggtagctgcgggcatctggggctttgtaaacaaagaccagatcgccaaggatgtgaagcagttctacgaccaggcccttcagcaggctgtgatggatgatgatgccaacaatgccaaggcagtggtgaagaccttccatgagacgctcaactgttgtggctccaatacgctgaccacactgaccaccgccgtgctgaggaacagcctgtgtccctcaagcagcaactcattcactcagcttctgaaggaagactgccatcagaaaatcgatgagctcttttctgggaagctgtacctcattggaattgcagccattgtggtagctgtcattatgatcttcgagatgattctgagcatggtgctgtgctgtggcatccggaacagctccgtgtactgaggcccttcgcttcgcaccagagggtccctggaatgaccagagggcaccttggggggcatggcctgtgtatatactatttctgtattactctgctacacttagcctttttacttttgagtcttttttgttctgaacttttcctgtcaccttttggagccgacaccacacataggtggcgtatgtggggacgtagaggtggagctggccctagctggcagggccctgtacttctgggacccctggagagttctgcctgctgagccaaacctcctccatagctacttgtccagaggccacatagccaagctagaaggccatgtccacccactctgcccactgtgggtcacatcgctcccatctttttaatccg

SEQ ID NO: 76 - protein, 81 aa, Rattus norvegicus
  MOBP-81 protein
MSQKVAKEGPRLSKNQKFSEHFSIHCCPPFTFLNSKREIVDRKYSICKSGCFYQKKEEDWICCACQKTRLRRRSRSTPRKK

SEQ ID NO: 77 - DNA, 2405 bp, Rattus norvegicus
  MOBP-81 protein
ctttcacagcagccaatacctgcagggcaacaaagaatcaaatgagagcgagacaagctgggaatgaagtcctggttgccagatgggagcttgaaaacacagtaagatgagtcaaaaagtggccaaggagggccccaggctctccaagaaccagaagttctcagagcacttcagcatccactgctgcccacccttcaccttcctcaactccaagcgtgagatcgtggaccgcaagtacagcatctgcaagagcggttgcttttaccagaagaaggaggaggactggatctgctgtgcctgccagaagaccagattgagaaggaggagcaggtcaaccccaagaaaaaagtgaccgagaagggacttctcgtgaatggacagcctctgcttgtggatttactgcttcaccgcaacccatctgccctcagacttatctggcctcgagtatgacgcaggggtggacacctgctgatgtaacaactacagatgccctcagtccccatggtgtgggagccttagggcagcctgcctggagcacttggacgatgccactccatcacctccctctataaccgccgaagaggcatctctgtccaccttctgaccctccatggttgttcagtctgaagattcctgtgtcttcagggatgtgcaaaacaaggtcatggtgtgcgaccatccaaatggcaaatataaaaaagaaattgtcaagtgttggcggtgacgcaagaaaaatcctagaagccacagatggggatttatacactgtcgcaacaccgttgagatgtactctggggacactatcaaggcaaagtatcttaaaacctctaatcaagcgactgtaattttaggcatccatcctggggaaaactcacacccttttagagaaaggagaaatgcccaaatttgtttaaaagatcgtggtttgtagttaagagaggtagaatgagattaaccaaaatgtcggtccacgggagagcaagagagtcaatcttggtatgtccacaaagtcctaagagcagttaaaagccaatctgtcagacagacccatgccgagcaggtaaatctagaaagcaaaccaaggactataaaagtcaagcaccaggcagtaaatccagaataagagcctatgcatacatattgacagctcacaatcctgtactggttattgcttacataaggagaagccacagatctcattctttcttctagaatgcaatagaatttaaaaatgacagcagaacaaaaggacactagaaattatcctctcagaaataaaagtataagaaataatttctggcttgaggaaataacccaggctaaaagctacacaggcctggcttaaccataccagcagggcatacctttgttaggagagggtcaattcgaagtttgtgaacagcctgggctgtttccgaggctgcataaatcttaaacaccaaaaatgtgtgttttgaattaactttgggtctggtctacagagtccggcttgtgatggctcctgacacgggagaaaaggggataagttaatataaggctctgggattttcctttgaggaacaggcaggtaggaaaggacactagaatgttggggaatgtggagggatgtaaaacagacagtattatgatggggggaactcaggaaggaacggtaaggacaccagaatggcctcgtgctgggagccagggtggccaagcccaggcagatgcacaaagaccccaaaggccaccattgcctcacaaccagacccagcaaagttcagatcaagtgcataaatgggggtcacgtcagtggaaactgtccggagcatggagcagggggtgaggaagacagtcagaaggaagcaagagatggcttagagtgttgcatggcagcaagaagaagcagtccaaatatcatcccaaggcagccatggcttcctctcccgaggacactgtggccctgtggaagtgccaaggcagaggcagaggaatttaaccgcacacaaactacatgcagaaactaaggtgctaaatgaacatctacgtgacaacctttctgaggaccaggttccagtgagcggcccagggccgttatcttcgcgatggtaactgagctgagaagaatgggaaccggataggagcacacagtagcccactggtgagacaaatccacgggcgagcctcaaccaacccactaaggaaacttcacgccttttcatctagttttcattttggcaaagcaaagccatcctgagtgcttgcttgtctccgccctccacgccacccccgagacacagagcatgcgcattaacccagagcgcgctaacacatgcacagcgttggacattagcctattaatgggaataaccagaaactactaaattttgtttgttgtagaaataaaacctggggatggatcaaaa

SEQ ID NO: 78 - protein, 590 aa, Homo sapiens
  Syntaxin binding protein 2
MAPSGLKAVVGEKILSGVIRSVKKDGEWKVLIMDHPSMRILSSCCKMSDILAEGITIVEDINKRREPIPSLEAIYLLSPTEKALIKDFQGTPTFTYKAAHIFFTDTCPEPLFSELGRSRLAKVVKTLKEIHLAFLPYEAQVFSLDAPHSTYNLYCPFRAEERTRQLEVLAQQIATLCATLQEYPAIRYRKGPEDTAQLAHAVLAKLNAFKADTPSLGEGPEKTRSQLLIMDRAADPVSPLLHELTFQAMAYDLLDIEQDTYRYETTGLSEAREKAVLLDEDDDLWVELRHMHIADVSKKVTELLRTFCESKRLTTDKANIKDLSQILKKMPQYQKELNKYSTHLHLADDCMKHFKGSVEKLCSVEQDLAMGSDAEGEKIKDSMKLIVPVLLDAAVPAYDKIRVLLLYILLRNGVSEENLAKLIQHANVQAHSSLIRNLEQLGGTVTNPGGSGTSSRLEPRERMEPTYQLSRWTPVIKDVMEDAVEDRLDRNLWPFVSDPAPTASSQAAVSARFGHWHKNKAGIEARAGPRLIVYVMGGVAMSEMRAAYEVTRATEGKWEVLIGSSHILTPTRFLDDLKALDKKLEDIALP

SEQ ID NO: 79 - DNA, 1888 bp, Homo sapiens
  Syntaxin binding protein 2
ggcacgaggggcggcggcgcccctcggggaagatggcgccctcggggctgaaggcggtggtgggggaaaaaattctgagcggagttattcggagtgtcaagaaggatggggagtggaaggtgcttatcatggatcacccaagcatgcgcatcttgtcttcctgctgcaaaatgtcagatatcctggctgagggcatcaccattgttgaagacatcaacaaacggcgggaacccattcccagtctggaggccatttatttgctgagccccacggagaaggccctgatcaaagacttccaggggaccccgactttcacctacaaagcggcccatatcttcttcaccgacacctgccccgagcccctgttcagtgagctaggccgctctcgtctggcaaaggtggtgaagacgttgaaggagattcaccttgccttcctcccctacgaggcccaggtgttctccctcgatgctccccacagcacctacaacctctactgccccttccgggcagaggagcgcacgcggcagctcgaggtgctggcccagcagattgccacgctgtgcgccaccctgcaggagtacccggccatccgctaccgcaagggcccagaggacacagcccagttggcccacgccgtcctggccaagctgaacgccttcaaggcagacactcccagtctgggcgagggcccagagaaaacccgctcccagctgctgataatggaccgggcagctgaccccgtgtccccactactgcatgagctcacgttccaggccatggcgtatgatctgctggacatagagcaggacacatacaggtatgagaccaccgggctgagcgaggcgcgggagaaggccgtcttgctggacgaggacgatgacttgtgggtggagcttcgccacatgcatatcgcagatgtgtccaagaaggtcacggagctcctgaggaccttctgtgagagcaagaggctgaccacggacaaggcgaacatcaaagacctatcccagatcctgaaaaagatgccgcagtaccagaaggagctgaataagtattctacgcacctgcatctagcagatgattgtatgaagcacttcaagggctcggtggagaagctgtgtagtgtggagcaggacctggccatgggctccgacgcagagggggagaagatcaaggactccatgaagctgatcgttccggtgctgctggacgcggcggtgcccgcctacgacaagatccgggtcctgctgctctacatcctccttcggaatggtgtgagtgaggagaacctggccaagctgatccagcatgccaatgtacaggcgcacagcagcctcatccgtaacctggagcagctgggaggcactgtcaccaaccccgggggctcggggacctccagccggctggagccgagagaacgcatggagcccacctatcagctgtcccgctggaccccggtcatcaaggatgtaatggaggacgccgtggaggaccggctggacaggaacctgtggcccttcgtatccgaccccgcccccacggccagctcccaggccgctgtcagtgcccgcttcggtcactggcacaagaacaaggctggcatagaagcccgggcgggcccccggctcatcgtgtatgtcatgggcggtgtggccatgtcagagatgagggccgcctacgaggtgaccagggccaccgagggcaagtgggaggtgctcattggctcctcacacatcctcaccccgacccgcttcctggatgacctgaaggcactggacaagaagctggaggacattgccctgccctgacccctggccccgccccctacccctccctttccagagaaataaactcttcccgtcgctctgccaaaaaaaaaaaaaaaaaaaaa

SEQ ID NO: 80 - protein, 505 aa, Rattus norvegicus
  Alpha-internexin
MSFGSEHYLCSASSYRKVFGDGSRLSARLSGPGASGSFRSQSLSRSNVASTAACSSASSLGLGLAYRRLPASDGLDLSQAAARTNEYKIIRTNEKEQLQGLNDRFAVFIEKVHQLETQNRALEAELAALRQRHAEPSRVGELFQRELRELRAQLEEASSARAQALLERDGLAEEVQRLRARCEEESRGREGAERALKAQQRDVDGATLARLDLEKKVESLLDELAFVRQVHDEEVAELLATLQASSQAAAEVDVAVAKPDLTSALREIRAQYESLAAKNLQSAEEWYKSKFANLNEQAARSTEAIRASREEIHEYRRQLQARTIEIEGLRGANESLERQILELEERHSAEVAGYQDSIGQLESDLRNTKSEMARHLREYQDLLNVKMALDIEIAAYRKLLEGEETRFSTSGLSISGLNPLPNPSYLLPPRILSSTTSKVSSAGLSLKKEEEEEEEEEEGASKEVTKKTSKVGESFEETLEETVVSTKKTEKSTIEEITTSSSQKM

SEQ ID NO: 81 - DNA, 4593 bp, Rattus norvegicus
  Alpha-internexin
gaattctgaactacaaagcaaagcgcttcacatttccctgacttgtgccataggctggaaggtaaactcagacaactgagccatccattgttcaggctctactctgtccttaccgggtgtggcgagggtggccagttctgtcagccgtgctcctgcttcactcctggaattgaatcaaagactaatccccacagaaaatggttctcccatctaattcaaaatggaatgtcaaacgcatacagggccagacagatgaaaagacgaggtggaaagggtagctctggagtgctgggagctgtgaggaacaccgcacttaaacaaaggcactcacttccgccctctgcagtcatcctaaaccttctactttcaagagtggcaggggctgggcattaaaaggtcaaaatcaatccagtctctttttagatgaaacctcctgactaaaaattcattaaaaaaaaacattttgaaatctattgttttgttggtttgggtctttttgtttaaaccaaagcctgcagactcctttctaccagcctgcggtcctgggatgcgcggttgattggaggtctagagatgaaccctgctaattctcaacgctttgctccttttccaggcctagcctctttccctccccgtaacccttcgcacccttggaaataagcctcttgttcttcaccaatgtccggagcatctcacgctctctgcgaatcctcccgtttactgtggctgcggagctgagagatgctctctgaagtggacccactttctccgaaaaggaccaggagacacctaagtccagaagataaagaggagggtgcttctccccctgaaaagggtggggacagggcctcacccttgggaactctgggatgcagatgagaacagagacacctttcagaaaaacctactctccttgggggataagcacagaaaaggaaggacaggctctaaagccatcaaagagctgagtgctctgggctgggaagccaaggttgttggggcactccctcccctccggtcattccctaggcagattcttggggcacaggcgcagcggccagggacggcccaggtcggtggcgcgcagctgcggtggcactgcgcttccccgcccccgcaggccccccttcctctccccgctagatctgaagatgaagctccacccctagcgcgtcgccccagccccgcgccttaaaagccccgcacaccgccccgccgcacccagccttgccgcaccttcgcgtcctcgccaggtccgccgcagccgcgcacccggccccgaccccggcaccatgagcttcggatcagagcactacttgtgctccgcctcctcctaccgcaaggtgttcggggatggctcgcgcctgtctgcgcgcctgtccgggccgggagcgtccggcagctttcgctcgcagtcgctgtcccgcagcaatgtggcctccacggccgcttgctcctcggcctcgtctctcggcctgggcctggcctaccgccgcctgccggcctccgacgggctggacctgagccaagcagcggcgcgcaccaacgagtacaagatcatccgcactaacgagaaggagcagctgcagggcctcaacgaccgcttcgcggtgttcatcgaaaaggtgcaccagctggagacgcagaaccgcgcgctcgaggccgagctggccgcgctgcgccagcgccacgccgagccgtcgcgcgtcggcgagctcttccagcgcgagctgcgcgagctgcgcgcgcagctggaggaggcgagctccgcgcgcgcgcaggccctgctggagcgcgacgggctggccgaggaggtgcagcgactgcgggcgcgctgcgaggaggagagccgcgggcgcgaaggcgcggagcgcgccctgaaggcgcagcagcgcgacgtggacggcgccactctggcccgcctggatctggagaagaaggtggagtcgctgctggacgagctggccttcgtgcgccaggtgcacgacgaggaggtggccgagctcctggccacgctgcaggcgtcttcgcaagccgccgccgaggtggacgtggctgtggctaaaccagacctgacttcggcgctgagggagatccgcgcccagtatgagtccttggccgctaagaacctgcagtcggccgaggagtggtacaagtccaagttcgccaacctgaacgagcaggctgcgcgcagcacggaagccatccgagccagccgagaggagatccacgagtaccggcgccagctccaggctcgtaccattgagatagagggcctgcgcggagccaatgaatccctggagaggcagatcttggaactggaggagcggcacagcgctgaggtggccggctaccaggatagcattgggcagctggagagtgacctgaggaacaccaagagcgagatggcccgccaccttcgggaataccaggacttgctcaatgtcaaaatggctcttgacattgagatagcagcttacaggaagctgctggaaggcgaagagacacgatttagcaccagcgggttaagcatctcagggctgaatccacttcccaatcccagttatttgctccctcctagaatccttagttctacaacctccaaagtctcatccgctgggctgtccctgaaaaaggaggaggaggaggaggaggaagaggaagaaggggcttctaaggaagtcactaagaaaacatccaaggtaggggaaagtttcgaagaaacactggaggaaacggtggtatctacgaagaaaaccgaaaagtcaactatagaagaaatcaccaccagttccagccaaaaaatgtaatgctgttgcttaaaaaaaaaaaacaaaaaaaggcgatgcctaagagggaatgcacttggcttattcacagcttactcctgaaccacacctaccaccgcaatagaatgtcttcaaggcttcaatgtcatagtctgtaccgaatcctcgctctctccagtaggaaggccactgtagctgggagcaaactgcagtgctggaggaggagcaggaggagctctctaagatcacagctttcctacctacagctcaggcctcatagtgacaggtgacccaggtgcagaggaggctcaaacgaaggtgctaaatctggtcatcgctcgctgtgagctgaaatcgaaacctgtttctccaccgtaccccgccagtacccagccatagaacctcactgtagatgtctaaaaatatacagtcactgccgaggacaagctagtgctccagacaccattctcccatccccctctgatagagctgagtcacaaaacgacggtcgtctctcatgggcaggtttccttcctctctcttcaatcccgtattgccatttctcccacctgagaggaaggaacaaagtttagtgtggtatccccttctagacacgcatagaacttacgccctttagtcctcactccgtgggtaaattagatgcatgttgaccattcccgtgatttgtataacgcacccttgtccatttcatgacctttcatgtccgtagcttaaaaaatagaaagtgattaatgataatggcagtgattggttttaagggttttcctaaaataatactaattataataataatcataaaagggatctctagactttaggctttcagcttatccagatccacttaattagagaggaaaaaaattaccttaaatgatgtagcatttcaccttaaatttagaggcacggtgatagttgccctactctgcgcttaagctgaccttagacctcgtatcgtaagtagagtgagtgagtgagtgctctgaaagggaaagaacaaggtctcctctgtgttggtttctatgtagtgctatagaattagctttctcatacccttgccctaggatctacataattcccttcccattagggtagtatgctatgtataggacattttatcaccagacacaacgctactgcccaaaagtgaaaaacacacacttctggtcaatcaacaccaccagcaaatatttaaaaaaaataaaaaaggaaggaagaaagaaagaaagaaagaaagaaagaaagagagagagagagagaaagagagaaagaaagagaaagattgattctcagagtcttttcctaacagggttacattgcttcagttggtggatgtccctcccctgaaggaaagacagaaacattattcagggtccaggaggggtatgcacacttctcccccacctcaagaaggcaggctggaaagtcagctcttcgtgcactcaactgtggcatccacatgacctgtctgcctcgcgtgccaaacttgatgcagggtttgccctgtctctgctcactcccaccaagctgtagaactgtctgtcctgtccccgcccaccccatcgcaagctctgtgctgataaggacatgatgcttctactgaattcacattctagcacgtgcttcaatatagctcaaggaaccggaaagacacttcgtttttcctgacccatcccctcttgaccccccccccacactgtaagacattagtaatattaagaaattaaaagtcattaatcctatgtggttcttgtctgtggtctctatagctcctgaggtgtgcatt

SEQ ID NO: 82 - protein, 320 aa, Mus musculus
  Beta-sarcoglycan
MAAAAAAAAATEQQGSNGPVKKSMREKAVERRNVNKEHNSNFKAGYIPIDEDRLHKTGLRGRKGNLAICVIVLLFILAVINLLITLVIWAVIRIGPNGCDSMEFHESGLLRFKQVSDMGVIHPLYKSTVGGRRNENLVITGNNQPIVFQQGTTKLSVEKNKTSITSDIGMQFFDPRTHNILFSTDYETHEFHLPSGVKSLNVQKASTERITSNATSDLNIKVDGRAIVRGNEGVFIMGKTIEFHMGGDVELKAENSIILNGTVMVSPTRLPSSSSGDQSGSGDWVRYKLCMCADGTLFKVQVTGHNMGCQVSDNPCGNTH

SEQ ID NO: 83 - DNA, 1237 bp, Mus musculus
  Beta-sarcoglycan
gagagcgcaagcgcgggctgcggcgggccaagatggcggcagcggcggcggcggcggcagcgaccgagcagcaagggtccaacgggccagtgaagaaatccatgcgggagaaggctgtggagcggaggaatgttaataaagagcacaacagcaatttcaaagctggatatatccccatcgatgaggaccggctccataagactgggctgagggggcgaaagggcaacttagccatctgcgtgatcgtcctcctgtttatcctggccgtcatcaatctactcattacacttgtcatctgggcggtgatccgcattgggccaaatgggtgtgatagcatggagttccacgagagcggtctgctgaggttcaagcaagtgtctgacatgggcgtcatccacccgctttataagagcacggtgggaggacggcggaatgaaaacctggtcatcactggcaacaaccagccaattgtcttccagcaagggacgaccaagctgagtgttgaaaagaacaagacctccatcaccagcgacatcgggatgcagttttttgacccaaggacacacaatatcctgttcagcacggactatgagacgcacgagtttcatctgccaagtggggtgaaaagtttgaacgttcagaaagcatcgactgaaaggattaccagcaatgctaccagtgacttaaacatcaaagtcgatgggcgtgcaattgtgcgaggcaacgaaggcgtcttcatcatgggcaaaaccattgaatttcacatgggaggagatgtggagttaaaagctgaaaacagcatcatcctcaacggcacggtgatggtcagccctacgcgcctgcccagttcctccagcggggaccagtcagggagtggtgactgggtgcgctacaagctctgcatgtgtgcagatggcacactcttcaaagtacaagtaacaggtcacaacatgggctgccaggtctcagacaacccttgtgggaacactcattagacggtgcatccgcaggctcagcgcgctcagatctgacttgaccttcccctttttaagaatgcatgtgaaccatgtttgtacagcgtttgccataacttgtaattattttacctggggagcaccactgtgtctattatgtactctccatatttaagggatcttgagtgcctacattctaatacattttagtaagttgcactgatactcaacagttctctaaaacaatgatgagaattaataaa

SEQ ID NO: 84 - protein, 251 aa, Homo sapiens
  CGI-78 protein
MGAAVFFGCTFVAFGPAFALFLITVAGDPLRVIILVAGAFFWLVSLLLASVVWFILVHVTDRSDARLQYGLLIFGAAVSVLLQEVFRFAYYKLLKKADEGLASLSEDGRSPISIRQMAYVSGLSFGIISGVFSVINILADALGPGVVGIHGDSPYYFLTSAFLTAAIILLHTFWGVVFFDACERRRYWALGLVVGSHLLTSGLTFLNPWYEASLLPIYAVTVSMGLWAFITAGGSLRSIQRSSCVRTDYLD

SEQ ID NO: 85 - DNA, 1967 bp, Homo sapiens
  CGI-78 protein
gtggggtcgcgttgccaccccacgcggactccccagctggcgcgcccctcccatttgcctgtcctggtcaggcccccaccccccttcccacctgaccagccatgggggctgcggtgtttttcggctgcactttcgtcgcgttcggcccggccttcgcgcttttcttgatcactgtggctggggacccgcttcgcgttatcatcctggtcgcaggggcatttttctggctggtctccctgctcctggcctctgtggtctggttcatcttggtccatgtgaccgaccggtcagatgcccggctccagtacggcctcctgatttttggtgctgctgtctctgtccttctacaggaggtgttccgctttgcctactacaagctgcttaagaaggcagatgaggggttagcatcgctgagtgaggacggaagatcacccatctccatccgccagatggcctatgtttctggtctctccttcggtatcatcagtggtgtcttctctgttatcaatattttggctgatgcacttgggccaggtgtggttgggatccatggagactcaccctattacttcctgacttcagcctttctgacagcagccattatcctgctccataccttttggggagttgtgttctttgatgcctgtgagaggagacggtactgggctttgggcctggtggttgggagtcacctactgacatcgggactgacattcctgaacccctggtatgaggccagcctgctgcccatctatgcagtcactgtttccatggggctctgggccttcatcacagctggagggtccctccgaagtattcagcgcagctcttgtgtaaggactgactacctggactgatcgcctgacagatcccacctgcctgtccactgcccatgactgagcccagccccagcccggggtccattgcccacattctctgtctccttctcgtcggtctaccccactacctccagggttttgctttgtccttttgtgaccgttagtctctaagctttaccaggagcagcctgggttcagccagtcagtgactggtgggtttgaatctgcacttatccccaccacctggggacccccttgttgtgtccaggactccccctgtgtcagtgctctgctctcaccctgcccaagactcacctcccttcccctctgcaggccgacggcaggaggacagtcgggtgatggtgtattctgccctgcgcatcccacccgaggactgagggaacctaggggggacccctgggcctggggtgccctcctgatgtcctcgccctgtatttctccatctccagttctggacagtgcaggttgccaagaaaagggacctagtttagccattgccctggagatgaaattaatggaggctcaaggatagatgagctctgagtttctcagtactccctcaagactggacatcttggtctttttctcaggcctgagggggaaccatttttggtgtgataaataccctaaactgcctttttttcttttttgaggtggggggagggaggaggtatattggaactcttctaacctccttgggctatattttctctcctcgagttgctcctcatggctgggctcatttcggtccctttctccttggtcccagaccttgggggaaaggaaggaagtgcatgtttgggaactggcattactggaactaatggttttaacctccttaaccaccagcatccctcctctccccaaggtgaagtggagggtgctgtggtgagctggccactccagagctgcagtgccactggaggagtcagactaccatgacatcgtagggaaggaggggagatttttttgtagtttttaattggggtgtgggaggggcggggaggttttctataaactgtatcattttctgctgagggtggagtgtcccatccttttaatcaaggtgattgtgattttgactaataaaaaagaatttgtaaaaaa

SEQ ID NO: 86 - protein, 885 aa, Homo sapiens
  KIAA0143 protein
AGGGSCRPLGCVTAGSASAPSTLRPSPFASSRPPRPSNGRHGAVGAPCAAPLSLGAAASAVEIAMPTRVCCCCSALRPRYKRLVDNIFPEDPKDGLVKTDMEKLTFYAVSAPEKLDRIGSYLAERLSRDVVRHRSGYVLIAMEALDQLLMACHSQSIKPFVESFLHMVAKLLESGEPKLQVLGTNSFVKFANIEEDTPSYHRRYDFFVSRFSAMCHSCHSDPEIRTEIRIAGIRGIQGVVRKTVNDELRATIWEPQHMDKIVPSLLFNMQKIEEVDSRIGPPSSPSATDKEENPAVLAENCFRELLGRATFGNMNNAVRPVFAHLDHHKLWDPNEFAVHCFKIIMYSIQAQYSHHVIQEILGHLDARKKDAPRVRAGIIQVLLEAVAIAAKGSIGPTVLEVFNTLLKHLRLSVEFEANDLQGGSVGSVNLNTSSKDNDEKIVQNAIIQTIGFFGSNLPDYQRSEIMMFIMGKVPVFGTSTHTLDISQLGDLGTRRIQIMLLRSLLMVTSGYKAKTIVTALPGSFLDPLLSPSLMEDYELRQLVLEVMHNLMDRHDNRAKLRGIRIIPDVADLKIKREKICRQDTSFMKKNGQQLYRHIYLGCKEEDNVQKNYELLYTSLALITIELANEEVVIDLIRLAIALQDSAIINEDNLPMFHRCGIMALVAAYLNFVSQMIAVPAFCQHVSKVIEIRTMEAPYFLPEHIFRDKCMLPKSLEKHEKDLYFLTNKIAESLGGSGYSVERLSVPYVPQVTDEDRLSRRKSIVDTVSIQVDILSNNVPSDDVVSNTEEITFEALKKAIDTSGMEEQEKEKRRLVIEKFQKAPFEEIAAQCESKANLLHDRLAQILELTIRPPPSPSGTLTITSGHAQYQSVPVYEMKFPDLCVY

SEQ ID NO: 87 - DNA, 5286 bp, Homo sapiens
  KIAA0143 protein
ggctggcggcggtagctgtcgcccgcttggttgcgtgaccgcggggtccgcgtccgctccctccacccttcgcccttcgcccttcgcctcgtcccggcctccgcggcccagcaacggccgtcatggtgccgtcggcgctccctgcgcggccccgctgagcctcggtgcggcggcgagcgcggtcgagatcgccatgcctacccgagtatgctgctgctgttccgctttgcgtcctcgctacaaacgcctggtggacaacatattccctgaagatccaaaagatggccttgtgaaaactgatatggagaaattgacattttatgcagtatctgctccagagaaactggatcgaattggttcttacctggcagaaaggttgagcagggatgttgtcagacatcgttctgggtatgttttgattgctatggaggcactggaccaacttctcatggcttgccattctcaaagcattaagccatttgtagaaagctttcttcatatggtggcaaagctgctggaatcgggggaaccaaagcttcaagttcttggaacaaattcttttgtcaaatttgcaaatattgaagaagacacaccatcctatcacagacgttatgacttttttgtgtctcgattcagtgccatgtgccattcctgtcatagtgatccagaaatacgaacagagatacgaattgctggaattagaggtattcaaggtgtggttcgcaaaacagtcaacgatgaacttcgggccaccatttgggaacctcagcatatggataagattgttccatccctcctgtttaacatgcaaaagatagaagaagttgacagtcgcataggccctccttcttctccttctgcaactgacaaagaagagaatcctgctgtgctggctgaaaactgtttcagagaactgctgggtcgagcaacttttgggaatatgaataatgctgttagaccagtttttgcgcatttagatcatcacaaactgtgggatcccaatgaatttgcagttcactgctttaaaattataatgtattccattcaggctcagtattctcaccatgtgatccaggagattctaggacaccttgatgctcgtaaaaaagatgctccccgggttcgagcaggtattattcaggttctgttagaggctgttgccattgctgctaaaggttccataggtccgacagtgctggaagtcttcaatacccttttgaaacatctgcgtctcagcgttgaattcgaagcaaatgatttacaggggggatctgtaggcagtgtcaacttaaatacaagttccaaagacaatgatgagaagattgtgcagaatgctatcatccaaacaataggattttttggaagtaacctaccagattatcagaggtcagaaatcatgatgttcattatggggaaagtacctgtctttggaacatctacccatactttggatatcagtcaactaggggatttgggaaccaggagaattcagataatgttgctgagatctttgcttatggtgacctctggatataaagcgaagacgattgttactgcactgccagggtctttcctggatcctttgttatcaccatctctcatggaggactacgaactgagacagttggtcttggaagtaatgcataatctcatggatcgtcatgacaatagggcaaagcttcgagggatcagaataataccggatgtagctgacctaaagataaaaagagaaaaaatttgcagacaagacacaagtttcatgaaaaagaatgggcaacagctgtatcggcacatatatttgggttgtaaagaggaagacaacgttcagaaaaactatgaactactttatacttctcttgctcttataactattgaactggctaatgaagaagtagttattgatctcattcgactggccattgctttacaggacagtgcaattatcaatgaggataatttgccaatgttccatcgttgtggaatcatggcactggttgcagcatacctcaactttgtaagtcagatgatagctgtccctgcattttgccagcatgttagcaaggttattgaaattcgaactatggaagccccttattttctaccagagcatatcttcagagataagtgcatgcttccaaaatctttagagaagcatgaaaaagatttgtactttctgaccaacaagattgcagagtcgctaggtggaagtggatatagtgttgagagattgtcagttccgtatgtaccacaagtaacagatgaagatcgactttctagaagaaaaagcattgtggacaccgtatccattcaggtggatattttatccaacaatgttccttctgatgatgtggttagtaacactgaagaaatcacttttgaagcattgaagaaagcaattgataccagtggaatggaagaacaggaaaaggaaaagaggcgtcttgtgatagagaaatttcagaaagcaccttttgaagaaatagcagcacagtgtgaatccaaagcaaatttgcttcatgatagacttgcccaaatattggaactcaccatacgtcctcctcccagtccatcaggaacactgaccattacttctgggcatgcccaataccaatctgtcccagtctatgagatgaagtttccagatctgtgtgtgtactgatcggcgcatgaagacctcagcatatgatttgtaaagcctaaaaattaaggccaagctgagctttcagggtttacttaatgtgtattaacatacttcttgaaaataatgatggaacatatctttaaccaaatgtttggcataccatattagaagttttggagctatatataatttcgagtactttcaaagatagatttatgccatgttaatttgctttgaggttcctgttgcctttttaagttgaacatgttttggtttcactttattccactgttaagtagtatgttttaaacttttcacaaatgtaatgttttttaaaaagtaagccttcagaggattgaaactgtataaattgtttatctcttaaacatctacacagccgcttagatgtagaatttttgttgttgttttctgcaaaggcagatacatttaaatatattcctagtcctggggctgcaaaactgttcggtggctttttgtccccatgctttagataagctgggataggcaccttgctattcagttactataataatatgtgataggcattcctcatcttcttcacaataataggacatctgttgaatagcattcctcgaatataaccctaaaaacgccatactttaaattgtctggttcttgtaatattgtgtttcctgccaagagtttgaccattctgcttgagaagtgtagagcttactcttggagtaccaaactgtgcaatatttttacatcataagatgtattagtttacaggctgtgctttgaaattatagtagtattttgctgtggctccattaattaaatgagatatatatttagtgcagaaaaaagacatttaaaacagctattagttcacctgtgaagagtctgtacattttgatttctattaagagcacatttatttacatttgtatattattttaaatcctcatagtcaaaaaattctccagatggacttttaatttgtaagatttgaactgtgacttgtatacatctgtttagaatcaattatatttgaaaagctgcctgtgttttaacagtcaagtgtgctaaagtttgtcaatttaagctgcttttgatttcagctaccaagattacaggtgcactctacacataacactgacagacccataacattacatacatcttagtgaattctatcacatggtaaaatgaacagctttctttgtaactcataaaattctcttaggacatttttataaagtcacctgtttatagttctatcttttcagattccatttctttttacataaaacagcatacatatcaaaaactgtagcctagaatacagtttaatttttggcttttgtttttgtttaaaaaattgcagtgaagaatgggatgtttgtgtttatggctatttgggcacctttagtagaaacagacaaaagtaaggaaacgataataggacaagcatacttgaaaatttctgaatacttaaacaaaagcgcaacatcttgaaaaccagtctagtcattgaaacctatgaaatgacactgaaagatcctgggctgcttcttaaataagtagatcagcaagacttgtttcagagtgacagtggagtcgttaccgctggaggactaaagggccctgtggcagctgtcactggaactttgcctcttgatcaggaaaaatgcttcaccagtccgtaaagccaagttgtattttttttattgcccttttttccttctgtatttttaaagaaggatgttaatttttgactatatattttaaaaaaatctaagcagggggacatgcaaaaacaatcatcatccacttggatgtcattttatagattaacactgtgtgcttttgtatggaaaaaatatatataatttaatagtataaaaaataaaatatatattcatttgcacttacgtgaaacacaaactttgctctacaaaatttcgtgtttcttagtgattttaaaatgcatgtattgcatgtaaaggaaaaccattacaattaatgtttatcacacctttatcttggtctttgttgatttgggttttgttggggtttttgttactgtttttaaattacagtaggcttctatatatcctggatttctgaactggtcttgttgacaaggattcccaagaaatggatcttttcactggctgactctcccatatctgcaagagattctgcaggaactgggtgtgcacacggtgttgtagccagttcaggtactgaatatttaggatttggtgaagttcactgtattgctatatttttgtagataaataaaactcaataaattgttaatcattctctttttgctgtatagaattgcttatatcactctttctttcatgacattggttaacatttaaatgttcctcctgtacttgtgttgtctgtgaccgcttatagagttttattgttattggtgtttacctgaatacctatgcgtacacacacacatatttccttaatacttctgaactcattatcttttagaataataatactactacactttaccagcaattaacttctccctacccaaaatgtttttcttcctgtctgaaaatggaactaatttgtcttattcgtgcttatatctgtattaaatgcaataaagttagtttttgaaatgt

SEQ ID NO: 88 - protein, 424 aa, Homo sapiens
  Septin 2
TDIARQVGEGCRTVPLAGHVGFDSLPDQLVNKSVSQGFCFNILCVGETGLGKSTLMDTLFNTKFEGEPATHTQPGVQLQSNTYDLQESNVRLKLTIVSTVGFGDQINKEDSYKPIVEFIDAQFEAYLQEELKIRRVLHTYHDSRIHVCLYFIAPTGHSLKSLDLVTMKKLDSKVNIIPIIAKADAISKSELTKFKIKITSELVSNGVQIYQFPTDDESVAEINGTMNAHLPFAVIGSTEELKIGNKMMRARQYPWGTVQVENEAHCDFVKLREMLIRVNMEDLREQTHTRHYELYRRCKLEEMGFKDTDPDSKPFSLQETYEAKRNEFLGELQKKEEEMRQMFVQRVKEKEAELKEAEKELHEKFDRLKKLHQDEKKKLEDKKKSLDDEVNAFKQRKTAAELLQSQGSQAGGSQTLKRDKEKKN

SEQ ID NO: 89 - DNA, 4612 bp, Homo sapiens
  Septin 2
cgaccgatatagctcgccaggtgggtgaaggttgccgaactgtccccctggctggacatgtggggtttgacagcttgcctgaccagctggtgaataagtccgtcagccagggcttctgcttcaacatcctgtgcgtgggagagacaggtttgggcaagtccaccctcatggacaccctgttcaacaccaaattcgaaggggagccagccacccacacacagccgggtgtccagctccagtctaatacctatgacctccaagagagcaacgtgaggctaaagctcacgatcgttagcacagttggctttggggaccagatcaacaaagaggacagctacaagcctatcgtggaattcatcgatgcacaattcgaggcctacctgcaggaagagctaaagatccgaagagtgctacacacctaccatgactcccgaatccatgtctgcttgtatttcattgcccccacgggtcattccctgaagtctctggacctagtgactatgaagaagctggacagtaaggtgaacatcatccccatcattgccaaagcagatgccatttcgaagagtgagctaacaaagttcaaaatcaaaatcaccagcgagcttgtcagcaacggagtccagatctatcagtttcctacagatgatgagtcggtggcagagatcaatggaaccatgaacgcccacctgccgtttgctgtcattggcagcacagaagaactgaagataggcaacaagatgatgagggcgcggcagtatccttggggcactgtgcaggttgaaaacgaggcccactgcgactttgtgaagctgcgggagatgctgattcgggtcaacatggaggatctgcgggagcagacccacacccggcactatgagctgtatcgccgctgtaagctggaggagatgggcttcaaggacaccgaccctgacagcaaacccttcagtttacaggagacatatgaggccaaaaggaacgagttcctaggggaactccagaaaaaagaagaggagatgagacagatgttcgtccagcgagtcaaagagaaagaagcggagctcaaagaggcagagaaagagctgcacgagaagtttgaccgtctgaagaaactgcaccaggacgagaagaagaaactggaggataagaagaaatccctggatgatgaagtgaatgctttcaagcaaagaaagacggcggctgagctgctccagtcccagggctcccaggctggaggctcacagactctgaagagagacaaagagaagaaaaattaactctgctgtttgctgcatgctgcatgagacccagggtcctgtttgggcttcctgtagacacccttttcctgcgcaacagagctgggcctccctttctctaatttcccccttaacatgcctggggggcatacaatccaacccgcgccctctcctctcttcctgccaaggtttatagaaacctgagaatctgagggtgatgtctggccgctggtcaagaagccaacagtcatgtggctcgcagatgcatcctgcatcccagtccccctcccagcacccccagccatcccccctgtcttcccccacatctttgccagaggtgtgacatggtcagggggcccatctgctactctttcccaccagctcccctgttccagttctggttgctgttagtttccctgaggtatttgcaaccaccatggctgggtaaccaccgatcagcacagctgtccccttggtctcctgtatcccagtcactagtcctccctggtccaccccaccctcatcctcaggagccacagccatttcttagagggtttcaaaaggacagcctttggcgccttttccttctaacctttgagtccagccctttccagttttcattcactcgaagtaactgcactcaagctgtgctcaaaatcggcaacgcatttatttacaccaagcccttcccataaaacacaactgctgaagaaaatagcagacgtttcccctctctctaactctgggtatcccacagatgcaaaagggagaataaacctgaatattattaccagcctagagtcttgaatgatagccttaccgaattcttcttgtgaggtatttcagcatctcggggggtaatttccggaagggctccatactgtcccaataaggtgaggccagtagcaggaataataaatcccactttgtaggctggaaaactgagctgtcaaaagaatcaagtgtttgggggtttgctctgatgagtcttctagttcatttggtgaatgtcatgatgatttttaacatgcattttgcatgcatcccccaataagaagagatgagactcggctggagagaagaaaaggcccttaactttctttccaatttaaggagttgagagtttaaaaatattccagccctaagtttttatcatgggtcccatctgatagtggctttgggaacctctgtgaagtagagagccctcccttgtcagggttatgaggcacagtggcctttggtgtttggccagtgacagtgtgagagatggagttgacctggcaatgatctgtggctaacatgccgtctctctgcccttcctttgcagtaatccatggctgtgtactgaatagtattccccgctacagctggactggactccatttagccttttaagccgaggttcctattttaactgacagctttcctttggggtgccaggcagcgaggccccccacccctatcctgccatgtacttcaagctcacttcttctttttgagttccgcaacttgctcctgcctcccagccccactggcactgaccatgaccacctacttctattttttttttagagtttctttttttgatcacttactttcaaagcacacagtcaaacaaggttatgccaaatttccaggcctttttgaagtattgagaaggggaaggggatttctcacttcaattatagatcataataggaagcaaaaagaaaaaaatgaaaagcaaacatatgcacgcacttttcttgttgacaaagcaagaatataggtttgctgtgtaggtttggtgctctattgattggtgagtgaccagagcaagtatgaaggtgatgctgccaaagcacaagcctttttgaagtattgagaaggggaaggggatttctcacttcaattatagatcataataggaagcaaaaagaaaaaaatgaaaagcaaacatatgcacgcacttttcttgttgacaaagcaagaatataggtttgctgtgtaggtttggtgctctattgattggtgagtgaccagagcaagtatgaaggtgatgctgccaaagcacaagccagtttcttgggaaaattcaagttacagtggagtatttttttgaagaccatatgcttggaggtagaaacaaaccaacgaccaaaaaaaaaaaaaaaaaatctgctcagatactcagccagtagctcagagagatgctgagttaggcctgtcaggtctccttgggaaaggcttcatatttgcaactttgatgattctatgtccagcttcagagctgctttcccagaaattcacgcttaaacaaccaaccggtaaccaccacttccccacaccgccgcccggtaattatttgcattacaaaccggaggcgccctcatttgcatttgtgtacagattaactagttaaggcttgagaagctctgaataattcaaaagtattagacccacacagccttggagagaccttcagaaactaaggaggagttttatattaagggagacattttagtcagtaagacgatataacctacttactccgtaaggggaaatgaaggcccagagaagggaagggacttgaccgaggtcccacttctgtttcgaggcagaagccagactaattttcatgcctcctgactcccaatcagtttcacaaagggattcaatctgtttatatacgttacattcctggatacgaggtcttttgatgttcagagtaactgactagttagtattagaagaccctcgaggtttttttccacagaaaaacatctgaagatggattgggtgagggctggcaaaacgaaggcatgccgggccagctccttaacccaatgacccagtgatgctgcaaggctggaacggggtccaggagactgtgtgtaacaggtgccctaggtgacccttataatcagggaagtttggtgaacaaaaatcgaacccatgagtgaacataaattaaaaagttgatcaacctattaaaatgtgtatttcattgggtagcttttctcactgtagacagattttttccttcttcaatgaaaaggcttttaaattagtacaactgttactatttaaaaaaaaaataccctaagtactctgtttacttctggtgaaacaaaaccagtcattagaaatggtctgtgcttttattttcccagactggagtggcttttctgaaacacacacacacacacacacacacacacacacgtacacacatccctcacttctcttaagccaagaagtttgctttccctagctgcagtgtagatggctcttgtttttgtttttttgttttaatcatttggcattcacatgtggctgttaatatgtgcttgtttttaattaaaacaagaagcttt

SEQ ID NO: 90 - protein, 459 aa, Rattus norvegicus
  Nucleobindin
MPTSVPRGAPFLLLPPLLMLSAVLAVPVDRAAPHQEDNQATETPDTGLYYHRYLQEVINVLETDGHFREKLQAANAEDIKSGKLSQELDFVSHNVRTKLDELKRQEVSRLRMLLKAKMDAKQEPNLQVDHMNLLKQFEHLDPQNQHTFEARDLELLIQTATRDLAQYDAAHHEEFKRYEMLKEHERRRYLESLGEEQRKEAERKLQEQQRRHREHPKVNVPGSQAQLKEVWEELDGLDPNRFNPKTFFILHDINSDGVLDEQELEALFTKELEKVYDPKNEEDDMREMEEERLRMREHVMKNVDTNQDRLVTLEEFLASTQRKEFGETAEGWKTVEMYPAYTEEELKRFEEELAAREAELNARAQRLSQETEALGRSQDRLEAQKRELQQAVLQMEQRKQQQQEQSAPPSQPDGQLQFRADTGDAPVPAPAGDQKDVPASEKKVPEQPPVLPQLDSQHL

SEQ ID NO: 91 - DNA, 2152 bp, Rattus norvegicus
  Nucleobindin
ccagaaacctggcggaggtcgcgggctgggaaaacgccctctgcgctaggagagaccagctacgatgcctacctctgtgccccgcggggcgcctttccttctcctaccacctttactgatgctgtctgctgtgctggcagtgcctgtggaccgcgcagcacctcatcaggaggacaaccaggccactgagaccccggacacaggcctgtactaccatcggtacctccaggaggtcatcaacgtgctagagacagatgggcacttccgggagaagctgcaagctgccaacgctgaggacattaagagtggaaagctgagtcaagagctggacttcgtcagccacaacgtccgcaccaagctggatgagctcaagcgacaggaggtatcaaggcttcggatgctgctcaaggccaagatggatgcaaagcaggagcctaacttgcaggtggaccacatgaaccttctgaagcagtttgaacacctggaccctcagaaccagcacacgtttgaggctcgggacctagagctgctgatccagacggccacccgagacctcgcccagtatgacgctgcacaccatgaagagttcaaacgctacgagatgctcaaggaacatgagcgaagacgttacctggagtctctgggagaggagcagcggaaggaggctgagaggaagctacaagagcagcagcgcagacaccgggaacaccccaaagtcaatgttcctggcagccaagcccagttgaaggaggtgtgggaggagctagatggattggaccccaacaggttcaaccccaagaccttcttcatactgcatgacatcaacagcgatggtgtcctagatgagcaagaactggaagctctgtttaccaaggagctggagaaggtgtacgacccgaagaacgaggaggacgacatgagggagatggaggaggagcggctgcgcatgcgggagcatgtgatgaagaacgtggacaccaaccaggaccgacttgtgaccctggaggaattcctggcatccacacagaggaaggagtttggggaaactgcggagggatggaagacagtggaaatgtatccagcctacacagaggaagagctgaagcgttttgaagaggagcttgctgcccgggaggctgagctaaatgccagagcccagcgcctcagccaggagacagaggccctggggcgctcccaggaccgcctagaggctcagaagagagagctgcagcaggccgttctgcagatggagcaaaggaagcagcaacagcaagagcagagcgctccgccttcccaacctgatgggcagctgcagttccgtgcagacacaggggatgctcctgtcccagctccagcaggtgaccagaaagacgtgcctgcttctgaaaagaaggttccagagcagccccctgtgctgccacagctggattctcagcatttataagccctcagagatcccggccctcaagttcccacagacagtgatggaagctggatgaagtgtccttgcagcctttctggagaccaaagccttcatggcctccaggggagaacagggccaccctgttcctcagatgcgcttctgcccatgtctaccagtctgttctcacagcctctgggtcccagtcctgccttcctccctcccttccttggcagggactcaactgcaggagcccctcaccccacctgagtgcacaccccaggcctgcctgccttccatgctttgctcttacctgtcccaggctcaacctttctgcttcctgtacctgtcagtgggtcacaggagcttgacttggtctctccaactctttgccttgtgagaggccaatgatctcaattccttttggtggctagaggccgccacaatgtggggtgtcctatcagctctgcctgggactcctgaactccaccaccaccccaccagccttaccctaaccacctgggcccaatacctacctgagggtcccagaaggggcccagatatggctacaccccttactgaggtccagcctcactcccacgtatgggggctcctgaaaattggttcacccacttccctggcttcaggccacactgctgtgtcctgctctgtgtcccagtctcatccaactatagcctggcacaaataaacattagatatctgtgtgaatgt

SEQ ID NO: 92 - protein, 160 aa, Rattus norvegicus
  Myelin structural protein SR 13
MLLLLLGILFLHIAVLVLLFVSTIVSQWLVGNGHRTDLWQNCTTSALGAVQHCYSSSVSEWLQSVQATMILSVIFSVLSLFLFFCQLFTLTKGGRFYITGVFQILAGLCVMSAAAIYTVRHSEWHVNNDYSYGFAYILAWVAFPLALLSGIIYVILRKRE

SEQ ID NO: 93 - DNA, 1736 bp, Rattus norvegicus
  Myelin structural protein SR 13
gggggaagccagcaacctagaggacgcccccgagtttgtgcctgaggctactccgctctgagccggctgtccctttgaactgaaagcaccgctccaccgagcccgagcccaactccagccaccatgcttctactcttgttggggatcctgttccttcacatcgcggtgctagtgttgctcttcgtctccaccatcgtcagccaatggctcgtgggcaatggacacaggactgatctctggcagaactgtaccacatccgccttgggagccgtccagcactgctactcctcatctgtgagcgaatggcttcagtctgtccaggccaccatgatcctgtctgtcatcttcagcgtcctgtccctgttcctgttcttctgccagctcttcactctcaccaaaggcggccgcttttacatcactggagtcttccaaatccttgctggtctgtgtgtgatgagtgcagcggccatctacacagtgagacacagtgagtggcatgtcaacaacgactactcctatggctttgcttacatcctggcctgggtggctttcccgctggccctccttagtggcatcatctacgtgatcctgcggaaacgcgaatgaggcgcccgacgcaccatccgtctaggctctgagcgtgcatagggtacacagggagggaggaaggaaaccagaaaaccaaaccaaccaacccaaaagagctagcccccaaacccaaacgcaagccaaaccaaacagaacacagttgagtggggattgctgtcgattgaagatgtatataatatctatggtttataaaacctatttataacactttttacatacatgtacataggattgtttgctttttatgttgaccgtcagcctcgtgttgaatcttaaacgactctacatcctaacactataaccaagctcagtattttcgttttgtttcgtttttttcatctttttgttttgctcagacataaaaaaaaaaaaaaaatccacgtggccccctttcatctgaaagcagatccctccctcccattcaacctcataggataaccaaagtgcggggacaaaccccagatggccagaggccttcacactatgggtgacccagtgaatttagcaggaataatccgctgcccgaatcaatgtgtgaagccctaagcactcacagacgaaacgccctgaccagagccctctgcgaaaccaatagctggtggctgcggaacacttgaccctgaaggcggagtactggggcacatgtttaaatgagacgtcagagacaagcaatctgtgaaatggtgctatagatttaccattccttgttattactaatcatttaaaccactcactggaaactcaattaacagttttatgacctacagcagaacagagacccgatacaaacggttcgtaactgctttcgtacatagctaggctgttgttattactacaataaataaatctcaaagccttcgtcactcccacagttttctcacggtcggagcatcaggacgagggtctagacccttgggactagcaaattccctggctttctgggtctagagtgttctgtgcctccaaggactgtctagcgatgacttgtattggccaccaactgtagatgtatatacggtgtccttctgatgctaagactccagacctttcttggttttgctggctttttctgattttataccaactgtgtggactaagatgcattaaaataaacatcagagtaactc

SEQ ID NO: 94 - protein, 375 aa, Rattus norvegicus
  Cytoplasmic beta-actin
MDDDIAALVVDNGSGMCKAGFAGDDAPRAVFPSIVGRPRHQGVMVGMGQKDSYVGDEAQSKRGILTLKYPIEHGIVTNWDDMEKIWHHTFYNELRVAPEEHPVLLTEAPLNPKANREKMTQIMFETFNTPAMYVAIQAVLSLYASGRTTGIVMDSGDGVTHTVPIYEGYALPHAILRLDLAGRDLTDYLMKILTERGYSFTTTAEREIVRDIKEKLCYVALDFEQEMATAASSSSLEKSYELPDGQVITIGNERFRCPEALFQPSFLGMESCGIHETTFNSIMKCDVDIRKDLYANTVLSGGTTMYPGIADRMQKEITALAPSTMKIKIIAPPERKYSVWIGGSILASLSTFQQMWISKQEYDESGPSIVHRKCF

SEQ ID NO: 95 - DNA, 4100 bp, Rattus norvegicus
  misc_feature 3859: n is a or g or c or t
acctcttcctcaactcacttctctctactctcacttttttttttcttcttcttttttttttttttttttttttttttttttgcaaaagaagggggtaaaaaaatgctgcactgtcgggcgaggccggtgagtgagcgagccggagccaatcagcgcccgccgttccgaaattgccttttatggctcgagtggccgctgtggcgtcctataaaacccggcggcgcaacgcgcgccactgtcgagtccgcgtccacccgcgagtacaacctccttgcagctcctccgtcccggtccacacccgccaccaggtaagcagggacgtcgggcccagcgggccccaactttaccttggccactacctcgctgcaggatcgtgaggaacactcagaagggacaccgtagaggggtggagcgtggtaccgggccgcggagcagctggcaaagcttaactttcccggcccctagggtgtagagtgtttgcagtcgtattcccgcggtgtggacactcgtgggtacgctcctgcttggtgcgcggggcttggggacacactagagtcgcggtgtgggcatttggagagccggtgcggctgcgggtgttaagccgcatctgtccaccttgaggggcaacagtattggggtcaggcgttacaatcacgctttgatggcctatgggtctttgtccaaaccggttttgccattcggcttggcgggcgcggcggggccgctcggccgggtgggggctgggatgccattgcgcgtgcgcgctctatcactgggcattggggccgtgcgcgctggggagggaactcttcctctccccctcttccgagttaagagttgcgcgtgcgtattgagactaggagcgcggccgccccgggttgggcgagggcgggccgtccaccggaaggggcggggtcgtagcggctaggcgcctgctcgcgcttcctgctgggtgtggtcgcctcccgcgcgcgcactagccgcccgtcgcctcagtgtaggcgggcctgtgcccgtttggggaggggcggaggcctggcttcctgccgtgggtccgcctccgggccagcgtttgccttttatggtaataatgcggctgtcctgcgcttcctttgtcccctgagcttgggcgcgcccctggcggctcgaggccgcggctcgccggaagtgggcagggcggcagcggctgctcttggcggctccgcggtgaccatagccctcttttgtgccttgatagttcgccatggatgacgatatcgctgcgctcgtcgtcgacaacggctccggcatgtgcaaggccggcttcgcgggcgacgatgctccccgggccgtcttcccctccatcgtgggccgccctaggcaccaggtaagtgaccctttactttgggagtggcagccctagggttttcttgggggtcgatgccagtgctgagaacgttgttctcctccgcagggtgtgatggtgggtatgggtcagaaggactcctacgtgggcgacgaggcccagagcaagagaggcatcctgaccctgaagtaccccattgaacacggcattgtaaccaactgggacgatatggagaagatttggcaccacactttctacaatgagctgcgtgtggcccctgaggagcaccctgtgctgctcaccgaggcccctctgaaccctaaggccaaccgtgaaaagatgacccaggtcagtatcctgggtgaccctccccttcttattgggtcaacttctcagcacgcccttctctaattgtctttcttctgccatgtcccataggactctcttctatgagctgagtctcccttggaactttgcagtttctgctctttcccagatgaggtcttttttttctctcgatcgcctttctgactaggtgttttaaacccctacagtgctgtgggtgtaggtactaacaatggctcgtgtgacaaagctaatgaggctggtgataaatggccttggagtgtgtattcagtagatgacagtaggtctaaatggagcccctgtcctgatactcccagcacacttaacttagctgtgttcttgccctctttgcatgtctcactcaaatctatccttacagtctcacctgccctgagtgtttcttgtggctttaggagcttgacaatactgtattcctttctctacagatcatgtttgagaccttcaacaccccagccatgtacgtagccatccaggctgtgttgtccctgtatgcctctggtcgtaccactggcattgtgatggactccggagacggggtcacccacactgtgcccatctatgagggttacgcgctccctcatgccatcctgcgtctggacctggctggccgggacctgacagactacctcatgaagatcctgaccgagcgtggctacagcttcaccaccacagctgagagggaaatcgtgcgtgacattaaagagaagctgtgctatgttgccctagacttcgagcaagagatggccactgccgcatcctcttcctccctggagaagagctatgagctgcctgacggtcaggtcatcactatcggcaatgagcggttccgatgccccgaggctctcttccagccttccttcctgggtaagttgtagtctcgtcccttctccatctaaaggtgaccaatgctggaggccacactgtaactctgatctcttgcttttcctttcaggtatggaatcctgtggcatccatgaaactacattcaattccatcatgaagtgtgacgttgacatccgtaaagacctctatgccaacacagtgctgtctggtggcaccaccatgtacccaggcatcgctgacaggatgcagaaggagattactgccctggctcctagcaccatgaagatcaaggtaagcagccttagcctggacccatagtggggtgtggtcagccctgtagttgtagccaactctcttggcttaaggaacaacccagcatccagaatgctcacaatcactgtcttgctttcttcagatcattgctcctcctgagcgcaagtactctgtgtggattggtggctctatcctggcctcactgtccaccttccagcagatgtggatcagcaagcaggagtacgatgagtccggcccctccatcgtgcaccgcaaatgcttctaggcggactgttactgagctgcgttttacaccctttctttgacaaaacctaacttgcgcagaaaaaaaaaatgagacatttggcatggctttattgtttttttgttttttgtttttgttttttttaaattttttttttaaaaaggttttttttttttgttttgttttggcgcttttgactcaaggatttaaaaactggaacggtgaaggcgaccgcagttggttggagcaaacatcccccaaagttctacaatgtggctgaggactttgattgtacattgttttttgtttttggtttttttaatagtcactccaagtatccacggcatagatggttacaggaagtccctcaccctcccaaaagccacccccaactcctaaggggaggatggctgcatccatgccctgagtccacaccgggaaggtgacagcattgcttctgtgtaaattatgtacttgcaaacatttttttaaatcttccgccttaatacttcatttttgtttttaatttctgaatggtcagccattcgtggcctgccccttttttttgtccccccaacttgatgtatgaaggctttggtctccctgggagtggtttgaggtgttgaggcgccagggctggcctgtacactgacgtgagaccgttttaataaaagtgcacaccttacaaacaagtttgtggctctgtggcttctactgggtgtggggagcaggctgggtgggtgtgnaactccacgtgggggaggggcaatttagggtgggctggccttgcctgatagctagtgggagggctaaaggatcatgatctttaatgaggtctcataaataccccaccttcggtcttgagggagggtgaggagttgggaaggtatctccctgacacctggccaagctggcctcatcaaccctactttcctcagccagggggaggtccacatcctcccacctgcatagcttgcgtctagaggc

SEQ ID NO: 96 - protein, 116 aa, Mus musculus
  Ly6/neurotoxin homolog
MTHLLTVFLVALMGLPVAQALECHVCAYNGDNCFKPMRCPAMATYCMTTRTYFTPYRMKVRKSCVPSCFETVYDGYSKHASATSCCQYYLCNGAGFATPVTLALVPALLATFWSLL

SEQ ID NO: 97 - DNA, 3925 bp, Mus musculus
  Ly6/neurotoxin homolog
taccaacaccgcacgaagtgtgtacagattcccagttagacagcaggagggacctgggagcggccagggggatgttttatctctaagagaccaagagctcaggcagggcttctgtgccctgcttcctccctggcttgagctggatcctggaccagctgctgacctcctgttcactctggcactgccctcacgtctccgtcatgacccatctgctcacagtgttcctggtggccctgatgggcctgcctgtggcccaggctctggagtgccacgtgtgtgcctacaatggagacaactgcttcaaacccatgcgctgcccagccatggccacctactgtatgaccacacgaacttacttcaccccataccggatgaaggtgaggaagtcctgtgtccccagctgctttgaaaccgtgtacgatggctattccaagcatgcatctgccacctcctgttgccagtactacctctgcaacggtgctggctttgctaccccggtgaccttggccctggtcccagcactcctagctaccttctggagcttgctgtaaagctcggttccccaagccagatccactcaaacgcaacactctcaaaaaacacagtttccctctctctcccaattcactccacccaacgctcttccttctgacactcctcaactaccacgaggtcccatggctacctacgaaagaactgatggcatccagatacctcactccaaggtcattttcagaaggctgacatgtggacctgtaatgtgcccacccatgggtggggcaggctgggcttctcctctacccaagatcaggggcatctgggagaatgtttatggaggaggggtcatcactcaagtcaaggagcactgatttgatagaattagtagccaaactccaccttcagaaccctgcctcagtctacccagtagaggatgggtctgctagaggtgaggggaggagagcggcggagaataacgagctggctagaagcagagaaagactcagcagggctgtctccgaagatcagcgcggcttgccagagcaaatgtgatgtggaagccatgtgaggaagccctttgtcatttccacttatctgaggaactctgccagacctgatgttgggatagccattggccaagggttcctagcaacggcgtcatttccataggccactgaaatccctccagccccagctcagcaggccccttgacctccactacagtccttcattcacacaccagctgctgggccttgaagttggcagggacttgggagcaggtgacccatgctattttttgtctggcctgttattctgggcatggcaagaagggatcagacgcaggtcagagcagggcagtagggcgactgagacagggaaacagacttcagccagtggcttcccaggtcccgtaggcagctcctacatccttcagtctcttgttacattcccgggagacaaatatacagggagccaagccgagtgctaggtgatgactgcctgtgaagtctattgtggccacagactgctgggtaccaagtctcaggagaacccagcctagatttaggagacacagatctgcctttcatgcagtgtagctgtccttgggagccttaccatgctctctaactagttcctcaactcacatgtcactgaggaaccccctaacactggcccagcccaggggtcgggatgctggccaatgtccatggagtgggactacccctggagagtccttgggtcatcacatcacaaatgttttattccaacctcccagtggtgagagctcgggacacaaaggtccatcctggggaccttcttcctggttctaggcagacctgaactctgtctgctgctagagctgatgtggttttccgcctcagtttcctcctccggggataggccaccggaggatttgggagggtggggagggcatcctgctgatgggctcgccgaggttctcaggaacaggaacgggcggggctttagtacacaggtgagttgggtgggaactggcccggagctgaggagacactgactgggcagagggaagatgagtctcaagggagggcaggaaaagggagggggagcgcgcatgcacatgtgcactcagtgcaggctacagagcccaaaaggcagcactggctgtggtgtcccctgaggcccaggcaagatgctaggaggaagccaatgctgcccccacctgagctcacatggaacatgcacaccaccagcagcagcagcaagcattgagactgacctgtggacgccatagggcactggcaaggagggtcagaggcgggtccctgactcagtgggtgaggcccgggaaacattatcctgttaccctgcgtgtgcaagatcattgtccccagctagatggcgtcctcaaccaaaactgagaggagccccagttcaggtcctccctcctaccacaagggggtggtgtggaggaggcttgattgcccttggagaagcaccggtactgcagagctgggggccagcttctttcatctgtgtctagacaccgaccagataggccccacagtggcaacactgccacacagtcctacaagaagccctgtgcctagctagcacagagccccaaaaggtgctcaattaatacagggccaagcctgccagtgggggggatgcagattaggggaacagacccagatggcctgtcctgaaccctgtctggggtggtgtgatgagcatctgtctagcccactgcaggtggctctacacactccacaacagttctgcaaaagtgtatgaggtggtcattactgcgcccctctcacaggtaaaggcactgaggcacggaggagtgaggcacttcattttcctgggccattcaactttccaggaccaacacattcaactatgggtactactccaatagctggggttctttgaggctgggccccctgaagatgatagtggcttcatcaaccagagaatttcagagtgcagtgttgtaggagcctatgaacctgaaatgtcagaactggaggtttgaggggctgaggggtaggccaggggtgtctggccccttgtgtggagacagagagagagggaacatgggatggggtagtagagagaagtgcaaaggagcgtcagcctttctcagggctaatgctgtcagggacgagggctcaagcctgtgagtgttctcacactgtgataaacagtggcccctcaacacagacggtgtccagagtggccggcagtggttatctagagttgcaatctggaagcctcttggtagtcactggagagaggccgcttgatgggacagcaccaaatgtgtgtgcttctgtgggatgtgaggaagctgggtcagcgcatgaagccaaagcgtccttcagagcagaggggtggctggtctagtccaccagagacaagctatccagtgagagtcatactctgtcaccgtctctgtgattaccttaccccaaagcagacggggacgggatgcagagcacccgtgtcttcatcttctgcggcaagcacgtgagttcacattctgaaactctagaaagatttccaggagtggggtgtgcctttgctttggtgcatggttacttcctggcaagcaccgtggcatcccgcagcactgagtgacctgggctcctcaagccatctcattggtgaaatgacagtgccagtaccctctcagctggctcttggaggcctgtgcatggggtctgcacagaggaggcccccaaactatgcatggacggacacgtgatgcctagcacttcccttggttgtgtctctgccaaccccaggctctcacccagcaaggaaatgaaatccacttttatgacacatctccctcccccagccagctccattcacctatatgccagggtggtccctttcaatgtctgtcccccattggatgaataaacaagcgaaggac

SEQ ID NO: 98 - protein, 130 aa, Rattus norvegicus
  Astrocytic phosphoprotein
MAEYGTLLQDLTNNITLEDLEQLKSACKEDIPSEKSEEITTGSAWFSFLESHNKLDKDNLSYIEHIFEISRRPDLLTMVVDYRTRVLKISEEDELDTKLTRIPSAKKYKDIIRQPSEEEIIKLAPPPKKA

SEQ ID NO: 99 - DNA, 2341 bp, Rattus norvegicus
  Astrocytic phosphoprotein
gcggaagaggcggcggcggcaggatcggcggcagcggtggccggagccaaggagcaggctccagacgctgcttaggaaccggggatccgggagtgcccgcaccctgagctctcagctccggaggcgtcatggcagagtacgggactctccttcaggacctgaccaacaacatcacccttgaagatctggaacagctcaagtcagcctgcaaggaggacatccccagtgagaagagtgaagagatcactacaggcagtgcctggtttagcttcctggagagccacaacaagctggacaaagacaacctctcctacattgagcacatctttgagatctcccgccgtcctgacctactcacgatggtggttgactacagaacccgtgtgctgaagatctctgaggaggatgagctggacaccaagctaacccgtatccccagcgccaagaagtacaaagacattatccggcagccctctgaggaagaaatcatcaaattggctcccccaccaaagaaggcctgagcaagggggaggaagaggaggaaggttggatcttcatcagaccactcccttccccatcctcaatgggaggggctagggcaacaccctgctccgtacccacttactaacttggtcctaacccttactatgcgcgtgtgtgtgcgtgtgcgcacgctctggctgtctgtctgtctagctcatctagttcctcctcctcatgaggggattggagtcaagggaggggggcttagctgcccccactgtaaggggaggtggacgctttttctatgtaaacagaaactggcacactcctcctcctctcctctcctccactcttccccacatctttaaagtatggaagcagaaagggcctgcgttttcccacactgaggagctatggtgaaaatgaggtatggcggaaatgattagatctaaagaaaaccagtggggaaggaaggtcaagaggctacccaacctccacctgaaggggctcctgagtgccagcgggccctcctttatgcccttgttacgggcctgggatgtggataagcagagatcctaatgagaagaccactagctcctttcctaggtcccaagatcaaaccccagtggagaagccagcttttgatgtccccaacttctgctctggagagactatgctgggaccatgcactgctgagcctgagccagggatgcaggcagagacaggcccactctcgctcctcagtttctagatacagactgttgggatatactgacccggagcctaggaattggggaatgagatcccttaggttttaaccgcaacctgcagagtcccaaaatgggtattgtaggcaacctttccactttccagttcggaattctccaagcaagagataagttaacgacagtgtttcctttgcactgactaccaccccattcaatccaggaagggactggtcacctttctcatttgggtttgtgaatgccacacagccaagccactagagtacagtgagtgaacccagcctcctccctgtcccaagatgcccttccccatcttgaccgtgctaactgtgtgtacatatatattctacatatatgtatattaaacccgcactgccatgtctgcccttttttgtggtttttagcattaacttattgtctaggccagagcgggagtgggaggggaatgccacagtaaagggagtggcagagccaaattgctaagttagtcaaaacagaaaacatttccaaccaatcacaacaaacattgcatttacatttgagctaagagacaatttagagaaagttgaaggtaggagcttttaggatgtgggagcaaaacttcacagggaagggaggaccagctgtcagaagagggctcttgaagccagactggctagagcaccctcacttcctagctgagcccagtttgcccatctcctctggccactgctgcagacacctgccttaacacacacacctctaagactccagttttgccttaaaggcccttcctaagtctccctagtcctgcctggcatctcctttgagacaagaaatccttgtacaactgggagaaaggaactatgggcaacatccttccatctggggtgttacgtgagagcaggagttaagacggaggctttccttatgaagacacaggagagagagagggtgcttctcagcagtttctctgtaaggcaaaaaacacaaaccttaaacacactaacctgccctaatacactcacctaccctgctaccctcgtgctgctgtgaggttgcccctagcctgtgttcctctgtctgcagcgtgcacggccttgttctaaccctggaataaaggtgactgactg

SEQ ID NO: 100 - protein, 582 aa, Mus musculus
  PLIC-1
MAESAESGGPPGAQDSAADSGPAEPKIMKVTVKTPKEKEEFAVPENSSVQQFKEEISKRFKSHIDQLVLIFAGKILKDQDTLSQHGIHDGLTVHLVIKTQNRPQDNSAQQTNTTGNSVTSSPAPDSNPTSGPAANSSFGLGGLGGLAGLSSLGLNTTNFFELQSQMQRQLLSNPEMMVQIMENPFVQSMLSNPDLMRQLIMANPQMQQLIQRDPEISHMLNNPDIMGQTLELARNPAMMQEMMRNQDRALSNVESIRGGYNALRRMYTDIQEPMINAAQEQFGGNPFGSLVSSPSSAEGTQPSRTENRDPLPNPWAPQTPQSSPAYGSSGSTNNTVSTSGGNATSTPAGQGTSGPKLVPGAGGSMFNTPGMQSLLQQITENPQLMQNMLSAPYMRSMLQSLSQNPDLAAQMMLNDPLFAGNPQLQEQMRQQLPTFLQQMQNPDTLSAMSNPRTMQALLQIQQGLQTLATEAPGLIPGFTPGLAAGNSGGPAGTTAPSTAPGEDTNPQGGAAEPGHQQFIQQMLQALAGVNPQLQSPEVRFQQQLEQLSAMGFLKREPNLQALIATGGDINAAIERLLGSQPS

SEQ ID NO: 101 - DNA, 1843 bp, Mus musculus
  PLIC-1
ccatggccgagagcgcagagagcggcggcccaccgggcgctcaggacagtgcggccgacagcggccccgcagagcccaagatcatgaaggtcacggtgaagacgcctaaagagaaggaagagttcgctgtgcccgagaacagctcggtccagcagttcaaagaggaaatctcaaaacgtttcaaatctcatattgaccaacttgtgttgatatttgctggaaaaattttaaaagatcaagatactttgagtcagcatgggattcatgatggacttacggttcaccttgtcatcaaaacccaaaacaggccgcaagataattcagctcagcaaacaaataccactggaaacagtgtgaccagttcaccagctcctgacagtaaccccacgtctgggcctgctgctaacagctcctttggtttaggtggacttggaggacttgcaggtcttagtagcttgggtttaaataccaccaacttctttgaactacagagccaaatgcagcggcaacttttgtctaaccctgaaatgatggtccagatcatggaaaacccctttgtgcagagcatgctctcaaaccctgacctgatgcggcagctgatcatggccaacccacagatgcagcagctgatacagagagacccagagatcagtcatatgcttaataacccagatataatgggacagacattggaacttgccagaaacccagcgatgatgcaggaaatgatgagaaaccaggaccgagccttgagcaacgtagaaagtatccgtggagggtacaacgccttacggcgcatgtacacagatattcaggagcccatgataaacgctgcgcaagagcagtttggtggtaatccatttggttccttagtgagcagtccgtcctcagcagaagggactcagccttctcgaacagaaaatagggatccgctacctaacccttgggctccacagactccccagagctctccagcctatggcagcagtggcagcaccaataacacagtgagcacctctggtggcaatgccaccagtaccccggcgggacagggcacctcagggcccaagcttgtgcctggagcaggaggtagtatgttcaacacccccggaatgcagagcctgctgcagcagataactgagaacccacagctcatgcagaacatgctgtctgccccatacatgaggagcatgctgcagtccctgagccagaaccccgaccttgctgcgcagatgatgctgaatgaccccctatttgctgggaaccctcagcttcaagagcaaatgagacaacagctcccaacgttcctccaacaaatgcagaaccccgacacattgtcagccatgtcgaaccctagaaccatgcaggccctgctgcaaatccagcagggcttgcagacgttggccacagaagctcccggcctcatcccagggtttactcctggcttggcagcgggcaattctggaggccctgcaggaaccactgcacctagcactgcacctggtgaggatacgaacccccaagggggcgctgctgagccaggccaccagcagtttatccagcaaatgctgcaggccctcgccggggtgaaccctcagctgcagagtccagaagtcagatttcaacagcaactggaacagctcagtgcaatgggattcttgaagcgtgaaccaaacttacaagcactgatagcaacagggggcgacatcaatgcggccattgaaaggttgctgggttcccagccgtcatagcagcatttctgtatcctgaaaaaatgtaatttatttttgataacggctcttaaatctttaaaataacctgctttatttcattgggattctgt

SEQ ID NO: 102 - protein, 618 aa, Rattus norvegicus
  Nfxl
MADEGKSYNEHDDRVSFPQRRKKGRGPFRWKCGVGNRRSGRGGSGIRSSRFEEDDGDVAMNDPQDGPRVRFNPYTTRPNRRRDTWHDRDRIHVTVRRDRAPQERGGAGTSQDGTTKNWFKITIPYGKKYDKMWLLSMIQSKCSVPFNPIEFHYENTRAHFFVENATTASALKAVNYKIQDRENGRISIIINSSAPPYIVQNELKPEQVEQLKLIMSKRYDGSQQALDLKGLRSDPDLVAQNIDVVLNRRGCMAAALRIIEENIPELLSLNLSNNRLYKLDDMSSIVQKAPNLKILNLSGNELKSEWELDKIKGLKLEELWLDRNPMCDTFLDQSTYISTIRERFPKLLRLDGHELPPPIAFDVEAPTMLPPCKGSYFGTENLKSLVLHFLQQYYAIYDSGDRQGLLDAYHDGACCSLSTPSNPQNPVRHNLAKYFNDSRNVKKIKDTTTRFRLLKHTRLNVVAFLNELPKTHHDVNSFVVDISAQTSTLLCFSVNGVFKEVDGKSRDSLRAFTRTFIAVPASNSGLCIVNDELFVRNASPEEIQRAFAMPAPTPSSSPVPTLSQEQQDMLQAFSTQSGMNLEWSQKCLQDNNWDYTRSAQAFTHLKAKGEIPEVAFMK

SEQ ID NO: 103 - DNA, 2304 bp, Rattus norvegicus
  Nfxl
attccatcctaatacgactcactatagggctcgagcggccgcccgggcaggtccaaggcgtccgggtgtttggtggcgtcaggttctgcgcctgagcccgcccctgtacctgcctgtgccatggcggacgaagggaagtcatacaacgaacatgatgaccgtgttagtttcccacaaagaagaaagaaaggtcgggggcctttccgatggaagtgtggtgtggggaatcgtaggtctggaagaggtggttctggtatacggtcttcccggtttgaggaagatgatggagatgtggcaatgaatgatccccaggatggcccccgagtgagattcaacccctataccacccgacctaaccgtcggagagatacttggcatgatcgagatcggattcatgttactgtacggagagacagagctcctcaagaaagaggaggtgctggcaccagtcaagatgggaccaccaagaactggttcaagattacaattccttatggcaaaaagtatgataaaatgtggcttctgagcatgattcaaagcaagtgcagtgtccccttcaaccccattgagtttcactatgaaaatacacgggcccatttttttgtggaaaatgccactaccgcttctgcattaaaggctgtcaactataagattcaggatcgagaaaacggaaggatatctatcatcatcaactcttctgctccaccctatattgtacagaatgaactgaagcctgaacaagtagagcagctcaagctgatcatgagcaaacgatatgatggctcccagcaagcacttgacctcaagggcctccgctcagacccagatttggtggcccagaacatcgatgttgttctaaaccgtagaggctgtatggcagccgccctgaggatcattgaagagaacattcctgagctattgtctttgaacttgagtaacaacaggctgtacaagcttgatgacatgtccagcattgtacagaaggcaccaaacttaaagatcctgaatctctctggaaacgaattgaagtccgagtgggaattagacaagataaaagggctgaagctggaagagctgtggcttgacagaaaccccatgtgtgacaccttcctagaccagtccacctatatcagcaccattcgcgaacgatttcccaagttattacgcctggatggccatgagctaccccctccaattgcctttgatgtcgaagcccctactatgttaccaccctgcaagggaagctattttggaacagagaacctaaagagtctggtcctgcacttcttgcagcagtactatgcaatttatgactcaggagaccgccagggtcttctggatgcctaccatgatggcgcctgttgctcgctaagcactccttccaaccctcagaaccctgtccgacacaacttagccaaatacttcaatgacagcagaaatgtgaagaagattaaagataccaccacgcggttccggctattaaagcatacacgtcttaatgtcgttgccttcctcaatgagttacctaaaactcaccatgacgttaactcctttgtggtagatataagtgcccaaacaagcacattgttgtgtttttctgtcaatggagtcttcaaggaagtggacgggaagtctcgagattctttacgtgctttcacgagaacgttcattgctgttcctgccagcaattcagggctatgtattgtaaatgacgaattatttgtgcggaacgccagccctgaagaaatccaaagagccttcgccatgcctgcacccacaccttcttccagcccagtacccacactctcccaagaacagcaggatatgttacaagcattttctacccagtctggcatgaacctcgagtggtctcagaagtgccttcaagacaataactgggactacaccagatctgcccaggccttcactcatctcaaggccaagggagagattccagaagtagcgtttatgaaatgatctcaagaaaagtccccttgtaaatagcccttggataatattgtctggctgtcatctgcggactcctgtccagcccgaggccacmctgtaactgtgactggggaggaagggctgcytcatccktcctcacctaccttctggaagmcttagcagaagacttccggaagattgagcctctatggtgccaggaagccaaagcttactttgtagagctdgacrctaaattaccggaaggacttaggtgctctgtgtacttaaccccagsattccctttactttttaagataaagagtgatattgtagtgtgtgtcccaaaaaaaaaaaaaa

SEQ ID NO: 104 - protein, 175 aa, Rattus norvegicus
  Alpha B-crystallin
MDIAIHHPWIRRPFFPFHSPSRLFDQFFGEHLLESDLFSTATSLSPFYLRPPSFLRAPSWIDTGLSEMRMEKDRFSVNLDVKHFSPEELKVKVLGDVIEVHGKHEERQDEHGFISREFHRKYRIPADVDPLTITSSLSSDGVLTVNGPRKQASGPERTIPITREEKPAVTAAPKK

SEQ ID NO: 105 - DNA, 6806 bp, Rattus norvegicus
  Alpha B-crystallin
gggaagagcagcctatgactttctgcccacacggacgccattcggtctaccaggttacagacaggagcagcacaaacaaggcccctagattttggtttatttagcctcacccagaattaaatcgaaggctacgattcatcaagggaccaaaccatacactgtggaccatgcacctatcccatctcccaggaatgctgctccctctggggtgtggtggatttctggaatcaccacgggaggtagacaggggattcaccgggttctgggtctggcagtcagggttcaactctggctacctcttcctcttcctcggggtcaggaggtgcaggaagcagggagatgtagacctcattgacttccgtgtccagatgccggccaccccgcggtgcctccaagttaaggatgccatcatgggagagagcagctctgacccgccaggggtccacatctgcaggcaggacataggttgcgacagaactctcgggacacgaagccgtggcgtccagacgttgggggtgtcgggcagacactctagcaggttgtccacagtcctcacggtcacctcatctggggtaaagtggctcacatccagaaatgcctggaacttgccttcactgagcctgagctctgaggcccctgccctgccgccctccccagctcctggcggcccggggccgaacatagtagccatggtagagggtgggggtcaggatctcttctggcaggaggcctgggagcagagaaagaaagagaaggcaagaggcaggatgaggaagaatgcagcgggcacagacagggctgaaaggcgatatctggagatatgggctagaagagcaggtgggaaactgtagaggggctgggaaaggagaggagatggttacggaaaaagcgagctggaactggaggacagaagcaaaaccagaaaatactctgaagcgtgcaaggcccggacaccgaaagccctgccgatctaaagtttggtcctgaagtctgttagcaagaccagagagagtcttgcttgaggggggagtctggggtgggagatggctcacctcaggagttcacttaagccccaggtagagtggaggccagtgaagctgggcagtgagcccagaatttccggaggtgggggtaggaagcatctgagccataccttctccgaagcgctgctcgccccaggcggctagggttggcaaattcgtactcggcagtggctgggtgggcgtgtggcactgtgcggcccgacatgactgctgttgcgactagtagccctgatcccaagtgtagccccaacaagatcaaggcgacccctccagcttcagagggagagccaggactcagtcagagtgggggcggggtctacaccacccaaaatagtgcagagcgtctgggggatgggggaggggtgggagcctaagtctagagtgcagctatgagggtgtgatcagcgctgcctggaccccctaagtcccctcctctgtatacccttcagctgtcgcataggcctggaagaacagctgagggtcctggtcctagagtaagctgggaagagaaactggtgcgctgcaggagggcaaggagaggcctggttgggcccttcaccaattgtacattccacatcaccctttgtccttatcagtctcaggcactgagcacattacttagaaaaaaaaaaaaaaagacaatgttctatgagccacagaacgtcaaaaatacaagaaacattttctgtctttttaatgtcagggtcttctgaacctagatcaactcggggttccagtcagacacctagttctgacatattggtggtcacagctctcctctgggactccacaaagagttaatgtccctgggctcagcccaggaagattccagcctctgcccaggcccaagatagttgctggctcaattcccctggcatgcaagactggagaggaggaggggcccaccagcagctgcttgggattccagaccctgtcctggctccagagaacaaggatggggtgggtgggtgccactaggtgtggacagagagctagtgaaacaagaccgtgacaagtcaccggccagctcagccctgccccgtgtttctcttttcttagctcagtgagtactgggtatgtgtcaccctgccaaatccctgatcacaagtccccatgaactgtcggggagctgggataataaaacccctgacatcaccgttccagaagcttcacaagactgcatatataaggggcaggctgtagcagcggctgaaggagttgaccggctaaccgactctacactcatctagccatcatggacatagccatccaccacccctggatccggcgtcccttctttcctttccactccccaagccgcctctttgaccagttcttcggagagcacctgttggagtctgacctcttctctacagccacttccctgagccccttctaccttcggccaccctccttcctgcgggcacctagctggattgacactgggctctcagaggtaagtctcccttgtgccagggcaggaatacgtatccttcctggtctctccattcctttgccctttcaccctgcctaagtcattttaggcatgtgcgtgtgcaaaggtacagagaaatgaggttactgttctcttcctgtggcacctgttctgttgatgctcctgttcctggtccattttctgtgcatggtgcctctttctcctctttcaactcctttacccacctctttaatagtcccagtcctggcagattttatcatctcaaactgtagagatcagatagaaacccactcattctgggcttttcctgtcttctttcctccccacttccttttgatggagctggatacatcaatcaatattctctctctctctctctctctctctctctctctctctctctctctcttccttggcctatgactttccgggttcctcggggctgagacagtgcattgacttcagtccatgccttaagaggaccgctcttgggaagccctgacttcttatctcagcagcgtgggagataagattaacagaggttgtcacagataacactgtggtttaaaaatattcgcgtgtgaggaaacaggagctgagtgagcaagggctttggaaggacagaacattccagatcgagtgggttggaaacttggcaggcaactgaacaagaaaaagggggcttttgttttatagacaaatgacaaagccagcactgggtagagaggcagcagatgccatccccagacccattcttgtgactagtctcctactgacccgtcttcttccctgtgcctctcggtaaagtttgggactgatggatgactaagaggtgtcttttgggtatcaccatggtggctcacatataccagtgtcactagtcaataagaggatttttcccccagcagccaggtagaggatggattaccagggcaggaagtaaatctcctggacgatgagccttctccagcttaatggacattctaccatcttttctcctccgcccccttgttattctagatgcgtatggagaaggacaggttctctgtgaacctggacgtgaagcacttctctccagaggaactcaaagtcaaggttctgggagacgtgattgaggtgcacggcaagcacgaagagcgccaggtgtgtggacctttctgccttctcttgtgaattcatttagtgccctgctggatgccagggactgccgccaccagcctctgaggctggcagaattccagtccccaaacataagactagatcaggagttatagatcaatggctgggaaacaagttagggctagatctgctgttagtgttgtggctgtgctgagagaactttcccctggatgttccttttgattgccttgtcttgtccagtgacgtcagaatagcagaagaattctcaacattcccaaccctcagcacatcctaggccaattgaatctgaagttttagggacatagctcaggcatcaacctttaaacaaacaaacaaacaaactcactccttgggtactttctgtaccccaaactggcagccattgctctaggcacagtgtgctgtacataatggctctgcgaaatgacagtcaccacggagtcgaaggcaataaaatctgtggcttttgaatgttcaggattccatgcctcttctgctcagtttaatgctatgctacttggtttgtctgcttttcagagcttcaggcttatggctgtgaggaagttaattgagatttgtttgtgtggatgcttggggtacttgggattcaataggtaacaggattagcatccacaattcctcttgcatttttagcatttttttttcttgacttcctaaaaaggtgttatgattgcttcttaaaaattaatgacttggggggctggggatttagctcagtggtagagcgcttacctaggaagcgcaaggtcctgggttcggtccccagctccgaaaaaaaagaaccaaaaaaaaaaaaattaatgacttggggttggggatttagctcagcggtagagcgtttgcctagcaagcgcaaggccctgggttcagtccccagctccgaaaaaaagaaaagaaaaaaaaattaatgacttagggcctggagagatggctcagcagttaaaagcactgaccgctcttccagaggtcctgcatacaattcccagcaaccacatggtggctcacaaccatctgtaatgggatccaatgccctcttctggcatgcaggtgtacatgcagatagagtgctcatacataaaattaaaaaaatcttaacaaaaaataactcagctgtgccgtggtggtgcatgcctttaatcccaacatttgggaggcagaggtagacagatctaagttagaggccagcttagactacagaatgagttccaggagagcaggagctcaacagagaaaccctacttatctcaaacaaaacaaaaaacaaaaatcaatacctcggttttgctcatgatttaggactggaccacagtgtcactgatagggtcaccttatataatatcagcaatattatactagaaacattctgagacattcctgagcaattagaacaatttatgggcttcctataagatacagacatactcggggttggggatttggctcagtggtagagcgtttgcctaggaagcgcaaggtcctgggttcggtccccagccctgaaaaaaagaaaaaagaaaaaaaaaaaagatacagacatactcaatgccaaagtgggagtctaaattccatggtccagtcaacatggattcattcctttgtggaaatgccctgaattgtagttgtgtctccgaatatgaagatctatgaaattcggggctgtttcagatttcctacactctgattggtaggtgtgtccatctggacagtttattctagccttaaacctggacaaacagctctggacttagctatctgcatatttacagtgttggaggtgaactcaggcctttcactactagactgaatccccagctccagatattttttccccccttagggccttttgttctaacccaagggcaggggatgtctagagtctggggcaagtggtggtatttcctgctcttatttctctcccttgcccttcattctctcggattaggacgaacatggcttcatctccagggagttccacaggaagtaccggatcccagccgacgtggatcctctcaccattacttcttccctgtcatcggatggagtcctcactgtgaatggaccaaggaaacaggcctctggccctgagcgcaccattcccatcacccgtgaagagaagcctgctgtcactgcagcccctaagaagtagattccctttcctcgttgcattttttaagacaaggaagtttcccatcagcgaatgaacatctgtgactagtgccgaagcttactaatgctaagggctggcccagattattaagctaataaaaaatatcgttcagcaacagacctgcctcgtctttgcaaacccagtgtgctttaaaccaatctgcaaacatagcagatctgctgatccccaccctgtgattcagggggctccgaggatacgtttggtcagagtgattggctccagaactttgtacagtacaaacccagggaaaaccattcactctcctggtattccagtcttcagtttataacacactgtactggttaggacagagtcttaggagtagactaagaagggagaaatggtgacgtcccagcagggtcatgatgaatcagctctggctaactccacataaaagacttcagtgcatttctgaagattaattacttcaccagggggacacggtggccttggtgttatttgaagagtgtcctcagctaattcacaggacagtaaatagctcgtgggtggtgactgtcgaacgagagagcctctcgggaatggcacagagtaatcaacagataccttttaaaccgagaccattctctaccaaacatgaaaatgacaataaagggtccagcaaaaaaacaaacaccaaaatcagaagaacatcttcctcctaaagttactaacccctggggtggggggaagcccacacaaaatagaaaccccccaaaaacctctctccattttaatgggtctagacatagtttctgtcagattacactgagccctgctacccgatatctggcaagcctgtagcaccagttctcatctctcctgtgttaactttaggttcacttgaggatgtgccttttgaagattttttttatttagtaattattatttttttataaaacttgataaaaaatagtatttccaactgtacagtcactag

SEQ ID NO: 106 - protein, 646 aa, Rattus norvegicus
  Heat shock cognate protein 70
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQAAKDAGTIAGLNVLRIINEPTAAAIAYGLDKKVRAERNVLIFDLGGGTFDVSILTTEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIISWLDKNQTAEKEEFEHQQKELEKVCNPIITKLYQSAGGMPGGMPGGFPGGGAPPSGGASSGPTIEEVD

SEQ ID NO: 107 - DNA, 2242 bp, Rattus norvegicus
  Heat shock cognate protein 70
atgcactttcaaagtagtgagaaaattttgatgttttctaatacatagaaatgatatcattaaaaggcagatgtggctcattgaacgcagagccagctctcgggggtctctgtgtggtctcgtcatcagcacagctgggcctacacgcaagcaaccatgtctaagggacctgcagttggcattgatcttggcaccacctactcctgtgtgggtgtcttccagcatggaaaggtggaaataattgccaatgaccagggtaaccgcaccacgccgagctatgttgctttcaccgacacagaacgattaattggggatgcggccaagaatcaggttgcaatgaaccccaccaacacagtttttgatgccaaacgtctgatcggacgtaggttcgatgatgctgttgttcagtctgacatgaagcactggcccttcatggtggtgaacgatgcaggcaggcccaaggtccaagtcgaatacaaaggggagacaaaaagtttctatcctgaggaagtgtcttcaatggttctgacaaaaatgaaggaaattgcagaagcttaccttggaaagactgttaccaatgccgtggtcaccgtgccagcttacttcaatgactctcagcgacaggcagcaaaagatgctggaactattgctggcctcaacgtacttcgaattatcaatgagccaactgctgctgctattgcctatggcttagataagaaggtcagggctgaaaggaatgtgctcatttttgacttgggaggtggcacttttgatgtgtcaattctgactactgaggatggaatttttgaagtcaaatcaacagctggagacacccacttgggcggagaagactttgacaaccgaatggtcaaccatttcattgctgagtttaagcgaaagcacaagaaggacatcagtgagaacaagagagctgtcaggcgtctccgcactgcctgtgagcgggccaagcgcaccctctcctccagcacccaggccagtattgagattgattctctctatgagggaattgacttctacacctccattacccgtgctcgatttgaggagttgaatgctgacctgttccgtggcacactggaccctgtagagaaggcccttcgagatgccaaactagacaagtcacagatccatgatattgtcctggtgggtggttctaccagaatccccaagatccagaaacttctgcaagacttcttcaatggaaaagagctgaataagagcattaaccccgatgaagctgttgcctatggtgcagctgtccaggcagccattctatctggagacaagtctgagaatgttcaggatttgctgctcttggatgtcactcctctttcccttgggattgaaactgctggtggagtcatgactgtcctcatcaagcgcaataccaccattcccaccaagcagacccagactttcaccacctactctgacaaccagccaggtgtactcatccaggtgtatgaaggtgaaagggccatgaccaaggacaacaacctgcttgggaagtttgagctcacaggcatacctccagcaccccgtggggttcctcagattgaggttacttttgacattgatgccaatggcatcctcaatgtttctgctgtagataagagcacaggaaaggagaacaagatcaccatcaccaatgacaagggccgcttgagtaaggaggatattgagcgcatggtccaagaagctgagaagtacaaagctgaggatgagaagcagagagataaggtttcctctaagaactcgctggagtcttatgctttcaacatgaaagcaactgttgaggatgagaaacttcaaggcaagatcaatgatgaagacaaacagaagattcttgacaagtgcaacgaaatcatcagctggctggataagaaccagactgcggagaaggaagaatttgagcatcagcagaaagaactggagaaggtctgcaaccctatcatcaccaagctgtaccagagtgctggtggcatgcctggaggaatgcctggtggcttccctggtggaggagctcctccatctggtggtgcttcttcaggccccaccattgaagaggtcgattaagtcaaagtagagggtgtagcattgttccacagggacccaaaacaagtaacatggaataataaaactatttaaattggcacaaaaaaaaaaaaaaaaacggagatgtggtgactcagctgtaatcccggcacttaggaagctgagg

SEQ ID NO: 108 - protein, 374 aa, Rattus norvegicus
  Tau microtubule associated protein
MAEPRQEFDTMEDQAGDYTMLQDQEGDMDHGLKAEEAGIGDTPNMEDQAAGHVTQARVAGVSKDRTGNDEKKAKGADGKTGAKIATPRGAATPGQKGTSNATRIPAKTTPSPKTPPGSGEPPKSGERSGYSSPGSPGTPGSRSRTPSLPTPPTREPKKVAVVRTPPKSPSASKSRLQTAPVPMPDLKNVRSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVHKSPVVSGDTSPRHLSNVSSTGSIDMVDSPQLATLADEVSASLAKQGL

SEQ ID NO: 109 - DNA, 5240 bp, Rattus norvegicus
  Tau microtubule associated protein
cctcccctggggaggttcgcgttcccgtggctcgcgcctgctgcccgccggcctcgagaacgcgctctctcggccgcggcgctcacagtctccgccacccaccagctccagcaccagcagcagcgccggcgccaccgcccaccttctgctgtcgccgccgccacaaccaccttcccctccgctgtcctcttctgtcctcgcctcctgtcgattatcaggctttgaagcagcatggctgaaccccgccaggagtttgacacaatggaagaccaggccggagattacactatgctccaagaccaagaaggagacatggaccatggcttaaaagctgaagaagcaggcatcggagacaccccgaacatggaggaccaagctgctgggcatgtgactcaagctcgagtggccggcgtaagcaaagacaggacaggaaatgacgagaagaaagcgaagggggccgatggcaaaacgggggcgaagatcgccacacctcggggagcagccactccgggccagaaaggcacatccaatgccaccaggatcccagccaagaccacacccagcccaaagactcctccaggatcaggtgaaccaccaaaatccggagaacgaagcggctacagcagccccggctcgcccggaacccctggcagtcgctcccgtaccccatccctaccaacgccgcccacccgagagcccaaaaaggtggcagtggttcgcactccccctaagtcaccgtctgccagtaagagccgcctacagactgcccctgtgcccatgccagacctaaagaacgtcaggtccaagattggctccactgagaacctgaagcaccagccgggaggcggcaaggtgcagataattaataagaagctggatcttagcaacgtccagtccaagtgtggctcaaaggacaatatcaaacacgtcccgggcggaggcagtgtgcaaatagtctacaagccagtggacctgagcaaggtgacctccaagtgtggttccttagggaacatccatcacaagccaggaggtggccaggtagaagtaaaatcagagaagctggacttcaaggatagagtccagtcgaagattggctccttggataacatcacccatgtccctggaggagggaataagaagattgaaacccacaagctgaccttcagggagaatgccaaagccaagacagaccatggagcagaaatcgtgcacaagtcacctgtggtgtctggggacacatctccacggcacctcagcaacgtctcctccacgggcagcatcgacatggtggactctccacagcttgccacgttagccgatgaagtgtccgcctctttggccaagcagggtttgtgatcaggcccctggggccgtcactgatcatggagagaagagagagtgagagtgtggaaaaaaaaaaaaaaagaatgacctggcccctcaccctctgccctccccgctgctcctcatagacaggctgaccagcttgtcacctaacctgcttttgtggctcgggtttggctcgggacttcaaaatcagtgatgggaaaaagtaaatttcatctttccaaattgatttgtgggctagtaataaaatatttttaaggaaggaaaaaaaaaacacgtaaaaccatggccaaacaaaacccaacatttccttggcaattgttattgaccccgcccccccctctgagttttagagggtgaaggaggctttggatagaggctgcttctggggattggctgagggactagggcaactaattgcccacagccccatcttaggggcatcagggacagcggcagacatgaaagacttgggacttggtgtgtttgtggagccgtaaggcgtatgttaactttgtgtgggtttgagggaggactgtgatagtgaaggctgagagatgggtgggctgggagtcagaggagagaggtgaggaagacaggttgggagagggggcattgcgtccttgccaaggagcttgggaagcacaggtagccctggctgcagcagtcttagctagcacagatgcctgcctgagaaagcacagtggggtacagtgggtgtgtgtgccccttctgaagggcagcccatgggagaaggggtattgggcagaaggaaggtaggccccagaaggtggcaccttgtagattggttctctgaaggctgaccttgccatcccagggcactgctcccaccctccaggaggaggtctgagctgaggagcttccttttcgatctcacaggaaaacctgtgttactgagttctgaagtttggaactacagccatgattttggccaccatacagacctgggactttagggctaaccagttctttgtaaggacttgtgcctcttgcgggaacatctgcctgttctcaagcctggtcctctggcacttctgcagtggtgagggatgggggtggtattctgggatgtgggtcccaggcctcccatccctcgcacagccactgtatcccctctacctgtcctatcatgcccacgtctgccacgagagccagtcactgccgtccgtacatcacgtctcaccgtcctgagtgcccagcctcccaagcccaatccctggacccctgggtagttatggccaatctgctctacactaggggttggagtccagggaaggcaaagatttgggccttggtctctagtcctacgttgccagaatccaaccagtgtgcctcccacaaggaaccttacaaccttgtttggtttgctccatcaggcgtttggcgccatcgtggatggagtccgtgtgtgcctggagattaccctggacacctctgctttttttttttttactttagcggttgcctcctaggcctgactccttcccatgttgaactggaggcagccaagttaggtgtcaatgtcctggcatcagtatgaacagtcagtagtcccagggcagggccacacttctcccatcttctgcttccaccccagcttgtgattgctagcctcccagagctcagccgccattaagtccccatgcacgtaatcagcccttccataccccaatttggggaacataccccttgattgaaatgttttccctccagtcctatggaagcggtgctgcctgcctgctggagcagccagccatctcagagacgcagccctttctctcctgtccgcaccctgctgcgctgtagtcggattcgtctgtttgtctgggttcaccagagtgactatgatagtgaaaagaaaaagaaaaagaaaaaagaaaaaagaaaaaaaaaaaaggacgcatgttatcttgaaatatttgtcaaaaggttgtagcccaccgcagggattggagggcctgatattccttgtcttcttcgtgacttaggtccaggccggtcgagtgctaccctgctggacatcccatgttttgaagggtttcttcttcatctgggacccctgcagacactggattgtgacattggaggtctatacattggccaaggctgaagcacaggacccgttagaggcagcaggctccgactgtcagggagagcttgtggctggcctgtttctctgagtgaagatggtcctctctaatcacaacttcaagtcccacagcagccctggcagacatctaagaactcctgcatcacaagagaaaaggacactagtaccagcagggagagctgtggccctagaaattccatgactctccactactatccgtgggtcctttccaagccttgcctcgtcaccaagggcttgggatggactgccccactgatgaaagggacatctttggagacccccttggtttccaaggcgtcagccccctgaccttgcatgacctcctacagctgaaggatgaggcctttaaagattaggaacctcaggcccaggtcggccactttgggcttgggtacagttagggacgatgcggtagaaggaggtggccaacctttccatataagagttctgtgtgcccagagctaccctattgtgagctccccactgctgatggactttagctgtccttagaagtgaagagtccaacggaggaaaaggaagtgtggtttgatggtctgtggtcccttcatcatggttacctgttgtggttttctctgtatacccccatttacccatcctgcagttcctgtccttgaataggggtgggggtactctgccatatctcttgtaggcagtcagcccccaagtcatagtttggagtgatctggtcagtgctaataggcagtttacaaggaattctggcttgttacttcagtgaggacaatcccccaaggccctggcacctgtcctgtctttccatggctctccactgcagagccaatgtctttgggtgggctagatagggtgtacaatttgcctggaacctccaagctcttaatccactttatcaatagttccatttaaattgacttcaatataagagtgtatccatttgagattgcttgtgttgtggggtaaaggggggaggaggaacatgttaagataattgacatgggcaaggggaagtcttgaagtgtagcagttaaaccatcttgtagccccattcatgatgttgaccacttgctagagagaagaggtgccataaggctagaacctagaggcttggctgtccaccaacaggcaggcttttgcaaggcagaggcagccagctaggtccctgacttcccagccaggtgcagctctaagaactgctcttgcctgctgccttcttgtggtgtccagagcccacagccaatgcctcctcaaaaccctggcttccttccttctaatccactggcacatcagcatcacctccggattgacttcagatccacagcctacactactagcagtgggtaagaccacttcctttgtccttgtctgttctccagaaaagtgggcatggaggcggtgttaataactataggtctgtggctttatgagccttcaaacttctctctagcttctgaaagggttacttttgggcagtattgcagtctcaccctccgatggctgtagcctgtgcagttgctgtactgggcatgatctccagtgcttgcaagtcccatgatttctttggtgttttgagggtggggggagggacatgaatcatcttagcttagcttcctgtctgtgaatgtccatatagtgtactgtgttttaacaaacgatttacactgactgttgctgtacaagtgaatttggaaataaagttattactctgattaaacaaaaaaaaaaaaaaaaaaaaaaaaaaaaaaaa

SEQ ID NO: 110 - protein, 248 aa, Rattus norvegicus
  Schwann cell peripheral myelin
MAPGAPSSSPSPILAALLFSSLVLSPTLAIVVYTDREVYGAVRSQVTLHCSFWSSEWVSDDISFTWRYQPEGGRDAISIFHYAKGQPYIDEVGTFKERIQWVGDPSWKDGSIVIHNLDYSDNGTFTCDVKNPPDIVGKTSQVTLYVFEKVPTRYGVVLGAVIGGILGVVLLLLLLFYLIRYCWLRRQAALQRRLSAMEKGKFHKSSKDSSKRGRQTPVLYAMLDHSRSTKAASEKKSKGLGESRKDKK

SEQ ID NO: 111 - DNA, 1029 bp, Rattus norvegicus
  Schwann cell peripheral myelin
attcggctgggcccttgcccctaccccagctatggctcctggggctccctcatccagccccagccctatcctggctgccctgctcttctcttctttggtgctgtccccaaccctggccattgtggtttacacggacagggaagtctatggtgctgtgcgctcccaggtcaccctgcactgctccttctggtccagtgaatgggtctcagatgacatctcttttacctggcgctaccagcctgaaggaggccgagatgccatttcaatcttccactatgccaagggtcaaccttacatcgatgaggtggggaccttcaaggagcgcatccagtgggtaggggaccctagctggaaggatggctccattgtcatacacaacctagactacagtgacaacggcactttcacatgtgatgtcaaaaacccaccggacatagtgggcaagacgtctcaggtcacgctctatgtctttgaaaaagtgcccactaggtatggggtggtgttgggagccgtgatcggtggcatcctcggggtggtgctgttgctgctgttgctcttctacctgatccggtactgctggctgcgcaggcaggctgccctgcagaggaggctcagtgccatggagaaggggaaatttcacaagtcttctaaggactcctcgaagcgcgggcggcagacgccagtgctgtatgccatgctggaccacagccgaagcaccaaagctgccagtgagaagaaatctaaagggctgggggagtctcgcaaggataagaaatagcggttagcgggccgggcggggggtcgggggtctgcgatggagtcttccaaaggctctcaggtggtggtcatcgagatggagcttcgcaaagatgagcagagctcggagccccggcctgcagtcaaatcccccagtagaaccagcctcaagaacgccctcaagaacatgatgggcctggactcgaacaagtgaccgtcacctccaggctctgtcagagcaggggacctaggctcctctttttcccagtctag

SEQ ID NO: 112 - protein, 444 aa, Rattus norvegicus
  Class 1 beta tubulin
MREIVHIQAGQCGNQIGAKFWEVISDEHGIDPTGTYHGDSDLQLDRISVYYNEATGGKYVPRAILVDLEPGTMDSVRSGPFGQIFRPDNFVFGQSGAGNNWAKGHYTEGAELVDSVLDVVRKEAESCDCLQGFQLTHSLGGGTGSGMGTLLISKIREEYPDRIMNTFSVVPSPKVSDTVVEPYNATLSVHQLVENTDETYCIDNEALYDICFRTLKLTTPTYGDLNHLVSATMSGVTTCLRFPGQLNADLRKLAVNMVPFPRLHFFMPGFAPLTSRGSQQYRALTVPELTQQVFDAKNMMAACDPRHGRYLTVAAVFRGRMSMKEVDEQMLNVQNKNSSYFVEWIPNNVKTAVCDIPPRGLKMAVTFIGNSTAIQELFKRISEQFTAMFRRKAFLHWYTGEGMDEMEFTEAESNMNDLVSEYQQYQDATAEEEEDFGEEAEEEA

SEQ ID NO: 113 - DNA, 2346 bp, Rattus norvegicus
  Class 1 beta tubulin
ctcgcatctctctgcagcagcctccggggctacgttccatcctatcagtctgagaccagcccagaagaaaaaaaccaaaaatccttaattatcttcttttgctcggtacctacattgggaccatcaaaaaaaaattattacagtaaaccgtagccatgagggaaatcgtgcacatccaggccggacagtgtggcaaccagatcggtgctaagttctgggaggtgataagcgatgaacacggcatcgaccccaccggcacctaccacggagacagcgacttgcagctggaccgaatctctgtgtactacaatgaagctacaggtggcaagtatgtccctcgagctatcttagtggatctagaacccgggactatggactccgttcgctcaggtccttttggccagatcttcagaccggacaactttgtttttggtcagtctggggcaggcaacaactgggctaagggtcactacacagagggagctgagctggttgactctgtcttggatgtggtgcggaaggaggcggagagctgtgactgcctgcaaggctttcagctgacccactcgctgggtggaggcacgggctctggcatgggcaccctgctcatcagcaagattcgagaagaataccccgaccgcatcatgaacaccttcagcgtggtgccctcacccaaagtgtctgacaccgtggttgagccctacaacgccaccctgtccgtccaccagttggtagagaacacagatgagacctactgcattgacaacgaggctctctacgacatctgcttccgtacccttaagctcaccacgccaacctacggagacctgaaccatctcgtctctgccaccatgagcggtgtcaccacctgcctccgtttcccgggccagctgaacgccgaccttcggaagctggctgtcaacatggtgcccttcccgcgtctccacttcttcatgcctggctttgcccctctcaccagccgtggaagccagcagtaccgggccctcactgtgcctgaactcacccagcaggtcttcgacgccaagaacatgatggccgcctgtgacccacgccacggccggtacctcacggttgcggctgtcttccgtggacggatgtccatgaaggaggtagatgagcaaatgctcaacgtgcagaataagaatagcagctacttcgtggaatggatccccaacaacgtcaagacggctgtctgtgacatcccaccgcgaggcctcaagatggcagtcaccttcatcggaaacagcacggccatccaggagctcttcaagcgcatctctgagcagtttacggcaatgttccgtcggaaggctttcctccactggtacacgggtgagggcatggacgagatggagttcaccgaagctgagagcaacatgaatgacctcgtctctgagtatcagcagtaccaggacgccaccgcagaagaggaagaggatttcggagaggaggcagaagaagaggcctaaggcggaagccctgcgtcacctcaggctgcttagttcccttagcttttttccaactgccctttgtttctttctgctgcctctgtcctgtttttgttccgttttgttttctcattggggggtaaacggtgcctggcacgtggcaggcgctcaataaatatttctttgtggaatgtctcctttctctttccgatctgacattctgagcgttaaccctgtgcttcctcctggtacccatttctttattatgactgtccagttaatattcttccaatatattctccaggaaccagcgttcggctccagacccggggtagaacccagcccagttctaagaactcaagagagtagccaccatcttaaagcaaagtagcgggtggggaaaaggtgggtagaggagatgctacttagggggtgggattttccagtcagggccatttagacacgggaatcgctggggctttcagtcgatggggctttcttttttccctgcttcatctctcggcctcaggagaggtattaacagtattatcaccatttatatcctagctgtcctgagccaaatctgccattggaggtgtcttcccctgtgttggttctccaagggacgttgtatggggatttgtgggggcagggcagcaaggccttgttctctaaatgtccagaaacactctaattaccatttccacctgggtgcctcacacactccccagaggggaggtaacatctctgccccatttccatcctgttccttggcttggtcattccctacctttctattttctgttaaacttggctttttttttttcatattgaaaagatgacattgccccgagagccaaaaataaatgggaattgg

SEQ ID NO: 114 - protein, 724 aa, Rattus norvegicus
  Heat shock protein 90
MPEEVHHGEEEVETFAFQAEIAQLMSLIINTFYSNKEIFLRELISNASDALDKIRYESLTDPSKLDSGKELKIDIIPNPQEATLTLVDTGIGMTKADLINNLGTIAKSGTKAFMEALQAGADISMIGQFGVGFYSAYLVAEKVVVITKHNDDEQYAWESSAGGSFTVRADHGEPIGRGTKVILHLKEDQTEYLEERRVKEVVKKHSQFIGYPITLYLEKEREKEISDDEAEEEKGEKEEEDKEDEEKPKIEDVGSDEEDDSGKDKKKKTKKIKEKYIDQEELNKTKPIWTRNPDDITQEEYGEFYKSLTNDWEDHLAVKHFSVEGQLEFRALLFIPRRAPFDLFENKKKKNNIKLYVRRVFIMDSCDDLIPEYLNFIRGVVDSEDLPLNISREMLQQSKILKVIRKNIVKKCLELFSELAEDKENYKKFYEAFSKNLKLGIHEDSTNRRRLSELLRYHTSQSGDEMTSLSEYVSRMKETQKSIYYITGESKEQVANSAFVERVRKRGFEVVYMTEPIDEYCVQQLKEFDGKSLVSVTKEGLELPEDEEEKKKMEESKARFENLCKLMKEILDKKVEKVTISNRLVSSPCCIVTSTYGWTANMERIMKAQALRDNSTMGYMMAKKHLEINPDHPIVETLRQKAEADKNDKAVKDLVVLLFETALSSLASHFRRPKTHSNRIYRMIKLGLGIDEDEVTAEEPSAAVPDEIPPLEGDEDASRMEEVD

SEQ ID NO: 115 - DNA, 2524 bp, Rattus norvegicus
  Heat shock protein 90
ggcctaggcttgccgtgcgagtcggacgtggtccgggcccaccctgctctgtactactactcggctttctcgtcaagatgcctgaggaagtgcaccatggagaggaagaggtggagaccttcgcctttcaggcagaaattgcccagctgatgtccctcatcatcaacactttttactcaaacaaagagattttcctccgggagttgatctctaatgcttctgatgccctggacaagattcggtatgagagcctgactgacccttccaagttggacagcgggaaagagctgaagattgacatcatccccaaccctcaggaagccacgctgactttggtggacacaggcattggcatgaccaaggctgacctcataaataacttgggaaccattgctaagtctggtacaaaggcgttcatggaggctctccaggctggtgcagacatctccatgattgggcagtttggtgtcggcttctactctgcctacctggtggcagagaaagtggttgtgatcacgaagcacaacgatgatgagcagtatgcctgggagtcttctgctggtggatccttcactgtccgtgcagaccacggtgagcccattggccggggtaccaaagtgatccttcacctcaaagaagaccagacagagtacttagaggagaggagggtcaaggaggtggtgaagaaacactcacagttcataggctaccccatcaccctctatctggagaaggaacgcgagaaggagatcagtgatgatgaggcagaggaagagaaaggggagaaggaggaggaagataaggaggatgaggagaagcctaagattgaggatgtgggatctgatgaggaggatgacagcggcaaagataagaaaaagaaaacaaagaagatcaaggagaagtacattgaccaggaagagctgaataagacgaagcccatctggaccagaaatcctgatgacatcactcaagaggaatatggtgaattctacaagagcctcaccaatgactgggaagaccacttggcagtcaagcacttctctgtagaagggcagttggaattcagggcattgctcttcattccccggcgagctccctttgacctctttgagaacaagaagaagaagaacaacatcaaattgtatgtccgtcgtgtgttcatcatggacagctgtgatgacctgatacctgagtacctcaacttcatccgtggtgtggttgattccgaggacctgcctctgaacatctcccgagaaatgctccagcagagcaagatcctgaaagtcatccgcaaaaacattgtgaagaagtgccttgagctcttctctgagttggctgaagacaaagagaactacaagaaattttatgaggcattctctaagaatttaaagcttggaatccatgaggattccactaaccgtcgccgcctctctgagctccttcgctaccatacctctcagtctggagatgagatgacttccctgtcagagtatgtgtctcgcatgaaggagacacagaagtccatctactatatcactggtgagagcaaagagcaggtggccaactctgccttcgtggagcgtgtgcggaaacggggctttgaggtggtatacatgactgagcccattgatgagtactgcgtacagcagctcaaggagttcgatggcaagagcctggtctccgtgaccaaggagggcctggagctaccagaggatgaggaagagaagaaaaaaatggaggagagcaaggcaaggtttgagaatctctgcaagctcatgaaggagatcttggacaagaaggttgaaaaagtgactatctccaataggcttgtgtcttccccctgctgcattgtgaccagcacctacggctggacagccaacatggaacggattatgaaggcccaggcactgcgggacaactcgacaatgggctacatgatggccaagaaacacctagagatcaaccctgaccaccccattgtggagactctgcggcagaaggctgaggcagacaaaaacgacaaagctgtcaaagacctggtggtgctgctgtttgagactgctctgtcttctctcgcttctcactttaggaggcccaaaacccactccaaccgcatctaccgcatgattaaactaggcctgggcattgatgaagatgaggtcactgcagaagagcccagtgctgctgttcccgatgagatccccccactggagggtgatgaggatgcctctcgcatggaagaagtggattaaaggctcctgggaagcccccgccctctgtatagtatccctgtggctcccccaacagccttgactggccctgacccacctgggctctctctgctcatgtctacaagaatctcttctatcctgtccttgtgccttaaggcaggaagatcccctcccacagatagcagggttgggtgttgtgtattgtgtttttttgtttgttttattttgttctgaaattaaaagtatgcaaaataaagatgatgcagttttaaaaaaaaaaaaaaaaaaaaagg

SEQ ID NO: 116 - protein, 541 aa, Homo sapiens
  Putative ribonuclease III
MRREVTVELSSQGFWKTGIRSDVCQHAMMLPVLTHHIRYHQCLMHLDKLIGYTFQDRCLLQLAMTHPSHHLNFGMNPDHARNSLSNCGIRQPKYGDRKVHHMHMRKKGINTLINIMSRLGQDDPTPSRINHNERLEFLGDAVVEFLTSVHLYYLFPSLEEGGLATYRTAIVQNQHLAMLAKKLELDRFMLYAHGPDLCRESDLRHAMANCFEALIGAVYLEGSLEEAKQLFGRLLFNDPDLREVWLNYPLHPLQLQEPNTDRQLIETSPVLQKLTEFEEAIGVIFTHVRLLARAFTLRTVGFNHLTLGHNQRMEFLGDSIMQLVATEYLFIHFPDHHEGHLTLLRSSLVNNRTQAKVAEELGMQEYAITNDKTKRPVALRTKTLADLLESFIAALYIDKDLEYVHTFMNVCFFPRLKEFILNQDWNDPKSQLQQCCLTLRTEGKEPDIPLYKTLQTVGPSHARTYTVAVYFKGERIGCGKGPSIQQAEMGAAMDALEKYNFPQMAHQKRFIERKYRQELKEMRWEREHQEREPDETEDIKK

SEQ ID NO: 117 - DNA, 1626 bp, Homo sapiens
  Putative ribonuclease III
atgagacgagaagtaacggtggagctaagtagccaaggattctggaaaactggcatccgttctgatgtctgtcagcatgcaatgatgctacctgttctgacccatcatatccgctaccaccaatgcctaatgcatttggacaagttgataggatatactttccaagatcgttgtctgttgcagctggccatgactcatccaagtcatcatttaaattttggaatgaatcctgatcatgccaggaattcattatctaactgtggaattcggcagcccaaatacggagacagaaaagttcatcacatgcacatgcggaagaaagggattaacaccttgataaatatcatgtcacgccttggccaagatgacccaactccctcgaggattaaccacaatgaacggttggaattcctgggtgatgctgttgttgaatttctgaccagcgtccatttgtactatttgtttcctagtctggaagaaggaggattagcaacctatcggactgccattgttcagaatcagcaccttgccatgctagcaaagaaacttgaactggatcgatttatgctgtatgctcacgggcctgacctttgtagagaatcggaccttcgacatgcaatggccaattgttttgaagcgttaataggagctgtttacttggagggaagcctggaggaagccaagcagttatttggacgcttgctctttaatgatccggacctgcgcgaagtctggctcaattatcctctccacccactccaactacaagagccaaatactgatcgacaacttattgaaacttctccggttctacaaaaacttactgagtttgaagaagcaattggagtaatttttactcatgttcgacttctggcaagggcattcacattgagaactgtgggatttaaccatctgaccctaggccacaatcagagaatggaattcctaggtgactccataatgcaactggtagccacagagtacttattcattcatttcccagatcatcatgaaggacacttaactttgttgcgaagctctttggtgaataatagaactcaggccaaggtagcggaggagctgggcatgcaggagtacgccataaccaacgacaagaccaagaggcctgtggcgcttcgcaccaagaccttggcggaccttttggaatcatttattgcagcgctgtacattgataaggatttggaatatgttcatactttcatgaatgtctgcttctttccacgattgaaagagttcattttgaatcaggattggaatgaccccaaatcccagcttcagcagtgttgcttgacacttaggacagaaggaaaagagccagacattcctctgtacaagactctgcagacagtgggcccatcccatgcccgaacctacactgtggctgtttatttcaagggagaaagaataggctgtgggaaaggaccaagtattcagcaagcggaaatgggagcagcaatggatgcgcttgaaaaatataattttccccagatggcccatcagaagcggttcatcgaacggaagtacagacaagagttaaaagaaatgaggtgggaaagagagcatcaagagagagagccagatgagactgaagacatcaagaaataa

(19) United States
(12) Patent Application Publication
(10) Pub. No.: US 2004/0058326 A1
(43) Pub. Date: Mar. 25, 2004

(54) Identification and use of molecules implicated in pain

(76) Inventors: Robert Alan Brooksbank, Cambridge (GB); Alistair Kerr Dixon, Cambridge (GB); Kevin Lee, Cambridge (GB); Robert Denham Pinnock, Ann Arbor, MI (US)

Correspondence Address: WARNER-LAMBERT COMPANY, 2800 PLYMOUTH RD, ANN ARBOR, MI, 48105, US

(21) Appl. No.: 10/205,331

(22) Filed: Jul. 24, 2002

(30) Foreign Application Priority Data
Feb. 7, 2002 (GB) 0202883.5
Jul. 27, 2001 (GB) 0118354.0

(51) Int. Cl. C12Q 1/68; G01N 33/53; G01N 33/567; A01K 67/027

(52) U.S. Cl. 435/6; 435/7.2; 800/3

(57) ABSTRACT

The invention relates to the use of:

(a) an isolated gene sequence that is down-regulated in the spinal cord of a mammal in response to mechanistically distinct first and second models of neuropathic or central sensitization pain;

(b) an isolated gene sequence comprising a nucleic acid sequence of any of Tables I to VI;

(c) an isolated gene sequence having at least 80% sequence identity with a nucleic acid sequence of any of Tables I to VI;

(d) an isolated nucleic acid sequence that is hybridizable to any of the gene sequences according to (a), (b) or (c) under stringent hybridisation conditions;

(e) a recombinant vector comprising a gene sequence or nucleic acid sequence according to any one of (a) to (d);

(f) a host cell containing the vector according to (e);

(g) a non-human animal having in its genome an introduced gene sequence or nucleic acid sequence or a removed or down-regulated gene sequence or nucleic acid sequence according to any one of (a) to (d);

(h) an isolated polypeptide comprising an amino acid sequence at least 90% identical to an amino acid sequence encoded by a nucleotide sequence according to any one of (a) to (d), or a variant polypeptide thereof with sequential amino acid deletions from the C terminus and/or the N-terminus;

(i) an isolated polypeptide encoded by a nucleotide sequence according to any one of (a) to (d); or

(j) an isolated antibody that binds specifically to a polypeptide according to (h) or (i);

in the screening of compounds for the treatment of pain, or for the diagnosis of pain.

The invention also relates to the use of naturally occurring compounds such as peptide ligands of the expression products of the above gene sequences and their associated signal transduction pathways for use in the treatment of pain.

This application claims the priority of United Kingdom application NO. 0118354.0, filed Jul. 27, 2001, and United Kingdom application NO. 0202883.5, filed Feb. 7, 2002; the entire contents of which applications are hereby incorporated by reference herein.

### FIELD OF THE INVENTION

The present invention relates to nucleic acids, their expression products and pathways involved in pain, and their use in screening for molecules that can alleviate pain. The invention further relates to methods for the assay and diagnosis of pain in patients.

### BACKGROUND TO THE INVENTION

Pain is currently classified into four general types. Post-operative acute pain can be successfully treated with existing pain medications of e.g. the opioid and non-steroidal anti-inflammatory (NSAID) types, and is usually short-term and self-limiting. A second type of pain, present e.g. in cancer and arthritis, is also responsive to medication initially with a NSAID and in its later stages with opioids. Neuropathic pain arises from damage to the central or peripheral nerve systems, and is more effectively treated with antidepressants or anticonvulsants. A fourth type of pain called central sensitization results from changes in the central nervous system as a result of chronic pain, these changes often being irreversible and difficult to treat. Nerve pain from shingles or diabetes falls into this and the neuropathic category. Changes occur where pain is at first poorly controlled and gradually progress to the point where a person is sensitive to stimuli which would not normally cause pain, for example a light touch. People with pain of this kind often describe a widening of the pain area to include areas which had originally not been injured or which were thought not to be involved in pain. This classification is, however based on clinical symptoms rather than on the underlying pain mechanisms.

Opiates such as morphine belong to a traditional class of pain-relieving compounds that are now recognized as binding to opiate receptors. Naturally occurring polypeptides have also been found to have opiate-like effects on the central nervous system, and these include β-endorphin, met-enkephalin and leu-enkephalin.

Salicin was isolated at the beginning of the 19th century, and from that discovery a number of NSAIDs such as aspirin, paracetamol, ibuprofen, flurbiprofen and naproxen were developed. NSAIDs are by far the most widely used pain-relieving compounds, but can exhibit side effects, in particular irritation of the GI tract that can lead to the formation of ulcers, gastrointestinal bleeding and anemia.

Interest in the neurobiology of pain is developing: see a colloquium sponsored by the US National Academy of Sciences in December 1998 concerning the neurobiology of pain and reviewed in The Scientist 13[1], 12, 1999. Many pain mechanisms were discussed including the role of the capsaicin receptors in pain, (M. J. Caterina et al., Nature, 389, 816-824, 1997). Large dosages of capsaicin were reported to disable that receptor, (W. R. Robbins et al., Anesthesia and Analgesia, 86, 579-583, 1998). Additionally, a tetrodotoxin-resistant sodium channel found in small diameter pain-sensing neurons (PN3) was discussed (A. N. Akopian et al., Nature, 379, 257-262, 1986) and L. Sangameswaran et al., Journal of Biological Chemistry, 271, 953-956, 1996). Its involvement in transmission and sensitization to pain signals has been reported, (S. D. Novakovic et al., Journal of Neuroscience, 18, 2174-2187, 1998). A further tetrodotoxin-resistant sodium channel has been reported (S. Tate et al., Nature Neuroscience, 1, 653-655 1998).

Second messenger systems have also been shown to be important since knockout-mice lacking protein kinase C (PKC) γ were reported to respond to acute pain e.g. from a hot surface, but not to respond to neuropathic pain when their spinal nerves are injured (Malmberg et al., Science, 278, 279-283 (1997).

Present methods for identifying novel compounds that relieve pain of one or more of the types indicated above suffer from the defect that they are dependent either on the relatively limited number of receptors known to be involved in pain or on the empirical identification of new receptors which is an uncertain process. In relation to known receptors, for example the opioid receptor, research directed to improved compounds offers the possibility of screenining compounds that have a better therapeutic ratio and fewer side effects. This does not lead naturally to compounds for different pain receptors that have new modes of action and new and qualitatively different benefits. Even when newly identified additional receptors are taken into account, known receptors revolve around tens of gene products. However, there are between 30,000 and 40,000 genes in the genome of an animal and more of them are concerned with nervous system function than with peripheral function. We therefore concluded that a large number of receptors and pathways are important to the transduction of pain, but up to now have remained unknown.

### SUMMARY OF THE INVENTION

It is an object of the invention to provide sequences of genetic material for which no role in pain has previously been disclosed, and which are useful, for example, in:

identifying metabolic pathways for the transduction of pain

identifying from said metabolic pathways compounds having utility in the diagnosis or treatment of pain

producing proteins and polypeptides with a role in the transduction of pain;

producing genetically modified non-human animals that are useful in the screening of compounds having utility in the treatment or diagnosis of pain.

Identifying ligand molecules for receptors involved in said metabolic pathways and having utility in the treatment of pain.

It is yet a further object of the invention to provide research tools, for example non-human animals and microorganisms, that can be used in screening compounds for pharmacological activity, especially pain-reducing activity.

The present invention is based on sequences that are down-regulated in two models of chronic pain, namely streptozocin-induced diabetes and chronic constrictive injury (CCI) to a nerve leading to the spine, for example the sciatic nerve.

In one aspect, the invention relates to the use in the screening of compounds that are effective in the treatment of pain, or in the diagnosis of pain, of:

(a) an isolated gene sequence that is down regulated in the spinal cord of a mammal in response to first and second models of pain, for example in response to streptozocin-induced diabetes and in response to a chronic constrictive injury to a nerve leading into the spine;

(b) an isolated gene sequence having at least 80% sequence identity with any of the nucleic acid sequences of Tables I-VI, preferably 85% sequence identity, more preferably 90%, increasingly preferably 95%, most preferably 99%.

(c) an isolated nucleic acid sequence comprising a sequence that is hybridizable to any of the gene sequences according to (a) or (b) under stringent hybridisation conditions;

(d) a recombinant vector comprising any of the gene sequences according to (a) to (c);

(e) a host cell containing the vector according to (d);

(f) a non-human animal, for use in the screening of compounds that are effective in the treatment of pain, or in the diagnosis of pain, having in its genome an introduced gene sequence or a removed or down-regulated nucleotide sequence, said sequence becoming down-regulated in the spinal cord of a mammal in response to first and second models of pain, particularly neuropathic or sensitisation pain, for example in response to streptozocin-induced diabetes and in response to a chronic constrictive injury to a nerve leading into the spine;

(g) an isolated polypeptide containing an amino acid sequence at least 90% identical to an amino acid sequence encoded by a nucleotide sequence according to any one of (a) to (d), or a variant thereof with sequential amino acid deletions from the C terminus and/or the N-terminus; or

(h) an isolated antibody that binds specifically to the isolated polypeptide according to (g).

The invention further provides a compound that is useful in the treatment or diagnosis of pain and that modulates the action of an expression product of a gene sequence that becomes down-regulated in the spinal cord of a mammal in response to first and second models of pain, for example being down-regulated both in response to streptozocin induced diabetes and in response to chronic constrictive injury to a nerve leading into the spine.

The invention also relates to the use of naturally occurring compounds such as peptide ligands of the expression products of the above gene sequences and their associated signal transduction pathways for the treatment of pain.

### DESCRIPTION OF PREFERRED EMBODIMENTS

Definitions

Within the context of the present invention:

“Comprising” means consisting of or including. Thus nucleic acid comprising a defined sequence includes nucleic acid that may contain a full-length gene or full-length cDNA. The gene may include any of the naturally occurring regulatory sequence(s), such as a transcription and translation start site, a promoter, a TATA box in the case of eukaryotes, and transcriptional and translational stop sites. Further, nucleic acid comprising a cDNA or gene may include any appropriate regulatory sequences for the efficient expression thereof in vitro.

“Isolated” requires that the material be removed from its original environment (e.g. the natural environment if it is naturally occurring). For example, a naturally occurring polynucleotide or a peptide present in a living animal is not isolated, but the same polynucleotide or peptide, separated from some or all of the coexisting materials in the natural system, is isolated. Such polynucleotide can be part of a vector and/or such polynucleotide or peptide can be part of a composition, and still be isolated in that the vector or composition is not a part of its natural environment.

“Mechanistically distinct” in relation to pain models implies that the pain is induced by mechanisms that differ in kind rather than being variants of a similar pain model. Thus diabetic pain and chronic constrictive pain models are mechanistically distinct, whereas spinal nerve ligation models and sciatic nerve ligation models which both work by ligation are not.

“Purified” does not require absolute purity; instead it is intended as a relative definition. Purification of starting materials or natural materials from their native environment to at least one order of magnitude, preferably two or three orders, and more preferably four or five orders of magnitude is expressly contemplated.

“Nucleic acid sequence” or “gene sequence” means a sequence of nucleotides or any variant or homologue thereof, or truncated or extended sequence thereof, and is preferably indicated by a Genebank accession number. Also within the scope of the present invention are down-regulated nucleic acid sequences which encode expression products which are components of signaling pathways. This invention also includes any variant or homologue or truncated or extended sequence of the down-regulated nucleotide sequence. Also within the scope of the present invention, the term “nucleic acid(s) product”, or “expression product” or “gene product” or a combination of these terms refers without being biased, to any, protein(s), polypeptide(s), peptide(s) or fragment(s) encoded by the down-regulated nucleotide sequence.

“Operably linked” refers to a linkage of polynucleotide elements in a functional relationship. For instance, a promoter or an enhancer is operably linked to a coding sequence if it regulates the transcription of the coding sequence. In particular, two DNA molecules (such as a polynucleotide containing a promoter region, and a polynucleotide encoding a desired polypeptide) are said to be “operably linked” if the nature of the linkage between the two polynucleotides does not (1) result in the introduction of a frame-shift mutation and (2) interfere with the ability of the polynucleotide containing the promoter to direct the transcription of the coding polynucleotide.

“Gene product” refers to polypeptide—which is interchangeable with the term protein—which is encoded by a nucleotide sequence and includes single-chain polypeptide molecules as well as multiple-polypeptide complexes where individual constituent polypeptides are linked by covalent or non-covalent means.

Polypeptides of the present invention may be produced by synthetic means (e.g. as described by Geysen et al., 1996) or by recombinant means.

The terms “variant”, “homologue”, “fragment”, “analogue” or “derivative” in relation to the amino acid sequence for the polypeptide of the present invention includes any substitution of, variation of, modification of, replacement of, deletion of or addition of one (or more) amino acid from or to the sequence providing the resultant polypeptide has the native gene product activity. In particular, the term “homologue” covers homology with respect to structure and/or function. With respect to sequence homology, there is at least 90%, more preferably at least 95% homology to an amino acid sequence encoded by the relevant nucleotide sequence shown in Tables I-VI, preferably there is at least 98% homology.

Typically, for the variant, homologue or fragment of the present invention, the types of amino acid substitutions that could be made should maintain the hydrophobicity/hydrophilicity of the amino acid sequence. Amino acid substitutions may include the use of non-naturally occurring amino acid analogues.

In addition, or in the alternative, the protein itself could be produced using chemical methods to synthesize a polypeptide, in whole or in part. For example, peptides can be synthesized by solid phase techniques, cleaved from the resin, and purified by preparative high performance liquid chromatography (e.g. Creighton (1983) Proteins Structures and Molecular Principles, W H Freeman and Co., New York, N.Y., USA). The composition of the synthetic peptides may be confirmed by amino acid analysis or sequencing (e.g. the Edman degradation procedure).

Direct peptide synthesis can be performed using various solid-phase techniques (Roberge J Y et al Science Vol 269 1995 202-204) and automated synthesis may be achieved, for example, using the ABI 431 A Peptide Synthesizer (Perkin Elmer) in accordance with the instructions provided by the manufacturer. Additionally, the amino acid sequence of a gene product, or any part thereof, may be altered during direct synthesis and/or combined using chemical methods with a sequence from other subunits, or any part thereof, to produce a variant polypeptide.

In another embodiment of the invention, a gene product natural, modified or recombinant amino acid sequence may be ligated to a heterologous sequence to encode a fusion protein. For example, for screening of libraries for compounds and peptide agonists and antagonists of gene product activity, it may be useful to encode a chimeric gene product expressing a heterologous epitope that is recognised by a commercially available antibody. A fusion protein may also be engineered to contain a cleavage site located between a gene product sequence and the heterologous protein sequence, so that the gene product may be cleaved and purified away from the heterologous moiety.

The gene product may also be expressed as a recombinant protein with one or more additional polypeptide domains added to facilitate protein purification. Such purification facilitating domains include, but are not limited to, metal chelating peptides such as histidine-tryptophan modules that allow purification on immobilised metals (Porath J, Protein Expr Purif Vol 3 1992 p263-281), protein A domains that allow purification on immobilised immunoglobulin, and the domain utilised in the FLAGS extension/affinity purification system (Immunex Corp, Seattle, Wash., USA). The inclusion of a cleavable linker sequence such as Factor XA or enterokinase (Invitrogen, San Diego, Calif., USA) between the purification domain and the gene product is useful to facilitate purification.

“Pain” includes chronic pain, neuropathic pain, pain arising from central sensitisation, and in particular diabetic pain.

“Stringent hybridization conditions” is a recognized term in the art and for a given nucleic acid sequence refers to those conditions which permit hybridization of that sequence to its complementary sequence and closely homologous sequences. Conditions of high stringency may be illustrated in relation to filter-bound DNA as for example 2×SCC, 65° C. (where SSC=0.15M sodium chloride, 0.015M sodium citrate, pH 7.2), or as 0.5M NaHPO₄, 7% sodium dodecyl sulfate (SDS), 1 mM EDTA, at 65° C., and washing in 0.1×SCC/0.1% SDS at 68° C. (Ausubel F. M. et al., eds, 1989, Current Protocols in Molecular Biology, Vol. I, Green Publishing Associates, Inc., and John Wiley & Sons Inc., New York, at p. 2. 10.3). Hybridization conditions can be rendered highly stringent by raising the temperature and/or by the addition of increasing amounts of formamide, to destabilize the hybrid duplex of non-homologous nucleic acid sequence relative to homologous and closely homologous nucleic acid sequences. Thus, particular hybridisation conditions can be readily manipulated, and will generally be chosen depending on the desired results.

“Variants or homologues” include (a) sequence variations of naturally existing gene(s) resulting from polymorphism(s), mutation(s), or other alteration(s) as compared to the above identified sequences, and which do not deprive the encoded protein of function (b) recombinant DNA molecules, such as cDNA molecules encoding genes indicated by the relevant Genebank accession numbers and (c) any sequence that hybridizes with the above nucleic acids under stringent conditions and encodes a functional protein or fragment thereof.

Identified Sequences

The inventors have identified nucleotide sequences that give rise to expression products listed in Tables I-VI, that become differentially expressed in the spinal cord in response to two distinct chronic pain stimuli, for example neuropathic and/or central sensitization pain stimuli, and that are believed to be involved in the transduction of pain. In Tables I-VI, * denotes more preferred nucleic acid sequences and ** denotes most preferred nucleic acid sequences. These nucleic acid sequences have not previously been implicated in the transduction of pain.

The validity of the present experimental procedure was confirmed by the fact that nucleotide sequences were obtained as a result of the investigation whose function in the transduction of pain has been previously confirmed and established. These nucleic acid sequences are not part of this invention. Any of the nucleic acid sequences and expression products can be used to develop screening technologies for the identification of novel molecules for the prevention or treatment of pain. These screening technologies could also be used to ascribe new pain therapeutic indications to molecules that have not previously been identified as being useful for the prevention or treatment of pain. Furthermore, the said nucleic acid sequences can be used as diagnostic tools and for the development of diagnostic tools.

TABLE I
Sequences whose expression products are kinases

| Expression | | | | | |
|---|---|---|---|---|---|
| product | Rat | Mouse | Human | | |
| or name | Accession | accession | Accession | | |
| (Reference) | Number | number | Number | Reaction | Assay |
| Pyruvate | M24359 | X97047 | X56494 | Tissue- | Kinase |
| kinase, M1 | (SEQ ID | | | specific | |
| and M2 | No's 1-3) | | | promoter; | |
| subunit s | | | | Carbo- | |
| (Refs: 1-2) | | | | hydrate | |
| | | | | kinase | |

TABLE II
Sequences whose expression products are receptors

| Expression | | | | | |
|---|---|---|---|---|---|
| Product or | Rat | Mouse | Human | | |
| Name | Accession | accession | Accession | | |
| (Reference) | Number | Number | Number | Reaction | Assay |
| Dopamine | I58000 | | | Dopamine | Receptor |
| receptor | (SEQ ID | | | receptor | |
| D.sub.1 ** | NO 4) | | | | |
| (Ref: 3) | | | | | |
| Putative | | AF114168 | | GABA-B | Receptor |
| GABA-B1a | | (SEQ ID | | Receptor | |
| receptor ** | | No's 5-6) | | | |
| (Ref: 4) | | | | | |

TABLE III
Sequences whose expression products are transporters

| Expression | Rat | Mouse | Human | | |
|---|---|---|---|---|---|
| Product or | Accession | Accession | Accession | | |
| Name | Number | Number | Number | Reaction | Assay |
| Differentiation- | AF271235 | | | Trans- | Transporter |
| associated Na- | (SEQ ID | | | membrane | |
| dependent | NO's 7-8) | | | phosphate | |
| inorganic | | | | transport | |
| phosphate | | | | | |
| cotransporter | | | | | |
| (Ref: N/A) | | | | | |
| Putative vacuolar | U87309 | | AAM47563.1 | Vacuolar | Transporter |
| assembly protein | | | (SEQ ID No | assembly | |
| VSP41 gene | | | 9) | and traffic | |
| (Ref: N/A) | | | | | |

TABLE IV
Sequences whose expression products are G-protein coupled receptor
proteins

| Expression | Rat | Mouse | Human | | |
|---|---|---|---|---|---|
| product or | Accession | Accession | Accession | | |
| Name | Number | Number | Number | Function | Assay |
| Git1 (G-protein- | AF085693 | | | Regulation of | Ligand binding |
| coupled receptor | | | | activity of | |
| kinase-interactor | (SEQ ID NO | | | ARF6 in | |
| 1; GPCR kinase- | 10-11) | | | phosphatidyl- | |
| associated ADP- | | | | inisitol 3-kinase | |
| ribosylation | | | | signalling | |
| factor) | | | | pathways; β2 | |
| (Ref: 5) | | | | adrenergic | |
| | | | | receptor | |
| | | | | regulation | |

TABLE V
Sequences whose expression products are DNA-binding proteins

| Expression | Rat | Mouse | Human | | |
|---|---|---|---|---|---|
| Product or | Accession | Accession | Accession | Re- | |
| Name | Number | Number | Number | action | Assay |
| Putative | | X91866 | M11354 | DNA | DNA |
| histone H3.3A | | | (SEQ ID NO | binding | binding |
| (Ref: 6) | | | 12-13) | | |

TABLE VI
Sequences whose expression products are other enzymes

| Expression | Rat | Mouse | Human | | |
|---|---|---|---|---|---|
| product or | Accession | Accession | Accession | | |
| Name | Number | Number | Number | Reaction | Assay |
| 3-Hydroxy 3- | X52625 | | | Cholesterol | Ligase |
| methylglutaryl | | | | biosynthesis | |
| coenzyme A | (SEQ ID | | | | |
| synthase, | No's 14-15) | | | | |
| cytosolic * | | | | | |
| (Ref: 7) | | | | | |
| Acyl-CoA | D360666 | | | Fatty acid | Ligase |
| synthetase II, | (N/A) | | | metabolism | |
| brain (Ref: N/A) | | | | | |
| Farnesyl | M34477 | | | Isoprene | Ligase |
| diphosphate | | | | biosynthesis | |
| synthase ** | (Seq ID No's | | | | |
| (Ref: 8) | 16-17) | | | | |
| Bendless protein | AB032739 | | E12457 | Protein | Ligase |
| (Ref: N/A) | (Seq ID No's | | | degradation | |
| | 18-19) | | | | |
| fatty acid | X62888 | | | Fatty acid | Ligase |
| synthase | (SEQ ID | | | synthesis | |
| (Ref: 9) | No's 20-21) | | | | |
| Glutamine | M91652 | | | Amino acid | Ligase |
| synthetase (EC | (SEQ ID | | | metabolism | |
| 6.3.1.2) ** | NO's | | | | |
| (Ref: 10) | 22-23) | | | | |
| Putative seryl- | | | X91257 | Ligase | Ligase |
| tRNA synthetase | | | (SEQ ID NO's | | |
| (Ref No: 11) | | | 24-25) | | |
| Enolase, alpha | X02610 | X52379 | M14328 | Glycolysis | Lyase |
| alpha, non- | | | (SEQ ID NO's | | |
| neuronal (NNE) | | | 26-27) | | |
| (REF NO 12) | | | | | |
| Aldose reductase, | X05884 | | | Reduces | Oxidoreductase |
| lens (AREC | (SEQ ID | | | carbonyl | |
| 11.1.21) | NO's 28-29) | | | | |
| (REF: 13) | | | | | |
| Cytochrome-c | S79304 | | | Mitochrondrial | Oxidoreductase |
| oxidase I, | (SEQ ID | | | energy | |
| mitochondrial | NO's 30-31) | | | metabloism | |
| (Ref: 14) | | | | | |
| Lactate | U07181 | X51905 | Y00711 | Glycolysis | Oxidoreductase |
| dehydrogenase-B | (SEQ ID | | | | |
| (LDH-B) | NO's 32-33) | | | | |
| (Ref: 15) | | | | | |
| Putative | | | x13923 | Mitochrondrial | Oxidoreductase |
| cytochrome c | | | | energy | |
| oxidase VIB (EC | | | (SEQ ID NO's | metabolism | |
| 1.9.3.1) | | | 34-35) | | |
| (Ref: 16) | | | | | |
| Putative NADH: | | | AF044953 | Mitochrondrial | Oxidoreductase |
| ubiquinone | | | (SEQ ID No's | energy | |
| oxidoreductasePG | | | 36-37) | metabolism | |
| IV subunit | | | | | |
| (Ref: 17) | | | | | |
| Putative succinate | | AF095938 | AF171022 | TCA cycle | Oxidoreductase |
| dehydrogenase | | (SEQ ID | | | |
| flavoprotein | | No's 38- | | | |
| (Ref: N/A) | | 39) | | | |
| Putative | | | J04973 | Mitochrondrial | Oxidoreductase |
| ubiquinol- | | | (SEQ ID No's | energy | |
| cytochrome-c | | | 40-41) | metabolism | |
| reductase (EC | | | | | |
| 1.10.2.2) core | | | | | |
| protein II | | | | | |
| (REF: 18) | | | | | |
| Stearoyl-coA | AB032243 | M26270 | | Fatty acid | Oxidoreductase |
| desaturase 2 * | (SEQ ID | | | biosynthesis | |
| (Ref: N/A) | NO's 42-43) | | | | |
| Ribophorin I * | X05300 | | | Glycosolation | Transferase |
| (Ref: 19) | (SEQ ID | | | | |
| | No's 44-45) | | | | |
| Sulfotransferase- | AF188699 | | | Transferase | Transferase |
| like protein | (SEQ ID | | | | |
| (REF: 20) | No's 46-47) | | | | |
| ATP synthase, | X56133 | | | Mitochrondrial | Hydrolase |
| H+, alpha | (SEQ ID | | | energy | |
| subunit, | No's 48-49) | | | metabolism | |
| mitochondrial | | | | | |
| (EC 3.6.1.34) | | | | | |
| (Ref: N/A) | | | | | |
| F1F0 ATPase | U00926 | | | Mitochrondrial | Hydrolase |
| delta subunit | (SEQ ID | | | energy | |
| (REF: 21) | No's 50-51) | | | metabolism | |
| Putative | | | D78013 | Pyrimidine | Hydrolase |
| dihydropyrimidin | | | (SEQ ID NO's | degradation | |
| ase related protein | | | 52-53) | | |
| * (REF: 22) | | | | | |
| Heat shock | 545392 | M18186 | M16660 | Cell protection | Hydrolase |
| protein 90 | (SEQ ID | | | | |
| (REF: 23) | NO's 114- | | | | |
| | 115) | | | | |
| Myelin basic | K00512 | | | Myelin | Ligand binding |
| protein S (MBP | SEQ ID No | | | structural | |
| S) (REF: 24) | 54) | | | protein | |
| Transferrin | D38380 | | | Iron transport | Ligand binding |
| (Ref 25) | (SEQ ID | | | | |
| | No's 55-56) | | | | |
| Neurofilament, | AF031880 | | | Cytoskeleton | Ligand binding |
| light molecular | (SEQ ID | | | | |
| weight (NF-L) | No's 57-58) | | | | |
| (Ref: 26) | | | | | |
| Myelin- | M16800 | M31811 | | Cell adhesion | Ligand binding |
| associayted | (SEQ ID | | | molecule for | |
| glycoprotein | NO's 59-60) | | | postnatal neural | |
| (MAG) | | | | development | |
| (Ref: 27) | | | | | |
| NF-M middle | M18628 | | | Cytoskeleton | Ligand binding |
| molecular weight | (SEQ ID | | | | |
| neurofilament | No's 61-62) | | | | |
| protein | | | | | |
| (Ref: 28) | | | | | |
| Neuro- | D32249 | | | Protein sorting; | Ligand binding |
| degeneration | (SEQ ID | | | synaptic | |
| associated- | No's 63-64) | | | communication | |
| protein 1 | | | | and plasticity | |
| (Ref: 29) | | | | | |
| S-100 protein β- | X01090 | | | Zinc and | Ligand binding |
| subunit | (SEQ ID No | | | calcium binding | |
| (REF: 30) | 65) | | | | |
| Microtubule- | X60370 | | L06237 | Cytoskeleton | Ligand binding |
| associated protein | (SEQ ID | | | protein, | |
| 1b (Map 1b) | No's 66-67) | | | neuronal | |
| (Ref: 31) | | | | growth/ | |
| | | | | regeneration; | |
| | | | | microtubule | |
| | | | | binding protein | |
| Putative cdc 37 | D26564 | | | Cell signalling; | Ligand binding |
| homolog | (Seq ID No's | | | cell cycle | |
| (Ref: 32) | 68-69) | | | protein | |
| Putative ras- | | | U11293 | Small GTP | Ligand binding |
| related protein | | | (SEQ ID No's | binding protein | |
| Rab-5c | | | 70-71) | | |
| (Ref: 33) | | | | | |
| Putative gelsolin | | J04953 | | Cytoskeleton | Ligand binding |
| (Ref: 34) | | (SEQ ID | | protein | |
| | | No's 72- | | | |
| | | 73) | | | |
| Cd81 antigen | U19894 | X59047 | M33680 | Regulator for | Ligand binding |
| (target of | (SEQ ID | | | neuron-induced | |
| antiproliferative | No's 74-75) | | | astrocyte | |
| antibody 1) | | | | differentiation | |
| (Ref: 35) | | | | microglial | |
| | | | | effector | |
| | | | | functions | |
| Mobp81 (Myelin- | X87900 | | | Myelin | Ligand binding |
| associated/Oligo- | (SEQ ID | | | compaction | |
| dendrocytic basic | N0's 76-77) | | | | |
| protein 81) | | | | | |
| (Ref: 36) | | | | | |
| Syntaxin binding | | | BC002869 | Neuro- | Ligand binding |
| protein n-sec1, | | | (Seq ID No's | transmission | |
| sec1 homolog | | | 78-79) | | |
| (Ref: N/A) | | | | | |
| Alpha-internexin | M73049 | | | Cytoskeleton | Ligand binding |
| (Ref: 37) | (SEQ ID | | | protein; | |
| | No's 80-81) | | | neuronal | |
| | | | | intermediate | |
| | | | | filament that | |
| | | | | can self- | |
| | | | | assemble | |
| Putative β- | | AB024921 | | Cytoskeleton | Ligand binding |
| sarcoglycan A3b | | (SEQ ID | | protein | |
| (Ref: N/a) | | No's 82- | | | |
| | | 83) | | | |
| CGI-78 protein | AF151835 | | AF151835 | Unknown | Ligand binding |
| Ref: 38) | | | (SEQ ID No's | | |
| | | | 84-85) | | |
| KIAA0143 | | | D63477 | Transmembrane | Ligand binding |
| (Ref: 39) | | | (SEQ ID NO's | protein | |
| | | | 86-87) | | |
| Septin 2 | | | D50918 | GTPase; | Ligand binding |
| (KIAA0128; | | | (SEQ ID No's | cytokinesis | |
| Ref 39) | | | 88-89) | | |
| Nucleobindin | Z36277 | | | Unknown | Ligand binding |
| (Ref: 40) | (SEQ ID | | | | |
| | No's 90-91) | | | | |
| Myelin protein | M69139 | S55427 | | Myelin | Ligand binding |
| SR13 | (SEQ ID | | | structural | |
| (Ref: 41) | NO's 92-93) | | | protein | |
| B-Actin, | V01217 | X03672 | | Structural | Ligand binding |
| cytoplasmic | (SEQ ID | | | protein | |
| (Ref: 42) | NO's 94-95) | | | | |
| Ly6/neurotoxin | | AF141377 | | Neuro- | Ligand binding |
| (Lynx1) homolog | | (SEQ ID | | transmitter | |
| (Ref: 43) | | No's 96- | | | |
| | | 97) | | modulator | |
| Astrocytic | AJ243949 | X86694 | | PKC substrate | Ligand binding |
| phosphoprotein, | (SEQ ID | | | | |
| PFA 15 gene | NO's 98- | | | | |
| (Ref: N/A) | 99) | | | | |
| PLIC-1 | | AF177345 | | Cytoskeleton | Ligand binding |
| (Ref: N/A) | | (SEQ ID | | interaction | |
| | | NO 100- | | | |
| | | 101) | | | |
| Nfx1 (tip | AF093139 | AF093140 | | Unknown | Logand binding |
| associating | (SEQ ID | | | | |
| protein (TAP) | NO's 102- | | | | |
| gene) (Ref: N/A) | 103) | | | | |
| Alpha-Crystallin | U04320 | M73741 | M28638 | Stress | Ligand binding |
| B (Ref N/A) | SEQ ID | | | response; heat | |
| | NO's 104- | | | shock element | |
| | 105) | | | | |
| Heat shock-like | X70065 | U73744 | Y00371 | Stress response | Ligand binding |
| protein 70 kD | (SEQ ID | | | | |
| (Ref: 44) | No's 106- | | | | |
| | 107) | | | | |
| Tau microtubule- | X79321 | | | Microtubule | Ligand binding |
| associated protein | (SEQ ID | | | associated | |
| (Ref: 45) | NO's 108- | | | protein | |
| | 109) | | | expressed in | |
| | | | | neurons | |
| Myelin, | K03242 | | | Myelin | Ligand binding |
| Schwann cell, | (SEQ ID | | | structural | |
| Peripheral (P-0) | No's 110- | | | protein | |
| (Ref: 46) | 111) | | | | |
| B-Tubulin class 1 | AB011679 | X04663 | AF14139 | Structural | Ligand binding |
| (Ref: 47) | (SEQ ID | | | protein | |
| | NO's 112- | | | | |
| | 113) | | | | |
| Putative | | | AF116910 | RNA hydrolysis | Hydrolase |
| ribonuclease III | | | (SEQ ID NO's | | |
| (Ref: N/A) | | | 116-117) | | |

Production of Polypeptides Nucleic Acids

Vectors

Recombinant expression vectors comprising a nucleic acid can be employed to express any of the nucleic acid sequences of the invention. The expression products derived from such vector constructs can be used to develop screening technologies for the identification of molecules that can be used to prevent or treat pain, and in the development of diagnostic tool for the identification and characterization of pain. The expression vectors may also be used for constructing transgenic non-human animals.

Gene expression requires that appropriate signals be provided in the vectors, said signals including various regulatory elements such as enhancers/promoters from viral and/or mammalian sources that drive expression of the genes or nucleic acid sequences of interest in host cells. The regulatory sequences of the expression vectors used in the invention are operably linked to the nucleic acid sequence encoding the pain-associated protein of interest or a peptide fragment thereof.

Generally, recombinant expression vectors include origins of replication, selectable markers, and a promoter derived from a highly expressed gene to direct transcription of a downstream nucleoide sequence. The heterologous nucleotide sequence is assembled in an appropriate frame with the translation, initiation and termination sequences, and if applicable a leader sequence to direct the expression product into the periplasmic space, the extra-cellular medium or cell membrane.

In a specific embodiment wherein the vector is adapted for expressing desired sequences in mammalian host cells, preferred vectors will comprise an origin of replication from the desired host, a suitable promoter and an enhancer, and also any necessary ribosome binding sites, polyadenylation site, transcriptional termination sequences, and optionally 5′-flanking non-transcribed sequences. DNA sequences derived from the SV40 or CMV viral genomes, for example SV40 or CMV origin, early promoters, enhancers, and polyadenylation sites may be used to provide the required non-transcribed genetic elements.

A recombinant expression vector used in the invention advantageously also comprises an untranscribed polynucleotide region located at the 3′end of the coding sequence (ORF), this 3′-UTR (untranslated region) polynucleotide being useful for stabilizing the corresponding mRNA or for increasing the expression rate of the vector insert if this 3′-UTR harbours regulation signal elements such as enhancer sequences.

Suitable promoter regions used in the expression vectors are chosen taking into account the host cell in which the nucleic acid sequence is to be expressed. A suitable promoter may be heterologous with respect to the nucleic acid sequence for which it controls the expression, or alternatively can be endogenous to the native polynucleotide containing the coding sequence to be expressed. Additionally, the promoter is generally heterologous with respect to the recombinant vector sequences within which the construct promoter/coding sequence has been inserted. Preferred promoters are the LacI, LacZ, T3 or T7 bacteriophage RNA polymerase promoters, the lambda PR, PL and Trp promoters (see EP-0 036 776), the polyhedrin promoter, or the p10 protein promoter from baculovirus (kit Novagen; Smith et al., (1983); O'Reilly et al. (1992)).

Preferred selectable marker genes contained in the expression recombinant vectors used in the invention for selection of transformed host cells are preferably dehydrofolate reductase or neomycin resistance for eukaryotic cell culture, TRP1 for S. cerevisiae or tetracycline, rifampicin or ampicillin resistance in E. coli, or Levamsaccharase for Mycobacteria, this latter marker being a negative selection marker.

Preferred bacterial vectors are listed hereafter as illustrative but not limitative examples: pQE70, pQE60, pQE-9 (Quiagen), pD10, phagescript, psiX174, p.Bluescript SK, pNH8A, pNH16A, pNH18A, pNH46A (Stratagene); pKK223-3, pKK233-3, pDR540, pRIT5 (Pharmacia); pWLNEO, pSV2CAT, pOG44, pXT1, pSG (Stratagene); pSVK3, pBPV, pMSG, pSVL (Pharmacia); pQE-30 (QIA express).

Preferred bacteriophage recombinant vectors of the invention are P1 bacteriophage vectors such as described by Sternberg N. L. (1992;1994).

A suitable vector for the expression of any of the pain associated polypeptides used in the invention or fragments thereof, is a baculovirus vector that can be propagated in insect cells and in insect cell-lines. A specific suitable host vector system is the pVL 1392/1393 baculovirus transfer vector (Pharmingen) that is used to transfect the SF9 cell line (ATCC N^{o} CRL 1711) that is derived from Spodoptera frugiperda.

The recombinant expression vectors of in the invention may also be derived from an adenovirus. Suitable adenoviruses are described by Feldman and Steig (1996) or Ohno et al. (1994). Another preferred recombinant adenovirus is the human adenovirus type two or five (Ad 2 or Ad 5) or an adenovirus of animal origin (Patent Application WO 94/26914)

Particularly preferred retrovirus for the preparation or construction of retroviral in vitro or in vivo gene delivery vehicles include retroviruses selected from the group consisting of Mink-Cell Focus Inducing Virus, Murine Sarcoma Virus, and Ross Sarcoma Virus. Other preferred retroviral vectors are those described in Roth et al. (1996), in PCT Application WO 93/25234, in PCT Application WO 94/06920, and also in Roux et al. (1989), Julan et al.(1992) and Nada et al. (1991).

Yet, another viral vector system that is contemplated is the Adeno Associated Viruses (AAV) such as those described by Flotte et al. (1992), Samulski et al. (1989) and McLaughlin et al. (1996).

Host Cells Expressing Pain Associated Polypeptides

Host cells that endogenously express pain associated polypeptides or have been transformed or transfected with one of the nucleic acid ssequences described herein, or with one of the recombinant vector described above, particularly a recombinant expression vector, can be used in the present invention. Also included are host cells that are transformed (prokaryotic cells) or are transfected (eukaryotic cells) with a recombinant vector such as one of those described above.

Preferred host cells used as recipients for the expression vectors used in the invention are the following:

(a) prokaryotic host cells: Escherichia coli, strains. (i.e. DH5-α, strain) Bacillus subtilis, Salmonella typhimurium and strains from species like Pseudomonas, Streptomyces and Staphylococcus for the expression of up and down-regulated nucleic acid sequence modulated by pain, characterized by having at least 80% sequence identity with any of the nucleic acid sequences of Tables I-VI. Plasmid propagation in these host cells can provide plasmids for transfecting other cells.

(b) eukaryotic host cells: HeLa cells (ATCC N^{o} CCL2; N^{o} CCL2.1; N^{o} CCL2.2), Cv 1 cells (ATCC N^{o} CCL70), COS cells (ATCC N^{o} CRL 1650; N^{o} CRL 1651), Sf-9 cells (ATCC N^{o} CRL 1711), C127 cells (ATCC N^{o} CRL-1804), 3T3 cells (ATCC N^{o} CRL-6361), CHO cells (ATCC N^{o} CCL-61), human kidney 293 cells (ATCC N^{o} 45504; N^{o} CRL-1573), BHK (ECACC N^{o}84100 501; N^{o}84111301), PC12 (ATCC N^{o} CRL-1721), NT2, SHSY5Y (ATCC N^{o} CRL-2266), NG108 (ECACC N^{o}88112302) and F11, SK-N-SH (ATCC N^{o} CRL-HTB-11), SK-N-BE(2) (ATCC N^{o} CRL-2271), IMR-32 (ATCC N^{o} CCL-127). A preferred system to which the nucleic acids of the invention can be expressed are neuronal cell lines such as PC12, NT2, SHSY5Y, NG108 and F11, SK-N-SH, SK-N-BE(2), IMR-32 cell lines, COS cells, 3T3 cells, HeLa cells, 292 cells and CHO cells. The above cell lines could be used for the expression of any of the nucleic acid sequences of Tables I-VI.

When a nucleic acid sequence of any of Tables I-X is expressed using a neuronal cell line, the sequence can be expressed through an endogenous promoter or native neuronal promoter, or an exogenous promoter. Suitable exogenous promoters include SV40 and CMV and eukaryotic promoters such as the tetracycline promoter. The preferred promoter when pain associated molecules are endogenously expressed is an endogenous promoter. A preferred promoter in a recombinant cell line is the CMV promoter.

In a specific embodiment of the host cells described above, these host cells have also been transfected or transformed with a polynucleotide or a recombinant vector for the expression of a natural ligand of any of the nucleic acid sequences of any of Tables I-VI or a modulator of these expression products.

Proteins, Polypeptides and Fragments

The expression products of the nucleic acid sequences of Tables I-VI or fragment(s) thereof can be prepared using recombinant technology, from cell lines or by chemical synthesis. Recombinant methods, chemical methods or chemical synthetic methods can be used to modify a gene in order to introduce into the gene product, or a fragment of the gene product, features such as recognition tags, cleavage sites or other modifications. For efficient polypeptide production, the endogenous expression system or recombinant expression system should allow the expression products to be expressed in a manner that will allow the production of a functional protein or fragment thereof which can be purified. Preferred cell lines are those that allow high levels of expression of polypeptide or fragments thereof. Such cell lines include cell lines which naturally express the nucleic acid sequence of Tables I-VI or common mammalian cell lines such as CHO cells or COS cells, etc, or more specific neuronal cell lines such as PC12. However, other cell types that are commonly used for recombinant protein production such as insect cells, amphibian cells such as oocytes, yeast and prokaryotic cell lines such as E. coli can also be used.

The expression products of Tables I-VI or fragments thereof can be utilized in screens to identify potential therapeutic ligands, either as a purified protein, as a protein chimera such as those produced in phage display, as a cell membrane (lipid or detergent) preparation, or in intact cells.

The invention also relates generally to the use of proteins, peptides and peptide fragments for the development of screening technologies for the identification of molecules for the prevention or treatment of pain, and the development of diagnostic tools for the identification and characterization of pain. These peptides include expression products of the nucleic acid sequences of Tables I-VI and purified or isolated polypeptides or fragments thereof having at least 90%, preferably 95%, more preferably 98% and most preferably 99% sequence identity with the any of the expression products of nucleic acid sequences of Tables I-VI. Expressed peptides and fragments of any of these nucleic acid sequences can be used to develop screening technologies for the identification of novel molecules for the prevention or treatment of pain. These screening technologies could also be used to ascribe new pain therapeutic indications to molecules, which have not previously been ascribed for the prevention or treatment of pain. Furthermore the said expressed peptides and fragments can be used as diagnostic tools and for the development of diagnostic tools.

Screening Methods

As discussed above, we have identified nucleic acid sequences whose expression is regulated by pain, particularly chronic pain and more particularly diabetic pain. The expression products of these nucleic acids can be used for screening ligand molecules for their ability to prevent or treat pain, and particularly, but not exclusively, chronic pain. The main types of screens that can be used are described below. The test compound can be a peptide, protein or chemical entity, either alone or in combination(s), or in a mixture with any substance. The test compound may even represent a library of compounds.

The expression products of any of the nucleic acid sequences of Tables I-VI or fragments thereof can be utilized in a ligand binding screen format, a functional screen format or invivo format. Examples of screening formats are provided.

A) Ligand Binding Screen

In ligand binding screening a test compound with is contacted with an expression product of one of the sequences of Tables I-VI, and the ability of said test compound to interact with said expression product is determined, e.g. the ability of the test compound(s) to bind to the expression product is determined. The expression product can be a part of an intact cell, lipidic preparation or a purified polypeptide(s), optionally attached to a support, such as beads, a column or a plate etc.

Binding of the test compound is preferably performed in the presence of a ligand to allow an assessment of the binding activity of each test compound. The ligand may be contacted with the expression product either before, simultaneously or after the test compound. The ligand should be detectable and/or quantifiable. To achieve this, the ligand can be labelled in a number of ways, for example with a chromophore, radioactive, fluorescent, phosphorescent, enzymatic or antibody label. Methods of labelling are known to those in the art. If the ligand is not directly detectable it should be amenable to detection and quantification by secondary detection, which may employ the above technologies. Alternatively the expression product or fragment thereof can be detectable or quantifiable. This can be achieved in a similar manner to that described above.

Binding of the test compound modifies the interaction of the ligand with its binding site and changes the affinity or binding of the ligand for/to its binding site. The difference between the observed amount of ligand bound relative to the theoretical maximum amount of ligand bound (or to the ligand bound in the absence of a test compound under the same conditions) is a reflection of the binding ability (and optionally the amount and/or affinity) of a test compound to bind the expression product.

Alternatively, the amount of test compound bound to the expression product can be determined by a combination of chromatography and spectroscopy. This can be achieved with technologies such as Biacore (Amersham Pharmacia). The amount of test compound bound to the expression product can also be determined by direct measurement of the change in mass upon compound or ligand binding to the expression product. Alternatively, the expression product, compound or ligand can be fluorescently labelled and the association of expression product with the test compound can be followed by changes in Fluorescence Energy Transfer (FRET).

The invention therefore includes a method of screening for pain alleviating compounds, comprising:

a) contacting a test compound or test compounds in the presence of a ligand with an expression product of any of the nucleic acid sequences of Tables I-VI or with cell expressing at least one copy of the expression product or with a lipidic matrix containing at least one copy of the expression product;

b) determining the binding of the test compound to the expression product, and

c) selecting test compounds on the basis of their binding abilities.

In the above method, the ligand may be added prior to, simultaneously with or after contact of the test compound with the expression product. Non limiting examples and methodology can be gained from the teachings of the Molecular Probes handbook and references therein (Molecular Probes, Inc., 4849 Pitchford Ave, Eugene, USA), Methods in neurotransmitter receptor analysis (Yamamura H I., Enna, S J., and Kuhar, M J., Raven Press New York, the Glaxo Pocket Guide to Pharmacology, Dr. Michael Sheehan, Glaxo Group Research Ltd, Ware, Herts SG12 0DP), Bylund D B and Murrin L C (2000, Life Sciences, 67 (24) 2897-911), Owicki J C (2000, J. biomol Screen (5) 297-306), Alberts et al (1994, Molecular Biology of the Cell, 3^{rd} Edn, Garland Publication Inc), Butler J E, (2000 Methods 22(1):4-23, Sanberg S A (2000, Curr Opin Biotechnol 11(1) 47-53), and Christopoulos A (1999, biochem Pharmacol 58(5) 735-48).

B) Functional Screening

(a) Kinase Assays

The expression product of any of the nucleic acid sequences of Tables I-VI which encode a kinase, and in particular the nucleic acid sequence listed in Table I, is amenable to screening using kinase assay technology.

Kinases have the ability to add phosphate molecules to specific residues in ligands such as binding peptides in the presence of a substrate such as adenosine triphosphate (ATP). Formation of a complex between the kinase, the ligand and substrate results in the transfer of a phosphate group from the substrate to the ligand. Compounds that modulate the activity of the kinase can be determined with a kinase functional screen. Functional screening for modulators of kinase activity therefore involves contacting one or more a test compounds with an expression product of one of the nucleic acid sequences of Tables I-VI which encodes a kinase, and determining the ability of said test compound to modulate the transfer of a phosphate group from the substrate to the ligand.

The expression product can be part of an intact cell or of a lipidic preparation or it can be a purified polypeptide(s), optionally attached to a support, for example beads, a column, or a plate. Binding is preferably performed in the presence of ligand and substrate to allow an assessment of the binding activity of each test compound.

The ligand should contain a specific kinase recognition sequence and it should not be phosphorylated at its phosphoryation site. The ligand and/or substrate may be contacted with the kinase either before, simultaneously or after the test compound. Optionally the substrate may be labelled with a kinase transferable labelled phosphate. The assay being monitored by the phosphorylation state of the substrate and/or the ligand. The ligand should be such that its phosphorylation state can be determined. An alternative method to do this is to label the ligand with a phosphorylation-state-sensitive molecule. To achieve this, the ligand can be labelled in a number of ways, for example with a chromophore, radioactive, fluorescent, phosphorescent, enzymatic or antibody label. If the ligand is not directly detectable it should be amenable to detection and quantification by secondary detection, which may employ the above technologies. Such technologies are known to those in the art.

Binding of the test compound to the kinase modifies its ability to transfer a phosphate group from the substrate to the ligand. The difference between the observed amount of phosphate transfer relative to the theoretical maximum amount of phosphate transfer is a reflection of the modulatory effect of the test compound. Alternatively, the degree of phosphate transfer can be determined by a combination of chromatography and spectroscopy. The extent of phosphorylation of the ligand peptide or dephosphorylation of the substrate can also be determined by direct measurement. This can be achieved with technologies such as Biacore (Amersham Pharmacia).

The invention also provides a method for screening compounds for the ability to relieve pain, which comprises:

(a) contacting one or more test compounds in the presence of ligand and substrate with an expression product of any of the sequences of Tables I-VI which is a kinase or with a cell containing at least 1 copy of the expression product or with a lipidic matrix containing at least 1 copy of an expression product;

(b) determining the amount of phosphate transfer from the substrate to the ligand; and

(c) selecting test compounds on the basis of their capacity to modulate phosphate transfer.

Optionally ligand, substrate and/or other essential molecules may be added prior to contacting the test compound with expression product of step (a) or after step (a). Non limiting examples and methodology can be gained from the teachings of the Molecular Probes handbook and references therein (Molecular Probes, Inc., 4849 Pitchford Ave, Eugene, USA), Methods in Molecular Biology 2000; 99: 191-201, Oncogene 2000 20; 19(49): 5690-701, and FASAB Journal, (10, 6, P55, P1458, 1996, Pocius D Amrein K et al).

b) Receptor Assays

An expression product of any of the nucleic acid sequences of Tables I-VI which encodes a receptor, and in particular any of the nucleic acid sequences listed in Table II, is amenable to screening using receptor assay technology.

Receptors are membrane associated proteins that initiate intracellular signalling upon ligand binding. Therefore, the identification of molecules for the prevention and treatment of pain can be achieved with the use of a ligand binding assay, as outlined above. Such an assay would utilize an endogenous or non-endogenous ligand as a component of the ligand binding assay. The binding of this ligand to the receptor in the presence of one or more test compounds would be measured as described above. Such is the nature of receptors that the assay is usually, but not exclusively performed with a receptor as an intact cell or membraneous preparation.

The invention therefore includes a method of screening for pain alleviating compounds, comprising:

a) contacting a test compound or test compounds in the presence of a ligand with cell expressing at least one copy of the expression product of any of the sequences of Tables I-VI which is a receptor or with a lipidic matrix containing at least one copy of the expression product;

b) determining the binding of the test compound to the expression product, and

c) selecting test compounds on the basis of their binding abilities.

c) Transporter Protein Assays

An expression product of any of the nucleic acid sequences of Tables I-VI which encodes a transporter protein, and in particular any of the nucleic acid sequences listed in Table III, is amenable to screening using transporter protein assay technology. Non limiting examples of technologies and methodologies are given by Carroll F I, et al (1995, Medical Research Review, Sep15 (5) p419-444), Veldhuis J D and Johnson M l (1994, Neurosci. Biobehav Rep., winter 18(4) 605-12), Hediger M A and Nussberger S (1995, Expt Nephrol, July-Aug 3(4) p211-218, Endou H and Kanai Y, (1999, Nippon Yakurigaku Zasshi, Oct. 114 Suppl 1:1p-16p), Olivier B et al (2000, Prog. Drug Res., 54, 59-119), Braun A et al (2000, Eur J Pharm Sci, oct 11, Suppl 2 S51-60) and Molecular Probes handbook and references therein (Molecular Probes, Inc., 4849 Pitchford Ave, Eugene, USA).

The main function of transporter proteins is to facilitate the movement of molecules across a cellular membrane. Compounds that modulate the activity of transporter proteins can be determined with a transporter protein functional screen. Functional screening for modulators of transporter proteins comprises contacting at least one test compound with an expression product as aforesaid which is a transporter protein and determining the ability of said test compound to modulate the activity of said transporter protein. The expression product can be part of an intact cell, or lipidic preparation, optionally attached to a support, for example beads, a column or a plate. Binding is preferably performed in the presence of the molecule to be transported, which should only able to pass through a cell membrane or lipidic matrix with the aid of the transporter protein. The molecule to be transported should be able to be followed when it moves into a cell or through a lipidic matrix. Preferably the molecule to be transported is labelled to aid in characterization, e.g. with a chromophore, radioactive, fluorescent, phosphorescent, enzymatic or antibody label. If the molecule to be transported is not directly detectable it should be amenable to detection and quantification by secondary detection, which may employ the above technologies. The molecule to be transported may be contacted with the transporter protein before, simultaneously with or after the test compound. If binding of the test compound to the transporter protein modifies its ability to transport molecules through a membraneous or lipidic matrix, then the difference between the observed amount of transported molecule in a cell/or through a lipidic matrix relative to the theoretical maximum amount is a reflection of the modulatory effect of the test compound.

The invention further provides a method for screening compounds for their ability to relieve pain, comprising

a) contacting at least one test compound in the presence of transporter molecules with a cell containing at least one copy of an expression product of any of the sequences of Tables I-VI which is a transporter protein or with a lipidic matrix containing at least one copy of the expression product;

c) measuring the movement of transported molecules into or from the cell, or across the lipidic matrix; and

d) selecting test compounds on the basis of their ability to modulate the movement of transported molecules.

d) G-Protein Coupled Receptor Protein Assays

An expression product of any of the nucleic acid sequences of Tables I-VI that encodes a G-protein coupled receptor protein, and in particular any of the nucleic acid sequences listed in Table IV, is amenable to screening using G-protein coupled receptor protein assay technology.

G-protein coupled receptor proteins (GPCRs) are membrane associated proteins whose main function is to transduce a signal through a cellular membrane. Upon ligand binding, GPCRs undergo a conformational change that allows complexing of the GPCRs with a G-protein. G-proteins possess a GTP/GDP binding site. The formation of the G-protein/ligand complex allows exchange of GTP for GDP, resulting in a conformational change of the G-protein. This conformational change initiates signal transduction.

Functional screening for modulators of GPCRs comprises contacting at least one test compound with an expression product as aforesaid which is a G-protein coupled receptor protein, and assessing the ability of the test compound(s) to modulate the exchange of GTP for GDP or the modulation of the GPCR signal transduction pathway. The expression product can be part of an intact cell or lipidic preparation, optionally attached to a support, for example beads, a column or a plate. Binding is preferably performed in the presence of a ligand, G-protein and GTP/GDP to allow an assessment of the binding activity of each test compound. Alternatively components of the signaling pathway are also included. In particular, in a preferred embodiment, a labeled GTP is used and the ability of the test compound(s) to modulate the exchange of GTP to GDP is determined. A further optional characteristic of the assay can be the inclusion of a reporter molecule that enables monitoring the regulation of the signaling pathway. The ligand may be contacted with the GPCR before, simultaneously with, or after the test compound. Binding of the test compound to the GPCR modifies its ability to modulate the exchange of GTP for GDP and hence the modulation of signal transduction. The difference between the observed amount of GTP exchanged for GDP relative to the theoretical maximum amount of GTP is a reflection of the modulatory effect of the test compound. Likewise the relative activities of signal transduction reporter molecules are also a reflection of the modulatory effect of the test compound. Non limiting examples of technologies and methodologies can be found in Molecular Probes handbook and references therein (Molecular Probes, Inc., 4849 Pitchford Ave, Eugene, USA), Glaxo Pocket Guide to Pharmacology, (Michael Sheehan, Pharmacology Division staff, Glaxo Group Research Ltd., Ware, Herts SC12 0DP) and Xing et al (2000, J. Recept. Signal. Transduct. Res. 20(40 189-210).

The invention provides a method of screening compounds for their ability to relieve pain, comprising:

a) contacting at least one test compound in the presence of a ligand, GTP/GDP, G-protein with a cell containing at least one copy of an expression product of a sequence of Tables I-VI as aforesaid which is a G-protein coupled receptor protein or with a lipidic matrix containing at least one copy of the expression product;

b) measuring the exchange of GTP for GDP, and

c) selecting test compounds on the basis of their ability to modulate said exchange.

In the above method, the ligand, GTP/GDP, G-protein and other essential molecules can be added before, simultaneously with or after the contacting of the test compound(s) with the cell line or lipidic matrix in step (a).

e) DNA-Binding Protein Assays

An expression product of any of the nucleic acid sequences of Tables I-VI that encodes a DNA-binding protein, and in particular any of the nucleic acid sequences listed in Table V, is amenable to screening using DNA-binding protein assay technology.

DNA binding proteins are proteins that are able to complex with DNA. The complexing of the DNA binding protein with the DNA in some instances requires a specific nucleic acid sequence. Screens can be developed in a similar manner to ligand binding screens as previously indicated and will utilise DNA as the ligand. DNA-binding protein assays can be carried using similar principles described in ligand binding assays as described above. Non limiting examples of methodology and technology can be found in the teachings of Haukanes B I and Kvam C (Biotechnology, 1993 Jan 11 60-63), Alberts B et al (Molecular Biology of the Cell, 1994, 3^{rd} Edn., Garland Publications Inc, Kirigiti P and Machida C A (2000 Methods Mol Biol, 126, 431-51) and Molecular Probes handbook and references therein (Molecular Probes, Inc., 4849 Pitchford Ave., Eugene, USA).

The invention therefore includes a method of screening for pain alleviating compounds, comprising:

a) contacting a test compound or test compounds in the presence of a plurality of nucleic acid sequences with an expression product of any of the nucleic acid sequences of Tables I-VI which is a DNA binding protein or with cells expressing at least one copy of the expression product or with a lipidic matrix containing at least one copy of the expression product;

b) determining the binding of the test compound to the expression product, and

c) selecting test compounds on the basis of their binding abilities.

In the above method, the plurality of nucleic acid sequence may be added prior to, simultaneously with or after contact of the test compound with the expression product.

f) Assays Using other Enzymes

Expression products of any of the nucleic acid sequences of Tables I-VI that encode other enzymes, e.g. ligases, lyases, oxidoreductases, transferases and hydrolases, and in particular any of the nucleic acid sequences listed in Table VI, is amenable to screening using appropriate assay technology.

Each class of enzyme has a defined function. Ligases have the property of being able to splice molecules together. This is achieved with the conversion of ATP substrate to AMP. Therefore, the activity of a ligase can be followed by monitoring the conversion of ATP to AMP. Such technologies are known to those in the art. Non limiting examples and methodologies are illustrated by Ghee. T Tan et al (1996,Biochem J. 314, 993-1000, Yang S W et al (1992, 15: 89(6) 2227-31 and refrences therein, and in Molecular Probes handbook and references therein (Molecular Probes, Inc., 4849 Pitchford Ave, Eugene, USA).

The invention also provides a methods of screening for pain alleviating compounds, comprising;

a) contacting one or more test compounds in the presence of ATP with an expression product of any of the sequences of Tables I-VI which is a ligase or with a cell expressing at least 1 copy of a expression product or with a lipidic matrix containing at least 1 copy of an expression product which is a ligase;

b) determining the amount of ATP converted to AMP, and

c) selecting test compounds on the basis of their ability to modulate said conversion.

Lyases are enzymes which catalyse the cleavage of by reactions other than hydrolysis. These enzymes can be grouped into seven groups according to type of bond cleaved. These groups are carbon-carbon lyases (E.C. No 4.1), carbon-oxygene lyases (E.C. No 4.2), carbon-nitrogen lyases (E.C. No 4.3), carbon-sulphur lyase (E.C. No 4.4) carbon-halide lyases (E.C. No 4.5), phosphorous-oxygene lyases (E.C. No 4.6) and other lyases (E.C. No 4.99) (Analytical Biochemistry 3^{rd} Edn, David J. Holme and Hazel Peck, Longman press). The enzyme commission number (E.C.) of the International Union of Biochemistry relates to the type of reaction catalysed by the enzyme. Further teachings on how to develop assays and screens for lyases can be obtained from Methods in Enzymology (Academic Press).

Oxidoreductases are enzymes that catalyse the transfer of hydrogen or oxygen atoms or electrons. These enzymes can by sub-grouped into twenty categories according to their specific mode of action. These groups are oxidoreductases acting on the CH—OH group of donors (E.C. No1.1), oxidoreductases acting on the aldehyde or oxo group of donors (E.C. No 1.2), oxidoreductases acting on the CH—CH group of the donor (E.C. No 1.3), oxidoreductases acting on the CH—NH2 group of donors (E.C. 1.4), oxidoreductases acting on the CH—NH group of donor (E.C. 1.5), oxidoreductases acting on the NADH or NADPH (E.C. No 1.6), oxidoreductases acting on other nitrogen compounds as donors (E.C. No 1.7), oxidoreductases acting on a sulphur group of donors (E.C. No 1.8), oxidoreductases acting on a haem group of donors (E.C. No1.9), oxidoreductases acting on diphenols and related substances as donors (E.C. 1.10), oxidoreductases acting on hydrogen peroxide as acceptor (E.C. No 1.11), oxidoreductases acting on hydrogen as donor (E.C. No 1.12), oxidoreductases acting on single doners with incorporation of molecular oxygen (E.C. No 1.13), oxidoreductases acting on paired donors with incorporation of molecular oxygen (E.C. No 1.14), oxidoreductases acting on superoxide radicals as acceptors (E.C. 1.15), oxidoreductases oxidizing metal ions (E.C. No 1.16), oxidoreductases acting on —CH2 groups (E.C. No 1.17), oxidoreductases acting on reduced ferredoxin as donor (E.C. No 1.18), oxidoreductases acting on reduced flavodoxin as donor (E.C. No 1.19) and other oxidoreductases (E.C. No 1.97) (Analytical Biochemistry 3^{rd} Edn, David J. Holme and Hazel Peck, Longman Press). The enzyme commission number (E.C.) of the International Union of Biochemistry relates to the type of reaction catalysed by the enzyme. Further teachings on how to develop assays and screens for oxidoreductases can be obtained from Methods in Enzymology (Academic Press) with special reference to volume 249.

Transferases are enzymes that catalyse the transfer of specific groups. They are classified into eight sub groups according to function, transferring one-carbon group (E.C. No 2.1), Transfering aldehyde or ketonic residues (E.C. No 2.2), Acetyltransferases (E.C. 2.3), glycosyltransferases (E.C. No 2.4), transferring alkyl or aryl groups other than methyl groups (E.C. No 2.5), transferring nitrogeneous groups (E.C. No 2.6), transferring phosphorous-containing groups (E.C. No 2.7) and transferring sulphur-containing groups (E.C. No 2.8) (Analytical Biochemistry 3^{rd} Edn, David J. Holme and Hazel Peck, Longman Press). The enzyme commission number (E.C.) of the International Union of Biochemistry relates to the type of reaction catalysed by the enzyme. Further teachings on how to develop assays and screens for transferases can be obtained from Methods in Enzymology (Academic Press).

Hydrolases are enzymes that catalyse hydrolytic reactions and are sub-grouped into eleven classes according to the type of reaction they carry out. Hydrolases acting on ester bonds (E.C. No 3.1), hydrolases acting on glycosyl compounds (E.C. No 3.2), hydrolases acting on ether bonds (E.C. No 3.3), hydrolases acting on peptide bonds (E.C. No 3.4), hydrolases acting on carbon-nitrogen bonds, other than peptide bonds (E.C. No 3.5), hydrolases acting on acid anhydrides (E.C. No 3.6), hydrolases acting on acid anhydrides (E.C. No 3.6), hydrolases acting on carbon-carbon bonds (E.C. No 3.7), hydrolases acting on halide bonds (E.C. No 3.8), hydrolases acting on phosphorous-nitrogen bonds (E.C. No 3.9), hydrolases acting on sulphur-nitrogen bonds (E.C. No 3.10) and hydrolases acting on carbon-phosphorous bonds (Analytical Biochemistry 3^{rd} Edn, David J. Holme and Hazel Peck, Longman Press). The enzyme commission number (E.C.) of the International Union of Biochemistry relates to the type of reaction catalysed by the enzyme. Further teachings on how to develop assays and screens for hydrolases can be obtained from Methods in Enzymology (Academic Press) with special reference to volume 249.

C) In Vivo Functional Screen

Any of the nucleotide sequences described in Tables I-VI or homologues thereof may be inserted by means of an appropriate vector into the genome of a lower vertebrate or of an invertebrate animal or may be inactivated or down regulated in the genome of said animal. The resulting genetically modified animal may be used for screening compounds for effectiveness in the regulation of pain. The invertebrate may, for example, be a nematode e.g. Caenorhabditis elegans, which is a favourable organism for the study of response to noxious stimuli. Its genome sequence has been determined, see Science, 282, 2012 (1998), it can be bred and handled with the speed of a micro-organism (it is a self-fertilizing hermaphrodite) and can therefore be used in a high throughput screening format (WO 00/63424, WO 00/63425, WO 00/63426 and WO 00/63427), and it offers a full set of organ systems, including a simple nervous system and contains many similarly functioning genes and signaling pathways to mammals. A thermal avoidance model based on a reflexive withdrawal reaction to an acute heat stimulus has been described by Wittenburg et al, Proc. Natl. Acad. Sci. USA, 96, 10477-10482 (1999), and allows the screening of compounds for the treatment of pain with the modulation of pain sensation as an endpoint.

The genome of C. elegans can be manipulated using homologous recombination technology which allows direct replacement of nucleic acids encoding C. elegans with their identified mammalian counterpart. Replacement of these nucleic acids with those nucleic acids outlined above would allow for the direct screening of test compound(s) with their expression products. Any of the pain-related genes described above may be ligated into a plasmid and introduced into oocytes of the worm by microinjection to produce germline transformants. Successful plasmid injection into C. elegans and expression of inserted sequences has been reported by Devgen B. V., Ghent, Belgium. It is also possible to produce by routine methods worms in which the target sequences are down-regulated or not expressed (knock-out worms). Further non limiting examples of methodology and technology can be found in the teachings of Hazendonk et al (1997, Nat genet. 17(1) 119-21), Alberts et al, (1994, Molecular Biology of the Cell 3^{rd} Ed. Garland Publishing Inc, Caenorhabditis elegans is anatomically and genetically simple), Broverman S et al, (1993, PNAS USA 15;90(10) 4359-63) and Mello et al (1991, 10(12)3959-70).

A further method for screening compounds for ability to modify response to pain, e.g. relieve pain, comprises:

(a) contacting one or more test compounds with at least one C elegans containing at least one copy of a sequence as set out above;

(b) subjecting the C. elegans to a nociceptive stimulus;

(c) observing the response of the C. elegans to said stimulus; and

d) selecting test compounds on the basis of their ability to modify the response of C. elegans to said stimulus.

Diagnostic Tools and Kits

Affinity Peptides, Ligands and Substrates

Pain associated polypeptides and fragments thereof can be detected at the tissue and cellular levels with the use of affinity peptides, ligands and substrates, which will enable a skilled person to define more precisely a patient's ailment and help in the prescription of a medicament. Such affinity peptides are characterized in that firstly they are able to bind specifically to a pain associated polypeptide, and secondly that they are capable of being detected. Such peptides can take the form of a peptide or polypeptide for example an antibody domain or fragment, or a peptide/polypeptide ligand or substrate, or a polypeptide complex such as an antibody.

The preparation of such peptides and polypeptides are known to those in the art. Antibodies, these may be polyclonal or monoclonal, and include antibodies derived from immunized animals or from hybridomas, or derivatives thereof such as humanized antibodies, Fab or F(ab′)2 antibody fragments or any other antibody fragment retaining the antigen binding specificity.

Antibodies directed against pain-associated gene product molecules may be produced according to conventional techniques, including the immunization of a suitable mammal with the peptides or polypeptides or fragment thereof. Polyclonal antibodies can be obtained directly from the serum of immunized animals. Monoclonal antibodies are usually produced from hybridomas, resulting from a fusion between splenocytes of immunized animals and an immortalized cell line (such as a myeloma). Fragments of said antibodies can be produced by protease cleavage, according to known techniques. Single chain antibodies can be prepared according to the techniques described in U.S. Pat. No. 4,946,778. Detection of these affinity peptides could be achieved by labeling. technologies which allow detection of peptides, such as enzymatic labeling, fluorescence labeling or radio-labeling are well known to those in the art. Optionally these affinity peptides, ligands and substrates could themselves be detected with the use of a molecule that has specific affinity to the peptides, ligands and substrates and is itself labeled.

The invention further provides a kit comprising;

(a) affinity peptide and/or ligand and/or substrate for an expression product of a gene sequence that is down-regulated in the spinal cord of a mammal in response to first and second models of neuropathic or central sensitization pain; and

(b) a defined quantity of an expression product of a gene sequence that is down-regulated in the spinal cord of a mammal both in response to first and second models of neuropathic or central sensitization pain,

for simultaneous, separate or sequential use in detecting and/or quantifying an expression product of a gene sequence that is down-regulated in the spinal cord of a mammal in response to first and second models of neuropathic or central sensitization pain.

Complimentary Nucleic Acids

Pain associated nucleic acid sequences can be characterized at the tissue and cellular levels with the use of complimentary nucleic acid sequences. Detection of the level of expression of pain associated nucleic acid sequences can help in the prognosis of a pain condition and the prescription of a medicament. These complimentary nucleic acids are characterized in that they can hybridize to a pain associated nucleic acid sequence and their presence can be detected through various techniques. Such techniques are known to those in the art and may include detection by polymerase chain reaction or detection by labeling of complimentary nucleic acid sequences by enzymatic labeling, affinity labeling fluorescent labeling or radio labeling. Complimentary strand nucleic acid sequences of this invention are 10 to 50 bases long, more preferably 15 to 50 bases long and most preferably 15 to 30 bases long, and hybridize to the coding sequence of the nucleic acid sequence.

A further aspect of this invention is a kit that comprises:

(a) nucleic acid sequences capable of hybridization to a nucleic acid sequence that is down-regulated in the spinal cord of a mammal in response to first and second models of neuropathic or central sensitization pain; and

(b) a defined quantity of one or more nucleic acid sequences capable of hybridization to a nucleic acid sequence that is down-regulated in the spinal cord of a mammal in response to first and second models of neuropathic or central sensitization pain, for simultaneous, separate or sequential use in detecting and/or quantifying a gene sequence that is down-regulated in the spinal cord of a mammal in response to first and second models of neuropathic or central sensitization pain.

Identification and Validation

Subtractive hybridization enables the identification of nucleic acid sequences whose expression profiles are modified by a stimulus. Upon stimulation of a system (in the case of this invention a nociceptive stimulus on an animal model) all observed changes in the level of nucleic acid sequence expression are due to the reaction of the system to the stimulus. Characterization of these changes in expression by way of identification of nucleic acid sequence and level of expression is both identification and validation.

The inventors have developed a four step process which allows for the simultaneous identification and validation of nucleic acid sequences whose expression are regulated by a pain stimulus, preferably a chronic pain stimulus, and more preferably a diabetic pain stimulus. This process may comprise the following steps:

(a) induction of a nociceptive stimulus in test animals;

(b) extraction of nucleic acids from specific neuronal tissue of test and control animals;

(c) selective amplification of differentially expressed nucleic acid sequences; and

(d) identification and characterization of differentially expressed gene products that are modulated by a nociceptive stimulus.

The above process is described in more detail below.

(a) Induction of Nociceptive Stimulus

The effect of the selected nociceptive stimulus on the test animal needs to be confirmable. The test subjects are therefore a species that has a “developed” nervous system, preferably similar to that of humans, most preferably rats or mice. Advantageously, the nociceptive stimulus is analogous to known pain paradigms in humans. One such paradigm of pain is the pain associated with diabetes, which can be induced in rodents with the use of streptozotocin (STZ). The present application requires the sequences to be down-regulated in two pain models which may be, but are not limited to models of neuropathic pain and/or central sensitization, and in which diabetic pain provides the first model and mechanical damage e.g. to a nerve leading into the spine can provide an appropriate second model.

Streptozotocin (STZ) induces hyperglycemia and Type 1 diabetes mellitus in rats. In particular, STZ contains a glucose analogue that allows it to be taken up by the glucose transporter 2 present on the surface of pancreatic β cells, the site of insulin synthesis. Once inside the cell, STZ causes a reduction in the level of nicotinamide adenine dinucleotide (NAD⁺). The decrease in NAD⁺ levels eventually leads to necrosis of the pancreatic β cell, causing a reduction in insulin levels and then diabetes, leading to neuropathy (diabetic) and neuropathic pain (R. B. Weiss, Cancer Treat. Rep., 66, 427-438 1982, Guy et al, Diabetologica, 28, 131-137 1985; Ziegler et al, Pain, 34, 1-10 1988; Archer et al, J. Neural. Neurosurgeon. Psychiatry, 46, 491-499 1983). The diabetic rat model has been shown to be a reliable model of hyperalgesia. We have used an STZ-induced diabetic rat model to create a state of hyperlagesia that can be compared with control animals (Courteix et al, Pain, 53, 81-88 1993).

Three models of neuropathic and/or central sensitization pain in rats, which involve nerve injury, may be used, see Ralston, DD (1998) Present models of neuropathic pain. Pain Rev. 5: 83-100. The injuries are caused by (1) loosely tying four chromic gut sutures around the sciatic nerve (CCI model developed by Bennett, G J and Y -K Xie, A peripheral mononeuropathy in rat that produces disorders of pain sensation like those seen in man, Pain 33: 87-107 (1988), (2) tightly ligating one third to one half of the fibers in the sciatic nerve (model developed by Seltzer, Z, R Dubner, Y Shire, A novel behavioral model of neuropathic pain disorders produced in rats by partial sciatic nerve injury, Pain 43: 205-218, 1990), and (3) tightly ligating the dorsal spinal nerve of a rat at the L5 or L5 and L6 levels (L5 model developed by Kim, S H and J M Chung, An experimental model for peripheral neuropathy produced by segmental spinal nerve ligation in the rat, Pain 50:355-363, 1992).

(b) Extraction of Nucleic Acids from Neuronal Tissue of Test and Control Animals

The inventors have determined that RNA extraction of whole spinal cord nervous tissue would provide a way of identifying nucleic acid sequences whose expression in spinal tissue is modulated by streptozotocin induced diabetes or by a mechanical nerve damage model for neuropathic and/or central sensitization pain e.g. CCI. Test (subjected to the nociceptive stimulus) and control animals were sacrificed, and the tissue to be studied e.g. neural tissue separated. Techniques for so doing vary widely from animal to animal and will be familiar to skilled persons.

A cDNA library can be prepared from total RNA extracted from neural tissue of the test and control animals. Where possible, however, it is preferred to isolate the mRNA from the total RNA of the test and control animals, by affinity chromatography on oligo (dT)-cellulose, and then reverse transcribe the mRNA from the test and control animals to give test and control cDNA. Converting mRNA from the test and control animals to corresponding cDNA may be carried out by any suitable reverse transcription method, e.g. a method as described by Gubler & Hoffman, Gene, 25, 263-269 (1983). If desired a proprietary kit may be used e.g. the CapFinder PCR cDNA Library Construction Kit (Life Technologies) which is based on long-distance PCR and permits the construction of cDNA libraries from nanograms of total RNA.

(c) Selective Amplification of Differentially Expressed Nucleic Acids

The reverse transcribed cDNA of the test and control animals is subjected to subtractive hybridisation and amplification so that differentially expressed sequences become selectively amplified and commonly expressed sequences become suppressed, so as to over-produce DNA associated with said nociceptic stimulus. A wide range of subtractive hybridisation methods can be used, but the preferred method is so-called suppression subtractive hybridisation, see U.S. Pat. No. 5,565,340 and Diatchenko et al, Proc. Nat. Acad. Sci. USA, 93, 6025-6030 (1996), the disclosures of which are herein incorporated by reference. Kits for carrying out this method are available from CLONTECH Laboratories, Inc.

(d) Cloning and Sequencing the Differentially Expressed cDNA

The differentially expressed cDNA is ligated into a cloning vector, after which cells of E. coli are transformed with the vector and cultured. Positive clones are selected and lysed to release plasmids containing the cDNA insert. The plasmids are primed using forward and reverse primers to either side of the cloning site and the cDNA insert is sequenced. Vector and adaptor sequences are then removed from the output data from the sequencer, leaving only the nucleotide sequence of the differentially expressed gene. The sequence is then checked against data held in a database for homology to known nucleotide sequences and genes, including expressed sequence tags (ESTs) and coding sequences for proteins.

(e) Validation of the Above Method

The importance of the sequences that we have identified in pain is confirmed by the fact that genes have been identified using this method that represent nucleic acid sequences which have previously been implicated in pain, including Calmodulin (pRCM1, Genebank X13933), Enkephalin (Genebank Y07503) and Neurotensin receptor type 2 (Genebank X97121).

The inventors have identified nucleic acid sequences of the MAP kinase pathway, a previously non pain-associated biological pathway. The inventors have subsequently shown that intra-spinal injection of a MEK inhibitor (MEK is part of the MAP kinase pathway) produces a powerful inhibition of pain (Patent application No. U.S. 60/144292). Subsequently, it has been shown that the MAP kinase is also implicated in acute inflammatory pain (Woolf et al, Nature Neuroscience 1999).

The invention will now be further described in the following Example.

### EXAMPLE

Induction of Diabetes

Diabetes was induced in adult (150-200 g) male Sprague-Dawley rats (n=6) as described by Courteix et al (supra). Animals were injected intraperitoneally with streptozotocin (STZ)(50 mg/kg) dissolved in distilled water. Control or sham animals (age-matched animals, n=6) were injected with distilled water only.

Chronic Constrictive Injury (CCI)

Rats were anaesthetized with i.p. sodium phenobarbital, after which the common left sciatic nerve was exposed at the level of the middle of the thigh by blunt dissection through the biceps femoris and proximal to the sciatic trifurcation. Four ligatures (4.0 braided silk) were tied loosely around it with about 1 mm spacing. The muscle was closed in layers and two wound clips were applied to close the skin incision. The wound was then covered with topical antibiotics.

Nociceptive Testing

Static allodynia (a form of hyperlagesia) was measured using a method described by Chaplan let al, “Quantitative assessment of tactile allodinya in the rat paw”, J. Neurosci. Methods, 53, 55-63 (1994). A series of von Frey filaments of different buckling weight (i.e. the load required for the filament to bend) were applied to the plantar surface of the right hand paw. The starting filament had a buckling weight of 20 g. Lifting of the paw was taken to be a positive result, in which case a filament with the next lowest buckling weight was used for the next measurement. The test was continued until a filament was found for which there was an absence of response for longer than 5 seconds whereas a re-test with the next heaviest filament gave a positive response. Animals were considered hyperalgesic if their thresholds were found to be <4 g of those of comparable untreated rats, see Calcutt & Chaplan, “Spinal pharmacology of tactile allodynia in diabetic rats”, British J. Pharmacol, 122, 1478-1482 (1997).

Tissue Extraction

STZ-treated, CCI-treated and control animals were anaesthetized with 4% halothane and perfused with ice-cold 0.9% saline containing 1% citric acid (pH adjusted to 7.4 with NaOH). The animals were decapitated and the lumbar spinal cord exposed. A 2-centimetre length of spinal cord ending at L6 (lumbar-6 forward) was removed from the spinal column. Attached dorsal root ganglia and contaminating spinal connective tissues were removed. The spinal cord tissue was snap frozen in dry ice and isopentane. In the experiments that follow, procedures on the streptozocin-treated and control groups of animals are disclosed. It will be understood that for identification of CCI-treated animals the same experiments are performed, but using tissues from the CCI-treated animals and from control animals.

Total RNA Extraction

Total RNA was extracted from the pooled male rat tissues of the streptozocin-treated and control groups using the TRIZOL Reagent Kit (Life Technologies). Briefly, tissue samples were homogenised fully using a Polytron homogenizer in 1 ml of TRIZOL reagent per 50-100 mg of tissue. Homogenized samples were then incubated at room temperature for 5 minutes and phase separated using 0.2 ml chloroform per 1 ml TRIZOL reagent followed by centrifugation at 3,000 g. The aqueous phase was transferred to a fresh tube and the RNA precipitated with an equal volume of isopropyl alcohol and followed by centrifugation at 10,000 g. The RNA pellet was washed in 75% ethanol and re-centrifuged. The pellet was then air dried and re-suspended in water.

mRNA Extraction

In contrast to ribosomal RNA and transfer RNA, the vast majority of mRNAs of mammalian cells carry tracts of poly(A⁺) at their 3′ termini. mRNAs can therefore be separated from the bulk of cellular RNA by affinity chromatography on oligo (dT)-cellulose. mRNA was extracted from Total RNA using the MESSAGEMAKER Kit (Life Technologies) in which mRNA (previously heated to 65° C. in order to disrupt secondary structures and so expose the poly (A⁺) tails) was bound to oligo(dT) cellulose under high salt concentrations (0.5M NaCl) in a filter syringe. Unbound RNA was then washed away and the poly(A⁺) mRNA eluted in distilled water. A tenth of the volume of 7.5 M Ammonium Acetate, 50 μg of glycogen/ml mRNA sample and 2 volumes of absolute alcohol were then added to the samples which were placed at −20° C. overnight. Following precipitation, the mRNA was spun down at 12,000 g for 30 minutes at 4° C. RNAase free water was used to re-suspend the pellets, which were then and stored at −80° C.

PCR Select

The technique used was based on that of the CLONTECH PCR Select Subtraction Kit. The following protocol was performed using STZ-treated lumbar spinal cord Poly A⁺ RNA as the ‘Tester’ and Sham lumbar spinal cord poly A⁺ RNA as the ‘Driver’ (Forward Subtraction). A second subtraction experiment using the Sham lumbar spinal cord mRNA as the ‘Tester’ and STZ treated lumbar spinal cord mRNA as the ‘Driver’ (Reverse Subtraction) was performed in parallel using the same reagents and protocol. As a control for both experiments, the subtraction was also carried out using human skeletal muscle mRNA that had been provided by the manufacturer.

First-Strand cDNA Synthesis

2 μg of PolyA⁺ RNA and 1 μl of cDNA synthesis primer (10 μM) were combined in a 0.5 ml Eppendorf tube and sterile H₂O was added where necessary to achieve a final volume of 5 μl. The contents were mixed gently and incubated in a thermal cycler at 70° C. for 2 min. The tubes were then cooled on ice for two minutes, after which 2 μl of 5×First-strand buffer, 1 μl of dNTP mix (10 mM each), sterile H₂O and 1 μl of AMV reverse transcriptase (20 units/μl) was also added. The tubes were then placed at 42° C. for 1.5 hr in an air incubator. First-strand cDNA synthesis was terminated by placing the tubes on ice. (the human skeletal muscle cDNA produced by this step was used as the ‘control driver’ in later steps).

Second-Strand cDNA Synthesis

48.4 μl of Sterile H₂O, 16.0 μl of 5×Second-strand buffer, 1.6 μl of dNTP mix (10 mM) and 4.0 μl of 20×Second-strand enzyme cocktail were added to each of the first-strand synthesis reaction tubes. The contents were then mixed and incubated at 16° C. in a thermal cycler for 2 hr. 6 units (2 μl) of T4 DNA polymerase was then introduced and the tubes were incubated for a further 30 min at 16° C. In order to terminate second-strand synthesis, 4 μl of 20×EDTA/glycogen mix was added. A phenol:chloroform extraction was then carried out using the following protocol:-

100 μl of phenol:chloroform:isoamyl alcohol (25:24:1) was added to the tubes which were then vortexed thoroughly and centrifuged at 14,000 rpm for 10 min at room temperature. The top aqueous layer was removed and placed in a fresh tube. 100 μl of chloroform:isoamyl alcohol (24:1) was then added to the aqueous layer and the tubes were again vortexed and centrifuged at 14,000 rpm for 10 min. 40 μl of 4 M NH₄OAc and 300 μl of 95% ethanol were then added and the tubes centrifuged at 14,000 rpm for 20 min. The supernatant was removed carefully, then 500 μl of 80% ethanol was added to the pellet. The tubes were centrifuged at 14,000 rpm for 10 min and the supernatant was removed so that the pellet could be air-dried. The precipitate was then dissolved in 50 μl of H₂O. 6 μl was transferred to a fresh microcentrifuge tube. The remainder of the sample was stored at −20° C. until needed.

Rsa I Digestion

All products of the above procedures were subjected to a restriction digest, using the restriction endonuclease Rsa I, in order to generate ds cDNA fragments that are short and thus are optimal for subtraction hybridisation due to the standard kinetics of the hybridisation. Also, as Rsa I makes a double stranded cut in the middle of a recognition sequence, ‘blunt ends’ of a known nucleotide sequence are produced allowing ligation of adaptors onto these ends in a later step. The following reagents were added to the 6 μl product of the second hybridisation (see above): 43.5 μl of ds cDNA, 5.0 μl 10×Rsa I restriction buffer and 1.5 μl of Rsa I (10 units/μl). The reaction mixture was incubated at 37° C. for 1.5 hr. 2.5 μl of 20×EDTA/glycogen mix was used to terminate the reaction. A phenol:chloroform extraction was then performed as above (second-strand cDNA synthesis section). The pellet produced was then dissolved in 5.5 μl of H₂O and stored at −20° C. until needed. The preparation of the experimental ‘Driver’ cDNAs and the control skeletal muscle cDNA was thus completed.

Adaptor Ligation

The adaptors were not ligated to the driver cDNA.

1 μl of each Rsa I-digested experimental cDNA (from the Rsa I Digestion above) was diluted with 5 μl of sterile water. Preparation of the control skeletal muscle tester cDNA was then undertaken by briefly centrifuging the tube containing control DNA (Hae III-digest of φX174 DNA [3 ng/μl]) and diluting 2 μl of the DNA with 38 μl of sterile water (to 150 ng/ml). 1 μl of control skeletal muscle cDNA (from the Rsa I Digestion) was then mixed with 5 μl of the diluted φX174/Hae III DNA (150 ng/ml) in order to produce the control skeletal muscle tester cDNA.

Preparation of the Adaptor-Ligated Tester cDNA

A ligation master mix was prepared by combining 3 μl of sterile water, 2 μl of 5×ligation buffer and 1 μl T4 DNA ligase (400 units/μl) per reaction. 2 μl of adaptor 1 (10 μM) was then added to 2 μl of the diluted tester cDNA. To this, 6 μl of the ligation master mix was also added. The tube was therefore labeled Tester 1-1. In a separate tube, 2 μl of the adaptor 2R (10 μM) was mixed with 2 μl of the diluted tester cDNA and 6 μl of the master mix. This tube was named Tester 1-2.

2 μl of Tester 1-1 and 2 μl of Tester 1-2 were then placed into fresh tubes. These would later be used as the unsubtracted tester control. The remainder of the contents of Tester 1-1 and Tester 1-2 tubes were then centrifuged briefly and incubated at 16° C. overnight. The ligation reaction was stopped by adding 1 μl of EDTA/glycogen mix and the samples were heated at 72° C. for 5 min in order to inactivate the ligase. In doing so, preparation of the experimental and control skeletal muscle adaptor-ligated tester cDNAs was complete.

1 μl from each unsubtracted tester control was then removed and diluted into 1 ml of water. These samples were set aside as they were to be used later for PCR (see below). All of the samples were stored at −20° C.

Analysis of Ligation Efficiency

1 μl of each ligated cDNA was diluted into 200 μl of water and the following reagents were then combined in four separate tubes:

Tube:

| Component | 1 | 2 | 3 | 4 |
|---|---|---|---|---|
| Tester 1-1 (ligated to Adaptor 1) | 1 | 1 | — | — |
| Tester 1-2 (ligated to Adaptor 2R) | — | — | 1 | 1 |
| G3PDH 3′ primer (10 μM) | 1 | 1 | 1 | 1 |
| G3PDH 5′ primer (10 μM) | — | 1 | — | 1 |
| PCR primer 1 (10 μM) | 1 | — | 1 | — |
| Total volume μl | 3 | 3 | 3 | 3 |

A master mix for all of the reaction tubes plus one additional tube was made up by adding 18.5 μl of sterile H₂O, 2.5 μl of 10×PCR reaction buffer, 0.5 μl of dNTP mix (10 mM), and 0.5 μl of 50×Advantage cDNA Polymerase Mix, per reaction, into a fresh tube. 22 μl of this master mix was then aliquotted into each of the 4 reaction tubes prepared above. The contents of the tubes were overlaid with 50 μl of mineral oil. The reaction mix was incubated in a thermal cycler at 75° C. for 5 min in order to extend the adaptors. The following protocol was then carried out immediately in a thermal cycler (Perkin-Elmer GeneAmp PCR Systems 2400): 94° C. for 30 sec (1 cycle), 94° C. 10 sec, 65° C. 30 sec and then 68° C. 2.5 min (25 cycles)

First Hybridisation

1.5 μl of the Adaptor 1-ligated Tester 1-1 was combined with 1.5 μl of the Rsa I-digested driver cDNA, prepared earlier and 1 μl of 4×Hybridisation buffer. This process was then repeated combining the Adaptor 2R-ligated Tester 1-2 with the Rsa I-digested driver cDNA and 4×hybridisation buffer. The samples were incubated in a thermal cycler at 98° C. for 1.5 min followed by incubation at 68° C. for 8 hr.

Second Hybridisation

1 μl of Driver cDNA (i.e. the Rsa I-digested cDNA (see above)), 1 μl 4×Hybridisation buffer and 2 μl Sterile H₂O were all combined in a fresh tube. 1 μl of this mix was then removed and placed in a new tube, overlaid with 1 drop of mineral oil and incubated at 98° C. for 1.5 min in order to denature the driver. The following procedure was used to simultaneously mix the driver with hybridisation samples 1 and 2 (prepared in the first hybridisation), thus ensuring that the two hybridisation samples were mixed together only in the presence of freshly denatured driver: A micropipettor was set at 15 μl. The pipette tip was then touched onto the mineral oil/sample interface of the tube containing hybridisation sample 2. The entire sample was drawn partway into the tip before it was removed from the tube in order to draw a small amount of air into the tip. The pipette tip was then touched onto the interface of the tube containing the freshly denatured driver (i.e. the tip contained both samples separated by a small pocket of air) before the entire mixture was transferred to the tube containing hybridisation sample 1. The reaction was then incubated at 68° C. overnight. 200 μl of dilution buffer was added to the tube, which was then heated in a thermal cycler at 68° C. for 7 min. The product of this second hybridisation was stored at −20° C.

PCR Amplification

Seven PCR reactions were set up: (1) The forward-subtracted experimental cDNA, (2) the unsubtracted tester control (see preparation of the adaptor ligated tester cDNA), (3) the reverse-subtracted experimental cDNA, (4) the unsubtracted tester control for the reverse subtraction, (5) the subtracted control skeletal muscle cDNA, (6) the unsubtracted tester control for the control subtraction, and (7) the PCR control subtracted cDNA (provided in the kit). The PCR control subtracted cDNA was required to provide a positive PCR control as it contained a successfully subtracted mixture of Hae III-digested φX174 DNA.

The PCR templates were prepared by aliquotting 1 μl of each diluted cDNA (i.e., each subtracted sample from the second hybridisation and the corresponding diluted unsubtracted tester control produced by the adaptor ligation see above) into an appropriately labeled tube. 1 μl of the PCR control subtracted cDNA was placed into a fresh tube. A master mix for all of the primary PCR tubes, plus one additional reaction, was then prepared by combining 19.5 μl of sterile water, 2.5 μl of 10×PCR reaction buffer, 0.5 μl of dNTP Mix (10 mM), 1.0 μl of PCR primer 1 (10 μM) and 0.5 μl of 50×Advantage cDNA Polymerase Mix. 24 μl of Master Mix was then aliquotted into each of the 7 reaction tubes prepared above and the mixture was overlaid with 50 μl of mineral oil, before being incubated in a thermal cycler at 75° C. for 5 min in order to extend the adaptors. Thermal cycling was then immediately started using the following protocol: 94° C. 25 sec (1 cycle), 94° C. 10 sec, 66° C. 30 sec and 72° C. 1.5 min (32 cycles).

3 μl of each primary PCR mixture was then diluted in 27 μl of H₂O, 1 μl of each of these dilutions was then placed into a fresh tube.

A master mix for the secondary PCRs, (plus an additional reaction) was set up by combining 18.5 μl of sterile water, 2.5 μl of 10×PCR reaction buffer, 1.0 μl of Nested PCR primer 1 (10 μM), 1.0 μl of Nested PCR primer 2R (10 μM), 0.5 μl of dNTP Mix (10 mM) and 0.5 μl of 50×Advantage cDNA Polymerase Mix per reaction. 24 μl of this Master Mix was then added into each reaction tube containing the 1 μl diluted primary PCR mixture. The following PCR protocol was then carried out: 94° C. 10 sec, 68° C. 30 sec and 72° C. 1.5 min (12 cycles). The reaction products were then stored at −20° C.

Ligation Into a Vector/Transformation & PCR

The products of the PCR amplification (enriched for differentially expressed cDNAs) were ligated into the pCR2.1-TOPO vector using a T/A cloning kit (Invitrogen), transformed into TOPO One Shot competent cells according to the manufacturers protocol and grown up on LB (Luria-Bertani) Agar plates overnight at 37° C. 1,000 colonies were then individually picked (using fresh sterile tips) and dipped into 5 μl of sterile water which had been aliquotted previously into 96 well PCR plates. The water/colonies were heated in a thermal cycler at 100° C. for 10 minutes in order to burst the cells, thus releasing the plasmids containing a differentially expressed cDNA insert. The 5 μl of water/plasmid was then used as a template in a PCR reaction (see below) using M13 Forward and Reverse primers (10 ng/μl), complementary to the M13 site present on either side of the cloning site on the vector. 5 μl of the PCR product was then run on a 2% agarose gel and stained by ethidium bromide. PCR products of an amplified insert were identified and 5 μl of the remainder of the PCR product (i.e. from the 15 μl that had not been run on the gel) was diluted 1/10 with water. 5 μl of the diluted PCR product was then used as a template in a sequencing reaction.

Sequencing

A sequencing reaction containing M13 primer (3.2 pmol/μl), ‘BigDye’ reaction mix (i.e. AmpliTaq® DNA polymerase, MgCl₂, buffer and fluorescent dNTPs [each of the four deoxynucleoside triphosphates is linked to a specific fluorescent donor dye which in turn is attached to a specific acceptor dye]) and cDNA template (diluted PCR product) was set up. The reaction was carried out on a thermal cycler for 25 cycles of 10 seconds at 96° C., 20 seconds at 50° C. and 4 minutes at 60° C. Each reaction product was then purified through a hydrated Centri-Sep column, and lyophilised. The pellets were resuspended in Template Supression Reagent and sequenced on an ABI Prism 310 Genetic Analyser. The analyser uses an ion laser to excite the specific donor dye that transfers its energy to the acceptor dye, which emits a specific energy spectrum that can be read by the sequencer.

The differentially expressed genes of the streptozocin-induced diabetes experiment and of the CCI experiment were sequenced at Parke-Davis, Cambridge and at the applicant's core sequencing facility in Ann Arbor, Mich., USA.

Bioinformatics

The sequencing results were analysed using the computer program CHROMAS in which the vector and adaptor sequences were clipped off, leaving only the nucleotide sequence of the differentially expressed gene. Each sequence was then checked for homology to known genes, Expressed Sequence Tags (ESTs) and Proteins using various Basic Local Alignment Search Tool (BLAST) searches against the Genbank sequence database at the National Centre for Biotechnology Information, Bethesda, Md., USA (NCBI).

Lists were derived called STZup and STZdown and CCIup and CCIdown that contain the nucleic acid sequences from the forward and back subtracted libraries respectively. In each list there are given accession numbers and descriptions for the known rat genes identified, and where available corresponding mouse or human genes. Sequences that are considered to be of interest and that are down-regulated both in a streptozocin-induced diabetes model and in a chronic constrictive injury pain model are identified in Tables I-VI above and are listed below.

References have been given where available for the sequences that have been found, and sequence listings have been given in the form in which they currently appear in publicly searchable databases e.g. the NCBI databaase (National Center for Biotechnology Information, National Library of Medicine, National Institutes of Health, Bethesda, Maryland, Md. 20894, USA, www.ncbi.nlm.nih.gov). These sequence listings are given for the purposes of identification only. The invention includes the use of subsequently revised versions of the above sequences (which may incorporate small differences to the version set out herein) and homologous sequences or similar proteins in other species as determined by a high percentage identity (e.g. above 50%, preferably above 90%), length of alignment and functional equivalence.

1. Noguchi, T., Inoue, H. and Tanaka, T., The M-1- and M-2-type isozymes of rat pyruvate kinase are produced from the same gene by alternative RNA splicing, J. Biol. Chem. 261, 13807-13812 (1986).

2. Takenaka, M., Noguchi, T., Inoue, H., Yamada, K., Matsuda, T. and Tanaka, T., Rat pyruvate kinase M gene: Its complete structure and characterization of the 5′-flanking region, J. Biol. Chem. 264, 2363-2367 (1989).

3. Zhou, Q. Y., Grandy, D. K., Thambi, L., Kushner, J. A., Van Tol, H. H., Cone R., Pribnow, D., Salon, J., Bunzow, J. R. and Civelli, O., Cloning and expression of human and rat D1 dopamine receptors, Nature, 347 (6288), 76-80 (1990)

4. Sullivan, R., Chateauneuf, A., Coulombe, N., Kolakowski, L. F. Jr., Johnson, M. P., Hebert, T. E., Ethier, N., Belley, M., Metters, K., Abramovitz, M., O'Neill, G. P. and Ng, G. Y. K., Coexpression of full-length gamma-aminobutyric acid(B) (GABA(B)) receptors with truncated receptors and metabotropic glutamate receptor 4 supports the GABA(B) heterodimer as the functional receptor, J. Pharmacol. Exp. Ther. 293 (2), 460-467 (2000)

5. Premont, R. T., Claing, A., Vitale, N., Freeman, J. L., Pitcher, J. A., Patton, W. A., Moss, J., Vaughan, M. and Lefkowitz, R. J., beta2-Adrenergic receptor regulation by GIT1, a G protein-coupled receptor kinase-associated ADP ribosylation factor GTPase-activating protein, Proc. Natl. Acad. Sci. U.S.A., 95 (24), 14082-14087 (1998).

6. Wells, D. and Kedes, L., Structure of a human histone cDNA: evidence that basally expressed histone genes have intervening sequences and encode polyadenylylated mRNAs, Proc. Natl. Acad. Sci. U.S.A., 82 (9), 2834-2838 (1985).

7. Ayte, J., Gil-Gomez, G. and Hegardt, F. G., Nucleotide sequence of a rat liver cDNA encoding the cytosolic 3-hydroxy-3-methylglutaryl coenzyme A synthase, Nucleic Acids Res., 18 (12), 3642 (1990).

8. Teruya, J. H., Kutsunai, S. Y., Spear, D. H., Edwards, P. A. and Clarke, C. F., Testis-specific transcriptional initiation sites of rat farnesyl pyrophosphate synthetase mRNA, Mol. Cell. Biol., 10, 2315-2326 (1990).

9. Beck, K. F., Schreglmann, R., Stathopulos, I., Klein, H., Hoch, J. and Schweizer, M., The fatty acid synthase (FAS) gene and its promoter in Rattus norvegicus, DNA Seq., 2 (6), 359-386 (1992).

10. Mill, J. F., Mearow, K. M., Purohit, H. J., Haleem-Smith, H., King, R. and Freese, E., Cloning and functional characterization of the rat glutamine synthetase gene, Brain Res. Mol. Brain Res., 9 (3), 197-207 (1991).

11. Vincent, C., Tarbouriech, N. and Hartlein, M., Genomic organization, cDNA sequence, bacterial expression, and purification of human seryl-tRNA synthase, Eur. J. Biochem., 250 (1), 77-84 (1997).

12. Giallongo, A., Feo, S., Moore, R., Croce, C. M. and Showe, L. C., Molecular cloning and nucleotide sequence of a full-length cDNA for human alpha enolase, Proc. Natl. Acad. Sci. U.S.A., 83 (18), 6741-6745 (1986).

13. Carper, D., Nishimura, C., Shinohara, T., Dietzchold, B., Wistow, G., Craft, C., Kador, P. and Kinoshita, J. H., Aldose reductase and p-crystallin belong to the same protein superfamily as aldehyde reductase, FEBS Lett. 220 (1), 209-213 (1987).

14. Ku, C. Y., Lu, Q., Ussuf, K. K., Weinstock, G. M. and Sanborn, B. M., Hormonal regulation of cytochrome oxidase subunit messenger RNAs in rat Sertoli cells, Mol. Endocrinol. 5 (11), 1669-1676 (1991).

15. Tsuji, S., Qureshi, M. A., Hou, E. W., Fitch, W. M. and Li, S. S. -L., Evolutionary relationships of lactate dehydrogenases from mammals, birds, an amphibian, fish, barley, and bacteria: LDH cDNA sequences from Xenopus, pig and rat, Proc. Natl. Acad. Sci. U.S.A. 91, 9392-9396 (1994).

16. Taanman, J. W., Schrage, C., Ponne, N., Bolhuis, P., de Vries, H. and Agsteribbe, E., Nucleotide sequence of cDNA encoding subunit VIb of human cytochrome c oxidase, Nucleic acids research. 17 (4), 1766 (1989).

17. Triepels, R., van den Heuvel, L., Loeffen, J., Smeets, R., Trijbels, F. and Smeitink, J., The nuclear-encoded human NADH: ubiquinone oxidoreductase NDUFA8 subunit: cDNA cloning, chromosomal localization, tissue distribution, and mutation detection in complex-I-deficient patients, Hum. Genet. 103 (5), 557-563 (1998).

18. Hosokawa, Y., Suzuki, H., Toda, H., Nishikimi, M. and Ozawa, T. Complementary DNA encoding core protein II of human mitochondrial cytochrome bc-1 complex: Substantial diversity in deduced primary structure from its yeast counterpart, J. Biol. Chem. 264, 13483-13488 (1989).

19. Harnik-Ort, V., Prakash, K., Marcantonio, E., Colman, D. R., Rosenfeld, M. G., Adesnik, M., Sabatini, D. D. and Kreibich, G. Isolation and characterization of cDNA clones for rat ribophorin I: complete coding sequence and in vitro synthesis and insertion of the encoded product into endoplasmic reticulum membranes, J. Cell Biol. 104(4), 855-863 (1987).

20. Falany, C. N., Xie, X., Wang, J., Ferrer, J. and Falany, J. L., Molecular cloning and expression of novel sulphotransferase-like cDNAs from human and rat brain, Biochem. J. 346 Pt 3, 857-864 (2000).

21. Pan, W., Ko, Y. H. and Pedersen, P. L., Delta subunit of rat liver mitochondrial ATP synthase: molecular description and novel insights into the nature of its association with the F1-moiety, Biochemistry 37 (19), 6911-6923 (1998).

22. Hamajima, N., Matsuda, K., Sakata, S., Tamaki, N., Sasaki, M. and Nonaka, M., A novel gene family defined by human dihydropyrimidinase and three related proteins with differential tissue distribution, Gene 180 (1-2), 157-163 (1996).

23. McGuire, J. A., Poellinger, L., Wikstrom, A. C. and Gustafsson, J. A., Cloning and regulation by glucocorticoid receptor ligands of a rat hsp90 J. Steroid Biochem. Mol. Biol. 42 (8), 813-822 (1992).

24. Roach, A., Boylan, K. B., Horvath, S., Prusiner, S. B. and Hood, L. E., Characterization of cloned cdna representing rat myelin basic protein: absence of expression in brain of shiverer mutant mice, Cell 34, 799-806 (1983)

25. Hoshino, A., Hisayasu, S. and Shimada, T., Complete sequence analysis of rat transferrin and expression of transferrin but not lactoferrin in the digestive glands, Comp. Biochem. Physiol. B 113, 491-497 (1996).

26. Chin, S. S. and Liem, R. K., Expression of rat neurofilament proteins NF-L and NF-M in transfected non-neuronal cells, Eur. J. Cell Biol. 50 (2), 475-490 (1989).

27. Lai, C., Brow, M. A. D., Nave, K. -A., Noronha, A. B., Quarles, R. H., Bloom, F. E., Milner, R. J. and Sutcliffe, J. G., Two forms of 1B236/myelin-associated glycoprotein, a cell adhesion molecule for postnatal neural development, are produced by alternative splicing, Proc. Natl. Acad. Sci. U.S.A. 84, 4337-4341 (1987).

28. Napolitano, E. W., Chin, S. S. M., Colman, D. R. and Liem, R. K. H., Complete amino acid sequence and in vitro expression of rat NF-M, the middle molecular weight neurofilament protein, J. Neurosci. 7, 2590-2599 (1987).

29. Nakayama, M., Miyake, T., Gahara, Y., Ohara, O. and Kitamura, T., A novel RING-H2 motif protein downregulated by axotomy: its characteristic localization at the postsynaptic density of axosomatic synapse, J. Neurosci. 15 (7 Pt 2), 5238-5248 (1995).

30. Kuwano, R., Usui, H., Maeda, T., Fukui, T., Yamanari, N., Ohtsuka, E., Ikehara, M. and Takahashi, Y., Molecular cloning and the complete nucleotide sequence of cDNA to mRNA for S-100 protein of rat brain, Nucleic Acids Res. 12 (19), 7455-7465 (1984).

31. Zauner, W., Kratz, J., Staunton, J., Feick, P. and Wiche, G., Identification of two distinct microtubule binding domains on recombinant rat MAP 1B, Eur. J. Cell Biol. 57 (1), 66-74 (1992).

32. Ozaki, T., Irie, K. and Sakiyama, S., Molecular cloning and cell cycle-dependent expression of a novel gene that is homologous to cdc37, DNA Cell Biol. 14 (12), 1017-1023 (1995).

33. Albertsen, H. M., Smith, S. A., Mazoyer, S. S., Fujimoto, E., Stevens, J., Williams, B., Rodriguez, P., Cropp, C. S., Slijepcevic, P. and Carlson, M., A physical map and candidate genes in the BRCA1 region on chromosome 17q12-21, Nature Genet. 7 (4), 472-479 (1994).

34. Dieffenbach, C. W., SenGupta, D. N., Krause, D., Sawzak, D. and Silverman, R. H., Cloning of murine gelsolin and its regulation during differentiation of embryonal carcinoma cells, J. Biol. Chem. 264, 13281-13288 (1989).

35. Geisert, E. E. Jr., Murphy, T. P., Irwin, M. H. and Larjava, H., A novel cell adhesion molecule, G-CAM, found on cultured rat glia , Neurosci. Lett. 133 (2), 262-266 (1991); Irwin, .H. and Geisert, .E. Jr., The upregulation of a glial cell surface antigen at the astrocytic scar in the rat, Neurosci. Lett. 154 (1-2), 57-60 (1993); Geisert, E. E. Jr., Yang, L. and Irwin, M. H., Astrocyte growth, reactivity, and the target of the antiproliferative antibody, TAPA, J. Neurosci. 16 (17), 5478-5487 (1996).

36. Holz, A., Schaeren-Wiemers, N., Schaefer, C., Pott, U., Colello, R. J. and Schwab, M. E., Molecular and developmental characterization of novel cDNAs of the myelin-associated/oligodendrocytic basic protein, J. Neurosci. 16 (2), 467-477 (1996).

37. Ching, G. Y. and Liem, R. K. H., Structure of the gene for the neuronal intermediate filament protein alpha-internexin and functional analysis of its promoter, J. Biol. Chem. 266, 19459-19468 (1991).

38. Lai, C. H., Chou, C. Y., Ch'ang, L. Y., Liu, C. S. and Lin, Wc., Identification of novel human genes evolutionarily conserved in caenorhabditis elegans by comparative proteomics, Genome Res. 10 (5), 703-713 (2000).

39. Nagase, T., Seki, N., Tanaka, A., Ishikawa, K. and Nomura, N., Prediction of the coding sequences of unidentified human genes. IV. The coding sequences of 40 new genes (KIAA0121-KIAA0160) deduced by analysis of cDNA clones from human cell line KG-1, DNA Res. 2 (4), 167-174 (1995).

40. Wendel, M., Sommarin, Y., Bergman, T. and Heinegard, D., Isolation, characterization, and primary structure of a calcium-binding 63-kDa bone protein, J. Biol. Chem. 270 (11), 6125-6133 (1995).

41. Welcher, A. A., Suter, U., De Leon, M., Snipes, G. J. and Shooter, E. M., A myelin protein is encoded by the homologue of a growth arrest-specific gene, Proc. Natl. Acad. Sci. U.S.A. 88, 7195-7199 (1991).

42. Nudel, U., Zakut, R., Shani, M., Neuman, S., Levy, Z. and Yaffe, D., The nucleotide sequence of the rat cytoplasmic beta-actin gene, Nucleic Acids Res. 11 (6), 1759-1771 (1983).

43. Miwa, J. M., Ibanez-Tallon, I., Crabtree, G. W., Sanchez, R., Sali, A., Role, L. W. and Heintz, N., lynx1, an endogenous toxin-like modulator of nicotinic acetylcholine receptors in the mammalian CNS, Neuron 23 (1), 105-114 (1999).

44. Rothermel, E., Detzler, E., Walter, L., Levan, G. and Gunther, E., Characterization and mapping of a highly conserved processed pseudogene and an intron-carrying gene of the heat shock cognate protein 70 (Hsc70) gene family in the rat, Mamm. Genome 6 (9), 602-606 (1995).

45. Sadot, E., Marx, R., Barg, J., Behar, L. and Ginzburg, I., Complete sequence of 3′-untranslated region of Tau from rat central nervous system. Implications for mRNA heterogeneity, J. Mol. Biol. 241 (2), 325-331 (1994).

46. Lemke, G. and Axel, R., Isolation and sequence of a cDNA encoding the major structural protein of peripheral myelin, Cell 40, 501-508 (1985).

1. Use of:
(a) an isolated gene sequence that is down-regulated in the spinal cord of a mammal in response to mechanistically distinct first and second models of neuropathic or central sensitization pain;
(b) an isolated gene sequence comprising a nucleic acid sequence of any of Tables I to VI;
(c) an isolated gene sequence having at least 80% sequence identity with a nucleic acid sequence of any of Tables I to VI;
(d) an isolated nucleic acid sequence that is hybridizable to any of the gene sequences according to (a), (b) or (c) under stringent hybridisation conditions;
(e) a recombinant vector comprising a gene sequence or nucleic acid sequence according to any one of (a) to (d);
(f) a host cell containing the vector according to (e);
(g) a non-human animal having in its genome an introduced gene sequence or nucleic acid sequence or a removed or down-regulated gene sequence or nucleic acid sequence according to any one of (a) to (d);
(h) an isolated polypeptide comprising an amino acid sequence at least 90% identical to an amino acid sequence encoded by a nucleotide sequence according to any one of (a) to (d), or a polypeptide variant thereof with sequential amino acid deletions from the C terminus and/or the N-terminus;
(i) an isolated polypeptide encoded by a nucleotide sequence according to any one of (a) to (d); or
(j) an isolated antibody that binds specifically to a polypeptide according to (h) or (i);
in the screening of compounds for the treatment of pain, or for the diagnosis of pain.

2. Use according to claim 1, wherein the isolated gene sequence is down-regulated both in response to streptozocin-induced diabetes and in response to surgical injury of a nerve leading to the spine.

3. Use according to claim 1 wherein the isolated gene sequence encodes a kinase.

4. Use according to claim 3, wherein the isolated gene sequence encodes an expression product or fragment thereof of pyruvate kinase, M1 and M2 subunits (M24359; X97047; X56494).

5. Use according to claim 1, wherein the isolated gene sequence encodes an expression product or fragment thereof of a receptor.

6. Use according to claim 5, wherein the isolated gene sequence encodes dopamine receptor D.sub.1 (158000).

7. Use according to claim 1, wherein the isolated gene sequence encodes a transporter.

8. Use according to claim 7, wherein the isolated gene sequence encodes differentiation-associated Na⁺-dependent inorganic phosphate cotransporter (AF271235) or putative vacuolar assembly protein VSP41 gene (U87309).

9. Use according to claim 1, wherein the isolated gene sequence encodes a G-protein coupled receptor protein.

10. Use according to claim 9, wherein the isolated gene sequence encodes Git1 (G-protein-coupled receptor kinase-interactor 1; GPCR kinase-associated ADP-ribosylation factor) (AF085693).

11. Use according to claim 1, wherein the isolated gene sequence encodes a DNA-binding protein.

12. Use according to claim 11, wherein the isolated gene sequence encodes putative histone H3.3A (X91866; M11354).

13. Use according to claim 1, wherein the isolated gene sequence encodes a ligase.

14. Use according to claim 13, wherein the isolated gene sequence encodes 3-Hydroxy 3-methylglutaryl coenzyme A synthase, cytosolic (X52625), acyl-CoA synthetase II, brain (D360666) farnesyl diphosphate synthase (M34477), bendless protein (AB032739; E12457), fatty acid synthase (X62888), glutamine synthetase (EC 6.3.1.2) (M91652), or putative seryl-tRNA synthetase (X91257).

15. Use according to claim 1, wherein the isolated gene sequence encodes a lyase.

16. Use according to claim 15, wherein the lyase is enolase, alpha alpha, non-neuronal (NNE) (X02610; X52379; M14328).

17. Use according to claim 1, wherein the isolated gene sequence encodes an oxidoreductase.

18. Use according to claim 17, wherein the isolated gene sequence encodes aldose reductase, lens (AREC 11.1.21) (X05884) cytochrome-c oxidase I, mitochondrial (S79304), lactate dehydrogenase-B (LDH-B) (U07181; X51905; Y00711), putative cytochrome c oxidase VIB (EC 1.9.3.1) (X13923), putative NADH: ubiquinone oxidoreductase PGIV subunit (AF044953), putative succinate dehydrogenase flavoprotein (AF095938; AF171022), putative ubiquinol-cytochrome-c reductase (EC 1.10.2.2) core protein II (J04973) or stearoyl-coA desaturase 2 (AB032243; M26270).

19. Use according to claim 1, wherein the isolated sequence encodes a transferase.

20. Use according to claim 19, wherein the isolated sequence encodes ribophorin I (X05300) or sulfotransferase-like protein (AF188699).

21. Use according to claim 1, wherein the isolated sequence encodes a hydrolase.

22. Use according to claim 21, wherein the isolated sequence encodes ATP synthase, H+, alpha subunit, mitochondrial (EC 3.6.1.34) (X56133), F1F0 ATPase delta subunit (U00926), putative dihydropyrimidinase related protein (D78013), heat shock protein 90 (S45392; M18186; M16660), or putative ribonuclease III (AF116910).

23. Use according to claim 1, wherein the isolated sequence encodes myelin basic protein S (MBP S) (K00512), transferring (D38380), neurofilament, light molecular weight (NF-L) (AF031880), myelin-associayted glycoprotein (MAG) (M16800; M31811), NF-M middle molecular weight neurofilament protein (M18628) neuro-degeneration associated-protein 1 (D32249), S-100 protein β-subunit (X01090), microtubule-associated protein 1b (Map 1b) (X60370; L06237), putative cdc 37 homolog (D26564), putative ras-related protein Rab-5c (U11293), putative gelsolin (J04953), Cd81 antigen (target of antiproliferative antibody 1) (U19894; X59047; M33680), Mobp81 (Myelin-associated/Oligo-dendrocytic basic protein 81) (X87900), syntaxin binding protein n-sec1, sec1 homolog, A-internexin (M73049), putative β-sarcoglycan A3b (AB024921), CGI-78 protein (AF151835), KIAA0143 (D63477), septin 2 (D50918), Nucleobindin (Z36277), myelin protein SR13 (M69139; S55427), B-Actin, cytoplasmic (V01217; X03672), ly6/neurotoxin (Lynx1) homolog (AD141377), astrocytic phosphoprotein; PFA 15 gene (AJ243949; X86694), PLIC-1 (AF177345), Nfx1 (tip associating protein (TAP) gene) (AF093139; AF093140), A-Crystallin B (U04320; M73741; M28638), heat shock-like protein 70 kD (X70065; U73744; Y00371), tau microtubule-associated protein (X79321), myelin, Scgwann cell, Perioheral (P-0) (K03242), B-Tubulin class 1 (ABO11679; X04663; AF14139), putative long-chain polyunsaturated fatty acid elongation enzyme (Helo1) or peptidylglycine alpha-amidating monooxygenase 1.

24. A non-human animal having in its genome an introduced gene sequence or a removed or down-regulated gene sequence, said sequence being down-regulated in the spinal cord of a mammal in response to first and second models of neuropathic or central sensitisation pain.

25. A non-human animal according to claim 24, wherein said gene sequence becomes down regulated both in response to streptozocin induced diabetes and in response to chronic constriction injury.

26. A non-human animal according to claim 24, wherein the introduced gene sequence encodes a kinase, an expression product or fragment thereof, a transporter, a G-protein coupled receptor, a DNA bidning protein, a ligase, a lyase, an oxidoreductase, transferase, or a hydrolase.

27. A non-human animal according to claim 24 which is C. elegans.

28. A kit comprising;
(a) affinity peptide and/or ligand and/or substrate for an expression product of a gene sequence that is down-regulated in the spinal cord of a mammal in response to a mechanistically distinct first and second models of neuropathic or central sensitization pain; and
(b) a defined quantity of an expression product of a gene sequence that is down-regulated in the spinal cord of a mammal both in response to first and second models of neuropathic or central sensitization pain,
for simultaneous, separate or sequential use in detecting and/or quantifying down-regulation of a gene sequence in the spinal cord of a mammal in response to first and second models of neuropathic or central sensitization pain.

29. A kit according to claim 28, wherein the gene sequence encodes a kinase, an expression product or fragment thereof, a transporter, a G-protein coupled receptor, a DNA bidning protein, a ligase, a lyase, an oxidoreductase, transferase, or a hydrolase.

30. A kit comprising:
(a) nucleic acid sequences capable of hybridization to a nucleic acid sequence that is down-regulated in the spinal cord of a mammal in response to first and second models of neuropathic or central sensitization pain; and
(b) a defined quantity of one or more nucleic acid sequences capable of hybridization to a nucleic acid sequence that is down-regulated in the spinal cord of a mammal in response to first and second models of neuropathic or central sensitization pain,
for simultaneous, separate or sequential use in detecting and/or quantifying down-regulation of a gene sequence in the spinal cord of a mammal in response to first and second models of neuropathic or central sensitization pain.

31. The kit of claim 30, wherein the gene sequence encodes a kinase, an expression product or fragment thereof, a transporter, a G-protein coupled receptor, a DNA bidning protein, a ligase, a lyase, an oxidoreductase, transferase, or a hydrolase.

32. A compound that modulates the action of an expression product of a gene sequence that is down-regulated in the spinal cord of a mammal in response to first and second models of neuropathic or central sensitization pain.

33. A compound according to claim 32 wherein the gene sequence is listed in Tables I to VI.

34. A compound according to claim 32 wherein the nucleotide sequence encodes a kinase, an expression product or fragment thereof, a transporter, a G-protein coupled receptor, a DNA bidning protein, a ligase, a lyase, an oxidoreductase, transferase, or a hydrolase.

35. A compound according to claim 32 for use as a medicament.

36. A compound according to claim 32 for the treatment or diagnosis of pain.

37. A pharmaceutical composition comprising a compound according to claim 32 and a pharmaceutically acceptable carrier or diluent.

38. Use of a compound according to claim 32 in the manufacture of a medicament for the treatment or diagnosis of pain.

39. Use of a compound according to any one of claims 32 in the manufacture of a medicament for the treatment or diagnosis of chronic pain.

40. A method of treatment of pain, which comprises administering to a patient an effective amount of a compound according to claim 32. protein coupled receptor, a DNA bidning protein, a ligase, a lyase, an oxidoreuctase, transferase, or a hydrolase.
